(19) United States
(12) Patent Application Publication
(10) Pub. No.: US 2017/0247762 A1
(43) Pub. Date: Aug. 31, 2017

(54) COMPOSITIONS, METHODS AND USE OF SYNTHETIC LETHAL SCREENING

(71) Applicants: THE BOARD INSTITUTE INC., Cambridge, MA (US); Massachusetts Institute of Technology, Cambridge, MA (US)

(72) Inventor: Myriam Heiman, Newton, MA (US)

(21) Appl. No.: 15/521,780

(22) PCT Filed: Oct. 27, 2015
(86) PCT No.: PCT/US2015/057567

§ 371 Date: Apr. 25, 2017

Related U.S. Application Data
(60) Provisional application No. 62/122,686, filed on Oct. 27, 2014.

(51) Int. Cl. C12Q 1/68; C12N 15/113; C07K 16/40; A01K 67/027; C12N 9/22

(52) U.S. Cl. CPC C12Q 1/6883; A01K 67/0278; C12N 9/22; C07K 16/40; C12Y 111/01009; C12N 15/113; A01K 2267/0318; C12Q 2600/118; C12Q 2600/158; C12Q 2600/136; C12N 2310/20; C07K 2317/34

(57) ABSTRACT

The present invention generally relates to methods of identifying modulators of central nervous system diseases and the use of the modulators in treatment and diagnosis. The methods utilize a novel high throughput screen that includes injection of a library of barcoded viral vectors expressing shRNA's, CRISPR/Cas systems or cDNA's into animal models of disease and detecting synthetic lethality.

### RELATED APPLICATIONS AND INCORPORATION BY REFERENCE

This application claims benefit of and priority to U.S. provisional patent application Ser. No. 62/122,686, filed Oct. 27, 2014.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FEDERAL FUNDING LEGEND

This invention was made with government support under grant number NS085880 awarded by the National Institutes of Health. The government has certain rights in the invention.

### FIELD OF THE INVENTION

The present invention generally relates to methods of identifying modulators of central nervous system diseases using a novel high throughput methodology that includes expressing CRISPR/Cas systems, shRNA's or cDNA's in animal models of disease.

### BACKGROUND OF THE INVENTION

Currently there are no cures or effective treatments for many neurodegenerative diseases. All of the major neurodegenerative diseases display characteristic nerve-cell (neuronal) vulnerability patterns, as well as an increased prevalence with advanced age. Many genes are involved in the pathogenesis of such diseases. As such, it is a challenge to find genes that are modulators of disease pathogenesis that can be used for diagnostic screening or effective treatments.

One such disease is Huntington's Disease. Huntington's disease, the most common inherited neurodegenerative disease, is characterized by a dramatic loss of deep-layer cortical and striatal neurons, as well as morbidity in mid-life. Huntington's disease is the most common genetic cause of abnormal involuntary writhing movements called chorea.

Symptoms of the disease can vary between individuals and even among affected members of the same family, but usually progress predictably. The earliest symptoms are often subtle problems with mood or cognition. A general lack of coordination and an unsteady gait often follows. As the disease advances, uncoordinated, jerky body movements become more apparent, along with a decline in mental abilities and behavioral symptoms. Physical abilities are gradually impeded until coordinated movement becomes very difficult. Mental abilities generally decline into dementia. Complications such as pneumonia, heart disease, and physical injury from falls reduce life expectancy to around twenty years from the point at which symptoms begin. There is no cure for Huntington's disease, and full-time care is required in the later stages of the disease.

Treatments for Huntington's disease are available to reduce the severity of some of its symptoms (Frank et al., (2010) Drugs 70 (5): 561-71). Tetrabenazine was approved in 2008 for treatment of chorea in Huntington's disease in the United States. Other drugs that help to reduce chorea include neuroleptics and benzodiazepines. Compounds such as amantadine are still under investigation but have shown preliminary positive results (Walker, (2007) Lancet 369 (9557): 218-28). Hypokinesia and rigidity, especially in juvenile cases, can be treated with anti-Parkinson drugs, and myoclonic hyperkinesia can be treated with valproic acid.

Huntington's disease is caused by a mutation in the Huntingtin gene. Expansion of a CAG (cytosine-adenine-guanine) triplet repeat stretch within the Huntingtin gene results in a mutant form of the protein, which gradually damages cells in the brain, through mechanisms that are not fully understood. The length of the trinucleotide repeat accounts for 60% of the variation in the age symptoms appear and the rate they progress. The remaining variation is due to environmental factors and other genes that influence the mechanism of the disease (Walker, (2007) Lancet 369 (9557): 218-28).

The diagnosis of Huntington's disease is suspected clinically in the presence of symptoms. The diagnosis can be confirmed through molecular genetic testing which identifies the expansion in the Huntingtin gene. Testing of adults at risk for Huntington disease who have no symptoms (asymptomatic) of the disease has been available for over ten years. However, this testing cannot accurately predict the age a person found to carry a Huntington disease causing mutation will begin experiencing symptoms, the severity or type of symptoms they will experience, or rate of disease progression. Other markers for disease progression are available, for example, loss of DARPP-32 striatal expression has been shown to be a molecular marker of Huntington's disease progression (Bibb et al., (2000) Proc Natl Acad Sci 6; 97(12):6809-14).

Human genetic studies led to the identification of huntingtin as the causative gene. Recent genomic advances have also led to the identification of hundreds of potential interacting partners for huntingtin protein, and many hypotheses as to the molecular mechanisms whereby mutant huntingtin leads to cellular dysfunction and death (Goehler et al., (2004) Mol. Cell 15 (6): 853-65). Huntingtin protein is expressed in all mammalian cells and interacts with proteins which are involved in transcription, cell signaling and intracellular transporting (Harjes et al., (2003) Trends Biochem. Sci. 28 (8): 425-33). However, the multitude of possible interacting partners and cellular pathways affected by mutant huntingtin has obfuscated research seeking to understand the etiology of this disease, and to date no curative therapeutic exists for the disease.

A high throughput screening method to discover modulators of diseases, such as Huntington's disease, is a powerful tool to identify new drug targets, new prognostic methods, and new treatments.

Citation or identification of any document in this application is not an admission that such document is available as prior art to the present invention.

### SUMMARY OF THE INVENTION

It is an object of the invention to provide a genetic screening platform that could be used in mammals to identify modulators of diseases of the central nervous system. It is another object of the invention that the modulators are used in treatments, as therapeutic targets and for diagnosing disease.

In a first aspect, the present invention provides a method of screening for modulators of a disease comprising: administering to each of a first and second mammal of the same species at least one vector, each vector comprising a regulatory element operably linked to a nucleotide sequence that is transcribed in vivo, wherein the first mammal is a model of a human disease and the second mammal is a normal control mammal not a model of a human disease, and wherein the nucleotide sequence encodes a protein coding gene, or a short hairpin RNA, or a CRISPR/Cas system; harvesting DNA from the first mammal and the second mammal; identifying the vectors by sequencing the harvested DNA; and comparing the representation of each vector from the first mammal and the second mammal, whereby a differential representation in the first mammal indicates that the protein coding gene, or short hairpin RNA target, or CRISPR/Cas system target is a modulator of the disease. Not being bound by a theory a synthetic lethal gene will be under represented in the first mammal that is a model of human disease. In a preferred embodiment, more than one vector is administered to each of a first and second mammal. In some embodiments, about 100, 500, 1000, 5000, 7000, 10,000, or 20,000 vectors may be administered to a mammal. The vectors may be administered stereotaxically. The nucleotide sequence that can be transcribed may target any gene within a genome or any sequence within a genome. The target sequence in the genome or target gene may be a regulatory sequence or any functional element in an RNA transcript or genomic locus, including, but not limited to a promoter, enhancer, repressor, polyadenylation signal, splice site, or untranslated regions. The gene may be any gene within a genome. The gene may be a peroxidase gene. The protein coding gene may be a cDNA, whereby a gene may be overexpressed. The vector may comprise a unique barcode sequence, and the method may further comprise identifying the barcodes during sequencing, whereby the identification of a barcode indicates the presence of a vector. A barcode can be any length nucleotide sequence within a polynucleotide that can be distinguished reliably by PCR, sequencing, or hybridization technology from similar length nucleotide sequences in another polynucleotide. The DNA sequencing may be any sequencing technique, preferably next generation sequencing, such as, Illumina sequencing. The barcodes may be identified by microarray analysis. Microarrays may be constructed such that cDNA complementary to the sequences of the barcodes are bound to the microarray. Harvested genomic DNA is hybridized to the bound cDNA to determine the amount of each barcode. Additionally, genomic DNA from the first mammal and second mammal are fluorescently labelled with different fluorescent dyes. For example one dye can fluoresce red and the other green. Both sets of labelled genomic DNA can then be hybridized to the same microarray and fluorescence can be compared to determine barcode representation.

The CRISPR/Cas system may comprise: a first regulatory element operably linked to a nucleotide sequence encoding a CRISPR-Cas system polynucleotide sequence comprising at least one guide sequence, a tracr RNA, and a tracr mate sequence, wherein the at least one guide sequence hybridizes with a target sequence; and a second regulatory element operably linked to a nucleotide sequence encoding a Type II Cas9 protein. The first and second mammals may be transgenic non-human mammals comprising Cas9 and the nucleotide sequence encoding a CRISPR/Cas system may comprise at least one guide sequence, a tracr RNA, and a tracr mate sequence, wherein the at least one guide sequence hybridizes with a target sequence. The expression of Cas9 may be inducible.

In one embodiment, the vector is configured to be conditional, whereby the vector targets only certain cell types. The vector may be a viral vector. The vector may be conditional by using a regulatory element that is cell or tissue specific. The regulatory element may be a promoter. The vector may be conditional by using a viral vector that infects a specific cell type. The vector may be any virus that efficiently targets cells of the central nervous system and does not illicit a strong immune reaction. The viral vector may be a lentivirus, an adenovirus, or an adeno associated virus (AAV). The virus envelope proteins may be chosen to cause the virus to have tropism towards a specific cell type. The vesicular stomatitis virus (VSV) envelope protein may be used to make a virus conditional.

The disease may be any nervous system disease where a model of disease exists or can be created. The screening method may be used to screen for modulators in Huntington's Disease, Alzheimer's disease, Parkinson's disease, and ALS. In preferred embodiments the disease is Huntington's Disease or Parkinson's Disease. The first mammal may be the R6/2 Huntington's disease model line.

In a second aspect, the present invention provides a method of treating a nervous system disease. The method may comprise activating expression of Gpx6 in the central nervous system of a subject in need thereof suffering from the disease. The activation may be by a small molecule or compound. The small molecule or compound may be identified using biochemical and cell based assays. Additionally, protein therapeutics could be used to activate Gpx6. Treatment may be a single dose, multiple doses over a period of time, or doses on schedule for life. The schedule may be e.g., weekly, biweekly, every three weeks, monthly, bimonthly, every quarter year (every three months), every third of a year (every four months), every five months, twice yearly (every six months), every seven months, every eight months, every nine months, every ten months, every eleven months, annually or the like.

The method may comprise expressing Gpx6 in the central nervous system of a subject in need thereof suffering from the disease. Gpx6 may be expressed by introduction of a plasmid by injection or by gene gun. Gpx6 may also be introduced by viral vector such as AAV, adenovirus, or lentivirus.

The method may comprise introducing into a subject in need thereof suffering from the disease a CRISPR-Cas9 based system configured to target Gpx6. The CRISPR/Cas system may comprise a functional domain that activates transcription of the Gpx6 gene. The functional domain may be an activator domain.

The disease may be any nervous system disease. The nervous system disease may be Huntington's Disease or Parkinson's Disease. Treating with a modulator by either effecting its expression or by introducing a vector to express the protein may not completely alleviate symptoms. Therefore, other drugs that specifically target the symptoms can be combined with that of a modulator. One may decrease the normal dose of the drug given due to the combination. The frequency of the drug may also be adjusted. The method may further comprise administering to a subject in need thereof suffering from the disease at least one of the drugs selected from the group consisting of Tetrabenazine, neuroleptics, benzodiazepines, amantadine, anti Parkinson's drugs, valproic acid, antioxidants, and Gpx mimetics. Central nervous system diseases are associated with oxidative stress, as well as, having neurological symptoms that lead to both mental and physical abnormalities. A combination therapy may be used to synergistically alleviate these symptoms. Antioxidants and Gpx mimetics may be used when a modulator involved in oxidative stress is identified.

In a third aspect, the present invention provides a method of determining a prognosis for a central nervous system disease comprising: obtaining a RNA sample from a patient suffering from a central nervous system disease; assaying the level of Gpx6 gene expression; and comparing the levels of Gpx6 gene expression to a control level determined by testing healthy subjects, wherein the prognosis is worse if Gpx6 gene expression is lower than the control level. The method may further comprise assaying the level of DARPP-32 gene expression; and comparing the levels of DARPP-32 gene expression to a control level determined by testing healthy subjects, wherein the prognosis is worse if DARPP-32 gene expression is lower than the control level.

In a fourth aspect, the present invention provides an antibody comprising a heavy chain and a light chain, wherein the antibody binds to an antigenic region of the Gpx6 protein comprising SEQ ID No: 1.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings, incorporated herein by reference wherein:

FIG. 1. Illustrates gene expression changes associated with normal aging in cortical and striatal dopaminoceptive cell types. Venn diagram showing the number and overlap of statistically significant gene expression changes in dopamine receptor 1a (Drd1a)- or dopamine receptor 2 (Drd2)-expressing cortical or striatal neurons, based on a comparison of mice aged 6 weeks of age versus 2 years, 6 weeks of age. Statistically significant changes are defined as genes displaying ≧1.2-fold change and a Benjamini-Hochberg adjusted p-value from Welch's t test of ≦0.05.

FIG. 2. Illustrates the Synthetic lethal in the CNS (SLIC) screen. Top: Lentiviral genome-wide overexpression or knockdown libraries are injected into the striatum, such that each neuron or glial cell receives on average of one element (schematized by different colors). Lentivirus integrates into the cell's genome and expresses either a cDNA or shRNA. Bottom: After incubation in vivo, cells that have received a synthetic lethal hit die and the representation of these library elements are lost (an event that can be revealed by sequencing of all of the lentiviruses still present in the brain). When injections are performed in a paired fashion, comparing disease model mice to wild-type littermates, genes that cause synthetic lethality only in combination with a disease-causing mutation can be identified.

FIG. 3. Illustrates the number of striatal cells transduced by the vesicular stomatitis virus G (VSV-G) coated lentivirus used in this study. EGFP cDNA-expressing lentivirus was injected into male mouse striatum 8 weeks of age and tissue was processed four days later for indirect immunofluorescent staining using antibodies directed toward GFP (marking transduced cells). By comparison of DAPI stained cells to EGFP-expressing cells, approximately 20% of cells in any rostrocaudal region of the striatum were transduced (EGFP positive). Based on a number of 1.4×10⁶ million striatal cells per animal (Fentress. Cowan et al., 1981), we thus calculate that the upper limit of transduction is 2.8×10⁵ striatal cells.

FIG. 4. Illustrates striatal cell types infected by the vesicular stomatitis virus G (VSV-G) coated lentivirus used in this study. EGFP cDNA-expressing lentivirus was injected into male mouse striatum 8 weeks of age and tissue was processed four days later for indirect immunofluorescent staining using antibodies directed toward GFP (marking transduced cells), NeuN (neuronal marker), and GFAP (astrocyte marker). Based on immunofluorescent staining with these markers, approximately 83% of transduced cells are neurons, 14% are astrocytes, and 3% are unidentified cells.

FIG. 5A-5C. Illustrates SLIC screening in mouse models of Huntington's disease. (A) Control small hairpin RNA (shRNA) representation in the striatum of wild-type animals, as determined by shRNA barcode sequencing, at 4 and 6 weeks after injection, each compared to a control 2 day time-point. A negative number reflects loss versus the control time-point. The positive control, a hairpin targeting the Psmd2 gene product, would be expected to cause cell death, leading to loss of its representation. Negative control shRNAs used (Table 9) had no known target in the genome. (B) shRNA barcode sequence representation at the first SLIC HD time-point. Graph represents log 2 fold changes in representation in the HD model at 4 weeks compared to the control 2-day time-point (R6/2 value, y axis), versus wild-type controls at the same two time-points (WT value, x axis). The positive control targeting the Psmd2 gene product is not plotted for the purposes of scaling. Diagonal line represents equal representation (x=y). Genes causing synthetic lethality are expected to be offset to the right of the diagonal in the bottom left quadrant of the graph. Gpx6 targeting shRNAs are denoted in red. (C) SLIC results for synthetic lethal hits that induce loss of representation, plotting % lentiviral element depletion seen in the HD model (R6/2) versus congenic wild-type animals at 4 weeks (left panel) and 6 weeks (right panel) of incubation. Controls are not represented. Gpx6 targeting shRNAs are denoted in red.

FIG. 6. Illustrates that Gpx6 expression is down-regulated in the brains of Huntington's disease model mice. RNA was purified from the striatum of male R6/2 and control mice aged 8 weeks, and messenger RNA (mRNA) was converted to cDNA and used for quantitative PCR to measure Gpx6 mRNA abundance. Average cycle threshold values relative to Eif4a2 (delta Cₜ) are plotted with standard deviation. A higher delta Cₜ value (closer to 0) signifies higher abundance. A two-tailed unpaired t-test reveals a significance in difference between the means, p=0.0002.

FIG. 7. Illustrates Gpx6 mRNA expression across mouse brain regions. A cDNA panel representing 13 brain regions, as well as whole mouse brain, was used for quantitative PCR to measure Gpx6 mRNA abundance in adult mouse brain (10 weeks of age). Average cycle threshold values relative to actin (delta C) are plotted with standard deviation. A lower delta C value signifies higher abundance.

FIG. 8. Illustrates Gpx6 expression across normal aging. RNA was purified from the noted brain regions of male mice aged 1.5, 11, and 18 months, and messenger RNA (mRNA) was converted to cDNA and used for quantitative PCR to measure Gpx6 mRNA abundance. Average cycle threshold values relative to actin (delta Cₜ) are plotted with standard deviation. A lower delta Cₜ value signifies higher abundance.

FIG. 9A-9B. Illustrates the results of over-expressing Gpx6 in Huntington's disease model mice (A) Rescue of open field motor behavior in Huntington's disease model mice overexpressing Gpx6. Huntington's disease model mice (R6/2) or wild-type (WT) congenic controls were injected in the striatum bilaterally with Gpx6 or control (TRAP construct expressing) AAV9 virus at 6 weeks of age. After two weeks of recovery, motor function was assessed by open field assay. Average performance is plotted ±SEM for each data point, reflecting total distance in cm travelled during a one-hour interval (R6/2+Gpx6 n=10; R6/2+control n=10; WT+Gpx6 n=12; WT+control n=11). R6/2+Gpx6 vs. R6/2+control p value=0.0165; WT+Gpx6 vs. WT+control p value=0.7826 (no significance). (B) Increased DARPP-32 expression in Huntington's disease model mice overexpressing Gpx6. Huntington's disease model mice (R6/2) or wild-type (WT) congenic controls were unilaterally injected with control (TRAP construct; left hemisphere) or Gpx6 overexpressing (right hemisphere) AAV9 virus at 6 weeks of age. After two weeks of recovery, mice were sacrificed and brain tissue was processed for indirect immunofluorescent staining. Top panel: representative images of R6/2 mice injected with Gpx6 and control AAV9. Bottom panel: quantitation of images (mean pixel intensity across imaging field) from equivalent points in the dorsal striatum, p value=0.0026. No significant difference between control and Gpx6-injected hemispheres was observed in wild-type congenic controls (data not shown). A.U. signifies arbitrary fluorescence units.

FIG. 10. Illustrates locomotor effects of Gpx6 overexpression in a Parkinson's disease model mouse line. Mice overexpressing mutant alpha-synuclein protein “PD” or wild type littermates were injected with a Gpx6 overexpression virus at 6 weeks of age. Motor phenotypes were tested by open field assay for 60 minutes at approximately 7 months of age. At this age, PD model mice exhibit hyperactivity before progressing to hypoactivity at a later age. Gpx6 overexpression rescued the PD model phenotype at this age.

### DETAILED DESCRIPTION OF THE INVENTION

The invention provides a method for identifying modulators of central nervous system diseases and for treating with agonists or antagonists of the modulators or with the modulators themselves. The invention also provides the use of the modulators in determining prognosis and diagnosis of a central nervous system disease and providing individualized or personalized treatment. The method may comprise: (a) stereotaxically administering to each of a first and second mammal of the same species at least one vector containing a barcode and a nucleic acid molecule that is transcribed in vivo, wherein the first mammal is a model of a human disease and the second mammal is a normal control mammal not a model of a human disease, and wherein the nucleic acid molecule is associated with a gene; (b) harvesting genomic DNA from the first mammal and the second mammal; (c) identifying the barcodes from the harvested genomic DNA; and (d) comparing the barcode representation from the first mammal and the second mammal, whereby a differential barcode representation in the first mammal indicates that the gene associated with the nucleic acid molecule is a modulator of the disease. In one embodiment, modulators are determined by a loss of barcode in the disease model mouse when compared to the control mouse. In another embodiment, modulators are determined by a gain of barcode in the disease model mouse when compared to the control mouse.

Several further aspects of the invention relate to screening for modulators associated with a wide range of central nervous system diseases which are further described on the website of the National Institutes of Health (website at http://rarediseases.info.nih.gov/gard/diseases-by-category/17/nervous-system-diseases). The central nervous system diseases may include but are not limited to Alzheimer's Disease, Huntington's Disease and other Triplet Repeat Disorders (see Table A), amyotrophic lateral sclerosis (ALS), and Parkinson's disease.

TABLE A
Trinucleotide repeat disorders
Polyglutamine (PolyQ) Diseases

| | | Normal PolyQ | Pathogenic |
|---|---|---|---|
| Type | Gene | repeats | PolyQ repeats |
| DRPLA | ATN1 or DRPLA | 6-35 | 49-88 |
| (Dentatorubropallidoluysian | | | |
| atrophy) | | | |
| HD (Huntington's disease) | HTT (Huntingtin) | 6-35 | 36-250 |
| SBMA (Spinobulbar muscular | Androgen receptor on | 9-36 | 38-62 |
| atrophy or Kennedy disease) | the X chromosome. | | |
| SCA1 (Spinocerebellar ataxia | ATXN1 | 6-35 | 49-88 |
| Type 1) | | | |
| SCA2 (Spinocerebellar ataxia | ATXN2 | 14-32 | 33-77 |
| Type 2) | | | |
| SCA3 (Spinocerebellar ataxia | ATXN3 | 12-40 | 55-86 |
| Type 3 or Machado-Joseph | | | |
| disease) | | | |
| SCA6 (Spinocerebellar ataxia | CACNA1A | 4-18 | 21-30 |
| Type 6) | | | |
| SCA7 (Spinocerebellar ataxia | ATXN7 | 7-17 | 38-120 |
| Type 7) | | | |
| SCA17 (Spinocerebellar ataxia | TBP | 25-42 | 47-63 |
| Type 17) | | | |

Non-Polyglutamine Diseases

| | | | Normal/wild | |
|---|---|---|---|---|
| Type | Gene | Codon | type | Pathogenic |
| FRAXA (Fragile X syndrome) | FMR1, on the X- | CGG | 6-53 | 230+ |
| | chromosome | | | |
| FXTAS (Fragile X-associated | FMR1, on the X- | CGG | 6-53 | 55-200 |
| tremor/ataxia syndrome) | chromosome | | | |
| FRAXE (Fragile XE mental | AFF2 or FMR2, on the | CCG | 6-35 | 200+ |
| retardation) | X-chromosome | | | |
| FRDA (Friedreich's ataxia) | FXN or X25, (frataxin- | GAA | 7-34 | 100+ |
| | reduced expression) | | | |
| DM (Myotonic dystrophy) | DMPK | CTG | 5-37 | 50+ |
| SCA8 (Spinocerebellar ataxia | OSCA or SCA8 | CTG | 16-37 | 110-250 |
| Type 8) | | | | |
| SCA12 (Spinocerebellar ataxia | PPP2R2B or SCA12 | nnn On 5′ | 7-28 | 66-78 |
| Type 12) | | end | | |

Additionally, the central nervous system diseases may include but are not limited to 2-methyl-3-hydroxybutyric aciduria, 2-methylbutyryl-CoA dehydrogenase deficiency, 22q11.2 deletion syndrome, 22q13.3 deletion syndrome, 3-alpha hydroxyacyl-CoA dehydrogenase deficiency, 6-pyruvoyl-tetrahydropterin synthase deficiency, Aarskog syndrome, Aase-Smith syndrome, Abetalipoproteinemia, Absence of septum pellucidum, Acanthocytosis, Aceruloplasminemia, Acrocallosal syndrome, Schinzel type, Acrofacial dysostosis Rodriguez type, Acute cholinergic dysautonomia, Acute disseminated encephalomyelitis, Adenylosuccinase deficiency, Adie syndrome, Adrenomyeloneuropathy, Advanced sleep phase syndrome, familial, AGAT deficiency, Agnosia, Aicardi syndrome, Aicardi-Goutieres syndrome type 5, Albinism deafness syndrome. Alexander disease, Alopecia, Alpers syndrome, Alpha-ketoglutarate dehydrogenase deficiency, Alpha-mannosidosis type 1, Alpha-thalassemia x-linked intellectual disability syndrome, Alternating hemiplegia of childhood, Aminoacylase 1 deficiency, Amish infantile epilepsy syndrome, Amish lethal microcephaly, Amyloid neuropathy, Amyloidosis cerebral, Anaplastic ganglioglioma, Andermann syndrome, Andersen-Tawil syndrome, Anencephaly, Angioma hereditary neurocutaneous, Aniridia renal agenesis psychomotor retardation, Apraxia, Arachnoid cysts, Arachnoiditis, Arthrogryposis dysplasia, Aspartylglycosaminuria, Ataxia telangiectasia, Atelosteogenesis, Athabaskan brainstem dysgenesis, Atkin syndrome, Atypical Rett syndrome, Bannayan-Riley-Ruvalcaba syndrome, Barth syndrome, Basal ganglia disease, biotin-responsive. Basilar migraine, Battaglia Neri syndrome, Batten disease, Becker muscular dystrophy, Behcet's disease, Bell's palsy, Benign familial neonatal-infantile seizures, Benign rolandic epilepsy (BRE), Bethlem myopathy, Bilateral frontal polymicrogyria, Bilateral frontoparietal polymicrogyria, Bilateral generalized polymicrogyria, Bilateral parasagittal parieto-occipital polymicrogyria, Bilateral perisylvian polymicrogyria, Binswanger's disease, Bird headed dwarfism Montreal type, Bixler Christian Gorlin syndrome, Blepharospasm, Bobble-head doll syndrome, Borjeson-Forssman-Lehmann syndrome, Boucher Neuhauser syndrome, Bowen-Conradi syndrome. Branchial arch syndrome X-linked, Brody myopathy, Brown-Sequard syndrome, Brown-Vialetto-Van Laere syndrome, Bullous dystrophy hereditary macular type, C syndrome, C-like syndrome, CADASIL, CAHMR syndrome, Camptodactyly arthropathy coxa vara pericarditis syndrome, CANOMAD syndrome, Cantu syndrome, Cardiocranial syndrome, Cardiofaciocutaneous syndrome, Carney complex, Cataract anterior polar dominant, Cataract ataxia deafness, Catel Manzke syndrome, Caudal regression syndrome, Central core disease, Central neurocytoma, Central post-stroke pain, Cerebellar ataxia, Cerebellar degeneration, Cerebellar hypoplasia, Cerebellum agenesis hydrocephaly, Cerebral autosomal recessive arteriopathy with subcortical infarcts and leukoencephalopathy, Cerebral cavernous malformation, Cerebral dysgenesis neuropathy ichthyosis and palmoplantar keratoderma syndrome, Cerebral folate deficiency, Cerebral gigantism jaw cysts, Cerebral palsy, Cerebral sclerosis similar to Pelizaeus-Merzbacher disease, Cerebro-oculo-facio-skeletal syndrome, Cerebrospinal fluid leak, Cerebrotendinous xanthomatosis, Ceroid lipofuscinosis neuronal, Cervical hypertrichosis peripheral neuropathy, Chanarin-Dorfman syndrome, Charcot-Marie-Tooth disease, Chediak-Higashi syndrome, Chiari malformation, Choreoacanthocytosis, Choroid plexus carcinoma, Choroid plexus papilloma, Christianson syndrome, Chromosome 19q13.11 deletion syndrome, Chromosome 1p36 deletion syndrome, Chronic lymphocytic inflammation with pontine perivascular enhancement responsive to steroids. Chudley Rozdilsky syndrome, Cleft palate short stature vertebral anomalies, COACH syndrome, Cockayne syndrome, Coenzyme Q10 deficiency, Coffin-Lowry syndrome, Coffin-Siris syndrome, Cohen syndrome, Complex regional pain syndrome, Congenital central hypoventilation syndrome, Congenital cytomegalovirus, Congenital disorder of glycosylation type 1B, Congenital disorder of glycosylation type 2C, Congenital fiber type disproportion, Congenital generalized lipodystrophy type 4, Congenital insensitivity to pain with anhidrosis, Congenital muscular dystrophy type 1A. Congenital myasthenic syndrome with episodic apnea, Congenital rubella, Convulsions benign familial infantile, Corneal hypesthesia familial, Cornelia de Lange syndrome, Corticobasal degeneration, Costello syndrome, Cowchock syndrome, Crane-Heise syndrome, Craniofrontonasal dysplasia, Craniopharyngioma, Craniotelencephalic dysplasia, Creutzfeldt-Jakob disease, Crisponi syndrome, Crome syndrome, Curry Jones syndrome, Cyprus facial neuromusculoskeletal syndrome, Cytomegalic inclusion disease, Dancing eyes-dancing feet syndrome, Dandy-Walker like malformation with atrioventricular septal defect, Danon disease. Dementia familial British, Dentatorubral-pallidoluysian atrophy, Dermatomyositis, Devic disease, Dihydropteridine reductase deficiency, Distal myopathy Markesbery-Griggs type. Distal myopathy with vocal cord weakness, Dopamine beta hydroxylase deficiency, Dravet syndrome, Duane syndrome, Dubowitz syndrome, Dwarfism, mental retardation and eye abnormality, Dykes Markes Harper syndrome, Dysautonomia like disorder, Dysequilibrium syndrome, Dyskeratosis congenita, Dyssynergia cerebellaris myoclonica, Dystonia, Early-onset ataxia with oculomotor apraxia and hypoalbuminemia, Emery-Dreifuss muscular dystrophy X-linked, Empty sella syndrome, Encephalitis lethargica, Encephalocraniocutaneous lipomatosis, Encephalomyopathy, Eosinophilic fasciitis, Epidermolysa bullosa simplex with muscular dystrophy, Epilepsy, Epiphyseal dysplasia hearing loss dysmorphism, Episodic ataxia with nystagmus, Erythromelalgia, Essential tremor, Fabry disease, Facial onset sensory and motor neuronopathy, Facioscapulohumeral muscular dystrophy, Fallot complex with severe mental and growth retardation, Familial amyloidosis, Finnish type, Familial congenital fourth cranial nerve palsy, Familial dysautonomia, Familial encephalopathy with neuroserpin inclusion bodies, Familial exudative vitreoretinopathy, Familial hemiplegic migraine, Familial idiopathic basal ganglia calcification, Familial transthyretin amyloidosis, Farber's disease, Fatal familial insomnia, Fatty acid hydroxylase-associated neurodegeneration, Fazio Londe syndrome, Febrile infection-related epilepsy syndrome, Feigenbaum Bergeron Richardson syndrome, Filippi syndrome. Fine-Lubinsky syndrome, Fitzsimmons Walson Mellor syndrome, Fitzsimmons-Guilbert syndrome, Floating-Harbor syndrome, Florid cemento-osseous dysplasia, Flynn Aird syndrome, Focal dermal hypoplasia, Fountain syndrome, Fragile X syndrome, Fragile XE syndrome, Franek Bocker kahlen syndrome, Friedreich ataxia, Frontometaphyseal dysplasia, Frontotemporal dementia, Fryns syndrome, Fucosidosis, Fukuyama type muscular dystrophy, Fumarase deficiency, Galactosialidosis, GAPO syndrome, Gaucher disease type, Gemignani syndrome, Geniospasm, Genoa syndrome, Gerstmann syndrome, Gerstmann-Straussler-Scheinker disease, Giant axonal neuropathy. Gillespie syndrome. Glucose transporter type 1 deficiency syndrome, Glutaric acidemia, Glycogen storage disease, GM1 gangliosidosis, Goldberg-Shprintzen megacolon syndrome, Gomez Lopez Hernandez syndrome, Granulomatosis with polyangiitis (Wegener's), Griscelli syndrome type 1, Grubben de Cock Borghgraef syndrome, GTP cyclohydrolase I deficiency, Guanidinoacetate methyltransferase deficiency, Guillain-Barre syndrome, Gurrieri syndrome, Hamanishi Ueba Tsuji syndrome, Hansen's disease, Harding ataxia, Harrod Doman Keele syndrome, Hartnup disease, Hashimoto's encephalitis, Hemangioblastoma, Hemicrania continua, Hemiplegic migraine, Hennekam syndrome, Hereditary angiopathy with nephropathy aneurysms and muscle cramps syndrome, Hereditary endotheliopathy retinopathy nephropathy and stroke, Hereditary hemorrhagic telangiectasia, Hereditary hyperekplexia, Hereditary neuropathy with liability to pressure palsy, Hereditary sensory and autonomic neuropathy type 2, Hereditary sensory neuropathy type 1, Hereditary spastic paraplegia, Homocysteinemia due to MTHFR deficiency, Homocystinuria due to CBS deficiency, Hoyeraal Hreidarsson syndrome, HTLV-1 associated myelopathy/tropical spastic paraparesis, Huntington disease, Hyde Forster Mccarthy Berry syndrome, Hydranencephaly, Hydrocephalus due to congenital stenosis of aqueduct of sylvius, Hydroxykynureninuria, Hyperkalemic periodic paralysis. Hyperphenylalaninemia due to dehydratase deficiency, Hyperprolinemia, Hypertrophic neuropathy of Dejerine-Sottas, Hypogonadism alopecia diabetes mellitus mental retardation and extrapyramidal syndrome, Hypokalemic periodic paralysis, Hypomyelination and congenital cataract, Hypomyelination with atrophy of basal ganglia and cerebellum, Hypoparathyroidism-retardation-dysmorphism syndrome, Hypospadias mental retardation Goldblatt type, Hypothalamic hamartomas, Ichthyosis alopecia eclabion ectropion mental retardation, Idiopathic spinal cord herniation, Inclusion body myopathy, Incontinentia pigmenti, Infantile axonal neuropathy, Infantile convulsions and paroxysmal choreoathetosis, familial, Infantile myofibromatosis, Infantile onset spinocerebellar ataxia, Infantile Parkinsonism-dystonia, Infantile spasms broad thumbs, Inherited peripheral neuropathy, Intellectual deficit, Internal carotid agenesis, Intraneural perineurioma, Isodicentric chromosome 15 syndrome, Johanson Blizzard syndrome, Johnson neuroectodermal syndrome, Joubert syndrome, Juberg Marsidi syndrome, Juvenile dermatomyositis, Juvenile primary lateral sclerosis, Kabuki syndrome. Kanzaki disease, Kapur Toriello syndrome, KBG syndrome, Kearns Sayre syndrome, Kennedy disease, Keutel syndrome, King Denborough syndrome, Kleine Levin syndrome, Klumpke paralysis, Kosztolanyi syndrome, Kuru, L-2-hydroxyglutaric aciduria, Laband syndrome, Lafora disease, Laing distal myopathy, Lambert Eaton myasthenic syndrome, LCHAD deficiency, Leigh syndrome, French Canadian type, Leisti Hollister Rimoin syndrome, Lennox-Gastaut syndrome, Lenz Majewski hyperostotic dwarfism, Lenz microphthalmia syndrome, Lesch Nyhan syndrome, Leukodystrophy with oligodontia, Leukodystrophy, dysmyelinating, and spastic paraparesis with or without dystonia. Levic Stefanovic Nikolic syndrome, Lhermitte-Duclos disease, Li-Fraumeni syndrome, Limb dystonia, Limb-girdle muscular dystrophy, Limited scleroderma, Lissencephaly, Localized hypertrophic neuropathy, Locked-in syndrome, Logopenic progressive aphasia, Lowe oculocerebrorenal syndrome, Lowry Maclean syndrome, Lujan Fryns syndrome, Mac Dermot Winter syndrome, Machado-Joseph disease, Macrogyria, pseudobulbar palsy and mental retardation, Macrothrombocytopenia progressive deafness, Mal de debarquement, Male pseudohermaphroditism intellectual disability syndrome, Verloes type, Malignant hyperthermia, Mannosidosis, beta A, lysosomal, Marchiafava Bignami disease, Marden-Walker syndrome, Marinesco-Sjogren syndrome, Martsolf syndrome, Maternally inherited Leigh syndrome, McDonough syndrome, McLeod neuroacanthocytosis syndrome, Meckel syndrome, Medrano Roldan syndrome, Medulloblastoma, Megalencephalic leukoencephalopathy with subcortical cysts, Mehes syndrome, Meier-Gorlin syndrome, Meige syndrome, Melnick-Needles syndrome, Meningioma, Meningioma, spinal, Menkes disease, Mental deficiency-epilepsy-endocrine disorders, Mental retardation, Meralgia paresthetica, Methionine adenosyltransferase deficiency, Methylcobalamin deficiency cbl G type, Microbrachycephaly ptosis cleft lip, Microcephalic osteodysplastic primordial dwarfism type 1, Microcephalic primordial dwarfism Toriello type, Microcephaly, Microphthalmia syndromic, Microscopic polyangiitis, Miller-Dicker syndrome, Miller-Fisher syndrome, Minicore myopathy with external ophthalmoplegia, Mitochondrial complex II deficiency, Mitochondrial encephalomyopathy lactic acidosis and stroke-like episodes, Mitochondrial myopathy, Mitochondrial neurogastrointestinal encephalopathy syndrome, Mitochondrial trifunctional protein deficiency, Mixed connective tissue disease, Miyoshi myopathy, Moebius syndrome, Molybdenum cofactor deficiency, Morse-Rawnsley-Sargent syndrome, Morvan's fibrillary chorea, Motor neuropathy peripheral with dysautonomia, Mousa Al din Al Nassar syndrome, Moyamoya disease, MPV17-related hepatocerebral mitochondrial DNA depletion syndrome, Mucopolysaccharidosis, Multifocal motor neuropathy, Multiple myeloma, Multiple sulfatase deficiency, Multiple system atrophy (MSA), Muscle eye brain disease, Muscular dystrophy white matter spongiosis, Muscular phosphorylase kinase deficiency, Myasthenia gravis, Myelocerebellar disorder, Myelomeningocele, Myhre syndrome, Myoclonic astatic epilepsy, Myoclonus, Myoglobinuria recurrent, Myopathy congenital multicore with external ophthalmoplegia, Myotonia congenita, Myotonic dystrophy, Nance-Horan syndrome, Narcolepsy, Native American myopathy. Nemaline myopathy 5, Neonatal adrenoleukodystrophy, Neonatal meningitis, Neonatal progeroid syndrome, Neu Laxova syndrome, Neuroaxonal dystrophy, infantile, Neuroblastoma, Neurocutaneous melanosis, Neurofaciodigitorenal syndrome, Neuroferritinopathy, Neurofibromatosis, Neuromyelitis optica spectrum disorder, Neuronal ceroid lipofuscinoses, Neuronal intranuclear inclusion disease, Neuropathy, Neuropathy, Neutral lipid storage disease with myopathy, Nevoid basal cell carcinoma syndrome, Nicolaides Baraitser syndrome, Niemann-Pick disease type B, Non 24 hour sleep wake disorder, Nondystrophic myotonia, Normokalemic periodic paralysis, Norrie disease, Northern Epilepsy, Occult spinal dysraphism, Oculocerebrocutaneous syndrome, Oculofaciocardiodental syndrome, Oculopharyngeal muscular dystrophy, Ohtahara syndrome, Okamoto syndrome, Oligoastrocytoma, Oliver syndrome, Olivopontocerebellar atrophy, Omphalocele cleft palate syndrome lethal. Optic atrophy 2, Ornithine transcarbamylase deficiency, Orofaciodigital syndrome, Osteopenia and sparse hair, Osteoporosis-pseudoglioma syndrome, Oto-palato-digital syndrome type 1, Ouvrier Billson syndrome, Pachygyria, Pallidopyramidal syndrome, Pallister W syndrome, Pallister-Killian mosaic syndrome, Pantothenate kinase-associated neurodegeneration, Paralysis agitans, juvenile, Paramyotonia congenital, Parenchymatous cortical degeneration of cerebellum, Paroxysmal hemicranias, Parsonage Turner syndrome, PEHO syndrome, Pelizaeus-Merzbacher disease, Pelizaeus-Merzbacher disease, late-onset type, Periventricular leukomalacia, Perry syndrome, Peters plus syndrome, Pfeiffer Mayer syndrome, Pfeiffer Palm Teller syndrome, PHACE syndrome, Phosphoglycerate kinase deficiency, Phosphoglycerate mutase deficiency, Photosensitive epilepsy, Pick's disease, Pitt-Hopkins syndrome, POEMS syndrome, Poliomyelitis, Polyarteritis nodosa, Polycystic lipomembranous osteodysplasia with sclerosing leukoencephalopathy, Polydactyly cleft lip palate psychomotor retardation, Polyglucosan body disease, adult, Polyneuropathy mental retardation acromicria premature menopause, Pontine tegmental cap dysplasia, Pontocerebellar hypoplasia, Post Polio syndrome, Posterior column ataxia, Potassium aggravated myotonia, PPM-X syndrome, Prader-Willi habitus, osteopenia, and camptodactyly, Primary amebic meningoencephalitis, Primary angiitis of the central nervous system, Primary basilar impression, Primary carnitine deficiency, Primary lateral sclerosis, Primary melanoma of the central nervous system, Primary progressive aphasia, Progressive bulbar palsy, Progressive hemifacial atrophy, Progressive non-fluent aphasia, Proteus syndrome, Proud Levine Carpenter syndrome, Pseudoaminopterin syndrome, Pseudoneonatal adrenoleukodystrophy, Pseudoprogeria syndrome, Pseudotrisomy 13 syndrome, Pseudotumor cerebri, Pudendal Neuralgia, Pure autonomic failure, Pyridoxal 5′-phosphate-dependent epilepsy, Pyridoxine-dependent epilepsy, Pyruvate dehydrogenase phosphatase deficiency, Qazi Markouizos syndrome, Radiation induced brachial plexopathy, Rasmussen encephalitis, Reardon Wilson Cavanagh syndrome, Reducing body myopathy, Refsum disease, Refsum disease, infantile form, Renal dysplasia-limb defects syndrome, Renier Gabreels Jasper syndrome, Restless legs syndrome, Retinal vasculopathy with cerebral leukodystrophy, Rett syndrome, Richards-Rundle syndrome, Rigid spine syndrome, Ring chromosome, Rippling muscle disease, Roussy Levy syndrome, Ruvalcaba syndrome, Sacral defect with anterior meningocele, Salla disease, Sandhoff disease, Sarcoidosis, Say Barber Miller syndrome, Say Meyer syndrome, Scapuloperoneal syndrome, neurogenic, Kaeser type, SCARF syndrome, Schimke immunoosseous dysplasia, Schindler disease, type 1, Schinzel Giedion syndrome, Schisis association, Schizencephaly, Schwannomatosis, Schwartz Jampel syndrome type 1, Scott Bryant Graham syndrome, Seaver Cassidy syndrome, Seckel syndrome, Segawa syndrome, autosomal recessive, Semantic dementia, Sensory ataxic neuropathy, dysarthria, and ophthalmoparesis, Sepiapterin reductase deficiency, Septo-optic dysplasia, SeSAME syndrome, Shapiro syndrome, Sharp syndrome, Short chain acyl CoA dehydrogenase deficiency, Shprintzen-Goldberg craniosynostosis syndrome, Sialidosis, Siderius X-linked mental retardation syndrome, Sideroblastic anemia and mitochondrial myopathy, Simpson-Golabi-Behmel syndrome, Single upper central incisor, Sjogren-Larsson syndrome, Slow-channel congenital myasthenic syndrome, Smith-Lemli-Opitz syndrome type 1, Smith-Magenis syndrome, Sneddon syndrome, Snyder-Robinson syndrome, Sonoda syndrome, Spasmodic dysphonia, Spastic ataxia Charlevoix-Saguenay type, Spastic diplegia, Spastic paraplegia, Spina bifida occulta, Spinal muscular atrophy, Spinal shock, Spinocerebellar ataxia, Spinocerebellar degeneration and corneal dystrophy, Split hand urinary anomalies spina bifida, Spondyloepiphyseal dysplasia congenital, Status epilepticus, Steinfeld syndrome, Stratton-Garcia-Young syndrome, Striatonigral degeneration infantile, Sturge-Weber syndrome, Subacute sclerosing panencephalitis, Subcortical band heterotopia, Subependymoma, Succinic semialdehyde dehydrogenase deficiency, Susac syndrome, Symmetrical thalamic calcifications, Tangier disease, Tarlov cysts, Tay-Sachs disease, Tel Hashomer camptodactyly syndrome, Temporal epilepsy, familial, Temtamy syndrome, Thalamic degeneration symmetrical infantile, Thalamic degeneration, symmetric infantile, Thoracic outlet syndrome, Thyrotoxic periodic paralysis, Toriello Carey syndrome, Torsion dystonia with onset in infancy, Tourette syndrome, Transverse myelitis, Trichinosis, Trichorhinophalangeal syndrome type 2, Trigeminal neuralgia, Triose phosphate-isomerase deficiency, Triple A syndrome, Tuberous sclerosis, Tubular aggregate myopathy, Tyrosinemia type 1, Ullrich congenital muscular dystrophy, Unverricht-Lundborg disease, Van Benthem-Driessen-Hanveld syndrome, Van Den Bosch syndrome, Variant Creutzfeldt-Jakob disease, Vein of Galen aneurysm, Vici syndrome, Viljoen Kallis Voges syndrome, VLCAD deficiency, Vogt-Koyanagi-Harada syndrome, Von Hippel-Lindau disease, Walker-Warburg syndrome. Warburg micro syndrome, Weaver syndrome, Welander distal myopathy, Swedish type, Wernicke-Korsakoff syndrome, West syndrome, Westphal disease, Whispering dysphonia, Wieacker syndrome, Williams syndrome, Wilson disease, Wittwer syndrome, Wolf-Hirschhorn syndrome, Wolman disease, Worster Drought syndrome, Wrinkly skin syndrome, X-linked Charcot-Marie-Tooth disease type 5, X-linked creatine deficiency, X-linked myopathy with excessive autophagy, X-linked periventricular heterotopia, Young Hughes syndrome, Zechi Ceide syndrome, and Zellweger syndrome.

In one embodiment the disease is monogenic, affects defined cell populations in an age-dependent manner, and the mouse model displays minimal cell loss. This latter feature is particularly advantageous to the screening scheme, as synthetic lethal screens require a mild phenotype around which to screen for an enhanced phenotype.

The screening method may be used to identify modulators for any central nervous system diseases where an animal model is available. Several animal models have been described for the most prominent of the central nervous system diseases (Harvey et al., (2011) J. Neural Transm.; 118(1): 27-45; Ribeiro et al., (2013) Rev Bras Psiquiatr. 35 Suppl 2:S82-91). In some methods of the invention the organism or subject is a non-human eukaryote or a non-human animal or a non-human mammal. A non-human mammal may be for example a rodent (preferably a mouse or a rat), an ungulate, or a primate. In a preferred embodiment, the animal model is a mouse.

In another embodiment the animal model is a Huntington's disease (HD) model line. Mouse models have been created with CAG repeats of different lengths that have an HD phenotype: R6/1 with 116 repeats, R6/2 with 144 repeats and R6/5 with a wider spectrum of repeats. R6/2 mice have been studied most and show choreiform-like movements, involuntary stereotypic movements, tremor, epileptic seizures and premature death (Mangiarini et al., (1996) Cell, 87:493-506). In R6/2 mice the age of onset is 9-11 weeks and the age of death is 10-13 weeks. R6/2 mice have huntingtin aggregates in the nucleus of neurons seen prior to developing a neurological phenotype (Davies et al., (1997) Cell., 90:537-548). Also, the mRNA for type 1 metabotropic glutamate receptors and for D1 dopamine receptors is already reduced at the age of 4 weeks (Cha et al., (1998) Proc Natl Acad Sci USA, 95:6480-6485). A transgenic rat model of HD, with a mutated huntingtin gene containing 51 CAG repeats, expresses adult-onset neurological phenotypes, cognitive impairments, progressive motor dysfunction and neuronal nuclear inclusions in the brain (von Horsten et al., (2003) Hum Mol Genet., 12:617-624). The transgenic rats have a late onset of phenotype and they die between 15 and 24 months. Transgenic HD rats have an age and genotype dependent deterioration of psychomotor performance and choreiform symptoms (Cao et al., (2006) Behav Brain Res., 170:257-261). Recently, HD was modeled in the rhesus macaque with a lentiviral vector (Cai et al., (2008) Neurodegener Dis., 5:359-366). Yang et al. injected rhesus oocytes with lentivirus expressing exon 2 of the human huntingtin gene with 84 CAG repeats and five transgenic monkeys carrying mutant huntingtin were produced (Yang et al., (2008) Proc Natl Acad Sci USA., 105:7070-7075). The monkeys showed the main features of HD disease including nuclear inclusions, neuropil aggregates and a behavioral phenotype but all of them died at an early stage of life. In a preferred embodiment the mouse model is the R6/2 Huntington's disease model line (Mangiarini et al., (1996) Cell, 87:493-506).

In another embodiment the methods are used to identify modulators of Alzheimer's disease (AD). Alzheimer's disease is the most prevalent of neurodegenerative diseases that causes progressive memory loss and dementia in affected patients. Diagnosis of AD occurs post-mortem by confirming the presence of neurofibrillary tangles (NFT) and amyloid plaques which are found in the several brain regions including the subiculum and entorhinal cortex. The NFT are intraneuronal microtubule bundles containing hyperphosphorylated forms of microtubule associated protein tau (MAPT). The amyloid plaques are extracellular deposits primarily consisting of the amyloid β peptide. To date, 16 genes or loci have been identified for AD (OMIM 104300). The presence of NFTs in post-mortem brain is one of the defining pathologies of AD. However, there is no direct correlation between the number of cortical plaques and cognitive deficit in AD patients, and many individuals have amyloid plaques without cognitive impairment or dementia (Duyckaerts et al., (2009) Acta Neuropathol., 118:5-36). Moreover, the amount and the topography of the senile plaques are not correlated with the severity of dementia, and the amyloid deposition seems to remain stable during the progression of the disease (Jack et al., (2010) Lancet Neurol., 9:119-28). As such, in one embodiment, Alzheimer's disease is screened for modulators that can be used for diagnosis and treatment. There have been several transgenic mice generated based on mutations in the human MAPT gene that have provided clear evidence for mutant tau in NFT pathology and dementia (McGowan et al., (2006) Trends Genet., 22:281-289). None of the transgenic rodent models based on single gene mutations have been able to fully recapitulate the features of AD. Combinations of transgenes have provided novel transgenic models that have a progressive pathology with behavioral deficits. Triple transgenic mice (3×Tg-AD) have been produced and progressively develop synaptic dysfunction, APP-containing plaques and NFTs (Oddo et al., (2003) Neurobiol Aging, 24:1063-1070). The 3×Tg-AD mouse has thus been the most widely used model of AD for evaluating potential therapies, examining environmental vulnerabilities and studying disease mechanism (Gimenez-Llort et al., (2007) Neurosci Biobehav Rev., 31:125-147; Foy et al., (2008) J Alzheimers Dis., 15:589-603). In addition to mouse models based on mutations found in human genes, there are non-transgenic models of AD in the rat, rabbit, dog and primate that offer the ability to conduct complementary studies for the evaluation of therapeutics and the understanding of disease mechanisms (Woodruff-Pak, (2008) J Alzheimers Dis., 15:507-521). In a preferred embodiment, the 3×Tg-AD mouse is used with the screening methods.

In another embodiment the methods are used to identify modulator's of amyotrophic lateral sclerosis (ALS). Amyotrophic lateral sclerosis is a neurodegenerative disease that results from the progressive loss of motor neurons in brain and spinal cord. Onset of disease typically occurs in middle adulthood but forms with juvenile onset also occur. Symptoms include asymmetrical muscle weakness and muscle fasciculations. The disease progresses rapidly after onset leading to paralysis and eventually death within 5 years. The first gene associated with ALS was the superoxide dismutase-1 (SOD1) gene encoding an enzyme capable of inactivating superoxide radicals (Rosen et al., (1993) Nature, 362:59-62). Gurney et al. reported that mice over-expressing a human SOD1 allele containing a G93A substitution developed spinal cord motor neuron loss and related paralysis (Gurney et al., (1994) Science, 264:1772-1775). Following that initial study with the G93A variant, 13 additional transgenic mice have been made that produced a broad range of outcomes but all exhibit some characteristics of the disease (Ripps et al., (1995) Proc Natl Acad Sci USA, 92:689-693; Wong et al., (1995) Neuron, 14:1105-1116; Bruijn et al., (1997) Neuron, 18:327-338; Wang et al., (2002) Neurobiol Dis., 10:128-138, (2003) Hum Mol Genet., 12:2753-2764, (2005) Hum Mol Genet., 14:2335-2347; Tobisawa et al., (2003) Biochem Biophys Res Commun., 303:496-503; Jonsson et al., (2005) Brain, 127:73-88 (2004), J Neuropathol Exp Neurol., 65:1126-1136 (2006); Chang-Hong et al., Exp Neurol., 194:203-211; Watanabe et al., (2005) Brain Res Mol Brain Res., 135:12-20; Deng et al., (2006) Proc Natl Acad Sci USA, 103:7142-7147). The SOD1 animal collection has produced several therapeutic strategies (e.g. arimoclomal, ceftriaxone, IGF-1, HDAC inhibitors) that are now in clinical trials. In a preferred embodiment, a G93A mouse model is used to screen for modulators.

In another embodiment the methods are used to identify modulator's of Parkinson's disease (PD). Parkinson's disease is a slow, progressive neurodegenerative disorder that is characterized pathologically by the loss of dopaminergic neurons in the pars compacta of the substantia nigra. There currently is no mouse model for Parkinson's disease based on a mutation. For example, even though the gene is linked to the disease, overexpressing of human α-synuclein or its mutated forms in transgenic mice is not sufficient to cause a complete Parkinsonian phenotype. In one embodiment this mouse is used to screen for modulators. In other embodiments, mouse knockouts for the Park genes are used. The so-called neurotoxin-based models of PD are the most effective in reproducing irreversible dopaminergic neuron death and striatal dopamine deficit in nonhuman primates and rodents. MPTP (1-methyl-4-phenyl-1,2,3,6-terahydropyridine), 6-OHDA (6-hydroxy-dopamine), and rotenone are so far the most widely used compounds. They are particularly attractive for inducing cytotoxicity by oxidative stress mechanisms, as brain from PD patients show decreased levels of reduced glutathione and oxidative modifications to DNA, lipids, and proteins (Pearce et al., (1997) J Neural Transm., 104:661-77; Floor et al., (1998) J Neurochem., 70:268-75). Interestingly, MPTP was accidently discovered during the investigations of the potential factors that led young addicts to develop PD-like symptoms. MPTP was found to be the heroin contaminant responsible for parkinsonism in these subjects (Ribeiro et al., (2013) Rev Bras Psiquiatr. 35 Suppl 2:S82-91). In a preferred embodiment, the neurotoxin based models are used to screen for modulators.

Among vectors that may be used in the practice of the invention, integration in the host genome of a central nervous system cell is possible with retrovirus gene transfer methods, often resulting in long term expression of the inserted transgene. In a preferred embodiment the retrovirus is a lentivirus. Additionally, high transduction efficiencies have been observed in many different cell types and target tissues. The tropism of a retrovirus can be altered by incorporating foreign envelope proteins, expanding the potential target population of target cells. A retrovirus can also be engineered to allow for conditional expression of the inserted transgene, such that only certain cell types are infected by the lentivirus. Additionally, cell type specific promoters can be used to target expression in specific cell types. Lentiviral vectors are retroviral vectors (and hence both lentiviral and retroviral vectors may be used in the practice of the invention). Moreover, lentiviral vectors are preferred as they are able to transduce or infect non-dividing cells and typically produce high viral titers. Selection of a retroviral gene transfer system may therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired nucleic acid into the target cell to provide permanent expression. Widely used retroviral vectors that may be used in the practice of the invention include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., (1992) J. Virol. 66:2731-2739; Johann et al., (1992) J. Virol. 66:1635-1640; Sommnerfelt et al., (1990) Virol. 176:58-59; Wilson et al., (1998) J. Virol. 63:2374-2378; Miller et al., (1991) J. Virol. 65:2220-2224; PCT/US94/05700).

Also useful in the practice of the invention is a minimal non-primate lentiviral vector, such as a lentiviral vector based on the equine infectious anemia virus (EIAV) (see, e.g., Balagaan, (2006) J Gene Med; 8: 275-285, Published online 21 Nov. 2005 in Wiley InterScience (www.interscience.wiley.com). DOI: 10.1002/jgm.845). The vectors may have cytomegalovirus (CMV) promoter driving expression of the target gene. Accordingly, the invention contemplates amongst vector(s) useful in the practice of the invention: viral vectors, including retroviral vectors and lentiviral vectors. In a preferred embodiment lentiviral vectors are used to insert short hairpin RNAs (shRNAs), seeking genes that, when knocked down, would enhance mutant huntingtin toxicity. In another preferred embodiment lentiviral vectors are used to insert cDNA, seeking genes that, when overexpressed, would enhance mutant huntingtin toxicity.

Also useful in the practice of the invention is an adenovirus vector. One advantage is the ability of recombinant adenoviruses to efficiently transfer and express recombinant genes in a variety of mammalian cells and tissues in vitro and in vivo, resulting in the high expression of the transferred nucleic acids. Further, the ability to productively infect quiescent cells, expands the utility of recombinant adenoviral libraries. In addition, high expression levels ensure that the products of the nucleic acids will be expressed to sufficient levels to screen for changes in viability of infected cells (see e.g., U.S. Pat. No. 7,029,848, hereby incorporated by reference). In addition libraries can utilize adeno associated virus as the vector, described herein.

Genetic screens, for example, for lethal events, can be carried out in a 96-well format where each well contains isolated cells and a different shRNA, cDNA, or CRISPR/Cas system encoding viral vector. However, this method cannot be performed in vivo. In another embodiment, a DNA barcoding strategy can be used in vivo with a pooled library of viral vectors. In one embodiment the viral vector can be identified by the barcode.

The term “barcode” as used herein, refers to any unique, non-naturally occurring, nucleic acid sequence that may be used to identify the originating source of a nucleic acid fragment. Such barcodes may be sequences including but not limited to, TTGAGCCT, AGTTGCTT, CCAGTTAG, ACCAACTG, GTATAACA or CAGGAGCC. Although it is not necessary to understand the mechanism of an invention, it is believed that the barcode sequence provides a high-quality individual read of a barcode associated with a viral vector, shRNA, or cDNA such that multiple species can be sequenced together.

DNA barcoding is a taxonomic method that uses a short genetic marker in an organism's DNA to identify it as belonging to a particular species. It differs from molecular phylogeny in that the main goal is not to determine classification but to identify an unknown sample in terms of a known classification. Kress et al., “Use of DNA barcodes to identify flowering plants” Proc. Natl. Acad. Sci. U.S.A. 102(23):8369-8374 (2005). Barcodes are sometimes used in an effort to identify unknown species or assess whether species should be combined or separated. Koch H., “Combining morphology and DNA barcoding resolves the taxonomy of Western Malagasy Liotrigona Moure, 1961” African Invertebrates 51(2): 413-421 (2010); and Seberg et al., “How many loci does it take to DNA barcode a crocus?” PLoS One 4(2):e4598 (2009). Barcoding has been used, for example, for identifying plant leaves even when flowers or fruit are not available, identifying the diet of an animal based on stomach contents or feces, and/or identifying products in commerce (for example, herbal supplements or wood). Soininen et al., “Analysing diet of small herbivores: the efficiency of DNA barcoding coupled with high-throughput pyrosequencing for deciphering the composition of complex plant mixtures” Frontiers in Zoology 6:16 (2009).

It has been suggested that a desirable locus for DNA barcoding should be standardized so that large databases of sequences for that locus can be developed. Most of the taxa of interest have loci that are sequencable without species-specific PCR primers. CBOL Plant Working Group, “A DNA barcode for land plants” PNAS 106(31): 12794-12797 (2009). Further, these putative barcode loci are believed short enough to be easily sequenced with current technology. Kress et al., “DNA barcodes: Genes, genomics, and bioinformatics” PNAS 105(8):2761-2762 (2008). Consequently, these loci would provide a large variation between species in combination with a relatively small amount of variation within a species. Lahaye et al., “DNA barcoding the floras of biodiversity hotspots” Proc Natl Acad Sci USA 105(8):2923-2928 (2008).

DNA barcoding is based on a relatively simple concept. For example, most eukaryote cells contain mitochondria, and mitochondrial DNA (mtDNA) has a relatively fast mutation rate, which results in significant variation in mtDNA sequences between species and, in principle, a comparatively small variance within species. A 648-bp region of the mitochondrial cytochrome c oxidase subunit 1 (CO1) gene was proposed as a potential ‘barcode’. As of 2009, databases of CO1 sequences included at least 620,000 specimens from over 58,000 species of animals, larger than databases available for any other gene. Ausubel, J., “A botanical macroscope” Proceedings of the National Academy of Sciences 106(31): 12569 (2009).

Software for DNA barcoding requires integration of a field information management system (FIMS), laboratory information management system (LIMS), sequence analysis tools, workflow tracking to connect field data and laboratory data, database submission tools and pipeline automation for scaling up to eco-system scale projects. Geneious Pro can be used for the sequence analysis components, and the two plugins made freely available through the Moorea Biocode Project, the Biocode LIMS and Genbank Submission plugins handle integration with the FIMS, the LIMS, workflow tracking and database submission.

Additionally other barcoding designs and tools have been described (see e.g., Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci USA. February 17; 106(7):2289-94).

An advantage of this invention is that one neuron in a brain region is used as a genetic screening vehicle, as opposed to one mouse being used as a screening vehicle. Additionally, many modulators of disease outcome can be isolated in a single experiment in contrast to single genes. A modulator is a gene that effects phenotype progression in a disease (disease outcome) (e.g., see example 3). In one embodiment the upper limit of elements that can be screened are shRNA's targeting whole genomes including non-coding RNA's. In one embodiment the upper limit of elements that can be screened are cDNA's expressing genes encoded within whole genomes. In one embodiment cDNA's expressing genes that are known biomarkers of oxidative stress are screened and in another embodiment these genes are targeted by shRNA (see e.g., BOSS (NIEHS), http://www.niehs.nih.gov/research/resources/databases/bosstudy/). In one embodiment viral genome-wide overexpression or knockdown libraries are injected into a section of the brain of a mammal. In another embodiment viral genome-wide overexpression or knockdown libraries are injected into the striatum of a mammal, such that each neuron or glial cell receives on average of one element. In this embodiment each virus expresses either a cDNA or shRNA. Each cDNA expresses a gene that potentially modulates disease outcome, while each shRNA causes repression of a gene that potentially modulates disease outcome. In one embodiment 2.8×10⁵ striatal cells are targeted per mouse, wherein over 80% of viral-transduced cells are neurons. In other mammals the number of cells targeted may be dependent on the size of the brain of the mammal. After incubation in vivo, cells that receive a synthetic lethal hit die and the representation of these library elements are lost. When injections are performed in a paired fashion, modulator's can be identified by comparing disease model mammals to wild-type littermates. Genes that cause synthetic lethality only in combination with a disease-causing mutation can be identified to be a modulator of disease. In contrast, in studies using mouse knockouts, a single gene in the entire mouse or cell type is deactivated.

In another embodiment a protein associated with oxidative stress is found to be a modulator of a central nervous system disease (see Example 2). There are two main families of proteins that detoxify peroxides (Day B J (2009) Biochemical pharmacology 77(3):285-296). Superoxide dismutases (SOD) and catalase are metalloproteins that catalyze “dismutation” reactions. Another class of endogenous catalytic H₂O₂ scavengers is the selenium-containing peroxidases. This is a broad group of enzymes that utilize H₂O₂ as a substrate along with an endogenous source of reducing equivalence. One of the best studied families of peroxidases are the glutathione peroxidases (GPx). The glutathione peroxidase family includes the eight known glutathione peroxidases (Gpx1-8) in humans. Mammalian Gpx1, Gpx2, Gpx3, and Gpx4 have been shown to be selenium-containing enzymes, whereas Gpx6 is a selenoprotein in humans with cysteine-containing homologues in rodents. Several existing studies discuss the observation that selenocysteine-containing enzymes are typically 100 to 1000-fold more active than corresponding mutants where selenocysteine (Sec) is replaced with cysteine (Cys) (Shchedrina et al., (2007) Proc Natl Acad Sci USA. 104(35): 13919-13924). This follows evidence that Sec is a more efficient redox catalyst than Cys. Thus, changing an enzyme's Sec to a Cys results in lower activity. In the case of some enzymes, changing their endogenous Cys to Sec, and adding a selenocysteine insertion sequence (SECIS) element, makes them more active in almost every case. The SECIS element is an RNA element around 60 nucleotides in length that adopts a stem-loop structure and directs the cell to translate UGA codons as selenocysteines. Adding a SECIS element may change enzyme activity. Thus, Cys containing enzymes might have different activity and substrate specificity. For example replacing Cys with Sec in MsrB2 and B3 led to inability to regenerate active enzymes by the natural electron donor. According to Kryukov et al., (2003) Science; 300(5624): 1439-43, Gpx6 is a close homologue of Gpx3, and the rat and mouse orthologs of Gpx6 contain Cys instead of Sec as is found in the human protein. They also note a lack of a functional SECIS unit in rodent Gpx6. Human Gpx6 is 72% homologous to mouse Gpx6. Therefore, in one embodiment the mouse homologue of a peroxidase protein is used in humans as a modulator of disease. In another embodiment a modulator that is a peroxidase protein can be mutated to contain a Cys instead of Sec or vice versa.

Studies have shown that Gpx6 levels correlate with dopamine levels in the brain, signifying that this gene may have relevance to other diseases linked to dopamine, including Parkinson's disease. Furthermore, Gpx6 levels correlate with aging (see Example 1). The other peroxidases, may also be modulators of central nervous system diseases, however the expression of these proteins do not show the same correlation as Gpx6.

In another embodiment a modulator may be involved in the regulation of dopamine signalling. Dopamine is a monoamine neurotransmitter that exerts its action on neuronal circuitry via dopamine receptors. As dopaminergic innervations are most prominent in the brain, dopaminergic dysfunction can critically affect vital central nervous system (CNS) functions, ranging from voluntary movement, feeding, reward, affect, to sleep, attention, working memory and learning (Carlsson, Beaulieu). Dysregulation of dopaminergic neurotransmission has been associated with multiple neurological and psychiatric conditions such as Parkinson's disease, Huntington's disease, attention deficit hyperactivity disorder (ADHD), mood disorders and schizophrenia (Carlsson, Ganetdinov and Caron), as well as various somatic disorders such as hypertension and kidney dysfunction (Missale, Beaulieu, Pharmacol. Rev. 2011, 63, 182).

In yet another aspect of the invention, the modulators of disease identified by the screening methods is used to treat a disease of the central nervous system by impeding phenotype progression of the disease. In one embodiment an agonist or antagonist of the biologic activity of the modulator is used to increase or decrease the activity of the modulator to improve disease outcome. The agonist or antagonist may be a small molecule or protein based therapeutic. Biochemical and cell based in vitro assays can be used to screen for the agonist or antagonist. The modulator can be purified or partially purified from cell extracts containing endogenous protein. This is advantageous in that the purified modulator includes its native post translational modifications and if it is part of a multiprotein complex, those associated proteins are copurified. Recombinant protein can also be expressed in mammalian cell culture, insect cells, bacteria, or yeast. This is advantageous in that the modulator can be tagged, facilitating purification. Such tags include, for example, hexahistidine tags, HA, MYC, and Flag. Recombinant protein can be generated using a DNA vector. Most preferably a plasmid encoding the protein sequence of the modulator is used. The plasmid contains functional elements required for its amplification in prokaryotic cells. The plasmid may contain elements required for the modulator gene to be incorporated into a virus. The plasmid may contain elements that allow expression of the gene in mammalian cells, such as a mammalian promoter. The plasmid may also contain elements for expression in insect or prokaryotic cells. Advantages of insect cells are high protein expression and post translational modifications associated with eukaryotic cells. In one embodiment the modulator protein is used in an in vitro assay that recapitulates its biological activity. In one embodiment Gpx6 peroxidase activity is reconstituted in vitro. Compounds or molecules are incubated at their effective concentrations in the in vitro reconstituted assay with the modulator to test effects on biological activity. In another embodiment, compounds or molecules are tested in cell based assays. In one embodiment reporter genes specific to a modulator can be incorporated into a mammalian cell. In one embodiment promoters of genes up or down regulated during oxidative stress could be incorporated into a reporter construct. The reporter construct may express a marker such as luciferase or GFP. Small molecules that activate Gpx6 activity in the presence of oxidative stress may be screened by assaying for the reporter expression. The modulator may also be overexpressed in such a cell based assay. In another embodiment a therapeutic molecule that activates or represses the expression of the modulator can be used to treat the disease. A cell based assay where a reporter gene is operably linked to the promoter of the modulator can be used. In a specific embodiment the Gpx6 promoter is used.

Many compound or small molecule libraries exist and can be used to screen for agonists and antagonists. Additionally, libraries can be selected, constructed, or designed specifically for a modulator. In one embodiment agonists or antagonists of modulators can be screened using, for example, the NIH Clinical Collections (see, http://www.nihclinicalcollection.com/). The Clinical Collection and NIH Clinical Collection 2 are plated arrays of 446 and 281, respectively, small molecules that have a history of use in human clinical trials. In another embodiment collections of FDA approved drugs are assayed. Advantages of these collections are that the clinically tested compounds are highly drug-like with known safety profiles. Additionally, agonists or antagonists can be modified based on known structures of the modulator and the small molecules.

In another embodiment molecules based on a modulator involved in oxidative stress can be used to treat the disease. The molecule may be a Gpx or peroxidase mimetic, catalase mimetic, or superoxide dismutase (SOD) mimetic (see e.g., Day B J (2009) Biochemical pharmacology 77(3):285-296). Gpx mimetics can be classified in three major categories: (i) cyclic selenenyl amides having a Se—N bond, (ii) diaryl diselenides, and (iii) aromatic or aliphatic monoselenides. Additionally, small molecules, such as the antioxidant ebselen, that acts as a glutathione peroxidase and phospholipid hydroperoxide glutathione peroxidase mimic could be used to treat a central nervous system disease. Ebselen has been shown to substantially reduce gray and white matter damage and neurological deficit associated with transient ischemia (Imai et al., (2001) Stroke; a journal of cerebral circulation 32(9):2149-2154). In other embodiments, drugs used to treat strokes are used to effect a modulator of disease. Molecules such as the antioxidant Coenzyme Q10 may also be used to treat a nervous system disease. In one embodiment the small molecules are administered to pre-symptomatic populations.

In another embodiment a protein based therapeutic may be an agonist or antagonist of a modulator. In one embodiment the therapeutic protein is an antibody or antigen binding fragment of an antibody. In one embodiment the antibody or antigen binding fragment may bind to an inhibitor of the modulator. In a preferred embodiment the antibody is humanized, chimeric, or fully humanized.

In another embodiment the modulator is introduced into a subject in need thereof to treat a central nervous system disease. Treatment may include over-expressing or repressing the modulator in the cells of patient in need thereof effected by the disease. In a more specific embodiment a vector could be used to introduce a nucleic acid that encodes the modulator (see Example 3). In one embodiment, the modulator is introduced by viral delivery. The nucleic acids encoding modulators discovered by the screening method can be delivered using adeno associated virus (AAV), lentivirus, adenovirus or other viral vector types, or combinations thereof. Plasmids that can be used for adeno associated virus (AAV), adenovirus, and lentivirus delivery have been described previously (see e.g., U.S. Pat. Nos. 6,955,808 and 6,943,019, and U.S. Patent application No. 20080254008, hereby incorporated by reference).

In terms of in vivo delivery, AAV is advantageous over other viral vectors due to low toxicity and low probability of causing insertional mutagenesis because it doesn't integrate into the host genome. AAV has a packaging limit of 4.5 or 4.75 Kb. Constructs larger than 4.5 or 4.75 Kb result in significantly reduced virus production. There are many promoters that can be used to drive nucleic acid molecule expression. AAV ITR can serve as a promoter and is advantageous for eliminating the need for an additional promoter element. For ubiquitous expression, the following promoters can be used: CMV, CAG, CBh, PGK, SV40, Ferritin heavy or light chains, etc. For brain expression, the following promoters can be used: SynapsinI for all neurons, CaMKIIalpha for excitatory neurons, GAD67 or GAD65 or VGAT for GABAergic neurons, etc. Promoters used to drive RNA can include: Pol III promoters such as U6 or H1. The use of a Pol II promoter and intronic cassettes can be used to express guide RNA (gRNA).

As to AAV, the AAV can be AAV1, AAV2, AAV5 or any combination thereof. One can select the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. The above promoters and vectors are preferred individually.

The virus may be delivered to the patient in need thereof in any way that allows the virus to contact the target cells in which delivery of the gene of interest is desired. Various means of delivery are described herein, and further discussed in this section. In some embodiments, the viral vector is delivered to the tissue of interest by, for example, an intramuscular or stereotaxic injection, while other times the viral delivery is via intravenous, transdermal, intranasal, oral, mucosal, or other delivery methods. In the provided method, the viral vector can be administered systemically. Such delivery may be either via a single dose, or multiple doses. One skilled in the art understands that the actual dosage to be delivered herein may vary greatly depending upon a variety of factors, such as the vector chosen, the target cell, organism, or tissue, the general condition of the subject to be treated, the degree of transformation/modification sought, the administration route, the administration mode, administration timing, the type of transformation/modification sought, etc.

In preferred embodiments, a suitable amount of virus is introduced into a patient in need thereof directly (in vivo), for example though injection into the body. In one such embodiment, the viral particles are injected directly into the patient's brain, for example, intracranial injection using stereotaxic coordinates may be used to deliver virus to the brain.

Such a delivery may further contain, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline or Hank's Balanced Salt Solution), a pharmaceutically-acceptable excipient, and/or other compounds known in the art. Such a dosage formulation is readily ascertainable by one skilled in the art. The dosage may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

In an embodiment herein the delivery is via an adenovirus, which may be at a single booster dose containing at least 1×10⁵ particles (also referred to as particle units, pu) of adenoviral vector. In an embodiment herein, the dose preferably is at least about 1×10⁶ particles (for example, about 1×10⁶-1×10¹² particles), more preferably at least about 1×10⁷ particles, more preferably at least about 1×10⁸ particles (e.g., about 1×10⁸-1×10¹¹ particles or about 1×10⁸-1×10¹² particles), and most preferably at least about 1×10⁹ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10⁹ articles). Thus, the dose may contain a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹¹ pu, about 2×10¹¹ pu, or about 4×10¹² pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In an embodiment herein, the delivery is via an AAV. A therapeutically effective dosage for in vivo delivery of the AAV to a human is believed to be in the range of from about 20 to about 50 ml of saline solution containing from about 1×10¹⁰ to about 1×10⁵⁰ functional AAV/ml solution. The dosage may be adjusted to balance the therapeutic benefit against any side effects. In an embodiment herein, the AAV dose is generally in the range of concentrations of from about 1×10⁵ to 1×10⁵ genomes AAV, from about 1×10⁸ to 1×10²⁰ genomes AAV, from about 1×10¹⁰ to about 1×10¹⁶ genomes, or about 1×10¹¹ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. In a preferred embodiment, AAV is used with a titer of about 2×10¹³ viral genomes/milliliter, and each of the striatal hemispheres of a mouse receives one 500 nanoliter injection. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for delivery to the Brain, see, e.g., US Patent Publication Nos. US20110293571; US20040013648, US20070025970, US20090111106 and U.S. Pat. No. 7,259,015. In another embodiment lentiviral vectors are used to deliver vectors to the brain of those being treated for a disease.

In an embodiment herein the delivery is via an lentivirus. Zou et al. administered about 10 μl of a recombinant lentivirus having a titer of 1×10⁹ transducing units (TU)/ml by an intrathecal catheter. These sort of dosages can be adapted or extrapolated to use of a retroviral or lentiviral vector in the present invention. For transduction in tissues such as the brain, it is necessary to use very small volumes, so the viral preparation is concentrated by ultracentrifugation. The resulting preparation should have at least 10⁸ TU/ml, preferably from 10⁸ to 10⁹ TU/ml, more preferably at least 10⁹ TU/ml. Other methods of concentration such as ultrafiltration or binding to and elution from a matrix may be used.

In other embodiments the amount of lentivirus administered may be 1×10 or about 1×10⁵ plaque forming units (PFU), 5×10⁵ or about 5×10⁵ PFU, 1×10⁶ or about 1×10⁶ PFU, 5×10⁶ or about 5×10⁶ PFU. 1×10⁷ or about 1×10⁷ PFU, 5×10⁷ or about 5×10⁷ PFU, 1×10⁸ or about 1×10⁸ PFU, 5×10⁸ or about 5×10⁸ PFU, 1×10⁹ or about 1×10⁹ PFU, 5×10⁹ or about 5×10⁹ PFU, 1×10¹⁰ or about 1×10¹⁰ PFU or 5×10¹⁰ or about 5×10¹⁰ PFU as total single dosage for an average human of 75 kg or adjusted for the weight and size and species of the subject. One of skill in the art can determine suitable dosage. Suitable dosages for a virus can be determined empirically.

In an embodiment herein the delivery is via a plasmid. In such plasmid compositions, the dosage should be a sufficient amount of plasmid to elicit a response. For instance, suitable quantities of plasmid DNA in plasmid compositions can be from about 0.1 to about 2 mg, from about 10 μg to about 1 mg, from about 1 μg to about 10 μg from about 10 ng to about 1 μg, or preferably from about 0.2 μg to about 20 μg.

Because the plasmid is the “vehicle” from which the protein is expressed, optimising vector design for maximal protein expression is essential (Lewis et al., (1999). Advances in Virus Research (Academic Press) 54: 129-88). Plasmids usually consist of a strong viral promoter to drive the in vivo transcription and translation of the gene (or cDNA) of interest (Mor, et al., (1995). Journal of Immunology 155 (4): 2039-2046). Promoters may be the SV40 promoter, Rous Sarcoma Virus (RSV) or the like. Intron A may sometimes be included to improve mRNA stability and hence increase protein expression (Leitner et al. (1997) Journal of Immunology 159 (12): 6112-6119). Plasmids also include a strong polyadenylation/transcriptional termination signal, such as bovine growth hormone or rabbit beta-globulin polyadenylation sequences (Alarcon et al., (1999). Adv. Parasitol. Advances in Parasitology 42: 343-410; Robinson et al., (2000). Adv. Virus Res. Advances in Virus Research 55: 1-74; Böhm et al., (1996). Journal of Immunological Methods 193 (1): 29-40).

DNA has been introduced into animal tissues by a number of different methods. The two most popular approaches are injection of DNA in saline, using a standard hypodermic needle, and gene gun delivery. A schematic outline of the construction of a DNA vaccine plasmid and its subsequent delivery by these two methods into a host is illustrated at Scientific American (Weiner et al., (1999) Scientific American 281 (1): 34-41).

Gene gun delivery ballistically accelerates plasmid DNA (pDNA) that has been adsorbed onto gold or tungsten microparticles into the target cells, using compressed helium as an accelerant (Alarcon et al., (1999). Adv. Parasitol. Advances in Parasitology 42: 343-410; Lewis et al., (1999). Advances in Virus Research (Academic Press) 54: 129-88).

Alternative delivery methods have included aerosol instillation of naked DNA on mucosal surfaces, such as the nasal and lung mucosa, (Lewis et al., (1999). Advances in Virus Research (Academic Press) 54: 129-88) and topical administration of pDNA to the eye and vaginal mucosa (Lewis et al., (1999). Advances in Virus Research (Academic Press) 54: 129-88).

The method of delivery determines the dose of DNA required. Saline injections require variable amounts of DNA, from 10 g-1 mg, whereas gene gun deliveries require 100 to 1000 times less DNA. Generally, 0.2 μg-20 μg are required, although quantities as low as 16 ng have been reported. These quantities vary from species to species, with mice, for example, requiring approximately 10 times less DNA than primates. (See e.g., Sedegah et al., (1994). Proceedings of the National Academy of Sciences of the United States of America 91 (21): 9866-9870; Daheshia et al., (1997). The Journal of Immunology 159 (4): 1945-1952; Chen et al., (1998). The Journal of Immunology 160 (5): 2425-2432; Sizemore (1995) Science 270 (5234): 299-302; Fynan et al., (1993) Proc. Natl. Acad. Sci. U.S.A. 90 (24): 11478-82).

In another embodiment a nucleic acid that specifically represses the modulator can be used to treat a patient in need thereof. Nucleic acids that lead to repression may utilize RNAi based methods or CRISPR-Cas9 based systems.

Modulators of central nervous system diseases can be targeted for treatment using the CRISPR-Cas9 system. In one embodiment, the sequences in Table 9 can be used as guide sequences to target a CRISPR enzyme to the genes. Such a system can be used for gene editing to knockout a gene or alter a mutated sequence. Additionally, CRISPR systems allow an increase in gene expression if fused to an activator of transcription. In an additional aspect of the invention, a Cas9 enzyme may comprise one or more mutations and may be used as a generic DNA binding protein with or without fusion to a functional domain. The mutations may be artificially introduced mutations or gain- or loss-of-function mutations. The mutations may include but are not limited to mutations in one of the catalytic domains (D10 and H840) in the RuvC and HNH catalytic domains, respectively. Further mutations have been characterized. In one aspect of the invention, the transcriptional activation domain may be VP64. In other aspects of the invention, the transcriptional repressor domain may be KRAB or SID4X. Other aspects of the invention relate to the mutated Cas 9 enzyme being fused to domains which include but are not limited to a transcriptional activator, repressor, a recombinase, a transposase, a histone remodeler, a demethylase, a DNA methyltransferase, a cryptochrome, a light inducible/controllable domain or a chemically inducible/controllable domain. In one embodiment, CRISPR is targeted to the Gpx6 gene. In another preferred embodiment, Gpx6 gene expression is increased.

In a further embodiment, the invention provides for methods to generate mutant tracrRNA and direct repeat sequences or mutant chimeric guide sequences that allow for enhancing performance of these RNAs in cells. Aspects of the invention also provide for selection of said sequences.

With respect to general information on CRISPR-Cas Systems, components thereof, and delivery of such components, including methods, materials, delivery vehicles, vectors, particles, AAV, and making and using thereof, including as to amounts and formulations, all useful in the practice of the instant invention, reference is made to: U.S. Pat. Nos. 8,999,641, 8,993,233, 8,945,839, 8,932,814, 8,906,616, 8,895,308, 8,889,418, 8,889,356, 8,871,445, 8,865,406, 8,795,965, 8,771,945 and 8,697,359; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); European Patents EP 2 784 162 B1 and EP 2 771 468 B1; European Patent Applications EP 2 771 468 (EP13818570.7), EP 2 764 103 (EP13824232.6), and EP 2 784 162 (EP14170383.5); and PCT Patent Publications PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667). WO 2014/093635 (PCT/US2013/074691), WO 2014/093655 (PCT/US2013/074736), WO 2014/093712 (PCT/US2013/074819). WO2014/093701 (PCT/US2013/074800). WO2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806). WO 2014/204728 (PCT/US2014/041808), WO 2014/204729 (PCT/US2014/041809). Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/836,127, 61/836,101, 61/836,080 and 61/835,973, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT Patent applications Nos: PCT/US2014/041803, PCT/US2014/041800, PCT/US2014/041809, PCT/US2014/041804 and PCT/US2014/041806, each filed Jun. 10, 2014; PCT/US2014/041808 filed Jun. 11, 2014; and PCT/US2014/62558 filed Oct. 28, 2014, and U.S. Provisional Patent Applications Ser. Nos. 61/915,150, 61/915,301, 61/915,267 and 61/915,260, each filed Dec. 12, 2013; 61/757,972 and 61/768,959, filed on Jan. 29, 2013 and Feb. 25, 2013; 61/835,936, 61/836,127, 61/836,101, 61/836,080, 61/835,973, and 61/835,931, filed Jun. 17, 2013; 62/010,888 and 62/010,879, both filed Jun. 11, 2014; 62/010,329 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/054,490, 62/055,484, 62/055,460 and 62/055,487, each filed Sep. 25, 2014; and 62/069,243, filed Oct. 27, 2014. Reference is also made to U.S. provisional patent applications Nos. 62/055,484, 62/055,460, and 62/055,487, filed Sep. 25, 2014; U.S. provisional patent application 61/980,012, filed Apr. 15, 2014; and U.S. provisional patent application 61/939,242 filed Feb. 12, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013. Reference is made to US provisional patent application U.S. Ser. No. 61/980,012 filed Apr. 15, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013.

Mention is also made of U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. application 62/091,462, 12 Dec. 2014, DEAD GUIDES FOR CRISPR TRANSCRIPTION FACTORS; U.S. application 62/096,324, 23 Dec. 2014, DEAD GUIDES FOR CRISPR TRANSCRIPTION FACTORS; U.S. application 62/091,456, 12 Dec. 2014. ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, RNA-TARGETING SYSTEM; U.S. application 62/096,656, 24 Dec. 2014, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/054,675, 24 Sep. 2014, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

Also with respect to general information on CRISPR-Cas Systems, mention is made of the following (also hereby incorporated herein by reference):
Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013). [Epub ahead of print];
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR/Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1): 102-6 (2015);
Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2): 139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546): 186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR/Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
   each of which is incorporated herein by reference, may be considered in the practice of the instant invention, and discussed briefly below:
   Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
   Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
   Wang et al. (2013) used the CRISPR/Cas system for the one-step generation of mice carrying mutations in multiple genes which were traditionally generated in multiple steps by sequential recombination in embryonic stem cells and/or time-consuming intercrossing of mice with a single mutation. The CRISPR/Cas system will greatly accelerate the in vivo study of functionally redundant genes and of epistatic gene interactions.
   Konermann et al. (2013) addressed the need in the art for versatile and robust technologies that enable optical and chemical modulation of DNA-binding domains based CRISPR Cas9 enzyme and also Transcriptional Activator Like Effectors
   Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
   Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and sgRNA can be titrated to minimize off-target modification. Additionally, to facilitate mammalian genome engineering applications, the authors reported providing a web-based software tool to guide the selection and validation of target sequences as well as off-target analyses.
   Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
   Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
   Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
   Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
   Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
   Hsu et al. (2014) is a review article that discusses generally CRISPR-Cas9 history from yogurt to genome editing, including genetic screening of cells.
   Wang et al. (2014) relates to a pooled, loss-of-function genetic screening approach suitable for both positive and negative selection that uses a genome-scale lentiviral single guide RNA (sgRNA) library.
   Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
   Swiech et al. demonstrate that AAV-mediated SpCas9 genome editing can enable reverse genetic studies of gene function in the brain.
   Konermann et al. (2015) discusses the ability to attach multiple effector domains, e.g., transcriptional activator, functional and epigenomic regulators at appropriate positions on the guide such as stem or tetraloop with and without linkers.
   Zetsche et al. demonstrates that the Cas9 enzyme can be split into two and hence the assembly of Cas9 for activation can be controlled.
   Chen et al. relates to multiplex screening by demonstrating that a genome-wide in vivo CRISPR-Cas9 screen in mice reveals genes regulating lung metastasis.
   Ran et al. (2015) relates to SaCas9 and its ability to edit genomes and demonstrates that one cannot extrapolate from biochemical assays. Shalem et al. (2015) described ways in which catalytically inactive Cas9 (dCas9) fusions are used to synthetically repress (CRISPRi) or activate (CRISPRa) expression, showing, advances using Cas9 for genome-scale screens, including arrayed and pooled screens, knockout approaches that inactivate genomic loci and strategies that modulate transcriptional activity.

End Edits
Shalem et al. (2015) described ways in which catalytically inactive Cas9 (dCas9) fusions are used to synthetically repress (CRISPRi) or activate (CRISPRa) expression, showing, advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR/Cas9 knockout and nucleotide preference at the cleavage site. The authors also found that the sequence preference for CRISPRi/a is substantially different from that for CRISPR/Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells.

Useful in the practice of the instant invention, reference is made to the article entitled BCL11A enhancer dissection by Cas9-mediated in situ saturating mutagenesis. Canver, M. C., Smith, E. C., Sher, F., Pinello, L., Sanjana, N. E., Shalem, O., Chen, D. D., Schupp, P. G., Vinjamur, D. S., Garcia, S. P., Luc, S., Kurita, R., Nakamura, Y., Fujiwara, Y., Maeda, T., Yuan, G., Zhang, F., Orkin, S. H., & Bauer, D. E. DOI:10.1038/nature15521, published online Sep. 16, 2015, the article is herein incorporated by reference and discussed briefly below:
Canver et al. involves novel pooled CRISPR-Cas9 guide RNA libraries to perform in situ saturating mutagenesis of the human and mouse BCL11A erythroid enhancers previously identified as an enhancer associated with fetal hemoglobin (HbF) level and whose mouse ortholog is necessary for erythroid BCL11A expression. This approach revealed critical minimal features and discrete vulnerabilities of these enhancers. Through editing of primary human progenitors and mouse transgenesis, the authors validated the BCL11A erythroid enhancer as a target for HbF reinduction. The authors generated a detailed enhancer map that informs therapeutic genome editing.

In addition, mention is made of PCT application PCT/US14/70057, Attorney Reference 47627.99.2060 and BI-2013/107 entitled “DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS (claiming priority from one or more or all of US provisional patent applications: 62/054,490, filed Sep. 24, 2014; 62/010,441, filed Jun. 10, 2014; and 61/915,118, 61/915,215 and 61/915,148, each filed on Dec. 12, 2013) (“the Particle Delivery PCT”), incorporated herein by reference, with respect to a method of preparing an sgRNA-and-Cas9 protein containing particle comprising admixing a mixture comprising an sgRNA and Cas9 protein (and optionally HDR template) with a mixture comprising or consisting essentially of or consisting of surfactant, phospholipid, biodegradable polymer, lipoprotein and alcohol; and particles from such a process. For example, wherein Cas9 protein and sgRNA were mixed together at a suitable, e.g., 3:1 to 1:3 or 2:1 to 1:2 or 1:1 molar ratio, at a suitable temperature, e.g., 15-30C, e.g., 20-25C, e.g., room temperature, for a suitable time, e.g., 15-45, such as 30 minutes, advantageously in sterile, nuclease free buffer, e.g., IX PBS. Separately, particle components such as or comprising: a surfactant, e.g., cationic lipid, e.g., 1,2-dioleoyl-3-trimethylammonium-propane (DOTAP); phospholipid, e.g., dimyristoylphosphatidylcholine (DMPC); biodegradable polymer, such as an ethylene-glycol polymer or PEG, and a lipoprotein, such as a low-density lipoprotein, e.g., cholesterol were dissolved in an alcohol, advantageously a C₁₆ alkyl alcohol, such as methanol, ethanol, isopropanol, e.g., 100% ethanol. The two solutions were mixed together to form particles containing the Cas9-sgRNA complexes. Accordingly, sgRNA may be pre-complexed with the Cas9 protein, before formulating the entire complex in a particle. Formulations may be made with a different molar ratio of different components known to promote delivery of nucleic acids into cells (e.g. 1,2-dioleoyl-3-trimethylammonium-propane (DOTAP), 1,2-ditetradecanoyl-sn-glycero-3-phosphocholine (DMPC), polyethylene glycol (PEG), and cholesterol) For example DOTAP:DMPC:PEG:Cholesterol Molar Ratios may be DOTAP 100, DMPC 0, PEG 0, Cholesterol 0; or DOTAP 90, DMPC 0, PEG 10, Cholesterol 0; or DOTAP 90, DMPC 0, PEG 5, Cholesterol 5. DOTAP 100, DMPC 0, PEG 0, Cholesterol 0. That application accordingly comprehends admixing sgRNA, Cas9 protein and components that form a particle; as well as particles from such admixing. Aspects of the instant invention can involve particles; for example, particles using a process analogous to that of the Particle Delivery PCT, e.g., by admixing a mixture comprising sgRNA and/or Cas9 as in the instant invention and components that form a particle, e.g., as in the Particle Delivery PCT, to form a particle and particles from such admixing (or, of course, other particles involving sgRNA and/or Cas9 as in the instant invention).

In general, the CRISPR-Cas or CRISPR system is as used in the foregoing documents, such as WO 2014/093622 (PCT/US2013/074667) and refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell. In some embodiments, direct repeats may be identified in silico by searching for repetitive motifs that fulfill any or all of the following criteria: 1. found in a 2 Kb window of genomic sequence flanking the type II CRISPR locus; 2. span from 20 to 50 bp; and 3. interspaced by 20 to 50 bp. In some embodiments, 2 of these criteria may be used, for instance 1 and 2, 2 and 3, or 1 and 3. In some embodiments, all 3 criteria may be used.

In embodiments of the invention the terms guide sequence and guide RNA, i.e. RNA capable of guiding Cas to a target genomic locus, are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In a classic CRISPR-Cas systems, the degree of complementarity between a guide sequence and its corresponding target sequence can be about or more than about 50%, 60%, 75%, 80%. 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and advantageously tracr RNA is 30 or 50 nucleotides in length. However, an aspect of the invention is to reduce off-target interactions, e.g., reduce the guide interacting with a target sequence having low complementarity. Indeed, in the examples, it is shown that the invention involves mutations that result in the CRISPR-Cas system being able to distinguish between target and off-target sequences that have greater than 80% to about 95% complementarity, e.g., 83%-84% or 88-89% or 94-95% complementarity (for instance, distinguishing between a target having 18 nucleotides from an off-target of 18 nucleotides having 1, 2 or 3 mismatches). Accordingly, in the context of the present invention the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In particularly preferred embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e. an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence.

The methods according to the invention as described herein comprehend inducing one or more mutations in a eukaryotic cell (in vitro, i.e. in an isolated eukaryotic cell) as herein discussed comprising delivering to cell a vector as herein discussed. The mutation(s) can include the introduction, deletion, or substitution of one or more nucleotides at each target sequence of cell(s) via the guide(s) RNA(s) or sgRNA(s). The mutations can include the introduction, deletion, or substitution of 1-75 nucleotides at each target sequence of said cell(s) via the guide(s) RNA(s) or sgRNA(s). The mutations can include the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 40, 45, 50, 75, 100, 200, 300, 400 or 500 nucleotides at each target sequence of said cell(s) via the guide(s) RNA(s) or sgRNA(s).

For minimization of toxicity and off-target effect, it will be important to control the concentration of Cas mRNA and guide RNA delivered. Optimal concentrations of Cas mRNA and guide RNA can be determined by testing different concentrations in a cellular or non-human eukaryote animal model and using deep sequencing the analyze the extent of modification at potential off-target genomic loci. Alternatively, to minimize the level of toxicity and off-target effect, Cas nickase mRNA (for example S. pyogenes Cas9 with the D10A mutation) can be delivered with a pair of guide RNAs targeting a site of interest. Guide sequences and strategies to minimize toxicity and off-target effects can be as in WO 2014/093622 (PCT/US2013/074667); or, via mutation as herein.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence.

The nucleic acid molecule encoding a Cas is advantageously codon optimized Cas. An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a Cas is codon optimized for expression in particular cells, such as eukaryotic cells. The eukaryotic cells may be those of or derived from a particular organism, such as a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way how the Cas transgene is introduced in the cell is may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences. Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus, such as for instance one or more oncogenic mutations, as for instance and without limitation described in Platt et al. (2014), Chen et al., (2014) or Kumar et al. (2009).

In some embodiments, the Cas sequence is fused to one or more nuclear localization sequences (NLSs), such as about or more than about 1, 2, 3, 4, 5, 6, 7, 8, 9, 10, or more NLSs. In some embodiments, the Cas comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In a preferred embodiment of the invention, the Cas comprises at most 6 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Non-limiting examples of NLSs include an NLS sequence derived from: the NLS of the SV40 virus large T-antigen, having the amino acid sequence PKKKRKV (SEQ ID NO: X); the NLS from nuclcoplasmin (e.g. the nucleoplasmin bipartite NLS with the sequence KRPAATKKAGQAKKKK) (SEQ ID NO: X); the c-myc NLS having the amino acid sequence PAAKRVKLD (SEQ ID NO: X) or RQRRNELKRSP (SEQ ID NO: X); the hRNPAI M9 NLS having the sequence NQSSNFGPMKGGNFGGRSSGPYGGGGQYFAKPRNQGGY(SEQ ID NO: X); the sequence RMRIZFKNKGKDTAELRRRRVEVSVELRKAKKDEQILKRRNV (SEQ ID NO: X) of the IBB domain from importin-alpha; the sequences VSRKRPRP (SEQ ID NO: X) and PPKKARED (SEQ ID NO: X) of the myoma T protein; the sequence POPKKKPL (SEQ ID NO: X) of human p53; the sequence SALIKKKKKMAP (SEQ ID NO: X) of mouse c-abl IV; the sequences DRLRR (SEQ ID NO: X) and PKQKKRK (SEQ ID NO: X) of the influenza virus NS1; the sequence RKLKKKIKKL (SEQ ID NO: X) of the Hepatitis virus delta antigen; the sequence REKKKFLKRR (SEQ ID NO: X) of the mouse Mxl protein; the sequence KRKGDEVDGVDEVAKKKSKK (SEQ ID NO: X) of the human poly(ADP-ribose) polymerase; and the sequence RKCLQAGMNLEARKTKK (SEQ ID NO: X) of the steroid hormone receptors (human) glucocorticoid. In general, the one or more NLSs are of sufficient strength to drive accumulation of the Cas in a detectable amount in the nucleus of a eukaryotic cell. In general, strength of nuclear localization activity may derive from the number of NLSs in the Cas, the particular NLS(s) used, or a combination of these factors. Detection of accumulation in the nucleus may be performed by any suitable technique. For example, a detectable marker may be fused to the Cas, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of CRISPR complex formation (e.g. assay for DNA cleavage or mutation at the target sequence, or assay for altered gene expression activity affected by CRISPR complex formation and/or Cas enzyme activity), as compared to a control no exposed to the Cas or complex, or exposed to a Cas lacking the one or more NLSs.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s) (e.g., sgRNAs); and, when a single vector provides for more than 16 RNA(s) (e.g., sgRNAs), one or more promoter(s) can drive expression of more than one of the RNA(s) (e.g., sgRNAs), e.g., when there are 32 RNA(s) (e.g., sgRNAs), each promoter can drive expression of two RNA(s) (e.g., sgRNAs), and when there are 48 RNA(s) (e.g., sgRNAs), each promoter can drive expression of three RNA(s) (e.g., sgRNAs). By simple arithmetic and well established cloning protocols and the teachings in this disclosure one skilled in the art can readily practice the invention as to the RNA(s) (e.g., sgRNA(s) for a suitable exemplary vector such as AAV, and a suitable promoter such as the U6 promoter, e.g., U6-sgRNAs. For example, the packaging limit of AAV is ˜4.7 kb. The length of a single U6-sgRNA (plus restriction sites for cloning) is 361 bp. Therefore, the skilled person can readily fit about 12-16, e.g., 13 U6-sgRNA cassettes in a single vector. This can be assembled by any suitable means, such as a golden gate strategy used for TALE assembly (www.genome-engineering.org/taleffectors/). The skilled person can also use a tandem guide strategy to increase the number of U6-sgRNAs by approximately 1.5 times, e.g., to increase from 12-16, e.g., 13 to approximately 18-24, e.g., about 19 U6-sgRNAs. Therefore, one skilled in the art can readily reach approximately 18-24, e.g., about 19 promoter-RNAs, e.g., U6-sgRNAs in a single vector, e.g., an AAV vector. A further means for increasing the number of promoters and RNAs, e.g., sgRNA(s) in a vector is to use a single promoter (e.g., U6) to express an array of RNAs, e.g., sgRNAs separated by cleavable sequences. And an even further means for increasing the number of promoter-RNAs, e.g., sgRNAs in a vector, is to express an array of promoter-RNAs, e.g., sgRNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53.short, www.nature.com/mt/joumal/v16/n9/abs/mt2008144a.html). In an advantageous embodiment, AAV may package U6 tandem sgRNA targeting up to about 50 genes. Accordingly, from the knowledge in the art and the teachings in this disclosure the skilled person can readily make and use vector(s), e.g., a single vector, expressing multiple RNAs or guides or sgRNAs under the control or operatively or functionally linked to one or more promoters-especially as to the numbers of RNAs or guides or sgRNAs discussed herein, without any undue experimentation.

The guide RNA(s), e.g., sgRNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the 3-actin promoter, the phosphoglycerol kinase (PGK) promoter, and the EF1α promoter. An advantageous promoter is the promoter is U6.

Mice used in experiments are about 20 g. From that which is administered to a 20 g mouse, one can extrapolate to scale up dosing to a 70 kg individual. In another preferred embodiment the doses herein are scaled up based on an average 70 kg individual to treat a patient in need thereof. The frequency of administration is within the ambit of the medical or veterinary practitioner (e.g., physician, veterinarian), or scientist skilled in the art.

In other embodiments, any of the proteins, antagonists, antibodies, agonists, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described herein. Using this approach, one may be able to achieve therapeutic efficacy with lower dosages of each agent, thus reducing the potential for adverse side effects. In a preferred embodiment, Huntington's Disease is treated by use of an identified modulator, as described herein, in conjunction with a known treatment. Treating with a modulator by either effecting its expression or by overexpressing the protein may not completely alleviate symptoms. Therefore, other drugs that specifically target the symptoms can be combined with that of a modulator. Central nervous system diseases are associated with oxidative stress as well as having neurological symptoms that lead to both mental and physical abnormalities. A combination therapy may be used to synergistically alleviate these symptoms. Antioxidants and Gpx mimetics may be used in combination with other known treatments when a modulator involved in oxidative stress is identified. The antioxidant ebselen may be used at about 300 mg per day. Such treatments may comprise Tetrabenazine, neuroleptics, benzodiazepines, amantadine, anti Parkinson's drugs and valproic acid. Tetrabenazine is used to treat Huntington's chorea (uncontrolled muscle movements) and can be given in doses of 12.5 mg orally weekly to a maximum dose of 37.5 to 50 mg daily. Preferably less than 25 mg is administered. In combination, the dosage may be less than 12.5 mg. Neuroleptics are used to treat psychotic disorders and may be given in a dose of 10 to 200 mg daily. Benzodiazepines are used as sedatives, hypnotics, anxiolytics, anticonvulsants and muscle relaxants. They may be administered in doses of between 3 to 6 mg/day. Amantadine is an antiviral medication and may be used in doses of 200 mg/day, up to 400 mg per day. Valproic acid is used to treat various types of seizure disorders and can be administered in doses of 5 to 60 mg/kg per day in divided doses. In one embodiment of the invention, the medicament may further comprise but is not limited to the following Parkinson's drugs: levodopa, dopamine agonists, catechol O-methyltransferase (COMT) inhibitors, monoamine oxidase B (MAO B) inhibitors, anticholinergic agents, or a combination thereof.

In another embodiment, antibodies are developed that bind specifically to the modulators using known methods in the art. In one embodiment the antibodies are polyclonal. In another embodiment the antibodies are monoclonal. In one embodiment the antibodies are generated against the full length protein. In another embodiment the antibodies are generated against antigenic fragments of the modulators. In one embodiment the antibodies are produced in sheep. In one embodiment the antibodies are produced in rabbits. In one embodiment the antibodies are produced in mice. In one embodiment the antibodies are produced in goats. In one embodiment the antibodies are used to study central nervous system diseases by staining tissue samples. In one embodiment the antibodies are used to determine protein quantity.

In another embodiment, modulators of central nervous system diseases can be used for diagnostic or prognostic screening. In one embodiment a modulator found to be synthetically lethal when knocked down in the screening method, would be a positive prognostic marker of disease outcome. In a preferred embodiment the modulator is Gpx6. In one embodiment a modulator found to be synthetically lethal when overexpressed in the screening method, would be a negative prognostic marker of disease outcome. In a preferred embodiment the protein expression of the modulator is determined. This may be performed with antibodies in western blots or in tissue staining. In another preferred embodiment gene expression is determined. This may be performed using microarrays, RT-PCR, quantitative PCR, or northern blot.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

The practice of the present invention employs, unless otherwise indicated, conventional techniques for generation of genetically modified mice. See Marten H. Hofker and Jan van Deursen, TRANSGENIC MOUSE METHODS AND PROTOCOLS, 2nd edition (2011).

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

Differential Gene Expression Profiling and Pathways Analysis

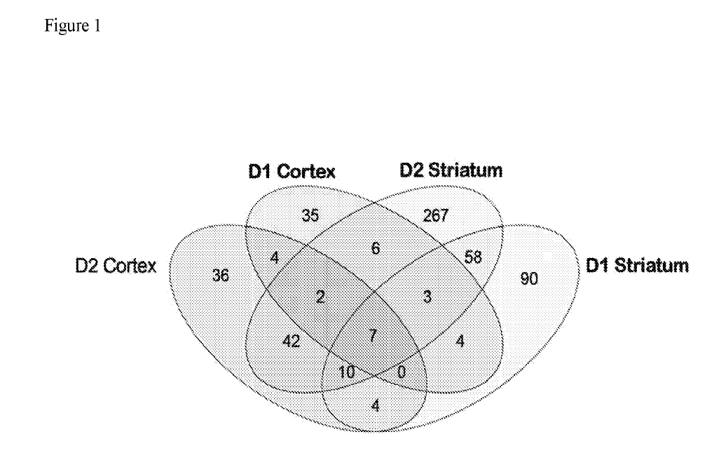

This Example Describes Cell-Type Specific Molecular Profiles of Cell Populations during normal mouse brain aging and normal age-associated molecular pathways in various neurodegenerative disease-relevant cell types (FIG. 1 and Tables 1-8). Applicants employed the translating ribosome affinity purification (TRAP) methodology (Heiman et al., (2008) Cell 135(4):738-748; Doyle et al., (2008) Cell 135(4):749-762) to create cell-type specific molecular profiles of cell populations during normal mouse brain aging. Mice aged 6 weeks or 2 years and 6 weeks from the Drd1::EGFP-L10a or Drd2::EGFP-L10a Bacterial Artificial Chromosome (BAC) transgenic lines (n=4 each group) were decapitated and brain tissue was immediately dissected and used for TRAP RNA purifications as previously described (Heiman et al., (2008). RNA was used to interrogate Affymetrix Mouse Exon Chips (Affymetrix, Santa Clara, Calif.) after amplification using the NuGEN Ovation protocol for probe preparation (NuGEN, San Carlos, Calif.). Genes differentially expressed across aging were identified as previously described (Heiman et al., (2008), Heiman et al., (2014) Nat Protoc. 2014; 9(6):1282-91) using Welch's t-test. Applicants defined significantly differentially expressed genes as those having any probe-sets with >1.2-fold change and a Benjamini-Hochberg adjusted p-value from Welch's t test of <0.05. For each comparison group, the set of statistically significant differentially expressed genes, independent of magnitude of change, was compared against the Wikipathways gene sets to compute overlaps. Statistical significance of gene set overlaps was assessed by a hypergeometric test.

Results.

Each cell type displayed a unique pattern of gene expression changes that was associated with aging (Tables 1-4 and FIG. 1). Only 5 genes, including 2 pseudogenes, displayed altered expression with aging in all cell types (Tnnt2, Gm5425, Rnd3, Pisd, and Pisd-ps3), indicating that there is not a general aging program across these cell types studied, but rather that even closely related cell types show distinct molecular changes during normal aging.

Pathways analysis of genes whose expression was altered revealed several molecular pathways altered with aging in each cell type (Tables 5-8) In Drd2-expressing striatal neurons, which displayed the most number of altered gene pathways during aging, “glutathione-mediated detoxification” and “glutathione redox reactions” were amongst the top gene pathways altered with age (including the genes Gsta3, Gsta4, Gstm1, Gstm6, Gpx1, Gpx2, and Gpx6). Oxidative damage has long been linked to aging (Harman et al., 1956). Given that oxidative damage to DNA, proteins, and lipids have all been reported to increase with age in the brain (Mecocci et al., (1993) Annals of neurology 34(4):609-616; Dei, Takeda, et al., (2002) Acta neuropathologica 104(2): 113-122; Smith, Carney et al., (1991) Proceedings of the National Academy of Sciences of the USA 88(23): 10540-10543), the increases to glutathione-dependent enzymes reported here likely reflect a homeostatic neuronal response to increased oxidative damage in this cell population.

### Example 2

### Synthetic Lethal Knockdown Screen for Genes Enhancing Huntingtin Toxicity

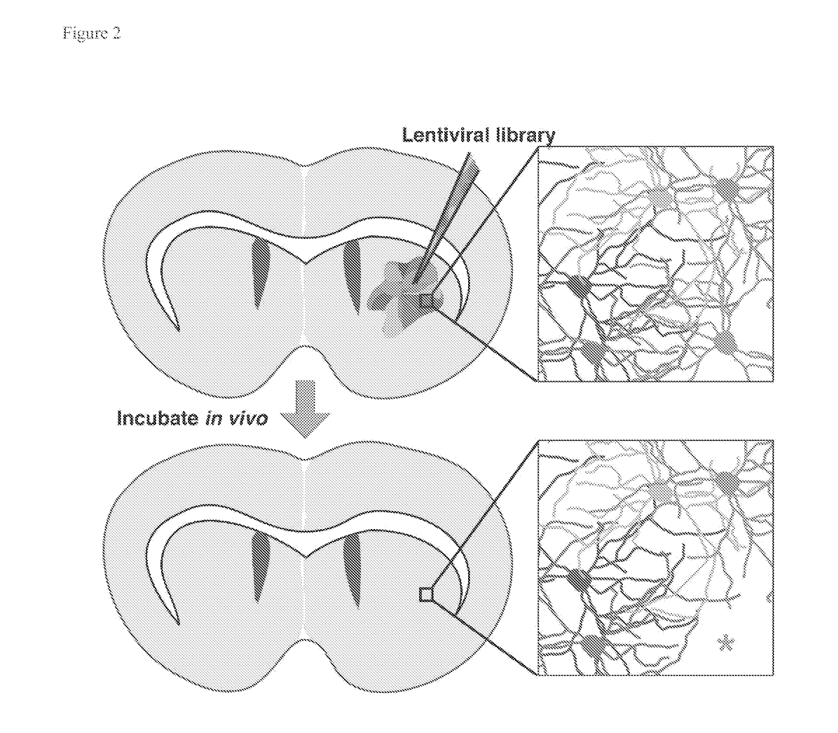

This example describes results of the SLIC genetic screening platform used in the mammalian nervous system. The SLIC screening platform utilizes individual neurons in a brain region as a genetic screening vehicle, as opposed to one mouse being used as a screening vehicle (FIG. 2). Specifically, genes were screened for synthetic lethality in a Huntington's disease mouse model that, when knocked down, would enhance mutant huntingtin toxicity. R6/2 mice (Mangiarini et al., (1996) Cell 87(3):493-506) or control littermates 6 weeks of age were anesthetized with a mixture of ketamine (Putney Inc., Portland, Me.) and xylazine (Lloyd Inc., Shenandoah, Iowa) and mounted on a Leica (Solms, Germany) mouse stereotaxic frame in a flat-skull position. Viral pools of lentiviruses carrying barcoded short hairpin RNAs (shRNAs) were injected bilaterally into mouse striata of disease and control littermates. One microliter of the barcoded lentiviral pools was injected at each of the following four coordinates (in mm relative to bregma, sagittal suture and dural surface): AP=0.3, L=2, DV=−3.7; AP=0.3, L=−2, DV=−3.7; AP-0.9, L=1.7, DV=−3.3; AP-0.9, L=−1.7, DV=−3.3. The lentiviruses carrying barcoded short hairpin RNAs (shRNAs) included 96 shRNA elements for the screen (Table 9), which included a positive control shRNA, negative control shRNAs, and experimental shRNAs that targeted 24 genes, with an average of 3.4 hairpins per gene. The 24 target genes were selected due to their high magnitude change in the aging TRAP study described in example 1 or else a previously reported link to Huntington's disease.

Two days, four weeks, or six weeks after lentiviral injections, mice were sacrificed and brain tissue was processed for genomic DNA extraction using a Qiagen kit (Qiagen, Hilden, Germany). Illumina sequencing and deconvolution were performed as previously described to determine lentiviral barcode representation (Ashton, Jordan, et al., 2012). (See also: http://www.broadinstitute.org/rnai/public/resources/protocols). Significance of screen results was calculated with the RIGER software as previously described (Luo, Cheung, Subramanian, et al. (2008). (See also: http://www.broadinstitute.org/cancer/software/GENE-E/).

Results.

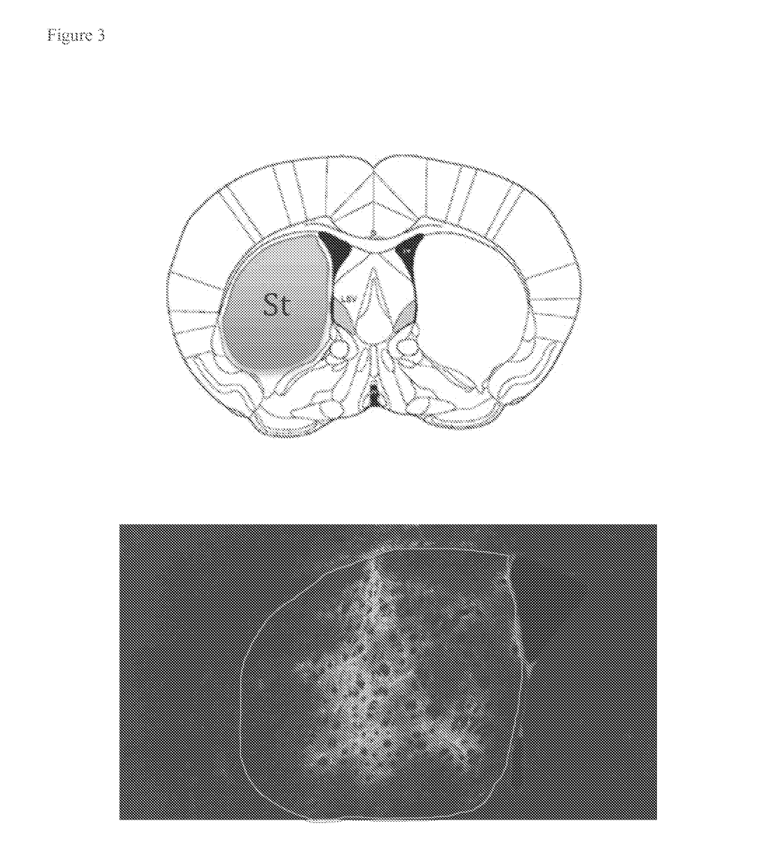

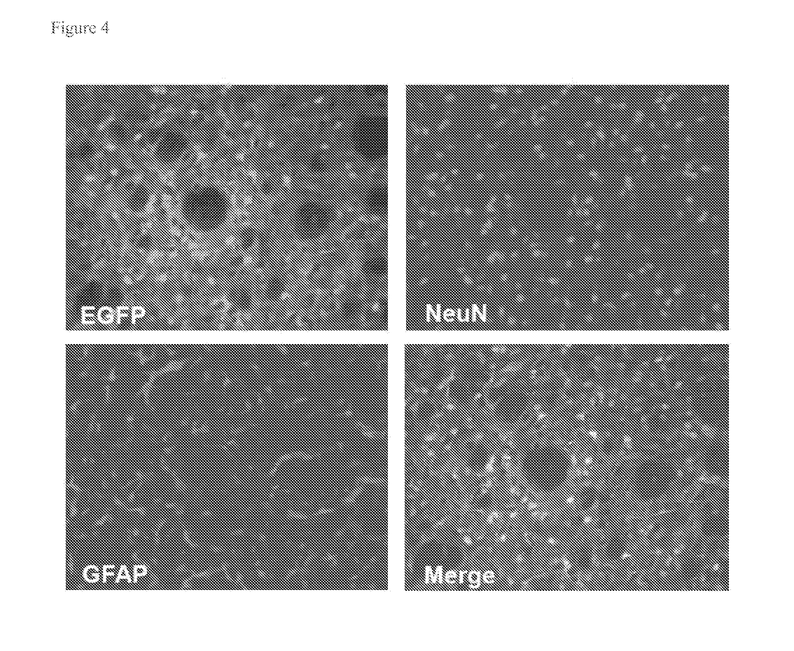

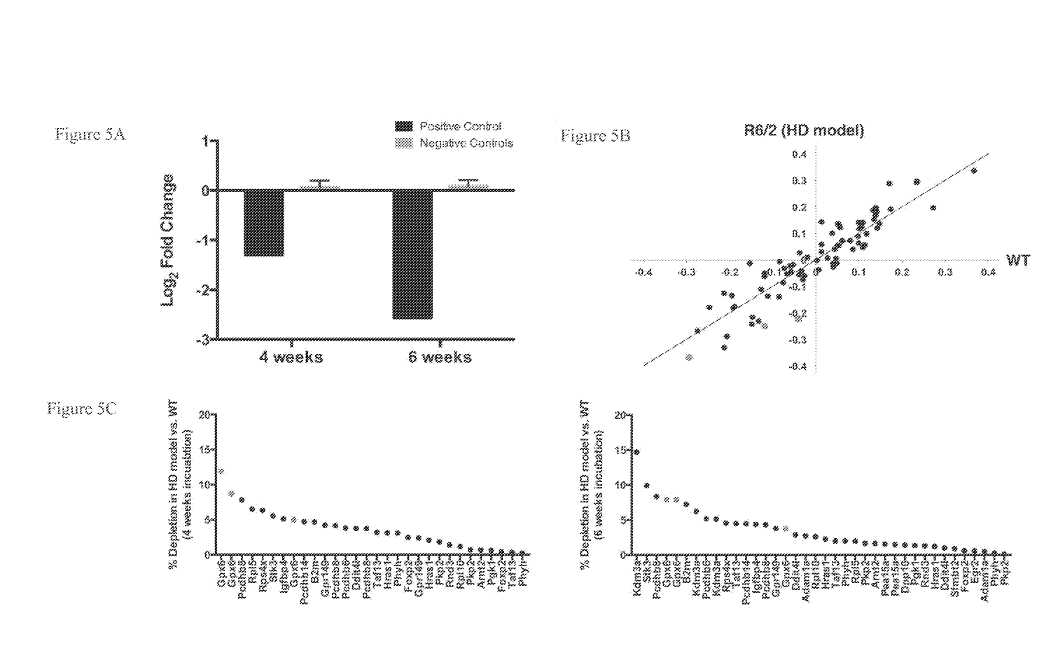

Based on test injections, Applicants calculate that up to 2.8×10⁵ striatal cells are targeted per mouse (FIG. 3), and that over 80% of viral-transduced cells are neurons (FIG. 4). Comparison of viral barcode representation in the wild-type control (non-model) mouse striatal samples at 4 weeks versus 2 days revealed that the positive control lentivirus, carrying an shRNA targeting the Psmd2 gene product (a proteasomal subunit, depletion of which is expected to lead to cell death), was greatly reduced in representation, while negative controls, which have no expected target in the mouse genome, were not reduced in representation (FIG. 5A). ShRNAs that led to enhanced cell death in R6/2 mice and not control mice revealed genes that display synthetic lethality with mutant huntingtin. Comparison of the R6/2 Huntington's disease model mice versus control littermates at the 4 and 6 weeks experimental time-points revealed that all shRNAs targeting Gpx6, a glutathione peroxidase that by homology is predicted to detoxify H2O2 to water, demonstrated synthetic lethality with mutant huntingtin (p value=0.0036 at 4 weeks of incubation; p value=0.0321 at 6 weeks of incubation) (FIGS. 5B and 5C and Tables 10, 11, and 12). No other targeted gene displayed statistically significant synthetic lethality at either screening time-point. Importantly, other shRNAs that affected general health of cells did not exhibit synthetic lethality with mutant huntingtin, and were lost approximately equally in both R6/2 mouse brain and controls (FIG. 5B).

### Example 3

### Gpx6 Function and Expression

This example describes Gpx6 function and expression. Applicants assessed Gpx6 distribution across brain region and age. Gpx6, high-titer adeno-associated virus serotype 9 (AAV9) was used to overexpress FLAG-tagged Gpx6 or the TRAP construct (control) in the striatum of the R6/2 model and control mice by bilateral injection at the following coordinates: AP=0.6, L=1.85, DV=−3.5; and AP=0.6, L=−1.85, DV=−3.5. AAV was used with a titer of about 2×10¹³ viral genomes/milliliter, and each of the striatal hemispheres received one 500 nanoliter injection in the Gpx6 over expression study. Virus vehicle was either phosphate-buffered saline or Hank's Balanced Salt Solution. Mice were 6 weeks of age upon injection with the AAV9 construct, and were tested in an open field assay at two weeks post injection. In a separate series of experiments, mice were also injected with AAV9. at the same coordinates, but with one striatal hemisphere receiving the FLAG-tagged Gpx6 AAV9 and one striatal hemisphere receiving the TRAP construct (control) AAV9. These mice were perfused for indirect immunofluorescent staining at two weeks post injection.

Results.

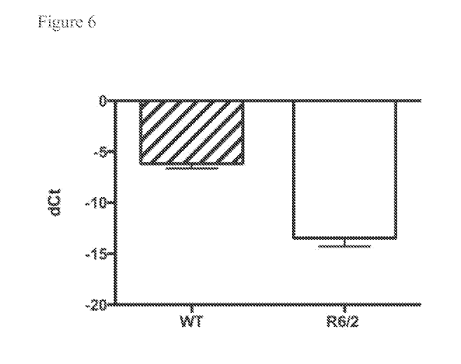

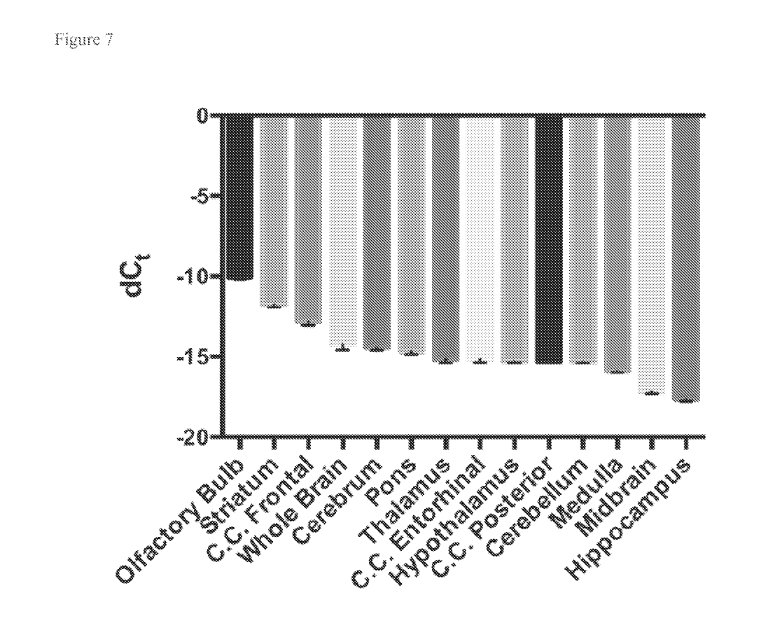

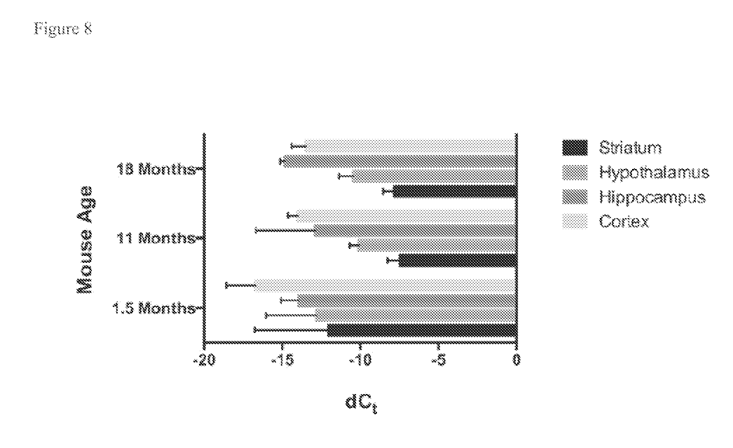

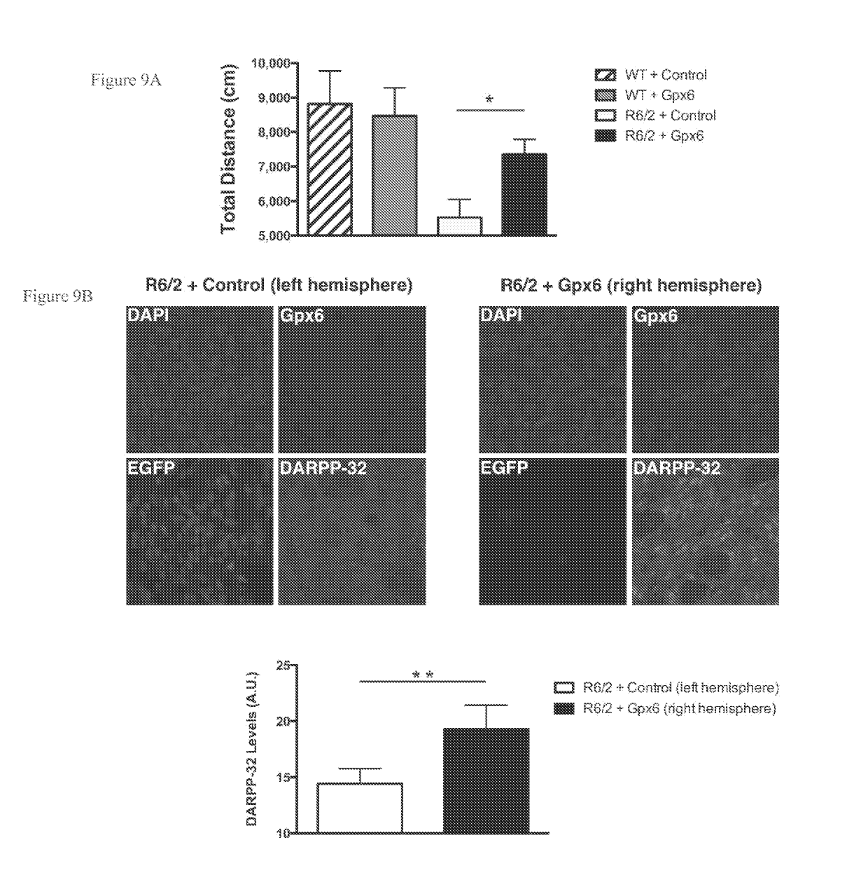

Applicants found that Gpx6 expression is down-regulated in the brains of Huntington's disease model mice (FIG. 6). Applicants also found Gpx6 to be highly expressed in the olfactory bulb, striatum, and frontal cerebral cortex (FIG. 7) and, confirming the TRAP results in example 1, observed that Gpx6 expression increases with age (FIG. 8). Over-expression of Gpx6 showed a therapeutic effect on phenotype progression in a Huntington's disease mouse model. Two weeks after viral injection, Applicants observed a dramatic rescue of open-field motor behavior in R6/2 mice, but no effect of viral transduction on motor behavior in wild-type mice (FIG. 9A). Finally, analysis of a molecular marker of Huntington's disease progression, loss of DARPP-32 striatal expression (Bibb et al., (2000) Proceedings of the National Academy of Sciences of the USA 97(12):6809-6814), revealed that Gpx6 over-expression also increases DARPP-32 expression in the R6/2 model (FIG. 9B).

### Example 4

### Effects of Gpx6 Overexpression on Parkinson's Disease Model Phenotype Progression

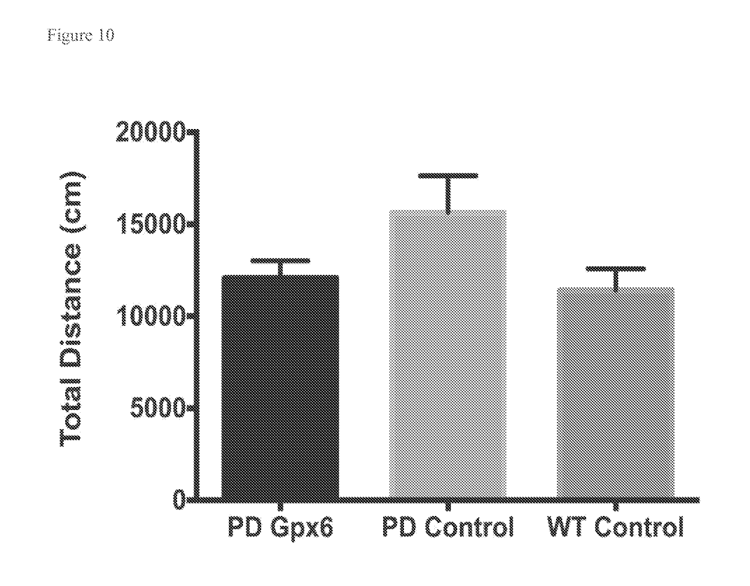

This example describes a decrease in phenotype progression in a Parkinson's disease mouse model after overexpression of Gpx6. Based on the ability of Gpx6 overexpression to delay the emergence of several Huntington's disease phenotypes in mouse models of the disease, Applicant's tested the effects of Gpx6 overexpression on a mouse model of Parkinson's disease (PD). The PD model overexpresses human alpha-synuclein that contains two PD-associated mutations, A30P and A53T (The Jackson Laboratories stock #008239). Starting at 2-3 months of age, these PD model mice are hyperactive, but then start to show a reduction in activity at approximately 16 months of age. In order to test the effect of Gpx6 overexpression on the disease course in this mouse model, Applicant's injected mice at 6 weeks of age with a control (TRAP construct) or Gpx6 overexpression virus, allowed the mice to recover, and aged them to a time-point where it would be expected to see a behavioral phenotype. The data shows that Gpx6 overexpression has a therapeutic benefit in this mouse model of PD, as Gpx6 overexpression reduced the hyperactivity seen at this age in this PD model (FIG. 10).

### Methods

Animal Usage.

All animal experiments were conducted with the approval of the Massachusetts Institute of Technology Animal Care and Use Committee. Mice were housed with food and water provided ad libitum. Experiments were conducted with Drd1::EGFP-L10a or Drd2::EGFP-L10a Bacterial Artificial Chromosome (BAC) transgenic (Heiman et al., 2008), adult (6 weeks old and 2 years, 6 weeks old) female mice on the C57BL/6J strain background, or with R6/2 model mice (Mangiarini et al., 1996) (B6CBA-Tg(HDexon1)62Gpb/1J, Jackson Laboratory stock #002810) at 5-12 weeks of age.

In Vitro Validation of Lentiviral Knockdown Efficiency.

HEK293T/17 cells (ATCC, Manassas, Va.) were grown in Dulbecco's Modified Eagle Medium (Invitrogen, Carlsbad, Calif.) supplemented with 10% (vol/vol) heat-inactivated fetal bovine serum (Invitrogen, Carlsbad, Calif.) and transfected with FLAG-tagged Gpx6 over-expression constructs (Origene, Rockville, Md.) using the FuGENE6HD reagent (Promega, Madison Wis.) following the manufacturer's instructions. One day after transfection, cells were transduced with Gpx6-targeting shRNA lentiviruses, and cell lysates were prepared for standard Western blotting two days later by lysing cells directly in Western blot sample buffer.

Indirect Immunofluorescent Staining.

Mouse brain tissue was prepared and stained as previously described (Heiman et al., 2008), using the following primary antibodies: DARPP-32 (Cell Signaling Technology, Beverly, Mass., antibody19A3, 1:1,000 dilution), GFP (Abcam, Cambridge, England, antibody ab6556, 1:5,000 dilution), NeuN (1:100 dilution), and GFAP (1:1,000 dilution).

Lentiviral Library Preparation.

Lentivirus was prepared and pooled as previously described (Root, Sabatini, et al., 2006). Lentivirus was concentrated by centrifugation at 20,000×g through a 20% sucrose cushion in a SW32Ti rotor (Beckman Coulter, Inc., Pasadena, Calif.), using an Optima L-90K centrifuge (Beckman Coulter, Inc., Pasadena, Calif.), and resuspended in Hank's Balanced Salt Solution (HBSS) to an approximate titer of 5×105 functional particles/μl before stereotaxic injection.

Open Field Behavioral Testing.

Mice were placed in a non-illuminated open field platform (19 in length×20 in width×15 in high; with 16 infrared beams each in the X and Y axis) housed within an environmental control chamber (both from Omnitech Electronic, Inc., Columbus, Ohio) during the first half of their light phase. Activity measurements captured by infrared beam breaks were collected in 10 min intervals, for a total of 60 min.

Quantitative PCR.

RNA was purified from aged and control mouse brain tissue using the RNeasy Lipid Tissue Mini Kit (Qiagen, Hilden, Germany). Complementary cDNA was produced using the SuperScript III kit (Invitrogen, Carlsbad, Calif.). Alternatively, to profile gene expression across brain regions, a commercially available mouse brain cDNA panel was used (Zyagen, San Diego, Calif.). Quantitative PCR was performed with 100 ng of cDNA, Taqman reagents and primers (Invitrogen, Carlsbad, Calif.), and a LightCycler480 (Roche, Basel Switzerland). Taqman primers used were as follows:

TaqMan Gene Expression Assay ID: Mm00607939_s1, Gene Symbol: Actb, mCG23209

### TaqMan Gene Expression Assay ID: Mm00513979_m1, Gene Symbol: Gpx6

Generation of a Gpx6 Polyclonal Antibody. As no commercial antibody that is specific for Gpx6 is available, Applicant's developed a rabbit polyclonal antibody to Gpx6 Covance (Denver, Pa.). Two polyclonal antibodies have been raised in rabbit hosts, each targeting the Gpx6-specific peptide “SDIMEYLNQ” (Seq ID No: 1) The antibodies are peptide affinity purified.

TABLE 1
Genes with significant changes (Benjamini-Hochberg adjusted p-values <
0.05) of at least 1.2-fold up or down in Drd1a-expressing striatal medium
spiny neurons at 2 years and 6 weeks of age, as compared to 6 weeks of age.

| | | | Fold | | |
|---|---|---|---|---|---|
| | p value | | change | | |
| Gene_ID | (corrected) | p value | (absolute) | Regulation | Gene Symbol |
| 6899520\|20194 | 0.04976691 | 5.15E−04 | 2.8210127 | up | S100a10 |
| 7023132\|236604 | 0.003987592 | 5.61E−07 | 2.3643906 | up | Pisd-ps3\|Pisd-ps1 |
| 6761825\|269109 | 0.003987592 | 3.56E−07 | 2.3027277 | up | Dpp10 |
| 6981113\|83436 | 0.031307697 | 1.72E−04 | 2.278604 | up | Plekha2 |
| 6886678\|74194 | 0.020062922 | 5.50E−05 | 2.2697477 | down | Rnd3 |
| 6841712\|320712 | 0.013143726 | 1.24E−05 | 2.2293866 | up | Abi3bp |
| 6850534\|27226 | 0.007663706 | 5.02E−06 | 2.1952941 | up | Pla2g7 |
| 6753402\|21956 | 0.007663706 | 5.44E−06 | 2.1893516 | up | Tnnt2 |
| 6777510\|73914 | 0.014433213 | 1.93E−05 | 2.15296 | up | Irak3 |
| 7013389\|237010 | 0.015460879 | 3.15E−05 | 2.1512105 | up | Klhl4 |
| 6768075\|12140 | 0.026665783 | 1.07E−04 | 2.069984 | down | Fabp7 |
| 6880670\|12010 | 0.005005356 | 1.24E−06 | 2.0678577 | up | B2m |
| 7017520\|14396 | 0.024065567 | 9.01E−05 | 2.0567203 | up | Gabra3 |
| 6967593\|110886 | 0.034732584 | 2.22E−04 | 2.0417027 | down | Gabra5 |
| 6861441\|328971 | 0.016631078 | 3.70E−05 | 2.0326183 | down | Spink10 |
| 6860204\|93890 | 0.017962048 | 4.52E−05 | 2.0307233 | down | Pcdhb19 |
| 6764133\|18611 | 0.005005356 | 1.92E−06 | 2.0271978 | up | Pea15a |
| 6900456\|57257 | 0.024065567 | 8.94E−05 | 1.9663367 | up | Vav3 |
| 6957263\|12444 | 0.007264868 | 4.09E−06 | 1.9645411 | down | Ccnd2 |
| 6937190, 702313 | 0.005005356 | 2.30E−06 | 1.9601252 | up | Pisd\|Pisd-ps3///Pisd-ps3\|Pisd-ps1 |
| 2\|320951 | | | | | |
| 6937190, 702313 | 0.005005356 | 2.30E−06 | 1.9601252 | up | Pisd\|Pisd-ps3///Pisd-ps3\|Pisd-ps1 |
| 2\|66776 | | | | | |
| 6768479\|13654 | 0.034732584 | 2.22E−04 | 1.9502792 | down | Egr2 |
| 6860170\|93877 | 0.04976691 | 4.83E−04 | 1.9439118 | up | Pcdhb6 |
| 6869570\|74055 | 0.005005356 | 2.46E−06 | 1.9408888 | up | Plce1 |
| 6824507\|67419 | 0.039366398 | 2.78E−04 | 1.8852826 | up | 3632451O06Rik |
| 6919895\|69352 | 0.013143726 | 1.57E−05 | 1.859933 | up | Necab1 |
| 6953887\|18575 | 0.031307697 | 1.66E−04 | 1.8485277 | up | Pde1c |
| 6919417\|252838 | 0.014433213 | 2.52E−05 | 1.8419087 | up | Tox |
| 6879646\|12509 | 0.042573277 | 3.29E−04 | 1.7996722 | up | Cd59a\|Cd59b |
| 6879646\|333883 | 0.042573277 | 3.29E−04 | 1.7996722 | up | Cd59a\|Cd59b |
| 6758223\|66297 | 0.007663706 | 5.93E−06 | 1.7777925 | down | 2610017I09Rik |
| 6805200\|75512 | 0.013143726 | 1.55E−05 | 1.7739272 | up | Gpx6 |
| 6919304\|56711 | 0.04630302 | 4.23E−04 | 1.7644565 | up | Plag1 |
| 7014941\|55936 | 0.014433213 | 2.28E−05 | 1.7513621 | up | Ctps2 |
| 6766409\|52906 | 0.014433213 | 2.54E−05 | 1.7504913 | up | Ahi1 |
| 6832146\|105859 | 0.024065567 | 8.76E−05 | 1.7445399 | up | Csdc2 |
| 6791494\|73635 | 0.013143726 | 1.39E−05 | 1.735179 | down | Rundc1\|1700113I22Rik\|Aarsd1 |
| 6830852, 683607 | 0.016631078 | 3.86E−05 | 1.7265527 | down | 9930014A18Rik\|Fam84b///Fam84b\|9930014 |
| 9\|320469 | | | | | A18Rik |
| 6830852, 683607 | 0.016631078 | 3.86E−05 | 1.7265527 | down | 9930014A18Rik\|Fam84b///Fam84b\|9930014 |
| 9\|399603 | | | | | A18Rik |
| 6837848\|54526 | 0.047644805 | 4.56E−04 | 1.7124188 | up | Syt10 |
| 6805383, 681169 | 0.014433213 | 2.18E−05 | 1.708725 | down | Hist1h3b\|Hist1h3c\|Hist1h3d\|Hist1h3e\|Hist1h |
| 7\|319148 | | | | | 3h\|Hist1h3i///Hist1h3b\|Hist1h3c\|Hist1h3d\|Hi |
| | | | | | st1h3e\|Hist1h3f\|Hist1h3h\|Hist1h3i |
| 6860188\|93885 | 0.024065567 | 8.95E−05 | 1.7023137 | up | Pcdhb14 |
| 6989222\|12903 | 0.027466808 | 1.16E−04 | 1.7015634 | down | Crabp1 |
| 6834890\|56274 | 0.024065567 | 8.70E−05 | 1.6866167 | up | Stk3 |
| 6784587\|11421 | 0.017962048 | 4.80E−05 | 1.6859602 | down | Ace\|Ace3 |
| 6784587\|217246 | 0.017962048 | 4.80E−05 | 1.6859602 | down | Ace\|Ace3 |
| 6843811\|74720 | 0.043797355 | 3.74E−04 | 1.6742324 | down | Tmem114 |
| 7015229\|11856 | 0.041084405 | 3.00E−04 | 1.66966 | up | Arhgap6 |
| 6908528\|114301 | 0.039366398 | 2.81E−04 | 1.6680608 | down | Palmd |
| 6809522\|20365 | 0.013143726 | 1.37E−05 | 1.6649965 | down | Serf1 |
| 6838460\|72393 | 0.024065567 | 7.84E−05 | 1.6563784 | up | Faim2 |
| 6978855\|56513 | 0.03619993 | 2.44E−04 | 1.6498939 | down | Pard6a |
| 6869068\|77125 | 0.04976691 | 5.03E−04 | 1.645941 | up | Il33 |
| 6768261, 687613 | 0.031307697 | 1.75E−04 | 1.6448121 | up | Gm5424\|Ass1///Ass1\|Gm5424 |
| 8\|432466 | | | | | |
| 6768261, 687613 | 0.031307697 | 1.75E−04 | 1.6448121 | up | Gm5424\|Ass1///Ass1\|Gm5424 |
| 8\|11898 | | | | | |
| 6792679\|30951 | 0.015102888 | 2.80E−05 | 1.6424714 | down | Cbx8 |
| 6759997\|20254 | 0.015102888 | 2.87E−05 | 1.6344112 | up | Scg2 |
| 6955137\|94282 | 0.023366889 | 7.06E−05 | 1.6341208 | down | Sfxn5 |
| 6781933\|276920 | 0.04635039 | 4.37E−04 | 1.6271018 | up | Ccdc42 |
| 6833331\|15370 | 0.04976691 | 5.26E−04 | 1.6336416 | down | Nr4a1 |
| 6805360\|319181 | 0.031008814 | 1.50E−04 | 1.6091015 | down | Hist1h2bg |
| 6799173\|217410 | 0.021131802 | 6.24E−05 | 1.6087055 | down | Trib2 |
| 6850191\|15937 | 0.021131802 | 6.23E−05 | 1.6082655 | up | Ier3 |
| 6954572\|104263 | 0.031335603 | 1.87E−04 | 1.6033699 | up | Kdm3a |
| 6903983\|241919 | 0.008627407 | 7.28E−06 | 1.5909182 | up | Slc7a14 |
| 6874631\|16922 | 0.039366398 | 2.82E−04 | 1.577109 | up | Phyh |
| 6755757\|72978 | 0.043715313 | 3.67E−04 | 1.5758797 | down | Cnih3 |
| 6805255, 680527 | 0.031335603 | 1.79E−04 | 1.573402 | down | Gm11277\|Gm13646\|Hist1h2bc\|Hist1h2bj\|His |
| 3, 6805370\|6802 | | | | | t1h2bk\|Hist1h2bl\|Hist3h2bm///Hist1h2bj\|Hist |
| 4 | | | | | 1h2bc\|Hist1h2bk\|Gm11277\|Gm13646///Hist1 |
| | | | | | h2bc\|Hist1h2bj\|Hist1h2bk\|Gm11277\|Gm136 |
| | | | | | 46 |
| 6756790\|17864 | 0.017962048 | 4.55E−05 | 1.5739444 | up | Mybl1 |
| 6834108\|12563 | 0.025328478 | 9.97E−05 | 1.5705953 | down | Cdh6 |
| 6815437\|238799 | 0.042669825 | 3.37E−04 | 1.5662972 | up | Tnpo1 |
| 6969021\|11864 | 0.014433213 | 2.43E−05 | 1.5643754 | up | Arnt2 |
| 6900404\|99730 | 0.024065567 | 7.75E−05 | 1.5643417 | down | Tafl3 |
| 6805255, 680527 | 0.031307697 | 1.76E−04 | 1.5641509 | down | Gm11277\|Gm13646\|Hist1h2bc\|Hist1h2bj\|His |
| 3, 6805370, 6811 | | | | | t1h2bk\|Hist1h2bl\|Hist1h2bm///Hist1h2bj\|Hist |
| 533\|665622 | | | | | 1h2bc\|Hist1h2bk\|Gm11277\|Gm13646///Hist1 |
| | | | | | h2bc\|Hist1h2bj\|Hist1h2bk\|Gm11277\|Gm136 |
| | | | | | 46///Gm11277\|Gm13646\|Hist1h2bj\|Hist1h2b |
| | | | | | k |
| 6805255, 680527 | 0.031307697 | 1.76E−04 | 1.5641509 | down | Gm11277\|Gm13646\|Hist1h2bc\|Hist1h2bj\|His |
| 3, 6805370, 6811 | | | | | t1h2bk\|Hist1h2bl\|Hist1h2bm///Hist1h2bj\|Hist |
| 533\|665596 | | | | | 1h2bc\|Hist1h2bk\|Gm11277\|Gm13646///Hist1 |
| | | | | | h2bc\|Hist1h2bj\|Hist1h2bk\|Gm11277\|Gm136 |
| | | | | | 46///Gm11277\|Gm13646\|Hist1h2bj\|Hist1h2b |
| | | | | | k |
| 6805255, 680527 | 0.031307697 | 1.76E−04 | 1.5641509 | down | Gm11277\|Gm13646\|Hist1h2bc\|Hist1h2bj\|His |
| 3, 6805370, 6811 | | | | | t1h2bk\|Hist1h2bl\|Hist1h2bm///Hist1h2bj\|Hist |
| 533\|319183 | | | | | 1h2bc\|Hist1h2bk\|Gm11277\|Gm13646///Hist1 |
| | | | | | h2bc\|Hist1h2bj\|Hist1h2bk\|Gm11277\|Gm136 |
| | | | | | 46///Gm11277\|Gm13646\|Hist1h2bj\|Hist1h2b |
| | | | | | k |
| 6805237, 680535 | 0.027739117 | 1.27E−04 | 1.5599499 | down | Hist1h3h\|Hist1h3b\|Hist1h3d\|Hist1h3e\|Hist1h |
| 7, 6805383, 6811 | | | | | 3g\|Hist1h3i\|Hist1h3a///Hist1h3b\|Hist1h3d\|Hi |
| 531, 6811697, 68 | | | | | st1h3e\|Hist1h3g\|Hist1h3h\|Hist1h3i///Hist1h3 |
| 11702\|319152 | | | | | b\|Hist1h3c\|Hist1h3d\|Hist1h3e\|Hist1h3h\|Hist1 |
| | | | | | h3i///Hist1h3i\|Hist1h3b\|Hist1h3d\|Hist1h3e\|H |
| | | | | | ist1h3g\|Hist1h3h///Hist1h3b\|Hist1h3c\|Hist1h |
| | | | | | 3d\|Hist1h3e\|Hist1h3f\|Hist1h3h\|Hist1h3i///His |
| | | | | | t1h3a\|Hist1h3b\|Hist1h3d\|Hist1h3e\|Hist1h3g\| |
| | | | | | Hist1h3h\|Hist1h3i |
| 6791641\|14580 | 0.024065567 | 7.86E−05 | 1.5587983 | up | Gfap |
| 6805237, 680535 | 0.042669825 | 3.51E−04 | 1.5567741 | down | Hist1h3h\|Hist1h3b\|Hist1h3d\|Hist1h3e\|Hist1h |
| 7, 6805364, 6805 | | | | | 3g\|Hist1h3i\|Hist1h3a///Hist1h3b\|Hist1h3d\|Hi |
| 383, 6811531, 68 | | | | | st1h3e\|Hist1h3g\|Hist1h3h\|Hist1h3i///Hist1h3 |
| 11697, 6811702\| | | | | | b\|Hist1h3d\|Hist1h3e\|Hist1h3i///Hist1h3b\|Hist |
| 319150 | | | | | 1h3c\|Hist1h3d\|Hist1h3e\|Hist1h3h\|Hist1h3i/// |
| | | | | | Hist1h3i\|Hist1h3b\|Hist1h3d\|Hist1h3e\|Hist1h |
| | | | | | 3g\|Hist1h3h///Hist1h3b\|Hist1h3c\|Hist1h3d\|Hi |
| | | | | | st1h3e\|Hist1h3f\|Hist1h3h\|Hist1h3i///Hist1h3a |
| | | | | | \|Hist1h3b\|Hist1h3d\|Hist1h3e\|Hist1h3g\|Hist1h |
| | | | | | 3h\|Hist1h3i |
| 6805237, 680535 | 0.042669825 | 3.51E−04 | 1.5567741 | down | Hist1h3h\|Hist1h3b\|Hist1h3d\|Hist1h3e\|Hist1h |
| 7, 6805364, 6805 | | | | | 3g\|Hist1h3i\|Hist1h3a///Hist1h3b\|Hist1h3d\|Hi |
| 383, 6811531, 68 | | | | | st1h3e\|Hist1h3g\|Hist1h3h\|Hist1h3i///Hist1h3 |
| 11697, 6811702\| | | | | | b\|Hist1h3d\|Hist1h3e\|Hist1h3i///Hist1h3b\|Hist |
| 319149 | | | | | 1h3c\|Hist1h3d\|Hist1h3e\|Hist1h3h\|Hist1h3i/// |
| | | | | | Hist1h3i\|Hist1h3b\|Hist\|h3d\|Hist1h3e\|Hist1h |
| | | | | | 3g\|Hist1h3h///Hist1h3b\|Hist1h3c\|Hist1h3d\|Hi |
| | | | | | st1h3e\|Hist1h3f\|Hist1h3h\|Hist1h3i///Hist1h3a |
| | | | | | \|Hist1h3b\|Hist1h3d\|Hist1h3e\|Hist1h3g\|Hist1h |
| | | | | | 3h\|Hist1h3i |
| 6805237, 680535 | 0.042669825 | 3.51E−04 | 1.5567741 | down | Hist1h3h\|Hist1h3b\|Hist1h3d\|Hist1h3e\|Hist1h |
| 7, 6805364, 6805 | | | | | 3g\|Hist1h3i\|Hist1h3a///Hist1h3b\|Hist1h3d\|Hi |
| 383, 6811531, 68 | | | | | st1h3e\|Hist1h3g\|Hist1h3h\|Hist1h3i///Hist1h3 |
| 11697, 6811702\| | | | | | b\|Hist1h3d\|Hist1h3e\|Hist1h3i///Hist1h3b\|Hist |
| 319153 | | | | | 1h3c\|Hist1h3d\|Hist1h3e\|Hist1h3h\|Hist1h3i/// |
| | | | | | Hist1h3i\|Hist1h3b\|Hist1h3d\|Hist1h3e\|Hist1h |
| | | | | | 3g\|Hist1h3h///Hist1h3b\|Hist1h3c\|Hist1h3d\|Hi |
| | | | | | st1h3e\|Hist1h3f\|Hist1h3h\|Hist1h3i///Hist1h3a |
| | | | | | \|Hist1h3b\|Hist1h3d\|Hist1h3e\|Hist1h3g\|Hist1h |
| | | | | | 3h\|Hist1h3i |
| 6805255, 680527 | 0.031335603 | 1.87E−04 | 1.555105 | down | Gm11277\|Gm13646\|Hist1h2bc\|Hist1h2bj\|His |
| 0, 6805273, 6805 | | | | | t1h2bk\|Hist1h2bl\|Hist1h2bm///Hist1h2bk///H |
| 370, 6811533\|31 | | | | | ist1h2bj\|Hist1h2bc\|Hist1h2bk\|Gm11277\|Gm |
| 9184 | | | | | 13646///Hist1h2bc\|Hist1h2bj\|Hist1h2bk\|Gm3 |
| | | | | | 1277\|Gm13646///Gm11277\|Gm13646\|Hist1h |
| | | | | | 2bj\|Hist1h2bk |
| 6971344\|66422 | 0.025328478 | 9.94E−05 | 1.5520357 | down | Dctpp1 |
| 6905746\|17035 | 0.043715313 | 3.68E−04 | 1.5490541 | up | Lxn |
| 6764526\|69726 | 0.04630302 | 4.30E−04 | 1.5490206 | up | Smyd3 |
| 6778528\|56418 | 0.027466808 | 1.16E−04 | 1.5428835 | down | Ykt6 |
| 6769343, 677353 | 0.014433213 | 2.54E−05 | 1.5391593 | down | Tdg\|Gm9855\|Gm5806 |
| 7, 6968533\|6247 | | | | | |
| 84 | | | | | |
| 6769343, 677353 | 0.014433213 | 2.54E−05 | 1.5391593 | down | Tdg\|Gm9855\|Gm5806 |
| 7, 6968533\|5451 | | | | | |
| 24 | | | | | |
| 6805358, 681168 | 0.017464902 | 4.17E−05 | 1.5370511 | down | Hist1h3f\|Hist1h3e///Hist1h3b\|Hist1h3c\|Hist1 |
| 1, 6811697\|2604 | | | | | h3d\|Hist1h3e\|Hist1h3f\|Hist1h3h\|Hist1h3i |
| 23 | | | | | |
| 6785684\|380684 | 0.031875465 | 1.93E−04 | 1.5352144 | up | Nefh |
| 6977139\|326618 | 0.024065567 | 9.03E−05 | 1.5304857 | down | Tpm4 |
| 6972181\|15461 | 0.021131802 | 6.21E−05 | 1.5240446 | down | Hras1 |
| 6910642\|229949 | 0.04635039 | 4.34E−04 | 1.5222185 | up | Ak5 |
| 6769343, 677353 | 0.015460879 | 3.15E−05 | 1.518443 | down | Tdg\|Gm9855\|Gm5806///Glt8d2\|Tdg |
| 7, 6775518, 6968 | | | | | |
| 533\|21665 | | | | | |
| 6808209\|94066 | 0.027553149 | 1.20E−04 | 1.5128382 | down | Mrpl36 |
| 6805237, 680535 | 0.047644805 | 4.54E−04 | 1.5121334 | down | Hist1h3h\|Hist1h3b\|Hist1h3d\|Hist1h3e\|Hist1h |
| 7, 6805358, 6805 | | | | | 3g\|Hist1h3i\|Hist1h3a///Hist1h3b\|Hist1h3d\|Hi |
| 364, 6805383, 68 | | | | | st1h3e\|Histih3g\|Histih3h\|Hist1h3i///Hist1h3 |
| 11531, 6811681, | | | | | f\|Hist1h3e///Hist1h3b\|Hist1h3d\|Hist1h3e\|Hist |
| 6811697, 681170 | | | | | 1h3i///Hist1h3b\|Hist1h3c\|Hist1h3d\|Hist1h3e\| |
| 2\|319151 | | | | | Hist1h3h\|Hist1h3i///Hist1h3i\|Hist1h3b\|Hist3 |
| | | | | | h3d\|Hist1h3e\|Hist1h3g\|Hist1h3h///Hist1h3b\| |
| | | | | | HLst1h3c\|Hist1h3d\|Hist1h3e\|Hist1h3f\|Hist1h |
| | | | | | 3h\|Hist1h3i///Hist1h3a\|Hist1h3b\|Hist1h3d\|Hi |
| | | | | | st1h3e\|Hist1h3g\|Hist1h3h\|Hist1h3i |
| 6893532\|12162 | 0.049941193 | 5.37E−04 | 1.5053798 | up | Bmp7 |
| 6918705\|230904 | 0.03619993 | 2.47E−04 | 1.5018022 | up | Fbxo2 |
| 6845139\|106264 | 0.015839854 | 3.34E−05 | 1.4911728 | down | 0610012G03Rik |
| 6747641\|240725 | 0.03460012 | 2.17E−04 | 1.4901471 | up | Sulf1 |
| 6805245, 681168 | 0.04976691 | 5.26E−04 | 1.4885166 | down | Hist1h2bn///Hist1h2be\|Hist1h1e\|Hist1h2bn |
| 6\|319187 | | | | | |
| 6860198\|93887 | 0.042669825 | 3.35E−04 | 1.4877453 | down | Pcdhb16 |
| 6819244\|12891 | 0.04196097 | 3.15E−04 | 1.4872487 | up | Cpne6 |
| 6764011\|107652 | 0.027558634 | 1.24E−04 | 1.4826605 | down | Uap1 |
| 6770160\|67603 | 0.031335603 | 1.84E−04 | 1.480635 | down | Dusp6 |
| 6753280\|98710 | 0.043797355 | 3.76E−04 | 1.4792016 | down | Rabif |
| 6922649\|66928 | 0.031307697 | 1.58E−04 | 1.4790272 | down | 3110001D03Rik\|LOC280487 |
| 6922649\|280487 | 0.031307697 | 1.58E−04 | 1.4790272 | down | 3110001D03Rik\|LOC280487 |
| 6748437\|170771 | 0.031891167 | 1.95E−04 | 1.4753839 | up | Khdrbs2 |
| 6840052, 690220 | 0.03595298 | 2.35E−04 | 1.4658682 | down | Gng5\|Gm3150///Gng5 |
| 4\|14707 | | | | | |
| 6966985\|12028 | 0.04630302 | 4.23E−04 | 1.461731 | down | Bax |
| 6984485\|114255 | 0.027553149 | 1.18E−04 | 1.458611 | down | Dok4 |
| 6995258\|21345 | 0.031307697 | 1.68E−04 | 1.4537994 | down | Tagln |
| 6994887\|72828 | 0.027249046 | 1.11E−04 | 1.4510411 | down | Ubash3b |
| 6871277\|20867 | 0.04630302 | 4.13E−04 | 1.4369333 | up | Stip1 |
| 6769637\|67282 | 0.024065567 | 9.14E−05 | 1.4348623 | down | Ccdc53 |
| 6765129\|16526 | 0.024065567 | 8.74E−05 | 1.4336265 | down | Kcnk2 |
| 6987331\|23988 | 0.04630302 | 4.24E−04 | 1.4327555 | down | Pin1\|Pin11 |
| 6987331\|241593 | 0.04630302 | 4.24E−04 | 1.4327555 | down | Pin1\|Pin11 |
| 6878655\|16410 | 0.027558634 | 1.24E−04 | 1.4282677 | up | Itgav |
| 6973588\|53333 | 0.03698042 | 2.55E−04 | 1.4254433 | down | Tomm40 |
| 6860259\|71302 | 0.04976691 | 5.32E−04 | 1.42541 | up | Arhgap26[Gm5820\|9630014M24Rik |
| 6860259\|545253 | 0.04976691 | 5.32E−04 | 1.42541 | up | Arhgap26\|Gm5820\|9630014M24Rik |
| 6860259\|381155 | 0.04976691 | 5.32E−04 | 1.42541 | up | Arhgap26\|Gm5820\|9630014M24Rik |
| 6768155\|19156 | 0.04976691 | 4.99E−04 | 1.4248804 | up | Psap |
| 6913531\|66536 | 0.04196097 | 3.19E−04 | 1.4231335 | down | Nipsnap3b |
| 6885447\|98766 | 0.043715313 | 3.69E−04 | 1.4347362 | down | Ubac1 |
| 6985655\|66531 | 0.031335603 | 1.81E−04 | 1.4146156 | down | 2310061C15Rik |
| 6844321\|27883 | 0.04976691 | 4.92E−04 | 1.4115212 | down | D16H22S680E\|Mir185\|Trmt2a |
| 6844321\|387180 | 0.04976691 | 4.92E−04 | 1.4115212 | down | D16H22S680E\|Mir185\|Trmt2a |
| 6905145\|67890 | 0.031307697 | 1.74E−04 | 1.4102932 | down | Ufm1 |
| 6823041\|12325 | 0.028634837 | 1.33E−04 | 1.4102247 | up | Camk2g\|Usp54 |
| 6951200\|66184 | 0.032929324 | 2.04E−04 | 1.4087964 | down | Rps4y2 |
| 6965076\|69752 | 0.04976691 | 4.84E−04 | 1.4064848 | down | Zfp511 |
| 6821431, 698987 | 0.028811546 | 1.38E−04 | 1.4051728 | down | Uchl3\|Uchl4///Uchl4\|Uchl3 |
| 3\|50933 | | | | | |
| 6821431, 698987 | 0.028811546 | 1.38E−04 | 1.4051728 | down | Uchl3\|Uchl4///Uchl4\|Uchl3 |
| 3\|93841 | | | | | |
| 6955034\|27369 | 0.03619993 | 2.43E−04 | 1.4038689 | down | Dguok |
| 6835065\|70790 | 0.03595298 | 2.38E−04 | 1.4013983 | up | Ubr5 |
| 6953587\|54353 | 0.042669825 | 3.41E−04 | 1.4010115 | up | Skap2 |
| 6941761\|207565 | 0.03595298 | 2.34E−04 | 1.3903749 | down | Camkk2 |
| 6866919\|68731 | 0.04196097 | 3.18E−04 | 1.3859518 | down | 1110032A13Rik |
| 6855669\|75564 | 0.04597941 | 4.01E−04 | 1.3830876 | up | Rsph9 |
| 6795889\|238247 | 0.04196097 | 3.10E−04 | 1.3828267 | up | Arid4a |
| 6754526\|73844 | 0.039366398 | 2.82E−04 | 1.3790938 | up | Ankrd45 |
| 6845559\|76916 | 0.04976691 | 5.07E−04 | 1.3761423 | down | 4930455C21Rik |
| 6881306\|110911 | 0.04550051 | 3.93E−04 | 1.358163 | up | Cds2 |
| 6916947\|170638 | 0.041004203 | 2.97E−04 | 1.3532506 | up | Hpcal4 |
| 6823724\|67011 | 0.042669825 | 3.42E−04 | 1.3496869 | down | Mettl6 |
| 6787525\|14406 | 0.04630302 | 4.29E−04 | 1.3473492 | up | Gabrg2 |
| 6845459\|207227 | 0.04976691 | 5.31E−04 | 1.3440369 | up | Stxbp51 |
| 6765596\|66084 | 0.04976691 | 5.22E−04 | 1.3427882 | down | Rmnd1\|Gm5512 |
| 6765596\|433224 | 0.04976691 | 5.22E−04 | 1.3427882 | down | Rmnd1\|Gm5512 |
| 6881100, 688110 | 0.04630302 | 4.10E−04 | 1.3219867 | up | Zc3h6 |
| 1\|78751 | | | | | |

TABLE 2
Genes with significant changes (Benjamini-Hochberg adjusted p-values <
0.05) of at least 1.2-fold up or down in Drd2-expressing striatal medium
spiny neurons at 2 years and 6 weeks of age, as compared to 6 weeks of age.
D2 Striatum.txt

| | | | Fold | | |
|---|---|---|---|---|---|
| | p value | | change | | |
| Gene_ID | (corrected) | p value | (absolute) | Regulation | Gene symbol |
| 6813284\|13488 | 1.10E−04 | 7.73E−09 | 5.3255243 | up | Drd1a |
| 6860176\|93879 | 1.40E−04 | 1.18E−07 | 4.5493364 | up | Pcdhb8 |
| 6880670\|12010 | 5.82E−04 | 1.15E−06 | 4.148419 | up | B2m |
| 6879087\|12672 | 1.40E−04 | 1.06E−07 | 3.5000043 | up | Chrm4 |
| 6877229\|16519 | 1.40E−04 | 1.15E−07 | 3.1247575 | up | Kcnj3 |
| 6905530\|229357 | 2.28E−04 | 2.41E−07 | 3.0322802 | up | Gpr149 |
| 6747641\|240725 | 2.28E−04 | 2.44E−07 | 2.9879596 | up | Sulf1 |
| 6764133\|18611 | 4.32E−04 | 6.07E−07 | 2.9562507 | up | Pea15a |
| 6998397\|22041 | 0.007923391 | 5.85E−05 | 2.8957565 | up | Trf |
| 6845079\|11815 | 0.008547507 | 6.73E−05 | 2.7908227 | up | Apod |
| 6761825\|269109 | 0.003589682 | 1.74E−05 | 2.7531443 | up | Dpp10 |
| 6805200\|75512 | 0.003589682 | 1.73E−05 | 2.7515676 | up | Gpx6 |
| 6886678\|74194 | 0.015283823 | 2.01E−04 | 2.741378 | down | Rnd3 |
| 6748020\|14859 | 0.003589682 | 1.70E−05 | 2.643068 | up | Gsta3 |
| 6943974\|21333 | 0.003656822 | 1.83E−05 | 2.6233518 | up | Tac1 |
| 6834890\|56274 | 0.002267633 | 7.65E−06 | 2.579052 | up | Stk3 |
| 6791494\|73635 | 0.00779995 | 5.70E−05 | 2.5706615 | down | Rundc1\|1700113I22Rik\| |
| | | | | | Aarsd1 |
| 6835759\|18606 | 0.004721215 | 2.63E−05 | 2.5419888 | up | Enpp2 |
| 6776577\|67405 | 0.020587178 | 4.19E−04 | 2.534108 | down | Nts |
| 6767537, 6822154\| | 0.002235683 | 7.23E−06 | 2.5077183 | down | Cd24a |
| 12484 | | | | | |
| 6824610\|29811 | 0.004966004 | 2.93E−05 | 2.4164193 | up | Ndrg2 |
| 6917180\|269582 | 3.81E−04 | 5.09E−07 | 2.3707016 | down | Clspn |
| 7023132\|236604 | 1.34E−04 | 3.86E−08 | 2.3301787 | up | Pisd-ps3\|Pisd-ps1 |
| 6811068\|56048 | 0.001255512 | 3.09E−06 | 2.2832966 | up | Lgals8 |
| 6860170\|93877 | 1.40E−04 | 8.96E−08 | 2.2796516 | up | Pcdhb6 |
| 6841712\|320712 | 6.50E−04 | 1.42E−06 | 2.2767649 | up | Abi3bp |
| 6753402\|21956 | 2.28E−04 | 2.57E−07 | 2.1717684 | up | Tnnt2 |
| 6819244\|12891 | 5.82E−04 | 9.67E−07 | 2.1581087 | up | Cpne6 |
| 7013389\|237010 | 0.005432868 | 3.59E−05 | 2.1391268 | up | Klhl4 |
| 6908078, 6908079\| | 0.001185273 | 2.76E−06 | 2.1385758 | up | Gstm1\|Gstm3///Gstm2\|Gstm1 |
| 14862 | | | | | |
| 6838460\|72393 | 0.002798558 | 1.18E−05 | 2.1031454 | up | Faim2 |
| 6855981\|20230 | 1.34E−04 | 7.55E−08 | 2.0987513 | down | Satb1\|5830444F18Rik\|C2300 |
| | | | | | 85N15Rik\|E430014B02Rik |
| 6855981\|320804 | 1.34E−04 | 7.55E−08 | 2.0987513 | down | Satb1\|5830444F18Rik\|C2300 |
| | | | | | 85N15Rik\|E430014B02Rik |
| 6855981\|320556 | 1.34E−04 | 7.55E−08 | 2.0987513 | down | Satb1\|5830444F18Rik\|C2300 |
| | | | | | 85N15Rik\|E430014B02Rik |
| 6855981\|320908 | 1.34E−04 | 7.55E−08 | 2.0987513 | down | Satb1\|5830444F18Rik\|C2300 |
| | | | | | 85N15Rik\|E430014B02Rik |
| 6805381\|50708 | 0.02441559 | 6.11E−04 | 2.0501385 | down | Hist1h1c |
| 6869068\|77125 | 5.88E−04 | 1.24E−06 | 2.0441618 | up | Il33 |
| 6807154\|14057 | 0.004926001 | 2.84E−05 | 2.0316029 | up | Sfxn1 |
| 6805360\|319181 | 2.63E−04 | 3.14E−07 | 2.0225863 | down | Hist1h2bg |
| 6815345\|15212 | 0.014639024 | 1.65E−04 | 2.0052912 | up | Hexb |
| 6937190, 7023132\| | 1.34E−04 | 5.77E−08 | 2.003108 | up | Pisd\|Pisd-ps3///Pisd-ps3\|Pisd- |
| 320951 | | | | | ps1 |
| 6937190, 7023132\| | 1.34E−04 | 5.77E−08 | 2.003108 | up | Pisd\|Pisd-ps3///Pisd-ps3\|Pisd- |
| 66776 | | | | | ps1 |
| 6974682\|320158 | 0.001867071 | 5.38E−06 | 1.9956818 | down | Zmat4 |
| 6798951\|26950 | 0.01630344 | 2.40E−04 | 1.9872415 | up | Vsnl1 |
| 6936702\|84652 | 0.00156325 | 4.29E−06 | 1.9783112 | up | Fam126a |
| 6994887\|72828 | 0.015052847 | 1.77E−04 | 1.9770834 | down | Ubash3b |
| 6996956\|20255 | 0.002493485 | 9.27E−06 | 1.9685587 | up | Scg3 |
| 6860188\|93885 | 0.002235683 | 7.21E−06 | 1.9678934 | up | Pcdhb14 |
| 6800468\|217517 | 0.001915143 | 5.66E−06 | 1.9609915 | up | Stxbp6 |
| 7015648\|71458 | 0.008480565 | 6.56E−05 | 1.9547465 | down | Bcor |
| 6805255, 6805273, | 5.82E−04 | 1.08E−06 | 1.9373631 | down | Gm11277\|Gm13646\|Hist1h2 |
| 6805370\|68024 | | | | | bc\|Hist1h2bj\|Hist1h2bk\|Hist1 |
| | | | | | h2bl\|Hist1h2bm///Hist1h2bj\| |
| | | | | | Hist1h2bc\|Hist1h2bk\|Gm112 |
| | | | | | 77\|Gm13646///Hist1h2bc\|Hist |
| | | | | | 1h2bj\|Hist1h2bk\|Gm11277\|G |
| | | | | | m13646 |
| 6908075, 6908077, | 0.003549275 | 1.62E−05 | 1.9293586 | up | Gstm6\|Gstm3///Gstm3///Gstm |
| 6908078\|14864 | | | | | 1\|Gstm3 |
| 6959584\|22177 | 0.042509187 | 0.001670174 | 1.926382 | up | Tyrobp |
| 6882307\|66405 | 0.001386939 | 3.61E−06 | 1.923774 | down | Mcts2 |
| 6805255, 6805273, | 5.82E−04 | 1.12E−06 | 1.9198099 | down | Gm11277\|Gm13646\|Hist1h2 |
| 6805370, 6811533\| | | | | | bc\|Hist1h2bj\|Hist1h2bk\|Hist1 |
| 665622 | | | | | h2bl\|Hist1h2bm///Hist1h2bj\| |
| | | | | | Hist1h2bc\|Hist1h2bk\|Gm112 |
| | | | | | 77\|Gm13646///Hist1h2bc\|Hist |
| | | | | | 1h2bj\|Hist1h2bk\|Gm11277\|G |
| | | | | | m13646///Gm11277\|Gm1364 |
| | | | | | 6\|Hist1h2bj\|Hist1h2bk |
| 6805255, 6805273, | 5.82E−04 | 1.12E−06 | 1.9198099 | down | Gm11277\|Gm13646\|Hist1h2 |
| 6805370, 6811533\| | | | | | bc\|Hist1h2bj\|Hist1h2bk\|Hist1 |
| 665596 | | | | | h2bl\|Hist1h2bm///Hist1h2bj\| |
| | | | | | Histih2bc\|Hist1h2bk\|Gm112 |
| | | | | | 77\|Gm13646///Hist1h2bc\|Hist |
| | | | | | 1h2bj\|Hist1h2bk\|Gm11277\|G |
| | | | | | m13646///Gm11277\|Gm1364 |
| | | | | | 6\|Hist1h2bj\|Hist1h2bk |
| 6805255, 6805273, | 5.82E−04 | 1.12E−06 | 1.9198099 | down | Gm11277\|Gm13646\|Hist1h2 |
| 6805370, 6811533\| | | | | | bc\|Hist1h2bj\|Hist1h2bk\|Hist1 |
| 319183 | | | | | h2b1\|Hist1h2bm///Hist1h2bj\| |
| | | | | | Hist1h2bc\|Hist1h2bk\|Gm112 |
| | | | | | 77\|Gm13646///Hist1b2bc\|Hist |
| | | | | | 1b2bj\|Hist1h2bk\|Gm11277\|G |
| | | | | | m13646///Gm11277\|Gm1364 |
| | | | | | 6\|Hist1h2bj\|Hist1b2bk |
| 6973587\|11816 | 0.040252663 | 0.001514109 | 1.9158078 | up | Apoe |
| 6899520\|20194 | 8.42E−04 | 1.89E−06 | 1.913387 | up | S100a10 |
| 6805255, 6805270, | 5.88E−04 | 1.20E−06 | 1.9044812 | down | Gm11277\|Gm13646\|Hist1h2 |
| 6805273, 6805370, | | | | | bc\|Hist1h2bj\|Hist1h2bk\|Hist1 |
| 6811533\|319184 | | | | | h2bl\|Hist1h2bm///Hist1h2bk// |
| | | | | | /Hist1h2bj\|Hist1h2bc\|Hist1h2 |
| | | | | | bk\|Gm11277\|Gm13646///Hist |
| | | | | | 1h2bc\|Hist1h2bj\|Hist1h2bk\|G |
| | | | | | m11277\|Gm13646///Gm1127 |
| | | | | | 7\|Gm13646\|Hist1h2bj\|Hist1h |
| | | | | | 2bk |
| 6880467\|214240 | 0.003312399 | 1.49E−05 | 1.9037254 | up | Disp2 |
| 6827410\|76965 | 0.003589682 | 1.72E−05 | 1.8947399 | up | Slitrk1 |
| 6780443\|13591 | 0.004721215 | 2.66E−05 | 1.8885926 | up | Ebf1 |
| 6928871\|20346 | 1.65E−04 | 1.50E−07 | 1.8852962 | up | Sema3a |
| 6944262\|114142 | 2.98E−04 | 3.77E−07 | 1.8727168 | up | Foxp2 |
| 6883533\|76829 | 0.013542953 | 1.44E−04 | 1.8723825 | down | Dok5 |
| 6930606\|20563 | 0.031038841 | 9.43E−04 | 1.8607357 | up | Slit2\|Mir218-1 |
| 6930606\|723822 | 0.031038841 | 9.43E−04 | 1.8607157 | up | Slit2\|Mir218-1 |
| 7002980, 7004901, | 0.01798404 | 2.89E−04 | 1.8528872 | up | Bcl2a1d\|Bcl2a1b\|Bcl2a1a///B |
| 7005644, 7006456\| | | | | | cl2a1a\|Bcl2a1c\|Bcl2a1d\|Bcl2 |
| 12047 | | | | | a1b |
| 7002980, 7004901, | 0.01798404 | 2.89E−04 | 1.8528872 | up | Bcl2a1d\|Bcl2a1b\|Bcl2a1a///B |
| 7005644, 7006456\| | | | | | cl2a1a\|Bcl2a1c\|Bcl2a1d\|Bcl2 |
| 12045 | | | | | a1b |
| 7002980, 7004901, | 0.01798404 | 2.89E−04 | 1.8528872 | up | Bcl2a1d\|Bcl2a1b\|Bcl2a1a///B |
| 7005644, 7006456\| | | | | | cl2a1a\|Bcl2a1c\|Bcl2a1d\|Bcl2 |
| 12044 | | | | | a1b |
| 6874631\|16922 | 0.01798404 | 2.91E−04 | 1.8519856 | up | Phyh |
| 6864444\|170459 | 0.001735423 | 4.88E−06 | 1.833533 | up | Stard4 |
| 6772476\|76157 | 0.026050128 | 6.84E−04 | 1.8292406 | up | Slc35d3 |
| 6756637\|58175 | 0.043834306 | 0.001858774 | 1.8150766 | down | Rgs20 |
| 7017520\|14396 | 0.038419306 | 0.001399085 | 1.8125371 | up | Gabra3 |
| 6863973\|106957 | 0.002037618 | 6.16E−06 | 1.809555 | up | Slc39a6 |
| 6880931\|26458 | 0.04682665 | 0.002093915 | 1.8083715 | up | Slc27a2 |
| 6940611\|13602 | 0.002789888 | 1.12E−05 | 1.8027624 | up | Sparcl1\|Scpppq1 |
| 6940611\|1002717 | 0.002789888 | 1.12E−05 | 1.8027624 | up | Sparcl1\|Scpppq1 |
| 04 | | | | | |
| 6989100\|19684 | 0.013600663 | 1.47E−04 | 1.7838393 | up | Rdx |
| 6820055\|13655 | 0.019556254 | 3.55E−04 | 1.7764342 | down | Egr3 |
| 6897908\|18441 | 0.002536604 | 9.63E−06 | 1.7762277 | up | P2ry1 |
| 6990685\|14860 | 0.021159004 | 4.42E−04 | 1.7742459 | up | Gsta4 |
| 6869570\|74055 | 0.004721215 | 2.62E−05 | 1.7690808 | up | Plce1 |
| 6916947\|170638 | 0.002319411 | 8.15E−06 | 1.76002 | up | Hpcal4 |
| 6949160\|74244 | 0.011257361 | 1.08E−04 | 1.7584462 | up | Atg7\|LOC100043926 |
| 6949160\|100043926 | 0.011257361 | 1.08E−04 | 1.7584462 | up | Atg7\|LOC100043926 |
| 7000764\|77226 | 0.03287614 | 0.001040165 | 1.7505 | down | 9330169L03Rik |
| 6884986\|74103 | 0.019693213 | 3.82E−04 | 1.7502115 | down | Neb1 |
| 6754867\|226610 | 0.00430831 | 2.33E−05 | 1.7439637 | down | Fam78b |
| 6756985\|72265 | 0.015268379 | 2.00E−04 | 1.7430842 | up | Tram1 |
| 6816708\|67053 | 0.0332504 | 0.001087987 | 1.7329823 | down | Rpp14 |
| 6862062\|71263 | 0.015283823 | 2.02E−04 | 1.7212113 | down | Mro |
| 6913009, 6921154\| | 0.001302391 | 3.30E−06 | 1.7166166 | down | Tesk1\|Cd72///Cd72 |
| 12517 | | | | | |
| 6900404\|99730 | 0.019556254 | 3.71E−04 | 1.706687 | down | Taf13 |
| 6813560\|56278 | 0.0281969 | 8.04E−04 | 1.7064552 | up | Gkap1 |
| 6908075\|14867 | 0.049958326 | 0.002381477 | 1.7012932 | up | Gstm6\|Gstm3 |
| 7011393\|236794 | 0.023736937 | 5.72E−04 | 1.6997313 | up | Slc9a6 |
| 6948759\|12661 | 0.006945028 | 4.93E−05 | 1.6881636 | up | Chl1 |
| 6954385\|13197 | 0.020429397 | 4.11E−04 | 1.6871984 | down | Gadd45a\|Gng12 |
| 6861689\|67064 | 0.011394512 | 1.10E−04 | 1.6851403 | down | Chmp1b |
| 6799173\|217410 | 0.017824696 | 2.74E−04 | 1.6835176 | down | Trib2 |
| 6763146\|74091 | 0.027805798 | 7.86E−04 | 1.6814463 | down | Npl |
| 6790317\|56405 | 0.022105824 | 4.82E−04 | 1.6790038 | down | Dusp14 |
| 6845978\|17470 | 0.04312894 | 0.001749659 | 1.6726958 | up | Cd200 |
| 6791641\|14580 | 0.011568548 | 1.12E−04 | 1.6671637 | up | Gfap |
| 6754138\|19734 | 0.033966344 | 0.001124808 | 1.6642561 | up | Rgs16 |
| 6763991\|19736 | 0.005185398 | 3.14E−05 | 1.6637514 | down | Rgs4 |
| 6778939\|211739 | 0.010969274 | 9.82E−05 | 1.6624225 | up | Vstm2a\|Hmgb1 |
| 6782694\|11676 | 0.044839386 | 0.001942 | 1.6547385 | up | Aldoc |
| 6957263\|12444 | 0.011084057 | 1.02E−04 | 1.6481607 | down | Ccnd2 |
| 6987109\|14608 | 0.029066546 | 8.46E−04 | 1.6443005 | up | Gpr83 |
| 7015229\|11856 | 0.002298735 | 7.92E−06 | 1.6439329 | up | Arhgap6 |
| 6898630\|68659 | 0.032440964 | 0.001005868 | 1.6438571 | down | Fam198b |
| 6768155\|19156 | 0.005023014 | 3.00E−05 | 1.6399189 | up | Psap |
| 6784371\|73293 | 0.019556254 | 3.71E−04 | 1.6378373 | down | Ccdc103\|4933439F11Rik |
| 6784371\|66784 | 0.019556254 | 3.71E−04 | 1.6378373 | down | Ccdc103\|4933439F11Rik |
| 6854453\|224624 | 0.010969274 | 9.66E−05 | 1.6355382 | down | Rab40c |
| 6946412\|11517 | 5.82E−04 | 1.09E−06 | 1.6319461 | up | Adcyap1r1 |
| 6758223\|66297 | 0.010677658 | 9.23E−05 | 1.6314174 | down | 2610017I09Rik |
| 6961010\|17984 | 0.04115921 | 0.001591617 | 1.6312153 | up | Ndn |
| 6748437\|170771 | 0.00285053 | 1.24E−05 | 1.6293082 | up | Khdrbs2 |
| 6824507\|67419 | 0.026061453 | 6.89E−04 | 1.6290909 | up | 3632451O06Rik |
| 6816288\|16392 | 0.047316674 | 0.002138365 | 1.6269062 | up | Isl1 |
| 6823068\|11750 | 0.015052847 | 1.84E−04 | 1.6259166 | up | Anxa7 |
| 6916089\|74754 | 0.01608881 | 2.28E−04 | 1.625083 | up | Dhcr24 |
| 7020407\|18675 | 0.013542953 | 1.46E−04 | 1.6249138 | down | Phex |
| 6869635\|12495 | 0.01811096 | 2.99E−04 | 1.6211282 | down | Entpd1\|Tctn3 |
| 6876072\|78617 | 0.036739744 | 0.001298216 | 1.6201752 | down | Cstad |
| 6963558\|11865 | 5.19E−04 | 7.66E−07 | 1.6189378 | down | Arnt1 |
| 6866643\|107029 | 0.020970276 | 4.31E−04 | 1.6186217 | down | Me2 |
| 6864327\|20983 | 0.008480565 | 6.43E−05 | 1.6172807 | up | Syt4 |
| 6872616\|19091 | 0.047316674 | 0.002129742 | 1.6146805 | up | Prkg1 |
| 7018897\|50887 | 0.049103312 | 0.0023131 | 1.6131068 | up | Hmgn5 |
| 6753397\|21952 | 0.004729526 | 2.69E−05 | 1.6111035 | down | Tnni1 |
| 6895790\|76897 | 0.045339916 | 0.001979617 | 1.6094204 | up | Raly1 |
| 6749115\|70676 | 0.037047874 | 0.001323231 | 1.6076359 | up | Gulp1 |
| 6912565\|12801 | 0.011134457 | 1.03E−04 | 1 6063108 | down | Cnr1 |
| 6916220\|69908 | 0.018251646 | 3.04E−04 | 1.6042217 | up | Rab3b |
| 6981113\|83436 | 0.02097058 | 4.33E−04 | 1.6026766 | up | Plekha2 |
| 6878655\|16410 | 0.012477017 | 1.23E−04 | 1.6024555 | up | Itgav |
| 6766409\|52906 | 0.00156325 | 4.29E−06 | 1.5939846 | up | Ahi1 |
| 6777286\|216363 | 0.016230881 | 2.33E−04 | 1.5891256 | down | Rab3ip |
| 6976395\|234290 | 0.011135913 | 1.05E−04 | 1.5849389 | down | BC030500 |
| 6834558\|432940 | 0.004089114 | 2.16E−05 | 1.5849028 | down | Fam105b |
| 6769343, 6773537, | 0.002493485 | 9.29E−06 | 1.5830903 | down | Tdg\|Gm9855\|Gm5806 |
| 6968533\|624784 | | | | | |
| 6769343, 6773537, | 0.002493485 | 9.29E−06 | 1.5830903 | down | Tdg\|Gm9855\|Gm5806 |
| 6968533\|545124 | | | | | |
| 6784345\|14824 | 0.002267633 | 7.52E−06 | 1.5818839 | up | Grn |
| 6842273\|74185 | 0.02970668 | 8.71E−04 | 1.578027 | down | Gbe1 |
| 6832146\|105859 | 0.005263386 | 3.29E−05 | 1.5721171 | up | Csdc2 |
| 6769445\|216198 | 0.043429643 | 0.001801553 | 1.5719231 | up | Tcp11l2 |
| 6878702\|241525 | 0.04797561 | 0.002202635 | 1.5712873 | up | Ypel4 |
| 6829659\|17181 | 0.001185273 | 2.83E−06 | 1.57119 | up | Matn2 |
| 6877356, 6886947\| | 0.029997475 | 8.86E−04 | 1.5697238 | up | Galnt5\|Ermn///Ermn |
| 77767 | | | | | |
| 6829123\|215654 | 0.04815002 | 0.002240778 | 1.5685539 | up | Cdh12 |
| 6791528\|72349 | 0.04337046 | 0.001785073 | 1.5679616 | down | Dusp3 |
| 6812770\|67046 | 0.025499985 | 6.60E−04 | 1.5675546 | down | Tbc1d7 |
| 6966198\|20733 | 0.01608881 | 2.26E−04 | 1.5670083 | up | Spint2 |
| 6892747\|19281 | 0.015322137 | 2.05E−04 | 1.5650489 | up | Ptprt |
| 7016726\|236781 | 0.002798558 | 1.15E−05 | 1.5631052 | down | Gpr119 |
| 6916219\|100087 | 0.017905615 | 2.77E−04 | 1.5617313 | down | Kti12 |
| 6871297\|70999 | 0.035202216 | 0.001204654 | 1.561103 | down | Naa40 |
| 6995454\|17967 | 0.01394069 | 1.54E−04 | 1.5575242 | up | Ncam1 |
| 6769343, 6773537, | 0.002537387 | 9.81E−06 | 1.5570186 | down | Tdg\|Gm9855\|Gm5806///Glt8 |
| 6775518, 6968533\| | | | | | d2\|Tdg |
| 21665 | | | | | |
| 6876570\|74192 | 0.010677658 | 9.23E−05 | 1.550878 | down | Arpc5l |
| 6764721\|12334 | 0.04193666 | 0.001627578 | 1.5500118 | up | Capn2 |
| 6997114\|235504 | 0.018667279 | 3.19E−04 | 1.5480542 | up | Slc17a5 |
| 6768261, 6876138\| | 0.014639024 | 1.67E−04 | 1.5479015 | up | Gm5424\|Ass1///Ass1\|Grn542 |
| 432466 | | | | | 4 |
| 6768261, 6876138\| | 0.014639024 | 1.67E−04 | 1.5479015 | up | Gm5424\|Ass1///Ass1\|Gm542 |
| 11898 | | | | | 4 |
| 6877954\|329421 | 0.04884973 | 0.00229772 | 1.5478375 | down | Myo3b |
| 6864518\|75533 | 0.04031455 | 0.001524941 | 1.5474952 | up | Nme5 |
| 6932930\|74596 | 0.015249936 | 1.97E−04 | 1.546657 | up | Cds1 |
| 6761256\|12043 | 0.028586583 | 8.22E−04 | 1.5446783 | down | Bcl2 |
| 6946339, 6953749\| | 0.022382699 | 5.04E−04 | 1.5439757 | up | Chn2\|9130019P16Rik///9130 |
| 69993 | | | | | 019P16Rik\|Chn2 |
| 6946339, 6953749\| | 0.022382699 | 5.04E−04 | 1.5439757 | up | Chn2\|9130019P16Rik/7/9130 |
| 100042056 | | | | | 019P16Rik\|Chn2 |
| 6784266, 6791494\| | 0.008480565 | 6.49E−05 | 1.5408274 | down | Rundc1///Rundc1\|1700113I2 |
| 217201 | | | | | 2Rik\|Aarsd1 |
| 6972294\|13033 | 0.044557586 | 0.001923529 | 1.5388831 | up | Ctsd |
| 6786045\|13195 | 0.015820324 | 2.16E−04 | 1.5372787 | up | Ddc |
| 6780961\|67966 | 0.01608881 | 2.28E−04 | 1.5367461 | down | Zcchc10 |
| 6825705\|20389 | 0.015137184 | 1.92E−04 | 1.5347135 | down | Sftpc |
| 6762321\|381290 | 0.02575794 | 6.68E−04 | 1.532662 | up | Atp2b4 |
| 6770201\|17311 | 0.012557453 | 1.24E−04 | 1.5317988 | down | Kitl |
| 6993067\|67469 | 0.020444345 | 4.13E−04 | 1.5317638 | down | Abhd5 |
| 6815511\|27220 | 0.016510215 | 2.48E−04 | 1.52917 | up | Cartpt |
| 6754149, 6861135\| | 0.018376803 | 3.09E−04 | 1.5289168 | up | Glul///Gramd3\|Glul |
| 14645 | | | | | |
| 7011581\|331424 | 0.010673828 | 8.84E−05 | 1.5289078 | down | C230004F18Rik\|C030023E2 |
| | | | | | 4Rik |
| 7011581\|320247 | 0.010673828 | 8.84E−05 | 1.5289078 | down | C230004F18Rik\|C030023E2 |
| | | | | | 4Rik |
| 6803780\|67236 | 0.010677658 | 9.00E−05 | 1.5288949 | down | Cinp |
| 6998305\|235542 | 0.03324496 | 0.001069841 | 1.5287542 | up | Ppp2r3a |
| 6800314\|16981 | 0.005284981 | 3.34E−05 | 1.5286509 | up | Lrrn3 |
| 6972181\|15461 | 0.005295044 | 3.39E−05 | 1.5272726 | down | Hras1 |
| 6808621\|723967 | 0.003656822 | 1.85E−05 | 1.5243323 | down | Mir9-2\|C130071C03Rik |
| 6808621\|320203 | 0.003656822 | 1.85E−05 | 1.5243323 | down | Mir9-2\|C130071C03Rik |
| 6988855\|54725 | 0.04953374 | 0.002343567 | 1.5239736 | up | Cadm1 |
| 6878548\|68082 | 0.038419306 | 0.001394552 | 1.5217838 | down | Dusp19 |
| 6768910\|20203 | 0.021932513 | 4.73E−04 | 1.5203797 | up | S100b |
| 6948964\|108073 | 0.002072472 | 6.41E−06 | 1.5164479 | up | Grm7 |
| 6949826\|30853 | 0.01984823 | 3.93E−04 | 1.5146208 | down | Mlf2 |
| 6861751\|52662 | 0.008547507 | 6.72E−05 | 1.5145016 | down | D18Ertd653e |
| 7014941\|55936 | 0.019081173 | 3.30E−04 | 1.5129799 | up | Ctps2 |
| 6750314\|320460 | 0.030360658 | 9.05E−04 | 1.5119076 | up | Vwc21 |
| 6964557\|66885 | 0.0332504 | 0.001078375 | 1.5105767 | up | Acadsb |
| 6885395\|68475 | 0.022202644 | 4.90E−04 | 1.5084188 | down | Ssna1 |
| 6933679\|77407 | 0.005520782 | 3.69E−05 | 1.5056041 | down | Rab35 |
| 6779845\|327900 | 0.019560797 | 3.77E−04 | 1.502835 | down | Ubtd2 |
| 6969021\|11864 | 0.00430831 | 2.33E−05 | 1.502225 | up | Arnt2 |
| 6988958\|235323 | 0.002835888 | 1.22E−05 | 1.5014262 | down | Usp28 |
| 6791230\|217151 | 0.019556254 | 3.56E−04 | 1.5006561 | down | Arl5c |
| 6854276\|76917 | 0.015052847 | 1.85E−04 | 1.4991108 | down | Flywch2 |
| 6899747, 6907247\| | 0.024054471 | 5.85E−04 | 1.497548 | down | Hist2h2aa1\|Hist2h2aa2\|Hist2 |
| 15267 | | | | | h2ac\|Hist2h3c1///Hist2h2aa1\| |
| | | | | | Hist2h2aa2\|Hist2h3c1 |
| 6899747, 6907247\| | 0.024054471 | 5.85E−04 | 1.497548 | down | Hist2h2aa1\|Hist2h2aa2\|Hist2 |
| 319192 | | | | | h2ac\|Hist2h3c1///Hist2h2aa1\| |
| | | | | | Hist2h2aa2\|Hist2h3c1 |
| 6973739\|20300 | 0.033489518 | 0.001101954 | 1.4958638 | down | Ccl25 |
| 6916708\|80509 | 0.018667279 | 3.19E−04 | 1.4954613 | down | Med8 |
| 6955034\|27369 | 0.00312503 | 1.38E−05 | 1.4942619 | down | Dguok |
| 6881306\|110911 | 0.002798558 | 1.17E−05 | 1.4928799 | up | Cds2 |
| 6937364\|16976 | 0.02366127 | 5.51E−04 | 1.4919764 | up | Lrpap1 |
| 6824195\|70561 | 0.021932513 | 4.71E−04 | 1.4907249 | up | Txndc16 |
| 6869932, 6873271\| | 0.005263386 | 3.28E−05 | 1.490423 | up | Scd2\|Scd1///Scd1\|Scd2 |
| 20250 | | | | | |
| 6869932, 6873271\| | 0.005263386 | 3.28E−05 | 1.490423 | up | Scd2\|Scd1///Scd1\|Scd2 |
| 20249 | | | | | |
| 6891493\|71436 | 0.021438045 | 4.52E−04 | 1.4899818 | up | Flrt3 |
| 6780844\|619293 | 0.01394069 | 1.53E−04 | 1.4898849 | down | Zfp354a\|Zfp354b\|9230009I0 |
| | | | | | 2Rik |
| 6982921\|66234 | 0.011613366 | 1.13E−04 | 1.4868916 | up | Sc4mol |
| 6972256\|101513 | 0.034334507 | 0.001144242 | 1.4851689 | down | 2700078K21Rik |
| 6990673\|68801 | 0.019556254 | 3.70E−04 | 1.4836878 | up | Elovl5 |
| 6831709\|117171 | 0.02606873 | 6.94E−04 | 1.4806932 | down | 1110038F14Rik |
| 6869436\|226098 | 0.036910944 | 0.00131315 | 1.4780036 | down | Hectd2 |
| 6803269\|71907 | 0.039544 | 0.001466365 | 1.4775524 | up | Serpina9 |
| 6891905\|13010 | 0.047668647 | 0.002171784 | 1.47457 | up | Cst3 |
| 6838469\|26934 | 0.015052847 | 1.81E−04 | 1.4744074 | up | Racgap1 |
| 6933491\|330164 | 0.04208484 | 0.00163976 | 1.4724283 | down | C130026L21Rik |
| 6937522\|22393 | 0.0281969 | 8.09E−04 | 1.470271 | up | Wfs1 |
| 6784412\|57778 | 0.015052847 | 1.78E−04 | 1.4695581 | down | Fmnl1 |
| 6903983\|241919 | 0.015137184 | 1.90E−04 | 1.4690902 | up | Slc7a14 |
| 6918814\|65945 | 0.004966004 | 2.90E−05 | 1.4689643 | up | Clstn1 |
| 69287591\|231014 | 0.02585882 | 6.73E−04 | 1.4681538 | up | 9330182L06Rik |
| 6933616, 69412181\| | 0.018114181 | 3.01E−04 | 1.4663692 | down | Ankrd13a///4930515G01Rik\| |
| 68420 | | | | | Ankrd13a |
| 6808997\|26556 | 0.004002512 | 2.08E−05 | 1.4656779 | down | Homer1\|C330006P03Rik |
| 6808997\|320588 | 0.004002512 | 2.08E−05 | 1.4656779 | down | Homer1\|C330006P03Rik |
| 6789325\|12514 | 0.022007378 | 4.78E−04 | 1.4655061 | down | Cd68 |
| 6902665\|209601 | 0.015204828 | 1.95E−04 | 1.4653959 | up | 4922501L14Rik |
| 6863645\|12558 | 0.030360658 | 9.02E−04 | 1.4628594 | up | Cdh2 |
| 6837805\|77980 | 0.020587178 | 4.20E−04 | 1.4586661 | up | Sbf1 |
| 6980052\|16337 | 0.016593723 | 2.51E−04 | 1.4583049 | up | Insr |
| 6990244\|235459 | 0.022598844 | 5.20E−04 | 1.4577506 | down | Gtf2a2 |
| 6957119\|14791 | 0.015983123 | 2.21E−04 | 1.4573512 | down | Emg1\|Lpcat3 |
| 6766705\|13822 | 0.024054471 | 5.87E−04 | 1.4570173 | down | Epb4.112 |
| 6880972\|109778 | 0.013242392 | 1.36E−04 | 1.4568212 | up | Blvra |
| 6752222\|241201 | 0.035368353 | 0.001219077 | 1.4561962 | up | Cdh7 |
| 6803136\|110616 | 0.031045154 | 9.45E−04 | 1.4553119 | up | Atxn3 |
| 6771581\|21334 | 0.022202644 | 4.93E−04 | 1.4538059 | up | Tac2 |
| 6866486\|80718 | 0.015052847 | 1.77E−04 | 1.453329 | down | Rab27b |
| 6989438\|20361 | 0.021438045 | 4.51E−04 | 1.453112 | down | Sema7a |
| 6885872\|73737 | 0.00533197 | 3.45E−05 | 1.4524046 | down | 1110008P14Rik |
| 6969818\|27276 | 0.027616503 | 7.63E−04 | 1.4516916 | up | Plekhb1 |
| 6956748\|67784 | 0.016593723 | 2.52E−04 | 1.4502109 | up | Plxnd1 |
| 6791995\|71795 | 0.006391116 | 4.31E−05 | 1.4501014 | down | Pitpnc1 |
| 7012006\|54411 | 0.028827934 | 8.33E−04 | 1.4465153 | up | Atp6ap1 |
| 6858134\|18189 | 0.03380979 | 0.001117247 | 1.446442 | up | Nrxn1 |
| 6801507\|94090 | 0.019246986 | 3.41E−04 | 1.4461541 | down | Trim9 |
| 6768151\|94214 | 0.015204828 | 1.95E−04 | 1.4460502 | up | Spock2 |
| 6938891\|11980 | 0.020970276 | 4.32E−04 | 1.4438521 | up | Atp8a1 |
| 6843340\|70028 | 0.04115921 | 0.001580126 | 1.4437007 | up | Dopey2 |
| 6929762\|277854 | 0.019152917 | 3.35E−04 | 1.4435827 | up | Depdc5 |
| 6950397, 6957687\| | 0.01601894 | 2.23E−04 | 1.4433552 | up | 8430419L09Rik///Gsg1\|8430 |
| 74525 | | | | | 419L09Rik |
| 6806444\|66154 | 0.017905615 | 2.80E−04 | 1.4423473 | down | Tmem14c |
| 6838257\|67760 | 0.015268379 | 1.99E−04 | 1.4420997 | up | Slc38a2 |
| 6949992\|101187 | 0.032736823 | 0.001031154 | 1.4404699 | down | Parpl1 |
| 6801807\|238271 | 0.029657012 | 8.67E−04 | 1.4399031 | up | Kcnh5 |
| 6785684\|380684 | 0.01910948 | 3.32E−04 | 1.4397109 | up | Nefh |
| 6792994\|382562 | 0.013328801 | 1.39E−04 | 1.4389725 | down | Pfn4 |
| 6986775\|22068 | 0.024526443 | 6.19E−04 | 1.4386616 | down | Trpc6 |
| 6769934\|77048 | 0.006945028 | 4.92E−05 | 1.4383348 | down | Ccdc41 |
| 6785367\|14387 | 0.032434884 | 9.99E−04 | 1.4367542 | up | Gaa |
| 6767850\|215085 | 0.028827934 | 8.33E−04 | 1.4356312 | up | Slc35f1 |
| 6845139\|106264 | 0.020325309 | 4.07E−04 | 1.4345336 | down | 0610012G03Rik |
| 6778528\|56418 | 0.037560377 | 0.001344177 | 1.434402 | down | Ykt6 |
| 6830852, 6836079\| | 0.043119576 | 0.001743216 | 1.4332331 | down | 9930014A18Rik\|Fam84b///Fa |
| 320469 | | | | | m84b\|9930014A18Rik |
| 6830852, 6836079\| | 0.043119576 | 0.001743216 | 1.4332331 | down | 9930014A18Rik\|Fam84b///Fa |
| 399603 | | | | | m84b\|9930014A18Rik |
| 6750557\|66821 | 0.02343562 | 5.44E−04 | 1.4325122 | down | Bcs11\|Zfp142 |
| 6885924\|99326 | 0.017736405 | 2.72E−04 | 1.4325033 | down | Garnl3 |
| 6831469\|19245 | 0.029353406 | 8.56E−04 | 1.4322174 | down | Ptp4a3 |
| 6904979\|73251 | 0.022598844 | 5.19E−04 | 1.4321386 | down | Setd7 |
| 6898477\|20713 | 0.022454733 | 5.10E−04 | 1.4302071 | up | Serpini1 |
| 6844567\|110197 | 0.01916445 | 3.37E−04 | 1.4295702 | down | Dgkg |
| 6960328\|20130 | 0.048653852 | 0.002277486 | 1.4291523 | down | Rras |
| 6754893\|56752 | 0.02606873 | 6.96E−04 | 1.42853 | up | Aldh9a1 |
| 6780882\|52626 | 0.04815002 | 0.002239924 | 1.426755 | up | Cdkn2aipnl |
| 6791212\|22658 | 0.012751671 | 1.29E−04 | 1.4259104 | up | Pcgf2 |
| 6838171\|54003 | 0.044442587 | 0.001914528 | 1.423863 | up | Nell2 |
| 6823302\|71228 | 0.029997475 | 8.84E−04 | 1.4204878 | up | Dlg5 |
| 6829598\|15529 | 0.019556254 | 3.65E−04 | 1.4202565 | up | Sdc2 |
| 6878511\|66861 | 0.015052847 | 1.81E−04 | 1.4187359 | up | Dnajc10 |
| 6821431, 6989873\| | 0.020308778 | 4.06E−04 | 1.4180315 | down | Uchl3\|Uchl4///Uchl4\|Uchl3 |
| 50933 | | | | | |
| 6821431, 6989873\| | 0.020308778 | 4.06E−04 | 1.4180315 | down | Uchl3\|Uchl4///Uchl4\|Uchl3 |
| 93841 | | | | | |
| 6952523\|243743 | 0.028027382 | 7.96E−04 | 1.4170537 | up | Plxna4 |
| 6860163\|93873 | 0.035458572 | 0.001231564 | 1.4162437 | up | Pcdhb2 |
| 6974762\|67207 | 0.010673828 | 8.86E−05 | 1.4154546 | down | Lsm1 |
| 6899374\|20200 | 0.044787455 | 0.001936602 | 1.4153138 | up | S100a6 |
| 6950391\|12576 | 0.048592288 | 0.002267077 | 1.414523 | down | Cdkn1b |
| 6934650\|12909 | 0.015052847 | 1.75E−04 | 1.4131835 | down | Crcp |
| 6986031\|11459 | 0.026061453 | 6.89E−04 | 1.4120103 | down | Acta1 |
| 6847540\|11820 | 0.006600066 | 4.53E−05 | 1.4108847 | up | App |
| 6965015\|52432 | 0.01798404 | 2.89E−04 | 1.4097495 | down | Ppp2r2d |
| 6989473\|319477 | 0.032513015 | 0.00101496 | 1.4095426 | down | 6030419C18Rik |
| 6766368\|26408 | 0.017905615 | 2.80E−04 | 1.4092246 | up | Map3k5 |
| 6764056\|66155 | 0.015204828 | 1.96E−04 | 1.4079518 | down | Ufc1 |
| 6898502\|213262 | 0.019556254 | 3.59E−04 | 1.4078732 | up | Fst15 |
| 6754403\|11899 | 0.010969274 | 9.78E−05 | 1.4076041 | up | Astn1 |
| 6938947\|243043 | 0.008920094 | 7.09E−05 | 1.4064586 | up | Kctd8 |
| 6838823\|58200 | 0.006600066 | 4.59E−05 | 1.406405 | down | Ppp1r1a |
| 6813536\|20745 | 0.04115921 | 0.001591286 | 1.405938 | up | Spock1 |
| 6808773\|13612 | 0.022202644 | 4.91E−04 | 1.4056443 | up | Edil3 |
| 6915929, 6915993\| | 0.015322137 | 2.04E−04 | 1.4053652 | down | Dab1\|Grn10304\|2900034C19 |
| 13131 | | | | | Rik\|AY512949\|LOC1005026 |
| | | | | | 04///Dab1 |
| 6817396\|11534 | 0.024154648 | 5.98E−04 | 1.4021187 | up | Adk |
| 6993890\|68743 | 0.01811096 | 2.98E−04 | 1.3999641 | up | Anln |
| 6995912\|110319 | 0.015137184 | 1.91E−04 | 1.3997213 | up | Mpi |
| 6940592\|246293 | 0.006600066 | 4.57E−05 | 1.3995645 | down | Klhl8 |
| 6963534\|320878 | 0.042509187 | 0.001677097 | 1.3995601 | down | Mical2 |
| 6842682\|17968 | 0.013328801 | 1.41E−04 | 1.3989094 | up | Ncam2 |
| 6992332\|14775 | 0.014431601 | 1.60E−04 | 1.3986729 | down | Gpx1 |
| 6891689\|241688 | 0.03991207 | 0.001487267 | 1.397334 | up | 6330439K17Rik |
| 6888751\|228355 | 0.018393353 | 3.12E−04 | 1.3969011 | up | Madd |
| 6891322\|59030 | 0.01630344 | 2.38E−04 | 1.3968654 | down | Mkks |
| 6940431, 6940432\| | 0.019232834 | 3.39E−04 | 1.3944072 | up | Wdfy3 |
| 72145 | | | | | |
| 6852358, 6925574\| | 0.0332504 | 0.001077326 | 1.3940427 | up | Hdac1 |
| 433759 | | | | | |
| 6816124, 6838415\| | 0.010677658 | 9.16E−05 | 1.3939478 | up | Il31ra\|Tuba1b\|Gm5620///Tub |
| 22143 | | | | | a1b\|Gm6682\|Gm5620 |
| 6838382\|69612 | 0.044053618 | 0.00187886 | 1.3932033 | down | 2310037I24Rik |
| 6793649\|50496 | 0.010673828 | 8.83E−05 | 1.3926133 | down | E2f6 |
| 6896519\|20482 | 0.019556254 | 3.70E−04 | 1.3922062 | down | Skil |
| 6918720\|20810 | 0.024526443 | 6.18E−04 | 1.3916972 | down | Srm |
| 6760754\|16560 | 0.021126166 | 4.38E−04 | 1.390481 | up | Kif1a |
| 6949797, 6957119\| | 0.021159004 | 4.41E−04 | 1.38964 | down | Lpcat3///Emg1\|Lpcat3 |
| 14792 | | | | | |
| 6867701\|56464 | 0.021438045 | 4.49E−04 | 1.3882275 | up | Ctsf |
| 6791418\|15114 | 0.018393353 | 3.11E−04 | 1.3881177 | up | Hap1 |
| 6918042\|69116 | 0.01984823 | 3.94E−04 | 1.3880422 | up | Ubr4\|C230096C10Rik |
| 6918042\|230866 | 0.01984823 | 3.94E−04 | 1.3880422 | up | Ubr4\|C230096C10Rik |
| 6803358, 6803364\| | 0.016230881 | 2.34E−04 | 1.3870988 | up | Atg2b |
| 76559 | | | | | |
| 6958256\|79362 | 0.011055893 | 1.00E−04 | 1.3868607 | up | Bhlhe41 |
| 6785943, 6978341\| | 0.01608881 | 2.27E−04 | 1.3867203 | down | Polr2c |
| 20021 | | | | | |
| 6793255, 6804226\| | 0.015052847 | 1.76E−04 | 1.3839858 | up | Wdr35///Wdr35\|Matn3 |
| 74682 | | | | | |
| 6952137\|320405 | 0.007270184 | 5.21E−05 | 1.3828329 | up | Cadps2 |
| 6891454\|75812 | 0.015441114 | 2.08E−04 | 1.3827794 | down | Tasp1 |
| 6775098, 6776193\| | 0.013542953 | 1.46E−04 | 1.3822339 | down | Mrpl42 |
| 67270 | | | | | |
| 6871837\|271564 | 0.022187717 | 4.85E−04 | 1.3803174 | up | Vps13a |
| 6955205\|66881 | 0.04115921 | 0.001587085 | 1.3796992 | up | Pcyox1 |
| 6964023\|28018 | 0.015322137 | 2.06E−04 | 1.3796805 | down | Ubfd1 |
| 6949361\|232337 | 0.043720026 | 0.001840079 | 1.3792504 | down | Zfp637 |
| 6996440\|235442 | 0.030360658 | 9.04E−04 | 1.3790128 | up | Rab8b |
| 6766110\|15273 | 0.015052847 | 1.80E−04 | 1.3786916 | down | Hivep2 |
| 6977075\|66498 | 0.039544 | 0.001470771 | 1.3776422 | down | Dda1 |
| 6992215\|56808 | 0.049109604 | 0.002316849 | 1.3775046 | up | Cacna2d2 |
| 6868032\|54525 | 0.024762 | 6.32E−04 | 1.3772434 | up | Syt7 |
| 6840923\|268890 | 0.040423766 | 0.001535315 | 1.3750381 | up | Lsamp |
| 6971344\|66422 | 0.04337046 | 0.001766213 | 1.3750355 | down | Dctpp1 |
| 6885482\|52838 | 0.022202644 | 4.92E−04 | 1.3748771 | down | Dnlz |
| 6767631\|209462 | 0.034334507 | 0.001142994 | 1.3747562 | down | Hace1 |
| 6964244\|26417 | 0.01984823 | 3.89E−04 | 1.3736368 | up | Mapk3 |
| 6968453\|64176 | 0.008398302 | 6.26E−05 | 1.3733315 | up | Sv2b |
| 7017600\|16728 | 0.019556254 | 3.56E−04 | 1.3732485 | up | L1cam |
| 6910621\|68830 | 0.007590169 | 5.50E−05 | 1.3714011 | down | Nexn |
| 7008100\|50918 | 0.019556254 | 3.65E−04 | 1.3707279 | up | Myadm\|Prkcc |
| 7008100\|18752 | 0.019556254 | 3.65E−04 | 1.3707279 | up | Myadm\|Prkcc |
| 6820088\|213484 | 0.04208484 | 0.001642205 | 1.3698381 | down | Nudt18 |
| 6983927\|66714 | 0.035799697 | 0.001245929 | 1.3697839 | down | 4921524J17Rik |
| 6913020\|230103 | 0.024526443 | 6.17E−04 | 1.3680389 | up | Nor2 |
| 6963211\|14356 | 0.043720026 | 0.001844338 | 1.3680122 | down | Fxc1\|Dnhd1\|Gm9571 |
| 6963211\|77505 | 0.043720026 | 0.001844338 | 1.3680122 | down | Fxc1\|Dnhd1\|Gm9571 |
| 6963211\|672646 | 0.043720026 | 0.001844338 | 1.3680122 | down | Fxc1\|Dnhd1\|Gm9571 |
| 6799524\|108089 | 0.01798404 | 2.92E−04 | 1.3673081 | down | Rnf144a |
| 6882521\|66734 | 0.032802183 | 0.001035519 | 1.3671783 | down | Map1lc3a |
| 6971688\|77938 | 0.0332504 | 0.001091005 | 1.3666912 | down | Fam53b |
| 6789401\|104457 | 0.01297834 | 1.32E−04 | 1.3666172 | down | 0610010K14Rik |
| 6899747, 6899750, | 0.024054471 | 5.86E−04 | 1.3645159 | down | Hist2h2aa1\|Hist2h2aa2\|Hist2 |
| 6899752, 6907246, | | | | | h2ac\|Hist2h3c1///Hist2h3c1\| |
| 6907247\|15077 | | | | | Hist2h3c2- |
| | | | | | ps///Hist2h3b\|Hist2h3c1\|Hist |
| | | | | | 2b3c2- |
| | | | | | ps///Hist2h3c1\|Hist2b3c2- |
| | | | | | ps\|Hist2b3b///Hist2h2aa1\|Hist |
| | | | | | 2h2aa2\|Hist2h3c1 |
| 6909139\|109676 | 0.027723162 | 7.74E−04 | 1.3641738 | up | Ank2\|Gm4392 |
| 6909139\|100043364 | 0.027723162 | 7.74E−04 | 1.3641738 | up | Ank2\|Gm4392 |
| 7017627\|27643 | 0.039544 | 0.001465421 | 1.36293 | down | Ubl4\|Slc10a3-ubl4 |
| 6985851\|18117 | 0.04533207 | 0.001965658 | 1.3628076 | down | Cox4nb |
| 6842933\|74112 | 0.03459776 | 0.001165178 | 1.3626226 | down | Usp16 |
| 6959133\|66071 | 0.013328801 | 1.40E−04 | 1.3620924 | up | Ethe1 |
| 6780844, 6788069\| | 0.034624055 | 0.001174939 | 1.3607397 | down | Zfp354a\|Zfp354b\|9230009I0 |
| 21408 | | | | | 2Rik///Zfp354b\|Zfp354a |
| 6780844, 6788069\| | 0.034624055 | 0.001174939 | 1.3607397 | down | Zfp354a\|Zfp354b\|9230009I0 |
| 27274 | | | | | 2Rik///Zfp354b\|Zfp354a |
| 6750868\|74205 | 0.0281969 | 8.07E−04 | 1.3601534 | up | Acsl3\|Utp14b |
| 6750868\|195434 | 0.0281969 | 8.07E−04 | 1.3601534 | up | Acsl3\|Utp14b |
| 6755222\|12847 | 0.019556254 | 3.53E−04 | 1 3600298 | up | Copa |
| 6812894\|20238 | 0.009567102 | 7.67E−05 | 1.3594275 | down | Atxn1 |
| 6775741\|28088 | 0.01798404 | 2.93E−04 | 1.3590493 | up | D10Wsu52e |
| 6917217\|242667 | 0.012750876 | 1.27E−04 | 1.3580503 | down | Dlgap3 |
| 6778583\|216527 | 0.01630344 | 2.39E−04 | 1.3575718 | down | Ccm2 |
| 6912213\|68493 | 0.022382699 | 5.02E−04 | 1.3574581 | down | Ndufaf4 |
| 6855051\|12268 | 0.026502775 | 7.17E−04 | 1.3574362 | up | C4b\|C4a |
| 6855051\|625018 | 0.026502775 | 7.17E−04 | 1.3574362 | up | C4b\|C4a |
| 6805245, 6811686\| | 0.030477278 | 9.11E−04 | 1.3565937 | down | Hist1h2bn///Hist1h2be\|Hist1h |
| 319187 | | | | | 1e\|Hist1h2bn |
| 6786991\|75572 | 0.013328801 | 1.39E−04 | 1.3564721 | down | Acyp2\|Ccdc47 |
| 6935370\|14086 | 0.036739744 | 0.001301056 | 1.3564117 | down | Fscn1 |
| 6995661\|330941 | 0.010969274 | 9.87E−05 | 1.3559855 | down | AI593442 |
| 6939985\|67111 | 0.038066395 | 0.001372992 | 1.3558711 | up | Naaa |
| 6998707\|74443 | 0.037047874 | 0.003321823 | 1.3553175 | up | P4htm |
| 6947760\|103963 | 0.028027382 | 7.95E−04 | 1.3548398 | up | Rpn1 |
| 6825371\|110265 | 0.033087827 | 0.001056168 | 1.3541646 | down | Msra |
| 6997077\|71538 | 0.014639024 | 1.65E−04 | 1.3531889 | down | Fbxo9 |
| 6785742\|64660 | 0.016530215 | 2.46E−04 | 1.3525581 | down | Mrps24 |
| 6880540\|228550 | 0.008480565 | 6.52E−05 | 1.3519324 | down | Itpka |
| 6848513\|68842 | 0.022382699 | 5.03E−04 | 1.3511229 | up | Tulp4 |
| 6925345\|66938 | 0.024154648 | 5.92E−04 | 1.350936 | down | 1700029G01Rik |
| 6888720\|66461 | 0.03315913 | 0.001060775 | 1.3498696 | down | Ptpmt1 |
| 6978291\|17748 | 0.018016174 | 2.95E−04 | 1.3498284 | up | Mt1 |
| 7010345\|236733 | 0.019556254 | 3.60E−04 | 1.3488789 | up | Usp11 |
| 6754014\|117198 | 0.00536721 | 3.51E−05 | 1.3487188 | down | Ivns1abp |
| 6935524\|264064 | 0.016230881 | 2.35E−04 | 1.3464878 | down | Cdk8 |
| 6929919\|231148 | 0.010677658 | 9.22E−05 | 1.346334 | down | Ablim2 |
| 6833138\|22146 | 0.042509187 | 0.001688896 | 1.3447404 | up | Tuba1c\|Gm6682\|Gm8973 |
| 6833138\|668092 | 0.042509187 | 0.001688896 | 1.3447404 | up | Tuba1c\|Gm6682\|Gm8973 |
| 6963264\|60510 | 0.04670013 | 0.002075124 | 1.3440274 | up | Syt9 |
| 6916797\|29871 | 0.02366127 | 5.54E−04 | 1.3433441 | down | Scmh1 |
| 6892193\|68559 | 0.023056254 | 5.33E−04 | 1.3426592 | down | Pdrg1 |
| 6941761\|207565 | 0.04337046 | 0.001796049 | 1.3405432 | down | Camkk2 |
| 6998396\|20818 | 0.031038841 | 9.40E−04 | 1.340059 | up | Srprb |
| 6852902\|17688 | 0.04679768 | 0.002089329 | 1.3396536 | up | Msh6\|Fbxo11 |
| 6852902\|225055 | 0.04679768 | 0.002089329 | 1.3396536 | up | Msb6\|Fbxo11 |
| 6883127\|57138 | 0.03864976 | 0.001417799 | 1.3395128 | up | Slc12a5 |
| 6761155\|27392 | 0.026663529 | 7.27E−04 | 1.339372 | up | Pign |
| 6788411\|11927 | 0.011055893 | 1.01E−04 | 1.3388278 | down | Atox1 |
| 6845459\|207227 | 0.024154648 | 5.95E−04 | 1.3388058 | up | Stxbp5l |
| 6771920\|270685 | 0.035340734 | 0.001215047 | 1.3384888 | up | Mthfd1l |
| 6966339\|56188 | 0.043720026 | 0.001841995 | 1.3383098 | up | Fxyd1 |
| 6864062\|108013 | 0.020886658 | 4.27E−04 | 1.337718 | up | Celf4 |
| 6945914\|66797 | 0.015983123 | 2.21E−04 | 1.3368968 | up | Cntnap2\|Ccni |
| 6811806\|22360 | 0.04193666 | 0.001624865 | 1.3367634 | up | Nrsn1 |
| 6782456\|19062 | 0.03324496 | 0.001070845 | 1.3359902 | up | Inpp5k |
| 6775310\|70294 | 0.04337046 | 0.001783667 | 1.3358172 | down | Rnf126 |
| 6840579\|22042 | 0.03840255 | 0.001390516 | 1.3344265 | down | Tfrc |
| 6975876\|192169 | 0.019556254 | 3.61E−04 | 1.3340727 | down | Ufsp2 |
| 6754137\|67792 | 0.017340807 | 2.65E−04 | 1.3336473 | down | Rgs8 |
| 6917790\|71665 | 0.03294127 | 0.001046858 | 1.3336054 | up | Fuca1 |
| 6850421\|17850 | 0.044442587 | 0.001907872 | 1.3334374 | up | Mut\|Cenpq |
| 6767258\|14360 | 0.016510215 | 2.48E−04 | 1.3333771 | down | Fyn |
| 6908146\|20912 | 0.04368416 | 0.001822909 | 1.3332828 | up | Stxbp3a |
| 6755173, 6764068\| | 0.01910948 | 3.33E−04 | 1.3316907 | down | Dedd///Nit1\|Dedd |
| 21945 | | | | | |
| 6896518\|18759 | 0.010822849 | 9.44E−05 | 1.3315817 | down | Prkci |
| 7014815\|110651 | 0.034624055 | 0.00116928 | 1.331539 | down | Rps6ka3 |
| 6807437\|75731 | 0.015854789 | 2.17E−04 | 1.3310698 | down | 5133401N09Rik |
| 6883013\|228858 | 0.0332504 | 0.001087326 | 1.3309959 | up | Gdap111 |
| 6827203\|72486 | 0.04312426 | 0.001746437 | 1.3300443 | up | Rnf219 |
| 7010647\|72693 | 0.043053027 | 0.001733175 | 1.3294554 | up | Zcchc12 |
| 6916125\|230584 | 0.020587178 | 4.20E−04 | 1.3290225 | up | Yipf1\|Rfc5 |
| 6868899\|22359 | 0.03324496 | 0.001071679 | 1.3275667 | up | Vldlr |
| 6966328\|22282 | 0.01394069 | 1.53E−04 | 1.3273046 | down | Usf2 |
| 6929719\|14284 | 0.027805798 | 7.85E−04 | 1.326915 | down | Fosl2 |
| 6992328\|66257 | 0.042509187 | 0.00168081 | 1.3260579 | up | Nicn1 |
| 6831592\|22701 | 0.04244813 | 0.00166235 | 1.3257983 | down | Zip41 |
| 6869635, 6873083\| | 0.01798404 | 2.92E−04 | 1.3250004 | down | Entpd1\|Tctn3///Tctn3 |
| 67590 | | | | | |
| 6749455\|227095 | 0.038871896 | 0.001429375 | 1.3242575 | up | Hibch |
| 6896593\|67414 | 0.04199467 | 0.001632782 | 1.3226247 | up | Mfn1 |
| 6818742\|93834 | 0.011135913 | 1.05E−04 | 1.3224422 | down | Peli2 |
| 6993465\|71946 | 0.04337046 | 0.001770237 | 1.320743 | up | Endod1 |
| 6884352\|50497 | 0.034411497 | 0.001156486 | 1.3202697 | down | Hspa14 |
| 6874080\|73442 | 0.025432337 | 6.54E−04 | 1.3201097 | up | Hspa12a |
| 6931961\|319387 | 0.023703147 | 5.68E−04 | 1.3191973 | up | Lphn3\|Dynlt1a\|A230055J12R |
| | | | | | ik |
| 6931961\|320314 | 0.023703147 | 5.68E−04 | 1.3191973 | up | Lphn3\|Dynlt1a\|A230055J12R |
| | | | | | ik |
| 6845559\|76916 | 0.043053027 | 0.001732094 | 1.3186158 | down | 4930455C21Rik |
| 6937073\|14208 | 0.032301586 | 9.90E−04 | 1.3180437 | up | Ppm1g |
| 6759718\|21961 | 0.022454733 | 5.11E−04 | 1.3180168 | down | Tns1 |
| 6869973\|226151 | 0.032512043 | 0.001010358 | 1.3166649 | up | Fam178a |
| 6787293\|23964 | 0.043053027 | 0.001730065 | 1.3164718 | up | Odz2 |
| 6757896\|320011 | 0.04584206 | 0.00202088 | 1.3162661 | up | Uggt1 |
| 6933812\|57816 | 0.016510215 | 2.45E−04 | 1.3148854 | down | Tesc |
| 6878657\|241520 | 0.043053027 | 0.001734471 | 1.3144302 | up | Fam171b |
| 6884183\|72075 | 0.026050128 | 6.84E−04 | 1.3144196 | down | Ogfr |
| 6935927\|13121 | 0.02441559 | 6.10E−04 | 1.314148 | up | Cyp51 |
| 6833185\|14555 | 0.034411497 | 0.001153731 | 1.3139409 | down | Gpd1 |
| 6792129\|217265 | 0.015052847 | 1.82E−04 | 1.3136501 | up | Abca5 |
| 6757120\|29819 | 0.023703147 | 5.65E−04 | 1.3135145 | down | Stau2\|C130013N14Rik |
| 6757120\|402742 | 0.023703147 | 5.65E−04 | 1.3135145 | down | Stau2\|C130013N14Rik |
| 6789979\|69713 | 0.026313707 | 7.05E−04 | 1.3133277 | down | Nlk\|Pin4 |
| 6776152\|67723 | 0.022454733 | 5.12E−04 | 1.3133212 | up | 4932415G12Rik |
| 6857310\|72722 | 0.017905615 | 2.82E−04 | 1.3130908 | down | Fam98a |
| 6966588\|19777 | 0.02366127 | 5.57E−04 | 1.3123834 | down | C80913 |
| 6774684\|211488 | 0.02650006 | 7.12E−04 | 1.3117256 | down | Ado |
| 6768323\|73132 | 0.042509187 | 0.00169164 | 1.3114651 | down | Slc25a16 |
| 6840019\|75826 | 0.022007378 | 4.78E−04 | 1.3112297 | down | Senp2 |
| 6964259\|233878 | 0.01566359 | 2.13E−04 | 1.3110644 | up | Sez6l2 |
| 6892364\|228812 | 0.019560797 | 3.74E−04 | 1.3108152 | up | Pigu |
| 6832719\|12805 | 0.043834306 | 0.001859543 | 1.3107749 | up | Cntn1 |
| 6768094\|19386 | 0.023703147 | 5.62E−04 | 1.3087014 | down | Ranbp2 |
| 6873254\|73689 | 0.019560797 | 3.77E−04 | 1.3083574 | down | Bloc1s2 |
| 6902661\|12972 | 0.019008702 | 3.27E−04 | 1.3077829 | up | Cryz |
| 6974039\|54126 | 0.022860363 | 5.27E−04 | 1.3058306 | down | Arhgef7 |
| 6896584, 6904047\| | 0.015137184 | 1.90E−04 | 1.305752 | down | 4930429B21Rik\|Zmat3///Zm |
| 22401 | | | | | at3 |
| 6966187\|73833 | 0.024154648 | 5.94E−04 | 1.3053551 | down | Rasgrp4\|Fam98c |
| 6797707\|73046 | 0.04815002 | 0.00224111 | 1.3049716 | down | Glrx5 |
| 6918705\|230904 | 0.03637223 | 0.001270969 | 1.3044555 | up | Fbxo2 |
| 6988773\|22687 | 0.03992887 | 0.001493508 | 1.3041425 | down | Zfp259 |
| 6969028\|14085 | 0.049958326 | 0.002381109 | 1.3041215 | up | Fah |
| 6810280\|268706 | 0.043053027 | 0.001727578 | 1.3038671 | up | Slc38a9 |
| 6853762\|26407 | 0.014973253 | 1.72E−04 | 1.3027297 | up | Map3k4 |
| 6789979, 6888496\| | 0.029707763 | 8.73E−04 | 1.3026756 | down | Nlk\|Pin4///Olfr1111\|Nlk |
| 18099 | | | | | |
| 6763652\|98376 | 0.048653852 | 0.002278244 | 1.3022286 | up | Gorab |
| 7017627, 7017628\| | 0.036739744 | 0.001300395 | 1.3021116 | down | Ubl4\|Slc10a3- |
| 100169864 | | | | | ubl4///Slc10a3\|Slc10a3-ubl4 |
| 6831994\|11911 | 0.022598844 | 5.16E−04 | 1.3008779 | down | Atf4 |
| 6770325\|103098 | 0.029003233 | 8.40E−04 | 1.3007712 | up | Slc6a15 |
| 6876173\|227723 | 0.018353892 | 3.07E−04 | 1.299692 | up | Bat21 |
| 6864678\|67199 | 0.034411497 | 0.001152159 | 1.2993454 | down | Pfdn1 |
| 6881771\|18549 | 0.03294127 | 0.001044944 | 1.2993256 | up | Pcsk2 |
| 6823041, 6823100, | 0.047668647 | 0.002167039 | 1.2989156 | up | Camk2g\|Usp54///Usp54 |
| 6823105\|78787 | | | | | |
| 6884721\|50755 | 0.012751671 | 1.28E−04 | 1.2987964 | down | Fbxo18 |
| 6917489\|66464 | 0.0358883 | 0.001251536 | 1.2987165 | down | Taf12 |
| 6966164\|24030 | 0.019755332 | 3.85E−04 | 1.2983397 | down | Mrps12 |
| 6877931\|73373 | 0.043834306 | 0.001858988 | 1.2981822 | down | Phospho2\|Rbm3 |
| 6788020\|12330 | 0.021695498 | 4.64E−04 | 1.2979654 | up | Canx |
| 6955766\|101351 | 0.035250623 | 0.001209471 | 1.2979203 | up | A130022J15Rik |
| 6823710\|64652 | 0.019556254 | 3.64E−04 | 1.2974981 | up | Nisch |
| 6966600\|12447 | 0.021552088 | 4.59E−04 | 1.2974267 | up | Ccne1 |
| 6954572\|104263 | 0.045719497 | 0.002007459 | 1.2972401 | up | Kdm3a |
| 6958995\|403187 | 0.027723162 | 7.76E−04 | 1.296651 | down | Opa3 |
| 6899585\|78523 | 0.039544 | 0.001469816 | 1.2958944 | down | Mrpl9 |
| 6782088, 6789369\| | 0.036654945 | 0.00128858 | 1.2950375 | down | Dullard\|Rai12///Rai12 |
| 54351 | | | | | |
| 6944432\|76522 | 0.046211697 | 0.002040424 | 1.2948897 | down | Naa38 |
| 6915745\|242557 | 0.047974896 | 0.002199229 | 1.2935965 | down | Atg4c\|Gm12689\|Gm10305 |
| 6915745\|1001370 | 0.047974896 | 0.002199229 | 1.2935965 | down | Atg4c\|Gm12689\|Gm10305 |
| 11 | | | | | |
| 6915745\|1000387 | 0.047974896 | 0.002199229 | 1.2935965 | down | Atg4c\|Gm12689\|Gm10305 |
| 27 | | | | | |
| 6840527\|66994 | 0.022202644 | 4.91E−04 | 1.2931771 | down | 1500031L02Rik |
| 6867642\|66990 | 0.040423766 | 0.001546125 | 1.2931631 | down | Tmem134 |
| 6949153\|232333 | 0.03481309 | 0.001184668 | 1.2929022 | up | Slc6a1 |
| 6945614, 6952941\| | 0.039544 | 0.001463646 | 1.2925439 | down | Mkrn1 |
| 54484 | | | | | |
| 6783654\|71452 | 0.019560797 | 3.78E−04 | 1.2917719 | down | Ankrd40 |
| 6791541\|268490 | 0.024805788 | 6.35E−04 | 1.2917227 | down | Lsm12 |
| 6929125\|330050 | 0.044053618 | 0.001880092 | 1.2914281 | up | Fam185a |
| 6864695\|24068 | 0.018772775 | 3.22E−04 | 1.2907568 | down | Sra1 |
| 6769213, 7008703\| | 0.04026438 | 0.001520212 | 1.290465 | up | Plk5///Plk5\|Spt1\|LOC236598 |
| 216166 | | | | | |
| 6899743\|64051 | 0.02606873 | 6.94E−04 | 1.2904557 | up | Sv2a |
| 6775372\|66594 | 0.03663139 | 0.001285176 | 1.2897568 | down | Uqcr11 |
| 6929457, 6929458\| | 0.04954692 | 0.002347931 | 1.289421 | up | Dpp6 |
| 13483 | | | | | |
| 6977142\|17274 | 0.04013421 | 0.00150401 | 1.289129 | down | Rab8a |
| 6958407\|387314 | 0.031038841 | 9.38E−04 | 1.2888066 | up | Tmtc1 |
| 6936116\|23857 | 0.044442587 | 0.001903648 | 1.2882978 | down | Dmtf1 |
| 6899613\|229584 | 0.031288665 | 9.55E−04 | 1.2878227 | up | Pogz |
| 6962925\|70974 | 0.022454733 | 5.08E−04 | 1.2877574 | up | Pgm2l1\|Gpx2-ps1 |
| 6938710\|68552 | 0.024872728 | 6.38E−04 | 1.2877141 | down | 1110003E01Rik |
| 6836237\|13196 | 0.04337046 | 0.00179329 | 1.2868526 | down | Asap1\|9130004J05Rik\|Gm10 |
| | | | | | 926\|LOC100039024 |
| 6836237\|71603 | 0.04337046 | 0.00179329 | 1.2868526 | down | Asap1\|9130004J05Rik\|Gm10 |
| | | | | | 926\|LOC100039024 |
| 6836237\|1001698 | 0.04337046 | 0.00379329 | 1.2868526 | down | Asap1\|9130004J05Rik\|Gm10 |
| 72 | | | | | 926\|LOC100039024 |
| 6836237\|1000390 | 0.04337046 | 0.00179329 | 1.2868526 | down | Asap1\|9130004J05Rik\|Gm10 |
| 24 | | | | | 926\|LOC100039024 |
| 6996269\|26395 | 0.030709505 | 9.20E−04 | 1.2838286 | up | Map2k1 |
| 6842326\|19876 | 0.04390929 | 0.001867758 | 1.2838104 | up | Robo1 |
| 6833186\|66379 | 0.044442587 | 0.003914988 | 1.28336 | down | 2310016M24Rik |
| 6924281\|56280 | 0.04115921 | 0.001585019 | 1.2821487 | down | Mrpl37 |
| 6852767\|19043 | 0.021552088 | 4.58E−04 | 1.2815548 | down | Ppm1b |
| 6788141\|76901 | 0.016510215 | 2.48E−04 | 1.2815293 | up | Phf15 |
| 6952900\|15258 | 0.04584206 | 0.002018127 | 1.2813956 | up | Hipk2 |
| 6975050\|66959 | 0.04953374 | 0.002343824 | 1.2809025 | down | Dusp26 |
| 6755233\|140559 | 0.021552088 | 4.59E−04 | 1.2806572 | up | Igsf8 |
| 6765307\|214791 | 0.019556254 | 3.64E−04 | 1.2801203 | down | Sertad4 |
| 6780767\|14584 | 0.04337046 | 0.001787895 | 1.2799969 | up | Gfpt2 |
| 6962930\|320452 | 0.046510797 | 0.00206017 | 1.2792466 | up | P4ha3 |
| 6750149\|66646 | 0.019556254 | 3.50E−04 | 1.2789862 | down | Rpe |
| 6801914, 6962925\| | 0.02474206 | 6.30E−04 | 1.2780323 | up | Gpx2\|Gpx2- |
| 14776 | | | | | ps1///Pgm2l1\|Gpx2-ps1 |
| 6801914, 6962925\| | 0.02474206 | 6.30E−04 | 1.2780323 | up | Gpx2\|Gpx2- |
| 14777 | | | | | ps1///Pgm2l1\|Gpx2-ps1 |
| 6991027\|21983 | 0.04880326 | 0.002292103 | 1.2778425 | up | Tpbg |
| 6816317\|52552 | 0.026050128 | 6.85E−04 | 1.2773947 | down | Parp8 |
| 6895393\|11308 | 0.029042374 | 8.43E−04 | 1.2773659 | down | Abi1 |
| 6970568\|68815 | 0.019560797 | 3.78E−04 | 1.2766405 | down | Btbd10 |
| 6768897\|103172 | 0.03806482 | 0.001366233 | 1.2742038 | down | Chchd10 |
| 6793253, 6804226\| | 0.042109743 | 0.001646138 | 1.2741894 | up | Matn3///Wdr35\|Matn3 |
| 17182 | | | | | |
| 6820237\|67381 | 0.016230881 | 2.33E−04 | 1.2731138 | down | Med4 |
| 6992367\|19087 | 0.047316674 | 0.002136069 | 1.2726023 | up | Prkar2a |
| 6754205, 7011852\| | 0.024154648 | 5.97E−04 | 1.2718637 | down | Stx6\|Hmgb3///Hmgb3 |
| 15354 | | | | | |
| 6989440\|13070 | 0.047316674 | 0.002129709 | 1.2717532 | down | Cyp11a1 |
| 6819928\|239157 | 0.03260038 | 0.001023769 | 1.2716821 | up | Pnnma2 |
| 6964329\|68961 | 0.027616503 | 7.65E−04 | 1.2709464 | down | Phkg2\|Gm166 |
| 6964329\|233899 | 0.027616503 | 7.65E−04 | 1.2709464 | down | Phkg2\|Gm166 |
| 6896770\|229211 | 0.04261202 | 0.001698729 | 1.2707958 | up | Acad9 |
| 6819694, 6825302\| | 0.015052847 | 1.86E−04 | 1.2706757 | up | Ctsb\|Fdft1///Fdft1\|Ctsb |
| 13030 | | | | | |
| 6819694, 6825302\| | 0.015052847 | 1.86E−04 | 1.2706757 | up | Ctsb\|Fdft1///Fdft1\|Ctsb |
| 14137 | | | | | |
| 6980270\|13642 | 0.021932513 | 4.73E−04 | 1.2703769 | up | Efnb2 |
| 6824779\|59049 | 0.0354481 | 0.001228634 | 1.2695707 | up | Slc22a17 |
| 6922895, 6922901\| | 0.038421385 | 0.001402961 | 1.2689745 | down | Ttc39b |
| 69863 | | | | | |
| 6942675\|100494 | 0.031003293 | 9.31E−04 | 1.268897 | down | Zfand2a |
| 6854541\|56409 | 0.026039083 | 6.79E−04 | 1.2682208 | down | Nudt3\|Anks1 |
| 6837470\|29859 | 0.042509187 | 0.001683426 | 1.2679896 | down | Sult4a1 |
| 6881337\|12653 | 0.044442587 | 0.00191544 | 1.2677501 | up | Chgb |
| 6823723\|24056 | 0.032360055 | 9.94E−04 | 1.2668406 | up | Sh3bp5\|Capn7 |
| 6759905\|13838 | 0.033567186 | 0.00110687 | 1.2665352 | up | Epha4 |
| 6875602\|74159 | 0.0469654 | 0.002106723 | 1.2665263 | down | Acbd5 |
| 6822891\|218772 | 0.02661468 | 7.22E−04 | 1.2661253 | down | Rarb\|Rpl23a |
| 6791233\|12295 | 0.017905615 | 2.82E−04 | 1.2653434 | down | Cacnb1 |
| 6764662\|226757 | 0.032434884 | 0.001001119 | 1.2649517 | down | Wdr26 |
| 6937844\|16826 | 0.03260038 | 0.00102078 | 1.2648244 | up | Ldb2 |
| 6754526\|73844 | 0.036409926 | 0.001274847 | 1.264413 | up | Ankrd45 |
| 6834745\|223455 | 0.040423766 | 0.001534566 | 1.2642958 | up | 6-Mar |
| 6792787\|209011 | 0.02366127 | 5.58E−04 | 1.2642294 | down | Sirt7 |
| 6837189\|66538 | 0.03840255 | 0.001389966 | 1.2636565 | down | Rps19bp1 |
| 6769192\|66043 | 0.02366127 | 5.59E−04 | 1.263041 | down | Atp5d |
| 6781561\|72795 | 0.047668647 | 0.002171312 | 1.2627603 | down | Ttc19 |
| 6785665\|66152 | 0.040423766 | 0.00154082 | 1.2617928 | down | Uqcr10 |
| 6980964\|18970 | 0.03806482 | 0.001370259 | 1.2614816 | down | Polb\|A930013F10Rik |
| 6980964\|68074 | 0.03806482 | 0.001370259 | 1.2614816 | down | Polb\|A930013F10Rik |
| 6760006\|69368 | 0.043720026 | 0.001832152 | 1.2613966 | up | Wdfy1 |
| 6754437, 6957465, | 0.027663546 | 7.68E−04 | 1.2612764 | down | Rfwd2\|Scarna3a///Csda\|Rfwd |
| 7011663, 7018291\| | | | | | 2///Ctag2\|Rfwd2///Asb12\|Rfw |
| 26374 | | | | | d2 |
| 6784785\|13929 | 0.026686419 | 7.31E−04 | 1.2611614 | down | Amz2 |
| 6883098\|52840 | 0.027805798 | 7.82E−04 | 1.2610734 | down | Dbndd2 |
| 7014836\|58194 | 0.025439167 | 6.56E−04 | 1.2605152 | down | Sh3kbp1\|Map3k15 |
| 6757634, 6873078, | 0.024367737 | 6.06E−04 | 1.260183 | down | Ptp4a1\|Gm13363///Gm13363\| |
| 6875459\|19243 | | | | | Ptp4a1///Etl4\|Gm13363\|Ptp4a |
| | | | | | 1\|Gm\|6495 |
| 6757634, 6873078, | 0.024367737 | 6.06E−04 | 1.260183 | down | Ptp4a1\|Gm13363///Gm13363\| |
| 6875459\|433406 | | | | | Ptp4a1///Etl4\|Gm13363\|Ptp4a |
| | | | | | 1\|Gm16495 |
| 6815255\|66549 | 0.032440964 | 0.001005171 | 1.260047 | down | Aggf1 |
| 6861350\|12322 | 0.03260038 | 0.001024564 | 1.2599939 | up | Camk2a |
| 6833138, 6838415, | 0.0332504 | 0.001091748 | 1.2594355 | up | Tuba1c\|Gm6682\|Gm8973///T |
| 6838417\|626534 | | | | | uba1b\|Gm6682\|Gm5620///Tu |
| | | | | | ba1a\|Gm6682\|Gm5620 |
| 6987128\|69137 | 0.026650216 | 7.25E−04 | 1.2593307 | up | 2200002K05Rik |
| 6942276\|212996 | 0.038421385 | 0.001404705 | 1.2593135 | down | Wbscr17 |
| 6972990\|22192 | 0.0332504 | 0.001091686 | 1.2590938 | down | Ube2m |
| 6901732\|108943 | 0.033052154 | 0.001052705 | 1.2589556 | down | Rg9mtd2 |
| 6782708\|55978 | 0.02366127 | 5.52E−04 | 1.2587875 | down | Ift20 |
| 6985355\|20340 | 0.045719497 | 0.002009048 | 1.2586819 | up | Glg1 |
| 7009774\|20977 | 0.032272834 | 9.87E−04 | 1.2579204 | up | Syp |
| 6788993\|70383 | 0.034411497 | 0.001154944 | 1.2574023 | down | Cox 10 |
| 6835104\|54375 | 0.01984823 | 3.91E−04 | 1.2570033 | down | Azin1 |
| 6798218\|17169 | 0.02474206 | 6.29E−04 | 1.2569531 | down | Mark3 |
| 6817229, 6822949\| | 0.040423766 | 0.001545631 | 1.255124 | down | Nkiras1///Ube2e1\|Nkiras1 |
| 69721 | | | | | |
| 6985984\|78779 | 0.040423766 | 0.001540842 | 1.2541198 | down | Spata2L |
| 6966425\|14751 | 0.040252663 | 0.001513779 | 1.2531556 | up | Gpi1 |
| 6949084\|68089 | 0.031038841 | 9.36E−04 | 1.2528758 | down | Arpc4 |
| 6814385\|18570 | 0.04533531 | 0.001976228 | 1.2527531 | down | Pdcd6 |
| 6926505\|71529 | 0.036739744 | 0.001301894 | 1.2519644 | down | 9030409G11Rik |
| 6994589\|109229 | 0.03402511 | 0.001129147 | 1.2509396 | down | Fam118b\|Srpr |
| 6886244, 6894961\| | 0.027616503 | 7.61E−04 | 1.2506616 | up | Lrp1b\|Ran///Lrp1b\|4631405J |
| 94217 | | | | | 19Rik |
| 6825888\|16432 | 0.0354481 | 0.001228708 | 1.2497038 | up | Itm2b |
| 6806831\|218215 | 0.04793611 | 0.00218734 | 1.2494488 | up | Rnf144b |
| 6849525, 6854541\| | 0.03324496 | 0.001072871 | 1.2488078 | down | Anks1///Nudt3\|Anks1 |
| 224650 | | | | | |
| 6908149\|66921 | 0.034624055 | 0.001175803 | 1.2484398 | up | Prpf38b |
| 6860133\|70791 | 0.04873302 | 0.002285377 | 1.2480017 | down | Hars2 |
| 6850552\|83965 | 0.04799745 | 0.002207012 | 1.247138 | up | Enpp5 |
| 6839957\|78408 | 0.026502775 | 7.14E−04 | 1 2471005 | down | Fam131a |
| 6847324, 6850940\| | 0.04811016 | 0.002220749 | 1.2462183 | down | Btg3\|Gm7334 |
| 12228 | | | | | |
| 6847324, 6850940\| | 0.04811016 | 0.002220749 | 1.2462183 | down | Btg3\|Gm7334 |
| 654432 | | | | | |
| 6943067\|74132 | 0.032513015 | 0.001013204 | 1.2455171 | down | Rnf6 |
| 6805241, 6805355\| | 0.04533531 | 0.001976078 | 1.245417 | down | Hist1h4b\|Hist1h4j\|Hist1h4k\| |
| 69386 | | | | | Gm11275///Hist1h4b |
| 6816124, 6838415, | 0.038419306 | 0.001399227 | 1.2448874 | up | Il31ra\|Tuba1b\|Gm5620///Tub |
| 6838417\|434428 | | | | | a1b\|Gm6682\|Gm5620///Tuba |
| | | | | | 1a\|Gm6682\|Gm5620 |
| 6970442\|67150 | 0.048592288 | 0.002268528 | 1.2442167 | down | Rnf141 |
| 6805252, 6805385, | 0.03864976 | 0.001418489 | 1.2436203 | down | Gm11275\|Hist1h4a\|Hist1h4b\| |
| 6811537, 6811564, | | | | | Hist1h4f\|Hist1h4i\|Hist1h4m// |
| 6811678, 6811692, | | | | | /Hist1h4a\|Hist1h4b\|Hist1h4c\| |
| 6811701\|319157 | | | | | Hist1h4f\|Hist1h4m///Hist1h4i |
| | | | | | \|Hist1h4f\|Hist1h4m\|Gm11275 |
| | | | | | ///Hist1h4a\|Hist1h4b\|Hist1h4 |
| | | | | | c\|Hist1h4f\|Hist1h4m\|Gm1127 |
| | | | | | 5///Hist1h4a\|Hist1h4c\|Hist1h |
| | | | | | 4f///Hist1h4a\|Hist1h4b\|Hist1h |
| | | | | | 4f\|Hist1h4m |
| 6898063\|16497 | 0.027723162 | 7.78E−04 | 1.2418925 | down | Kcnab1 |
| 6903454\|13123 | 0.043608375 | 0.001815099 | 1.2418736 | up | Cyp7b1 |
| 6867650\|19045 | 0.023736937 | 5.72E−04 | 1.2415985 | down | Ppp1ca |
| 6965901\|232975 | 0.026686419 | 7.32E−04 | 1.241252 | up | Atp1a3 |
| 6968647\|67308 | 0.04337046 | 0.001792819 | 1.2392359 | down | Mrpl46 |
| 6942655\|19085 | 0.04811016 | 0.002225725 | 1.2391682 | up | Prkar1b\|9330169B04Rik |
| 6942655\|319999 | 0.04811016 | 0.002225725 | 1.2391682 | up | Prkar1b\|9330169B04Rik |
| 6782454\|18738 | 0.04368416 | 0.001824396 | 1.2384719 | down | Pitpna |
| 6883526\|109054 | 0.042509187 | 0.003680835 | 1.2382039 | down | Pfdn4\|Cyp24a1 |
| 6919195\|140500 | 0.036743402 | 0.001304607 | 1.2380875 | down | Acap3 |
| 6777305\|64050 | 0.02606873 | 6.95E−04 | 1.2364374 | down | Yeats4 |
| 6819425\|67840 | 0.03992887 | 0.001491343 | 1.2361919 | down | Mrp63 |
| 6805252, 6805385, | 0.043608375 | 0.001812688 | 1.2355359 | down | Gm11275\|Hist1h4a\|Hist1h4b\| |
| 6811528, 6811537, | | | | | Hist1h4f\|Hist1h4i\|Hist1h4m// |
| 6811678, 6811692, | | | | | /Hist3h4a\|Hist1h4b\|Hist1h4c\| |
| 6811701\|326619 | | | | | Hist1h4f\|Hist1h4m///Hist1h4 |
| | | | | | a\|Hist1h4b\|Hist1h4j\|Hist1h4k\| |
| | | | | | Hist1h4m///Hist1h4a\|Hist1h4 |
| | | | | | b\|Hist1h4c\|Hist1h4f\|Hist1h4 |
| | | | | | m\|Gm11275///Hist1h4a\|Hist1 |
| | | | | | h4c\|Hist1h4f///Hist1h4a\|Hist1 |
| | | | | | h4b\|Hist1h4f\|Hist1h4m |
| 6975209\|75029 | 0.035077687 | 0.001196139 | 1.2351745 | down | Purg |
| 6805252, 6811537, | 0.035202216 | 0.001205335 | 1.2343416 | down | Gm11275\|Hist1b4a\|Hist1h4b\| |
| 6811564\|319158 | | | | | Hist1h4i\|Hist1h4i\|Hist1h4m// |
| | | | | | /Hist1h4i\|Hist1h4f\|Hist1h4m\| |
| | | | | | Gm11275 |
| 6973683\|140482 | 0.04048394 | 0.001551273 | 1.234045 | up | Zfp358 |
| 6998583\|109652 | 0.043834306 | 0.001861487 | 1.2332873 | down | Acy1 |
| 6867748\|69860 | 0.0393026 | 0.001447976 | 1.2314234 | down | Eif1ad\|Sart1 |
| 6864330\|67453 | 0.046591923 | 0.00206704 | 1.2267478 | down | Slc25a46 |
| 6849973\|66416 | 0.030015303 | 8.88E−04 | 1.2261399 | down | Ndufa7 |
| 6837428\|109754 | 0.049655594 | 0.002356572 | 1.2259744 | up | Cyb5r3 |
| 6767460\|54198 | 0.0332504 | 0.001090932 | 1.2253067 | down | Snx3 |
| 6789483\|103712 | 0.044442587 | 0.001914789 | 1.2245939 | up | 6330403K07Rik |
| 6876310\|227743 | 0.047316674 | 0.002145768 | 1.2222756 | down | Mapkap1\|5830434F19Rik\|49 |
| | | | | | 30414H07Rik |
| 6876310\|76034 | 0.047316674 | 0.002145768 | 1.2222756 | down | Mapkap1\|5830434F19Rik\|49 |
| | | | | | 30414H07Rik |
| 6876310\|73869 | 0.047316674 | 0.002145768 | 1.2222756 | down | Mapkap1\|5830434F19Rik\|49 |
| | | | | | 30414H07Rik |
| 6860049\|56550 | 0.04629016 | 0.002047143 | 1.222038 | down | Ube2d2 |
| 6917283\|107271 | 0.042509187 | 0.001680853 | 1.2182815 | down | Yars |
| 6951756\|101148 | 0.04815002 | 0.002235392 | 1.2171097 | down | B630005N14Rik |
| 7012681\|17698 | 0.049823217 | 0.00236803 | 1.2154173 | up | Msn |
| 6833184\|83797 | 0.04026438 | 0.001519246 | 1.2153908 | down | Smarcd1 |
| 6839932\|11773 | 0.035368353 | 0.00122097 | 1.2150815 | down | Ap2m1 |
| 6762234\|21367 | 0.045408387 | 0.001985799 | 1.2126511 | up | Cntn2 |
| 6853197\|76781 | 0.04337046 | 0.001784526 | 1.210731 | down | Mettl4 |
| 6764138\|98660 | 0.04533531 | 0.00197496 | 1.2105742 | up | Atp1a2 |
| 6794073\|380752 | 0.045719497 | 0.002007306 | 1.2088413 | down | Tssc1 |
| 6965153\|330671 | 0.043053027 | 0.001731272 | 1.2070053 | up | B4galnt4 |
| 6749572\|19070 | 0.046761967 | 0.002081159 | 1.2048521 | down | Mobkl3 |
| 6947596\|21802 | 0.0469654 | 0.0021037 | 1.2038522 | down | Tgfa |

TABLE 3
Genes with significant changes (Benjamini-Hochberg
adjusted p-values <0.05) of at least 1.2-fold up or down in Drd1a-expressing
cortical neurons at 2 years and 6 weeks of age, as compared to 6 weeks of age.

| | p value | | Fold change | | |
|---|---|---|---|---|---|
| Gene_ID | (corrected) | p value | (absolute) | Regulation | Gene symbol |
| 7023132\|236604 | 1.30E−04 | 1.92E−08 | 3.5778549 | up | Pisd-ps3\|Pisd-ps1 |
| 6845079\|11815 | 0.047086563 | 2.74E−04 | 2.9835136 | up | Apod |
| 6998397\|22041 | 0.002609346 | 1.33E−06 | 2.6085126 | up | Trf |
| 6972168\|66141 | 0.002609346 | 1.85E−06 | 2.5289943 | up | Ifitm3 |
| 6937190, 7023132\|320951 | 1.30E−04 | 2.74E−08 | 2.4953797 | up | Pisd\|Pisd-ps3///Pisd-ps3\|Pisd- |
| | | | | | ps1 |
| 6937190, 7023132\|66776 | 1.30E−04 | 2.74E−08 | 2.4953797 | up | Pisd\|Pisd-ps3///Pisd-ps3\|Pisd- |
| | | | | | ps1 |
| 6791465\|19183 | 0.03733478 | 1.49E−04 | 2.4761345 | up | Psmc3ip |
| 6784526\|17896 | 0.032506455 | 6.81E−05 | 2.2456028 | down | Myl4\|Lin52\|Gm7020 |
| 6817978\|21924 | 0.032506455 | 6.86E−05 | 2.1837645 | down | Tnnc1 |
| 6823429\|66039 | 0.03972272 | 1.65E−04 | 2.117844 | down | D14Ertd449e |
| 6784526, 6796606\|217708 | 0.028130708 | 5.04E−05 | 2.056382 | down | Myl4\|Lin52\|Gm7020///Lin52\| |
| | | | | | Gm7020 |
| 6784526, 6796606\|629959 | 0.028130708 | 5.04E−05 | 2.056382 | down | Myl4\|Lin52\|Gm7020///Lin52\| |
| | | | | | Gm7020 |
| 6862827\|12405 | 0.047086563 | 2.66E−04 | 2.0361567 | down | Cbln2 |
| 6899683\|13040 | 0.003809435 | 3.48E−06 | 1.9884881 | up | Ctss |
| 6782484\|74230 | 0.004386073 | 4.63E−06 | 1.9319992 | down | 1700016K19Rik |
| 6959584\|22177 | 0.040753897 | 1.83E−04 | 1.9228017 | up | Tyrobp |
| 6753402\|21956 | 0.004028092 | 3.96E−06 | 1.8955778 | up | Tnnt2 |
| 6983999\|12404 | 0.047086563 | 2.98E−04 | 1.8022048 | down | Cbln1 |
| 6869068\|77125 | 0.028130708 | 5.05E−05 | 1.7979872 | up | Il33 |
| 6995918\|235416 | 0.002609346 | 1.30E−06 | 1.7442707 | down | Lman11\|Cplx3 |
| 6995918\|235415 | 0.002609346 | 1.30E−06 | 1.7442707 | down | Lman11\|Cplx3 |
| 6768261, 6876138\|432466 | 0.002609346 | 2.00E−06 | 1.6745123 | up | Gm5424\|Ass1///Ass1\|Gm5424 |
| 6768261, 6876138\|11898 | 0.002609346 | 2.00E−06 | 1.6745123 | up | Gm5424\|Ass1///Ass1\|Gm5424 |
| 6988976\|13489 | 0.03615578 | 1.37E−04 | 1.6427418 | up | Drd2 |
| 6957352\|232400 | 0.040753897 | 1.83E−04 | 1.6084235 | down | BC048546 |
| 6967593\|110886 | 0.042847566 | 2.11E−04 | 1.5844014 | down | Gabra5 |
| 6945335\|109624 | 0.028130708 | 5.14E−05 | 1.5766602 | up | Cald1 |
| 6747478\|76982 | 0.035836473 | 1.04E−04 | 1.5673733 | down | 3110035E14Rik |
| 6992215\|56808 | 0.035454802 | 9.40E−05 | 1.5502318 | up | Cacna2d2 |
| 6993890\|68743 | 0.005008051 | 5.63E−06 | 1.5448154 | up | Anln |
| 6900928\|66789 | 0.019637536 | 2.35E−05 | 1.5300947 | down | Alg14 |
| 7016409\|245386 | 0.03400331 | 7.41E−05 | 1.5258399 | up | Fam70a\|Zbtb33 |
| 6954385\|13197 | 0.035454802 | 8.96E−05 | 1.5098312 | down | Gadd45a\|Gng12 |
| 6864456\|27528 | 0.040753897 | 1.77E−04 | 1.5033208 | down | D0H4S114 |
| 6836358\|17988 | 0.028130708 | 4.14E−05 | 1.4998 | up | Ndrg1 |
| 6811068\|56048 | 0.035836473 | 1.21E−04 | 1.4984856 | up | Lgals8 |
| 6869570\|74055 | 0.047086563 | 2.79E−04 | 1.4937183 | up | Plce1 |
| 6883533\|76829 | 0.040753897 | 1.79E−04 | 1.4935141 | down | Dok5 |
| 6872916\|15925 | 0.028428873 | 5.41E−05 | 1.4907689 | down | Ide |
| 6764721\|12334 | 0.03733478 | 1.48E−04 | 1.4689611 | up | Capn2 |
| 6769343, 6773537, 6968533\|624784 | 0.002609346 | 2.02E−06 | 1.4675822 | down | Tdg\|Gm9855\|Gm5806 |
| 6769343, 6773537, 6968533\|545124 | 0.002609346 | 2.02E−06 | 1.4675822 | down | Tdg\|Gm9855\|Gm5806 |
| 6748020\|14859 | 0.047086563 | 3.00E−04 | 1.4651384 | up | Gsta3 |
| 6752222\|241201 | 0.03615578 | 1.34E−04 | 1.4480729 | up | Cdh7 |
| 7010762, 7016409\|56805 | 0.035454802 | 9.28E−05 | 1.4420869 | up | Zbtb33///Fam70a\|Zbtb33 |
| 6946785, 6954385\|14701 | 0.035454802 | 9.47E−05 | 1.4353529 | down | Gng12///Gadd45a\|Gng12 |
| 6769343, 6773537, 6775518, 6968533\| | 0.0037269 | 3.34E−06 | 1.4327823 | down | Tdg\|Gm9855\|Gm5806///Glt8d2\| |
| 21665 | | | | | Tdg |
| 6783321\|18952 | 0.035836473 | 1.13E−04 | 1.4307998 | up | Sept4\|LOC100503535 |
| 6783321\|100503535 | 0.035836473 | 1.13E−04 | 1.4307998 | up | Sept4\|LOC100503535 |
| 6758223\|66297 | 0.043042764 | 2.19E−04 | 1.4281554 | down | 2610017I09Rik |
| 6755559\|68226 | 0.043042764 | 2.24E−04 | 1.4218508 | down | Efcab2 |
| 6822729\|54713 | 0.040753897 | 1.79E−04 | 1.4151258 | down | Fezf2 |
| 6886678\|74194 | 0.043677434 | 2.33E−04 | 1.4130437 | down | Rnd3 |
| 6766409\|52906 | 0.035454802 | 9.42E−05 | 1.4032575 | up | Ahi1 |
| 6782702\|22370 | 0.035836473 | 1.27E−04 | 1.4028069 | up | Vtn |
| 6913901\|72479 | 0.035454802 | 8.00E−05 | 1.3892306 | up | Hsdl2 |
| 6909375\|66357 | 0.02716284 | 3.63E−05 | 1.3778921 | down | Ostc |
| 6923525\|74519 | 0.028428873 | 5.60E−05 | 1.3728458 | up | Cyp2j9 |
| 6912245, 6920276\|14348 | 0.03615578 | 1.37E−04 | 1.3615227 | down | Fut9 |
| 6931790\|57357 | 0.047086563 | 2.69E−04 | 1.359849 | down | Srd5a3 |
| 6872646\|54447 | 0.035836473 | 3.12E−04 | 1.3592392 | up | Asah2 |
| 6862102\|52538 | 0.03733478 | 1.52E−04 | 1.3579103 | up | Acaa2 |
| 6959459\|51798 | 0.03733478 | 1.52E−04 | 1.355676 | up | Ech1 |
| 6816413\|18115 | 0.024438534 | 3.09E−05 | 1.3556129 | up | Nnt |
| 6995076\|71732 | 0.04191086 | 2.03E−04 | 1.3523128 | up | Vps11 |
| 6977260\|15368 | 0.04191086 | 2.01E−04 | 1.343893 | up | Hmox1 |
| 6959265\|13086 | 0.049245864 | 3.25E−04 | 1.3420637 | up | Cyp2a4\|Cyp2a5 |
| 6959265\|13087 | 0.049245864 | 3.25E−04 | 1.3420637 | up | Cyp2a4\|Cyp2a5 |
| 6796053\|238266 | 0.028130708 | 4.59E−05 | 1.3297588 | down | Syt16 |
| 6823849\|26419 | 0.035836473 | 1.29E−04 | 1.3211268 | down | Mapk8 |
| 6789475\|216877 | 0.028130708 | 4.66E−05 | 1.3187447 | up | Dhx33 |
| 6767387\|53599 | 0.04191086 | 2.02E−04 | 1.3120232 | down | Cd164 |
| 6974490\|52123 | 0.043677434 | 2.33E−04 | 1.3106182 | down | Agpat5 |
| 6761964\|72160 | 0.047086563 | 2.94E−04 | 1.2954209 | down | Tmem163\|Mgat5 |
| 6853910\|72057 | 0.035836473 | 1.25E−04 | 1.294048 | down | Phf10\|1600012H06Rik\|LOC106740 |
| 6853910\|106740 | 0.035836473 | 1.25E−04 | 1.294048 | down | Phf10\|1600012H06Rik\|LOC106740 |
| 6750547\|227292 | 0.041231222 | 1.91E−04 | 1.2933345 | up | Ctdsp1 |
| 6864326\|19762 | 0.035454802 | 9.20E−05 | 1.2931432 | up | Rit2 |
| 6836298, 6849523\|20630 | 0.03548006 | 9.73E−05 | 1.2924098 | down | Snrpc |
| 6924832\|12795 | 0.035836473 | 1.25E−04 | 1.283983 | down | Plk3 |
| 6922649\|66928 | 0.047086563 | 2.68E−04 | 1.2812592 | down | 3110001D03Rik\|LOC280487 |
| 6922649\|280487 | 0.047086563 | 2.68E−04 | 1.2832592 | down | 3110001D03Rik\|LOC280487 |
| 6825445\|19229 | 0.043042764 | 2.19E−04 | 1.2802857 | up | Ptk2b |
| 6941685\|11669 | 0.047086563 | 2.91E−04 | 1.2797663 | up | Aldh2 |
| 6991261\|19417 | 0.035836473 | 1.28E−04 | 1.2791666 | up | Rasgrf1 |
| 6998987\|74100 | 0.047086563 | 2.94E−04 | 1.2663616 | down | Arpp21\|Mir128-2 |
| 6998987\|723815 | 0.047086563 | 2.94E−04 | 1.2663616 | down | Arpp21\|Mir128-2 |
| 6925587\|66264 | 0.047666077 | 3.08E−04 | 1.2616228 | down | Ccdc28b\|2510006D16Rik |
| 6760754\|16560 | 0.035836473 | 1.01E−04 | 1.2583791 | up | Kif1a |
| 6791015\|18604 | 0.040834192 | 1.87E−04 | 1.2575148 | up | Pdk2 |
| 6783029\|70439 | 0.047086563 | 3.01E−04 | 1.2506527 | down | Taf15 |
| 6988962\|26951 | 0.047086563 | 2.80E−04 | 1.2481672 | up | Zw10 |
| 6848806, 6853910\|67912 | 0.043042764 | 2.21E−04 | 1.2481549 | down | 1600012H06Rik///Phf10\|1600012H06Rik\| |
| | | | | | LOC106740 |
| 7010345\|236733 | 0.047086563 | 2.90E−04 | 1.2121428 | up | Usp11 |

TABLE 4
Genes with significant changes (Benjamini-Hochberg
adjusted p-values <0.05) of at least 1.2-fold up or down in Drd2-expressing
cortical neurons at 2 years and 6 weeks of age, as compared to 6 weeks of age.

| | p value | | Fold change | | |
|---|---|---|---|---|---|
| Gene_ID | (corrected) | p value | (absolute) | Regulation | Gene symbol |
| 6998397\|22041 | 0.015955767 | 1.91E−05 | 4.5832944 | up | Trf |
| 6813284\|13488 | 0.008502543 | 5.38E−06 | 3.3188136 | up | Drd1a |
| 6845079\|11815 | 0.010140898 | 8.28E−06 | 2.5178335 | up | Apod |
| 7023132\|236604 | 0.003949368 | 5.46E−07 | 2.387355 | up | Pisd-ps3\|Pisd-ps1 |
| 6776577\|67405 | 0.003949368 | 1.20E−06 | 2.2947934 | down | Nts |
| 6817978\|21924 | 0.04098089 | 2.38E−04 | 2.2747989 | down | Tnnc1 |
| 6754149, 6861135\|14645 | 0.01982543 | 3.35E−05 | 2.2733164 | up | Glul///Gramd3\|Glul |
| 6877356, 6886947\|77767 | 0.032421894 | 1.41E−04 | 2.2179747 | up | Galnt5\|Ermn///Ermn |
| 6908075, 6908077, 6908078\|14864 | 0.048785735 | 4.86E−04 | 2.0753336 | up | Gstm6\|Gstm3///Gstm3///Gstm1\| |
| | | | | | Gstm3 |
| 6791494\|73635 | 0.030455668 | 1.11E−04 | 2.0665221 | down | Rundc1\|1700113I22Rik\|Aarsd1 |
| 6937190, 7023132\|320951 | 0.003949368 | 1.39E−06 | 2.0144777 | up | Pisd\|Pisd-ps3///Pisd-ps3\|Pisd- |
| | | | | | ps1 |
| 6937190, 7023132\|66776 | 0.003949368 | 1.39E−06 | 2.0144777 | up | Pisd\|Pisd-ps3///Pisd-ps3\|Pisd- |
| | | | | | ps1 |
| 6943974\|21333 | 0.030455668 | 1.07E−04 | 1.9952383 | up | Tac1 |
| 7013389\|237010 | 0.042421777 | 3.34E−04 | 1.8928168 | up | Klhl4 |
| 6993890\|68743 | 0.032484267 | 1.46E−04 | 1.884146 | up | Anln |
| 6898477\|20713 | 0.03124751 | 1.34E−04 | 1.8833878 | up | Serpini1 |
| 6880670\|12010 | 0.041485418 | 2.56E−04 | 1.875716 | up | B2m |
| 6944262\|114142 | 0.003949368 | 6.80E−07 | 1.8566908 | up | Foxp2 |
| 6748020\|14859 | 0.032484267 | 1.45E−04 | 1.8344905 | up | Gsta3 |
| 6811068\|56048 | 0.042421777 | 3.38E−04 | 1.8010027 | up | Lgals8 |
| 6997555\|382090 | 0.01285925 | 1.36E−05 | 1.7201661 | up | 4922501C03Rik |
| 6904297\|11747 | 0.030455668 | 1.20E−04 | 1.6902814 | up | Anxa5 |
| 6862062\|71263 | 0.043850936 | 3.92E−04 | 1.671545 | down | Mro |
| 6838811, 6917301\|17357 | 0.042421777 | 3.05E−04 | 1.6639088 | down | Marcksl1\|BC048502///Marcksl1 |
| 6964527\|56213 | 0.045647837 | 4.30E−04 | 1.6607143 | up | Htra1 |
| 6788025\|216724 | 0.022754725 | 5.07E−05 | 1.6575161 | up | Rufy1 |
| 6973587\|11816 | 0.010140898 | 9.98E−06 | 1.6567526 | up | Apoe |
| 6899520\|20194 | 0.03124751 | 1.33E−04 | 1.6538708 | up | S100a10 |
| 6878655\|16410 | 0.039288376 | 2.13E−04 | 1.64845 | up | Itgav |
| 6834890\|56274 | 0.042421777 | 2.95E−04 | 1.6482366 | up | Stk3 |
| 6836991\|12300 | 0.030455668 | 1.19E−04 | 1.6434618 | down | Cacng2 |
| 6989222\|12903 | 0.008459655 | 4.76E−06 | 1.639584 | down | Crabp1 |
| 6971344\|66422 | 0.0259438 | 6.93E−05 | 1.6349066 | down | Dctpp1 |
| 6884986\|74103 | 0.046546645 | 4.49E−04 | 1.6318291 | down | Nebl |
| 6797969\|17263 | 0.022754725 | 5.76E−05 | 1.6306723 | down | Meg3\|Dlk1\|Mir1906 |
| 6797969\|100316809 | 0.022754725 | 5.76E−05 | 1.6306723 | down | Meg3\|Dlk1\|Mir1906 |
| 6764138\|98660 | 0.048785735 | 4.85E−04 | 1.6294948 | up | Atp1a2 |
| 6899747, 6907247\|15267 | 0.010140898 | 9.80E−06 | 1.6212646 | down | Hist2h2aa1\|Hist2h2aa2\|Hist2h2ac\| |
| | | | | | Hist2h3c1///Hist2h2aa1\|Hist2h2aa2\| |
| | | | | | Hist2h3c1 |
| 6899747, 6907247\|319192 | 0.010140898 | 9.80E−06 | 1.6212646 | down | Hist2h2aa1\|Hist2h2aa2\|Hist2h2ac\| |
| | | | | | Hist2h3c1///Hist2h2aa1\|Hist2h2aa2\| |
| | | | | | Hist2h3c1 |
| 6961010\|17984 | 0.00482493 | 2.04E−06 | 1.6177676 | up | Ndn |
| 6926936\|110208 | 0.042801354 | 3.71E−04 | 1.6117427 | up | Pgd |
| 6861751\|52662 | 0.033511773 | 1.53E−04 | 1.6040033 | down | D18Ertd653e |
| 6823068\|11750 | 0.016177624 | 2.05E−05 | 1.5961775 | up | Anxa7 |
| 6913009, 6921154\|12517 | 0.042801354 | 3.69E−04 | 1.5803419 | down | Tesk1\|Cd72///Cd72 |
| 6885395\|68475 | 0.02753681 | 7.94E−05 | 1.5702732 | down | Ssna1 |
| 6972710\|57776 | 0.04098089 | 2.43E−04 | 1.5661737 | down | Ttyh1 |
| 7000764\|77226 | 0.030455668 | 1.24E−04 | 1.565689 | down | 9330169L03Rik |
| 6961650, 6968387\|100038347 | 0.017847234 | 2.71E−05 | 1.564204 | down | Fam174b |
| 6753402\|21956 | 0.00988544 | 6.95E−06 | 1.5561305 | up | Tnnt2 |
| 6872646\|54447 | 0.008459655 | 4.26E−06 | 1.5533785 | up | Asah2 |
| 6988194\|66279 | 0.044974487 | 4.15E−04 | 1.5444175 | down | Tmem218 |
| 6973472\|243833 | 0.018898552 | 3.06E−05 | 1.5440156 | up | Zfp128 |
| 6824507\|67419 | 0.042421777 | 2.94E−04 | 1.5365113 | up | 3632451O06Rik |
| 6883013\|228858 | 0.042421777 | 3.33E−04 | 1.5346153 | up | Gdap111 |
| 6779845\|327900 | 0.022754725 | 5.74E−05 | 1.5159067 | down | Ubtd2 |
| 6762321\|381290 | 0.042421777 | 3.39E−04 | 1.5038337 | up | Atp2b4 |
| 6964250\|68952 | 0.030455668 | 1.26E−04 | 1.5031539 | down | Fam57b |
| 6962751\|381903 | 0.04098089 | 2.44E−04 | 1.4986535 | down | Alg8 |
| 6805360\|319181 | 0.02733521 | 7.69E−05 | 1.4921204 | down | Hist1h2bg |
| 6848581\|106489 | 0.038548224 | 2.03E−04 | 1.4837484 | down | Sft2d1\|T2\|Gm12166 |
| 6848581\|100039624 | 0.038548224 | 2.03E−04 | 1.4837484 | down | Sft2d1\|T2\|Gm12166 |
| 6872980\|19662 | 0.022754725 | 4.55E−05 | 1.4830675 | down | Rbp4 |
| 6983838\|101966 | 0.022754725 | 4.20E−05 | 1.4753313 | down | D8Ertd738e |
| 6768261, 6876138\|432466 | 0.022754725 | 5.64E−05 | 1.473841 | up | Gm5424\|Ass1///Ass1\|Gm5424 |
| 6768261, 6876138\|11898 | 0.022754725 | 5.64E−05 | 1.473841 | up | Gm5424\|Ass1///Ass1\|Gm5424 |
| 6840887\|207683 | 0.042421777 | 3.47E−04 | 1.4678116 | down | Igsfl1 |
| 6797969, 6797978\|13386 | 0.0259438 | 6.84E−05 | 1.4611462 | down | Meg3\|Dlk1\|Mir1906///Dlk1 |
| 6852887\|17685 | 0.04284105 | 3.77E−04 | 1.4595807 | up | Msh2 |
| 6791995\|71795 | 0.038548224 | 1.89E−04 | 1.4416575 | down | Pitpnc1 |
| 6803780\|67236 | 0.042421777 | 3.14E−04 | 1.4332547 | down | Cinp |
| 6752571\|70829 | 0.022754725 | 5.59E−05 | 1.4327077 | up | Ccdc93 |
| 6877822\|26877 | 0.043799955 | 3.88E−04 | 1.425908 | down | B3galt1 |
| 6750351\|108147 | 0.042421777 | 2.98E−04 | 1.4252509 | up | Atic |
| 6883267\|110750 | 0.021531083 | 3.78E−05 | 1.4230214 | up | Cse11 |
| 6886678\|74194 | 0.030455668 | 1.22E−04 | 1.4227502 | down | Rnd3 |
| 6909629\|67006 | 0.014083494 | 1.58E−05 | 1.4220327 | down | Cisd2 |
| 6788815\|11671 | 0.030455668 | 9.64E−05 | 1.4178725 | up | Aldh3a2 |
| 6954385\|13197 | 0.038548224 | 1.87E−04 | 1.4168825 | down | Gadd45a\|Gng12 |
| 6769445\|216198 | 0.042421777 | 3.55E−04 | 1.4158584 | up | Tcp11l2 |
| 6798418\|217944 | 0.022754725 | 4.49E−05 | 1.4127954 | up | Rapgef5 |
| 6796305\|56217 | 0.038548224 | 1.91E−04 | 1.4115229 | up | Mpp5 |
| 6848581, 6848584\|21331 | 0.03972801 | 2.18E−04 | 1.4081773 | down | Sft2d1\|T2\|Gm12166///T2 |
| 6902665\|209601 | 0.042226546 | 2.71E−04 | 1.4075357 | up | 4922501L14Rik |
| 6751538\|67921 | 0.038548224 | 1.85E−04 | 1.4075081 | down | Ube2f\|Gm5434 |
| 6916947\|170638 | 0.030455668 | 1.10E−04 | 1.4067643 | up | Hpcal4 |
| 6937047\|67695 | 0.030455668 | 1.18E−04 | 1.4062134 | down | Ost4\|Agbl5 |
| 6972970\|319748 | 0.017847234 | 2.76E−05 | 1.4012991 | down | Zfp865\|Zfp784\|4632433K11Rik |
| 6972970\|654801 | 0.017847234 | 2.76E−05 | 1.4012991 | down | Zfp865\|Zfp784\|4632433K11Rik |
| 6972970\|77043 | 0.017847234 | 2.76E−05 | 1.4012991 | down | Zfp865\|Zfp784\|4632433K11Rik |
| 6853388\|70544 | 0.042226546 | 2.67E−04 | 1.400874 | down | 5730437N04Rik |
| 6985850\|68918 | 0.030455668 | 1.26E−04 | 1.3967563 | down | 1190005I06Rik |
| 6896584\|67576 | 0.042421777 | 2.85E−04 | 1.3956859 | down | 4930429B21Rik\|Zmat3 |
| 6899747, 6899750, 6899752, 6907246, | 0.049234077 | 5.02E−04 | 1.3945229 | down | Hist2h2aa1\|Hist2h2aa2\|Hist2h2ac\| |
| 6907247\|15077 | | | | | Hist2h3c1///Hist2h3c1\|Hist2h3c2- |
| | | | | | ps///Hist2h3b\|Hist2h3c1\|Hist2h3c2- |
| | | | | | ps///Hist2h3c1\|Hist2h3c2- |
| | | | | | ps\|Hist2h3b///Hist2h2aa1\|Hist2h2aa2\| |
| | | | | | Hist2h3c1 |
| 6912947\|108816 | 0.045647837 | 4.36E−04 | 1.3941127 | down | 4933409K07Rik\|Gm3893\|Gm7819 |
| 6912947\|100042539 | 0.045647837 | 4.36E−04 | 1.3941127 | down | 4933409K07Rik\|Gm3893\|Gm7819 |
| 6912947\|665845 | 0.045647837 | 4.36E−04 | 1.3941127 | down | 4933409K07Rik\|Gm3893\|Gm7819 |
| 6890638\|320961 | 0.042801354 | 3.66E−04 | 1.392631 | down | Gabpb1\|A630026N12Rik |
| 6929651, 6937047\|231093 | 0.030455668 | 1.21E−04 | 1.388592 | down | Agbl5///Ost4\|Agbl5 |
| 6805380\|319178 | 0.022754725 | 4.72E−05 | 1.3881029 | down | Hist1h2bb |
| 6782277\|55984 | 0.039288376 | 2.12E−04 | 1.3869212 | up | Camkk1 |
| 6918382, 6918560\|100503000 | 0.042421777 | 3.53E−04 | 1.3848228 | up | Gm13051\|Zfp534\|1700029I01Rik\| |
| | | | | | Gm13251\|Zfp600\|Gm13242\| |
| | | | | | Rex2\|Gm13138\|Gm13139\| |
| | | | | | Gm13225\|Gm13151\|Gm13235\| |
| | | | | | Gm13212\|LOC100503000/// |
| | | | | | 1700029I01Rik\|Gm13251\|Zfp534\| |
| | | | | | Gm13139\|Gm13151\|2610305D13Rik\| |
| | | | | | LOC100503000 |
| 6839934\|27406 | 0.030455668 | 1.20E−04 | 1.379296 | up | Abcf3 |
| 6996440\|235442 | 0.041485418 | 2.57E−04 | 1.3785135 | up | Rab8b |
| 6777309, 6777310\|17105 | 0.048785735 | 4.90E−04 | 1.3751312 | up | Lyz2\|Lyz1///Lyz1\|Lyz2 |
| 6777309, 6777310\|17110 | 0.048785735 | 4.90E−04 | 1.3751312 | up | Lyz2\|Lyz1///Lyz1\|Lyz2 |
| 6896584, 6904047\|22401 | 0.030455668 | 1.20E−04 | 1.3749123 | down | 4930429B21Rik\|Zmat3///Zmat3 |
| 6900404\|99730 | 0.040309925 | 2.27E−04 | 1.3731047 | down | Tafl3 |
| 6917217\|242667 | 0.042226546 | 2.73E−04 | 1.3728224 | down | Dlgap3 |
| 6882768\|228852 | 0.044817124 | 4.10E−04 | 1.3703306 | down | Ppp1r16b |
| 6751538, 6794491\|432649 | 0.04098089 | 2.45E−04 | 1.3591425 | down | Ube2f\|Gm5434///Gm5434 |
| 6833308\|56149 | 0.042421777 | 3.23E−04 | 1.3591031 | down | Grasp |
| 6797707\|73046 | 0.044817124 | 4.08E−04 | 1.3571836 | down | Glrx5 |
| 6949826\|30853 | 0.040628925 | 2.31E−04 | 1.3515993 | down | Mlf2 |
| 6836699\|23936 | 0.042801354 | 3.66E−04 | 1.3503007 | down | Lynx1 |
| 6900239\|81600 | 0.030455668 | 1.15E−04 | 1.3502584 | up | Chia\|1810022K09Rik |
| 6900239\|69126 | 0.030455668 | 1.15E−04 | 1.3502584 | up | Chia\|1810022K09Rik |
| 6778425\|11764 | 0.049234077 | 5.09E−04 | 1.3486375 | up | Ap1b1 |
| 6758663\|70396 | 0.049234077 | 5.02E−04 | 1.3484918 | down | Asnsd1 |
| 6997077\|71538 | 0.038548224 | 1.93E−04 | 1.343926 | down | Fbxo9 |
| 6918382, 6918397, 6918560\|100043100 | 0.042421777 | 3.14E−04 | 1.3402557 | up | Gm13051\|Zfp534\|1700029I01Rik\| |
| | | | | | Gm13251\|Zfp600\|Gm13242\| |
| | | | | | Rex2\|Gm13138\|Gm13139\| |
| | | | | | Gm13225\|Gm13151\|Gm13235\| |
| | | | | | Gm13212\|LOC100503000/// |
| | | | | | Gm13157\|1700029I01Rik\|Gm13251\| |
| | | | | | Zfp534\|Rex2\|Gm13138\| |
| | | | | | Gm13212\|Gm13225\|Gm13151\| |
| | | | | | Gm13235\|Gm13154///1700029I01Rik\| |
| | | | | | Gm13251\|Zfp534\|Gm13139\| |
| | | | | | Gm13151\|2610305D13Rik\| |
| | | | | | LOC100503000 |
| 6970138\|55992 | 0.042421777 | 3.24E−04 | 1.3331729 | up | Trim3 |
| 6979144, 6985389\|170737 | 0.026766581 | 7.34E−05 | 1.3291724 | down | Znrf1///Ldhd\|Znrf1 |
| 6966328\|22282 | 0.042421777 | 2.89E−04 | 1.3234341 | down | Usf2 |
| 6933409\|71782 | 0.042421777 | 2.93E−04 | 1.3136151 | up | Ankle2 |
| 6770923\|327824 | 0.042421777 | 3.49E−04 | 1.3079915 | down | 5330438D12Rik\|LOC100504423 |
| 6770923\|100504423 | 0.042421777 | 3.49E−04 | 1.3079915 | down | 5330438D12Rik\|LOC100504423 |
| 6978855\|56513 | 0.046546645 | 4.52E−04 | 1.307966 | down | Pard6a |
| 6992219\|56289 | 0.038548224 | 2.02E−04 | 1.3071496 | down | Rassf1 |
| 6764048\|641376 | 0.030455668 | 1.04E−04 | 1.3021629 | down | Tomm40l |
| 6882521\|66734 | 0.030455668 | 1.14E−04 | 1.3008461 | down | Map11c3a |
| 6873158\|66583 | 0.044974487 | 4.17E−04 | 1.293808 | down | Exosc1 |
| 6842933\|74112 | 0.042421777 | 3.23E−04 | 1.2911134 | down | Usp16 |
| 6921030, 6921081, 7003070, 7003163\| | 0.04014609 | 2.23E−04 | 1.2910843 | down | Ccl27a///Gm13306\|Ccl27a///Zar1\| |
| 20301 | | | | | Ccl27a |
| 6921081, 7003163\|100039863 | 0.041485418 | 2.51E−04 | 1.2884337 | down | Gm13306\|Ccl27a |
| 6980271\|234023 | 0.042421777 | 3.41E−04 | 1.2866849 | down | Arglu1 |
| 6915929, 6915993\|13131 | 0.042421777 | 3.38E−04 | 1.2848579 | down | Dab1\|Gm10304\|2900034C19Rik\| |
| | | | | | AY512949\|LOC100502604 |
| | | | | | ///Dab1 |
| 6878995\|56428 | 0.042226546 | 2.68E−04 | 1.2837576 | down | Mtch2 |
| 6996704\|21406 | 0.042421777 | 3.02E−04 | 1.2834076 | up | Tcfl2 |
| 6775250\|28169 | 0.044817124 | 4.08E−04 | 1.2755362 | down | Agpat3 |
| 6963972\|59052 | 0.038548224 | 1.88E−04 | 1.2750655 | down | Mettl9 |
| 6941761\|207565 | 0.038548224 | 1.98E−04 | 1.268037 | down | Camkk2 |
| 6771546, 6777915\|14421 | 0.042421777 | 3.22E−04 | 1.2680281 | down | B4galnt1\|Slc26a10///Slc26a10\| |
| | | | | | B4galnt1 |
| 6771546, 6777915\|216441 | 0.042421777 | 3.22E−04 | 1.2680281 | down | B4galnt1\|Slc26a10///Slc26a10\| |
| | | | | | B4galnt1 |
| 6964241\|66162 | 0.042801354 | 3.73E−04 | 1.2623248 | down | Bola2 |
| 6913863\|72429 | 0.049234077 | 5.16E−04 | 1.2596972 | down | Dnajc25\|Gng10 |
| 6913863\|14700 | 0.049234077 | 5.16E−04 | 1.2596972 | down | Dnajc25\|Gng10 |
| 6893279\|18019 | 0.042421777 | 2.98E−04 | 1.2566174 | down | Nfatc2 |
| 6771538, 6777902\|12567 | 0.048785735 | 4.85E−04 | 1.2456908 | down | Cdk4///Tspan31\|Cdk4 |
| 6935486\|56443 | 0.049234077 | 5.15E−04 | 1.2079331 | up | Arpc1a |

TABLE 5
Enriched pathways from Wikipathways altered with age in
Drd1a-expressing striatal medium spiny neurons.

| | | | Total |
|---|---|---|---|
| | | Matched | Pathway |
| Pathway | p value | Entities | Entities |
| Mm_XPodNet_-_protein- | 2.85E−05 | 13 | 836 |
| protein_interactions_in_the_podocyte_expanded_by_STRING_WP2309_72004 | | | |
| Mm_Chemokine_signaling_pathway_WP2292_72463 | 9.62E−05 | 6 | 193 |
| Mm_PodNet-_protein-protein_interactions_in_the_podocyte_WP2310_72005 | 2.48E−04 | 7 | 315 |
| Mm_IL-7_Signaling_Pathway_WP297_69128 | 7.19E−04 | 3 | 44 |
| Mm_B_Cell_Receptor_Signaling_Pathway_WP274_67072 | 0.003729099 | 4 | 156 |
| Mm_G_Protein_Signaling_Pathways_WP232_71315 | 0.005608093 | 3 | 91 |
| Mm_Integrin-mediated_Cell_Adhesion_WP6_72138 | 0.006962605 | 3 | 101 |
| Mm_Striated_Muscle_Contraction_WP216_72052 | 0.012084 | 2 | 45 |
| Mm_MAPK_signaling_pathway_WP493_71754 | 0.024514528 | 3 | 159 |
| Mm_Purine_metabolism_WP2185_71316 | 0.02668426 | 3 | 178 |
| Mm_Primary_Focal_Segmental_Glomerulosclerosis_FSGS_WP2573_72201 | 0.02678989 | 2 | 73 |
| Mm_Kit_Receptor_Signaling_Pathway_WP407_69079 | 0.030982522 | 2 | 67 |
| Mm_IL-5_Signaling_Pathway_WP151_69175 | 0.032727577 | 2 | 69 |

TABLE 6
Enriched pathways from Wikipathways altered with age in Drd2-expressing
striatal medium spiny neurons.

| | | | Total |
|---|---|---|---|
| | | Matched | Pathway |
| Pathway | p value | Entities | Entities |
| Mm_XPodNet_-_protein- | 6.20E−10 | 51 | 836 |
| protein_interactions_in_the_podocyte_expanded_by_STRING_WP2309_72004 | | | |
| Mm_EGFR1_Signaling_Pathway_WP572_71756 | 3.89E−06 | 14 | 176 |
| Mm_PodNet-_protein-protein_interactions_in_the_podocyte_WP2310_72005 | 1.12E−05 | 18 | 315 |
| Mm_MAPK_signaling_pathway_WP493_71754 | 1.07E−04 | 11 | 159 |
| Mm_Myometrial_Relaxation_and_Contraction_Pathways_WP385_72108 | 1.43E−04 | 11 | 157 |
| Mm_Hypothetical_Network_for_Drug_Addiction_WP1246_69102 | 1.72E−04 | 5 | 32 |
| Mm_Calcium_Regulation_in_the_Cardiac_Cell_WP553_73390 | 3.56E−04 | 10 | 150 |
| Mm_IL-6_signaling_Pathway_WP387_72091 | 4.08E−04 | 8 | 99 |
| glutathione redox reactions I | 4.82E−04 | 3 | 9 |
| Mm_B_Cell_Receptor_Signaling_Pathway_WP274_67072 | 5.18E−04 | 10 | 156 |
| Mm_Primary_Focal_Segmental_Glomerulosclerosis_FSGS_WP2573_72201 | 9.00E−04 | 6 | 73 |
| glutathione-mediated detoxification | 9.06E−04 | 4 | 24 |
| Mm_IL-7_Signaling_Pathway_WP297_69128 | 0.001013867 | 5 | 44 |
| Mm_ErbB_signaling_pathway_WP1261_71282 | 0.001013867 | 5 | 46 |
| Mm_G_Protein_Signaling_Pathways_WP232_71315 | 0.001068723 | 7 | 91 |
| Mm_Estrogen_signalling_WP1244_73501 | 0.001363316 | 6 | 74 |
| Mm_Kit_Receptor_Signaling_Pathway_WP407_69079 | 0.001363316 | 6 | 67 |
| Mm_Amino_Acid_metabolism_WP662_71177 | 0.001488036 | 7 | 95 |
| Mm_MAPK_Cascade_WP251_71729 | 0.001646583 | 4 | 29 |
| Mm_Integrin-mediated_Cell_Adhesion_WP6_72138 | 0.001687754 | 7 | 101 |
| Mm_Insulin_Signaling_WP65_71726 | 0.001848077 | 9 | 159 |
| Mm_Splicing_factor_NOVA_regulated_synpatic proteins_WP1983_71717 | 0.002140725 | 4 | 42 |
| Mm_Cholesterol_Biosynthesis_WP103_71741 | 0.002402918 | 3 | 15 |
| gluconeogenesis I | 0.002917192 | 3 | 17 |
| GDP-mannose biosynthesis I | 0.003268231 | 2 | 6 |
| GDP-mannose biosynthesis | 0.003268231 | 2 | 6 |
| Mm_Oxidative_Damage_WP1496_75225 | 0.00380393 | 4 | 41 |
| Mm_Urea_cycle_and_metabolism_of_amino_groups_WP426_72149 | 0.004844879 | 3 | 37 |
| Mm_G1_to_S_cell_cycle_control_WP413_72012 | 0.004965064 | 5 | 62 |
| Mm_Selenium_Micronutrient_Network_WP1272_73551 | 0.005622589 | 3 | 31 |
| Mm_TGF-beta_Receptor_Signaling_Pathway_WP258_73847 | 0.006082267 | 8 | 150 |
| Mm_Eukaryotic_Transcription_Initiation_WP567_69915 | 0.006176465 | 4 | 41 |
| Mm_Folic_Acid_Network_WP1273_74467 | 0.006470848 | 3 | 27 |
| Mm_Tryptophan_metabolism_WP79_73389 | 0.007349561 | 4 | 44 |
| fatty acid Beta-oxidation I | 0.007391186 | 3 | 24 |
| Mm_Wnt_Signaling_Pathway_and_Pluripotency_WP723_69165 | 0.007550718 | 6 | 97 |
| spermine biosynthesis II | 0.008820865 | 2 | 8 |
| superpathway of D-myo-inositol (1,4,5)-trisphosphate metabolism | 0.008820865 | 2 | 8 |
| Mm_Exercise-induced_Circadian_Regulation_WP544_69890 | 0.011720306 | 4 | 49 |
| Mm_Metapathway_biotransformation_WP1251_69747 | 0.011818458 | 3 | 143 |
| Mm_IL-2_Signaling_Pathway_WP450_67368 | 0.011883704 | 5 | 76 |
| pyrimidine ribonucleotides interconversion | 0.013834631 | 2 | 10 |
| Mm_miRNAs_involved_in_DNA_damage_response_WP2085_74241 | 0.013834631 | 2 | 49 |
| pyrimidine ribonucleotides de novo biosynthesis | 0.016704416 | 2 | 12 |
| CDP-diacylglycerol biosynthesis I | 0.016704416 | 2 | 13 |
| Mm_Regulation_of_Actin_Cytoskeleton_WP523_71326 | 0.017078303 | 7 | 151 |
| Mm_Prostaglandin_Synthesis_and_Regulation_WP374_69204 | 0.019096008 | 3 | 31 |
| Mm_Cell_cycle_WP190_71755 | 0.01963693 | 5 | 88 |
| phosphatidylglycerol biosynthesis II (non-plastidic) | 0.019803159 | 2 | 14 |
| Mm_Glycogen_Metabolism_WP317_70007 | 0.020789187 | 3 | 34 |
| Mm_Signaling_of_Hepatocyte_Growth_Factor_Receptor_WP193_69178 | 0.022562083 | 3 | 34 |
| starch degradation | 0.023121472 | 2 | 14 |
| colanic acid building blocks biosynthesis | 0.023121472 | 2 | 14 |
| fatty acid Beta-oxidation II (core pathway) | 0.023121472 | 2 | 15 |
| Mm_SIDS_Susceptibility_Pathways_WP1266_69139 | 0.024744025 | 4 | 61 |
| tRNA charging pathway | 0.026346961 | 3 | 37 |
| glycolysis III | 0.026650239 | 2 | 14 |
| Mm_T_Cell_Receptor_Signaling_Pathway_WP480_69149 | 0.027621077 | 6 | 133 |
| glycolysis I | 0.030380595 | 2 | 16 |
| Mm_PluriNetWork_WP1763_72003 | 0.035232157 | 10 | 291 |
| Mm_Striated_Muscle_Contraction_WP216_72052 | 0.037194125 | 3 | 45 |
| Mm_IL-3_Signaling_Pathway_WP373_69196 | 0.037842713 | 5 | 100 |
| Mm_Nucleotide_Metabolism_WP87_71749 | 0.047149517 | 2 | 19 |
| Mm_Glutathione_metabolism_WP164_71334 | 0.047149517 | 2 | 19 |
| Mm_Wnt_Signaling_Pathway_NetPath_WP539_71716 | 0.04984857 | 5 | 109 |
| Mm_Selenium_metabolism-Selenoproteins_WP108_69772 | 0.049974676 | 3 | 48 |

TABLE 7
Enriched pathways from Wikipathways altered with age in Drd1a-
expressing cortical neurons.

| | | | Total |
|---|---|---|---|
| | | Matched | Pathway |
| Pathway | p value | Entities | Entities |
| Mm_Striated_Muscle_Contraction_WP216_72052 | 1.49E−04 | 3 | 45 |
| Mm_Keap1-Nrf2_WP1245_71125 | 4.95E−04 | 2 | 14 |
| Mm_Fatty_Acid_Biosynthesis_WP336_71737 | 0.001443061 | 2 | 22 |
| Mm_Signaling_of_Hepatocyte_Growth_Factor_Receptor_WP193_69178 | 0.003238718 | 2 | 34 |
| bupropion degradation | 0.003435435 | 2 | 35 |
| nicotine degradation III | 0.005203122 | 2 | 43 |
| nicotine degradation II | 0.006749277 | 2 | 49 |
| Mm_B_Cell_Receptor_Signaling_Pathway_WP274_67072 | 0.006916409 | 3 | 156 |
| Mm_Myometrial_Relaxation_and_Contraction_Pathways_WP385_72108 | 0.007299635 | 3 | 157 |
| Mm_Primary_Focal_Segmental_Glomerulosclerosis_FSGS_WP2573_72201 | 0.010716978 | 2 | 73 |
| Mm_IL-2_Signaling_Pathway_WP450_67368 | 0.015482554 | 2 | 76 |
| Mm_XPodNet_-_protein- | 0.015918477 | 6 | 836 |
| protein_interactions_in_the_podocyte_expanded_by_STRING_WP2309_72004 | | | |
| Mm_IL-6_signaling_Pathway_WP387_72091 | 0.025211193 | 2 | 99 |

TABLE 8
Enriched pathways from Wikipathways altered with age in Drd2-expressing
cortical neurons.

| | | | Total |
|---|---|---|---|
| | | Matched | Pathway |
| Pathway | p value | Entities | Entities |
| Mm_XPodNet_-_protein- | 9.61E−05 | 12 | 836 |
| protein_interactions_in_the_podocyte_expanded_by_STRING_WP2309_72004 | | | |
| Mm_B_Cell_Receptor_Signaling_Pathway_WP274_67072 | 0.003393002 | 4 | 156 |
| glutathione-mediated detoxification | 0.004241159 | 2 | 24 |
| Mm_Prostaglandin_Synthesis_and_Regulation_WP374_69204 | 0.007014286 | 2 | 31 |
| Mm_Retinol_metabolism_WP1259_74433 | 0.010410202 | 2 | 39 |
| Mm_Striated_Muscle_Contraction_WP216_72052 | 0.011489692 | 2 | 45 |
| Mm_Adipogenesis_genes_WP447_73875 | 0.01569575 | 3 | 133 |
| Mm_G1_to_S_cell_cycle_control_WP413_72012 | 0.024738263 | 2 | 62 |
| Mm_Chemokine_signaling_pathway_WP2292_72463 | 0.034508925 | 3 | 193 |
| Mm_Cell_cycle_WP190_71755 | 0.04576582 | 2 | 88 |

TABLE 9
Lentiviruses used in this study
95 shRNA lentiviruses targeting 76 distinct target sequences

| | | | | | | NCBI | | |
|---|---|---|---|---|---|---|---|---|
| | Vector | Transcript | Gene | Target | Reasom Gene | Gene | Target | |
| Hairpin ID | Name | Targeted | Symbol | type | was Chosen | ID | Region | Target Sequences |
| TRCN0000072261 | pLKO.1 | promegaLuc.1 | Luciferase | Control | Negative | | CDS | CACTCGGATATTTGATA |
| | | | | | Control | | | TGTG |
| TRCN0000072250 | pLKO.1 | promegaLuc.1 | Luciferase | Control | Negative | | CDS | AGAATCGTCGTATGCAG |
| | | | | | Control | | | TGAA |
| TRCN0000066072 | pLKO.1 | NM_134101.1 | Psmd2 | Control | Positive | 21762 | CDS | CGCCAGTTAGCTCAATA |
| | | | | | Control | | | TCAT |
| TRCN0000207065 | pLKO.1 | clonetechGfp.1 | GFP | Control | Negative | | CDS | GCGATCACATGGTCCTG |
| | | | | | Control | | | CTGG |
| TRCN0000072231 | pLKO.1 | lacZ.1 | LacZ | Control | Negative | | CDS | CGCTAAATACTGGCAGG |
| | | | | | Control | | | CGTT |
| TRCN0000072209 | pLKO.1 | rfp.1 | RFP | Control | Negative | | CDS | CTCAGTTCCAGTACGGC |
| | | | | | Control | | | TCCA |
| TRCN0000231782 | pLKO_ | None | None | Control | Negative | None | Non- | ACAGTTAACCACTTTTTG |
| | TRC021 | | | | Control | | shRNA | AAT |
| | | | | | | | trans- | |
| | | | | | | | cript | |
| TRCN0000428544 | pLKO_ | NM_053139.3 | Rcdhb14 | Experi- | Upregulated | 93885 | CDS | GTAGTGCAACCATCACGT |
| | TRC005 | | | mental | with age in | | | ATT |
| | | | | | Drd1a- and | | | |
| | | | | | Drd2- | | | |
| | | | | | expressing | | | |
| | | | | | medium | | | |
| | | | | | spiny neurons | | | |
| | | | | | (Tables S1 | | | |
| | | | | | and S2) | | | |
| TRCN0000435247 | pLKO_ | NM_053139.3 | Rcdhb14 | Experi- | Upregulated | 93885 | CDS | AGGCAAGTGACCGCCATT |
| | TRC005 | | | mental | with age in | | | ATC |
| | | | | | Drd1a- and | | | |
| | | | | | Drd2- | | | |
| | | | | | expressing | | | |
| | | | | | medium | | | |
| | | | | | spiny neurons | | | |
| | | | | | (Tables S1 | | | |
| | | | | | and S2) | | | |
| TRCN0000419614 | pLKO_ | NM_053139.3 | Rcdhb14 | Experi- | Upregulated | 93885 | 3UTR | CATGATACTGGTAGTCAT |
| | TRC005 | | | mental | with age in | | | TT |
| | | | | | Drd1a- and | | | |
| | | | | | Drd2- | | | |
| | | | | | expressing | | | |
| | | | | | medium | | | |
| | | | | | spiny neurons | | | |
| | | | | | (Tables S1 | | | |
| | | | | | and S2) | | | |
| TRCN0000426134 | pLKO_ | NM_053139.3 | Rcdhb14 | Experi- | Upregulated | 93885 | CDS | TCAGTACTTATCAGCGAA |
| | TRC005 | | | mental | with age in | | | ATT |
| | | | | | Drd1a- and | | | |
| | | | | | Drd2- | | | |
| | | | | | expressing | | | |
| | | | | | medium | | | |
| | | | | | spiny neurons | | | |
| | | | | | (Tables S1 | | | |
| | | | | | and S2) | | | |
| TRCN0000320173 | pLKO_ | NM_010517.3 | Igfbp4 | Experi- | IGF-1 has | 16010 | CDS | CATTCCAAACTGTGACCG |
| | TRC005 | | | mental | neuropro- | | | CAA |
| | | | | | tective | | | |
| | | | | | effects in | | | |
| | | | | | HD (Humbert | | | |
| | | | | | et al., 2002) | | | |
| | | | | | and Igfb4 is | | | |
| | | | | | striated- | | | |
| | | | | | enriched | | | |
| | | | | | (Heiman et | | | |
| | | | | | al., 2008) | | | |
| TRCN0000350214 | pLKO_ | NM_010517.3 | Igfbp4 | Experi- | IGF-1 has | 16010 | CDS | GCTGCGGTTGTTGCGCCA |
| | TRC005 | | | mental | neuropro- | | | CTT |
| | | | | | tective | | | |
| | | | | | effects in | | | |
| | | | | | HD (Humbert | | | |
| | | | | | et al., 2002) | | | |
| | | | | | and Igfb4 is | | | |
| | | | | | striated- | | | |
| | | | | | enriched | | | |
| | | | | | (Heiman et | | | |
| | | | | | al., 2008) | | | |
| TRCN0000114798 | pLKO.1 | NM_010517.2 | Igfbp4 | Experi- | IGF-1 has | 16010 | CDS | GACAAGGATGAGAGCGAA |
| | | | | mental | neuropro- | | | CAT |
| | | | | | tective | | | |
| | | | | | effects in | | | |
| | | | | | HD (Humbert | | | |
| | | | | | et al., 2002) | | | |
| | | | | | and Igfb4 is | | | |
| | | | | | striated- | | | |
| | | | | | enriched | | | |
| | | | | | (Heiman et | | | |
| | | | | | al., 2008) | | | |
| TRCN0000114797 | pLKO.1 | NM_010517.2 | Igfbp4 | Experi- | IGF-1 has | 16010 | CDS | CATTCCAAACTGTGACC |
| | | | | mental | neuropro- | | | GCAA |
| | | | | | tective | | | |
| | | | | | effects in | | | |
| | | | | | HD (Humbert | | | |
| | | | | | et al., 2002) | | | |
| | | | | | and Igfb4 is | | | |
| | | | | | striated- | | | |
| | | | | | enriched | | | |
| | | | | | (Heiman et | | | |
| | | | | | al., 2008) | | | |
| TRCN0000114800 | pLKO.1 | NM_010517.2 | Igfbp4 | Experi- | IGF-1 has | 16010 | CDS | GCTGCGGTTGTTGCGCC |
| | | | | mental | neuropro- | | | ACTT |
| | | | | | tective | | | |
| | | | | | effects in | | | |
| | | | | | HD (Humbert | | | |
| | | | | | et al., 2002) | | | |
| | | | | | and Igfb4 is | | | |
| | | | | | striated- | | | |
| | | | | | enriched | | | |
| | | | | | (Heiman et | | | |
| | | | | | al., 2008) | | | |
| TRCN0000320111 | pLKO_ | NM_010517.3 | Igfbp4 | Experi- | IGF-1 has | 16010 | CDS | GACAAGGATGAGAGCGA |
| | TRC005 | | | mental | neuropro- | | | ACAT |
| | | | | | tective | | | |
| | | | | | effects in | | | |
| | | | | | HD (Humbert | | | |
| | | | | | et al., 2002) | | | |
| | | | | | and Igfb4 is | | | |
| | | | | | striated- | | | |
| | | | | | enriched | | | |
| | | | | | (Heiman et | | | |
| | | | | | al., 2008) | | | |
| TRCN0000288175 | pLKO_ | NM_011063.2 | Pea15a | Experi- | Upregulated | 18611 | CDS | CAAAGACAACCTCTCCT |
| | TRC005 | | | mental | with age in | | | ACAT |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000105789 | pLKO.1 | NM_011603.1 | Pea15a | Experi- | Upregulated | 18611 | CDS | CCTGACCAACAACATCA |
| | | | | mental | with age in | | | CCCT |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000105787 | pLKO.1 | NM_011603.1 | Pea15a | Experi- | Upregulated | 18611 | CDS | CAAAGACAACCTCTCCC |
| | | | | mental | with age in | | | TACAT |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000288240 | pLKO_ | NM_011063.2 | Pea15a | Experi- | Upregulated | 18611 | CDS | CCTGACCAACAACATCA |
| | TRC005 | | | mental | with age in | | | CCCT |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000307569 | pLKO_ | NM_011063.2 | Pea15a | Experi- | Upregulated | 18611 | CDS | ACACCAAGCTAACCCGT |
| | TRC005 | | | mental | with age in | | | ATTC |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000096379 | pLKO.1 | NM_007488.2 | Arnt2 | Experi- | Upregulated | 11864 | 3UTR | CGCTATTATCATGCCAT |
| | | | | mental | with age in | | | AGAT |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000096382 | pLKO.1 | NM_007488.2 | Arnt2 | Experi- | Upregulated | 11864 | CDS | CCTACTCTGATGAGATC |
| | | | | mental | with age in | | | GAGT |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN00000323726 | pLKO_ | NM_007488.2 | Arnt2 | Experi- | Upregulated | 11864 | 3UTR | CGCYATTATCATGCCAT |
| | TRC005 | | | mental | with age in | | | AGAT |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000323788 | pLKO_ | NM_007488.2 | Arnt2 | Experi- | Upregulated | 11864 | CDS | CCTACTCTGATGAGATC |
| | TRC005 | | | mental | with age in | | | GAGT |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000374677 | pLKO_ | NM_007488.2 | Arnt2 | Experi- | Upregulated | 11864 | CDS | TGTCGGACAAGGCAGTA |
| | TRC005 | | | mental | with age in | | | AATA |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN000023132 | pLKO_ | NM_001038695.1 | Kdm3a | Experi- | Upregulated | 104263 | CDS | CACGATCAGAGCTGGTA |
| | TRC005 | | | mental | with age in | | | TTTA |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000252744 | pLKO_ | NM_001038695.2 | Kdm3a | Experi- | Upregulated | 104263 | CDS | TGCGGGTAGAAGGCTTC |
| | TRC005 | | | mental | with age in | | | TTAA |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000252745 | pLKO_ | NM_001038695.2 | Kdm3a | Experi- | Upregulated | 104263 | 3UTR | CTGCGAAGTTTCGTTGGA |
| | TRC005 | | | mental | with age in | | | TTT |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000252747 | pLKO_ | NM_001038695.2 | Kdm3a | Experi- | Upregulated | 104263 | CDS | GAAGTTCCTGAGCAAGT |
| | TRC005 | | | mental | with age in | | | TATT |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000295705 | pLKO_ | NM_009735.3 | B2m | Experi- | Upregulated | 12010 | 3UTR | CCAGTTTCTAATATGCT |
| | TRC005 | | | mental | with age in | | | ATAC |
| | | | | | Drd1a- and | | | |
| | | | | | Drd2- | | | |
| | | | | | expressing | | | |
| | | | | | medium spiny | | | |
| | | | | | well as Drd2- | | | |
| | | | | | expressing | | | |
| | | | | | cortical | | | |
| | | | | | cells (Tables | | | |
| | | | | | S1, S2, and | | | |
| | | | | | S4) | | | |
| TRCN0000295762 | pLKO_ | NM_009735.3 | B2m | Experi- | Upregulated | 12010 | CDS | TAAAGTAGAGATGTCAG |
| | TRC005 | | | mental | with age in | | | ATAT |
| | | | | | Drd1a- and | | | |
| | | | | | Drd2- | | | |
| | | | | | expressing | | | |
| | | | | | medium spiny | | | |
| | | | | | well as Drd2- | | | |
| | | | | | expressing | | | |
| | | | | | cortical | | | |
| | | | | | cells (Tables | | | |
| | | | | | S1, S2, and | | | |
| | | | | | S4) | | | |
| TRCN0000288438 | pLKO_ | NM_009735.3 | B2m | Experi- | Upregulated | 12010 | CDS | GCCGAACATACTGAACT |
| | TRC005 | | | mental | with age in | | | GCTA |
| | | | | | Drd1a- and | | | |
| | | | | | Drd2- | | | |
| | | | | | expressing | | | |
| | | | | | medium spiny | | | |
| | | | | | well as Drd2- | | | |
| | | | | | expressing | | | |
| | | | | | cortical | | | |
| | | | | | cells (Tables | | | |
| | | | | | S1, S2, and | | | |
| | | | | | S4) | | | |
| TRCN0000066424 | pLKO.1 | NM_009735.3 | B2m | Experi- | Upregulated | 12010 | CDS | GCCGAACATACTGAACT |
| | | | | mental | with age in | | | GCTA |
| | | | | | Drd1a- and | | | |
| | | | | | Drd2- | | | |
| | | | | | expressing | | | |
| | | | | | medium spiny | | | |
| | | | | | well as Drd2- | | | |
| | | | | | expressing | | | |
| | | | | | cortical | | | |
| | | | | | cells (Tables | | | |
| | | | | | S1, S2, and | | | |
| | | | | | S4) | | | |
| TRCN0000329356 | pLKO_ | NM_177386.4 | Sfmbt2 | Experi- | Previously | 353282 | CDS | CCCTCTGACCACACCAT |
| | TRC005 | | | mental | shown to | | | ATAA |
| | | | | | change in | | | |
| | | | | | published | | | |
| | | | | | HD studies | | | |
| | | | | | (Becanovic | | | |
| | | | | | et al., 2010) | | | |
| TRCN0000329354 | pLKO_ | NM_177386.4 | Sfmbt2 | Experi- | Previously | 353282 | CDS | CGGATGTGGTACGATTC |
| | TRC005 | | | mental | shown to | | | ATTA |
| | | | | | change in | | | |
| | | | | | published | | | |
| | | | | | HD studies | | | |
| | | | | | (Becanovic | | | |
| | | | | | et al., 2010) | | | |
| TRCN0000329357 | pLKO_ | NM_177386.4 | Sfmbt2 | Experi- | Previously | 353282 | 3UTR | CCTATTTGATAGTCCTA |
| | TRC005 | | | mental | shown to | | | TATT |
| | | | | | change in | | | |
| | | | | | published | | | |
| | | | | | HD studies | | | |
| | | | | | (Becanovic | | | |
| | | | | | et al., 2010) | | | |
| TRCN | pLKO_ | NM_177386.4 | Sfmbt2 | Experi- | Previously | 353282 | CDS | TTCGTCAACCACCGGTGT |
| | TRC005 | | | mental | shown to | | | TTC |
| | | | | | change in | | | |
| | | | | | published | | | |
| | | | | | HD studies | | | |
| | | | | | (Becanovic | | | |
| | | | | | et al., 2010) | | | |
| TRCN0000337555 | pLKO_ | NM_030143.4 | Ddit41 | Experi- | Previously | 73284 | 3UTR | CCCTAATGAGTGGATA |
| | TRC005 | | | mental | shown to | | | ATAAA |
| | | | | | change in | | | |
| | | | | | published | | | |
| | | | | | HD studies | | | |
| | | | | | (Becanovic | | | |
| | | | | | et al., 2010) | | | |
| TRCN0000276917 | pLKO_ | NM_030143.4 | Ddit41 | Experi- | Previously | 73284 | CDS | GATTTCGACTACTGGGAT |
| | TRC005 | | | mental | shown to | | | TAT |
| | | | | | change in | | | |
| | | | | | published | | | |
| | | | | | HD studies | | | |
| | | | | | (Becanovic | | | |
| | | | | | et al., 2010) | | | |
| TRCN0000176976 | pLKO.1 | NM_030143.2 | Ddit41 | Experi- | Previously | 73284 | CDS | GATTTCGACTACTGGGA |
| | | | | mental | shown to | | | TTAT |
| | | | | | change in | | | |
| | | | | | published | | | |
| | | | | | HD studies | | | |
| | | | | | (Becanovic | | | |
| | | | | | et al., 2010) | | | |
| TRCN0000276918 | pLKO_ | NM_030143.3 | Ddit41 | Experi- | Previously | 73284 | CDS | TCGCTTCTCCTCAGGCC |
| | TRC005 | | | mental | shown to | | | TTAA |
| | | | | | change in | | | |
| | | | | | published | | | |
| | | | | | HD studies | | | |
| | | | | | (Becanovic | | | |
| | | | | | et al., 2010) | | | |
| TRCN0000183203 | pLKO.1 | NM_010726.1 | Phyh | Experi- | Upregulated | 16922 | 3UTR | GAGGACATCAAAGCAAA |
| | | | | mental | with age in | | | GAAA |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000183360 | pLKO.1 | NM_010726.1 | Phyh | Experi- | Upregulated | 16922 | 3UTR | GCTCTTCCTTATAATT |
| | | | | mental | with age in | | | CCTTT |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000314263 | pLKO_ | NM_0107262.2 | Phyh | Experi- | Upregulated | 16922 | 3UTR | GAGGACATCAAAGCAAA |
| | TRC005 | | | mental | with age in | | | GAAA |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000314262 | pLKO_ | NM_0107262.2 | Phyh | Experi- | Upregulated | 16922 | 3UTR | GCTCTTCCTTATAATT |
| | TRC005 | | | mental | with age in | | | CCCTTT |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000221761 | pLKO.1 | NM_008828.1 | Pgk1 | Experi- | Randomly | 18655 | CDS | CATCAAATTCTGCTTGG |
| | | | | mental | chosen | | | ACAA |
| | | | | | housekeeping | | | |
| | | | | | target gene | | | |
| TRCN0000104502 | pLKO.1 | NM_009094.1 | Rps4x | Experi- | Proteins | 20102 | CDS | CCCTGACTGGAGATGAA |
| | | | | mental | involved in | | | GTAA |
| | | | | | translation | | | |
| | | | | | have been | | | |
| | | | | | shown to be | | | |
| | | | | | associated | | | |
| | | | | | with | | | |
| | | | | | Huntingtin | | | |
| | | | | | protein | | | |
| | | | | | (Culver et | | | |
| | | | | | al., 2012) | | | |
| TRCN | pLKO.1 | NM_016980.1 | Rpl5 | Experi- | Proteins | 100503670 | CDS | CCCTCATAGTACCAAA |
| | | | | mental | involved in | | | CGATT |
| | | | | | translation | | | |
| | | | | | have been | | | |
| | | | | | shown to be | | | |
| | | | | | associated | | | |
| | | | | | with | | | |
| | | | | | Huntingtin | | | |
| | | | | | protein | | | |
| | | | | | (Culver et | | | |
| | | | | | al., 2012) | | | |
| TRCN0000311277 | pLKO_ | NM_009483.1 | Kdm6a | Experi- | Upregulated | 22289 | 3UTR | CTATGCCAGGACTCTCG |
| | TRC005 | | | mental | with age | | | TAAA |
| | | | | | when Drd1a- | | | |
| | | | | | and Drd2- | | | |
| | | | | | expressing | | | |
| | | | | | medium spiny | | | |
| | | | | | gene | | | |
| | | | | | expression | | | |
| | | | | | data are | | | |
| | | | | | pooled | | | |
| | | | | | (analysis | | | |
| | | | | | not shown) | | | |
| TRCN0000305239 | pLKO_ | NM_009483.1 | Kdm6a | Experi- | Upregulated | 22289 | CDS | AGTTAGCAGTGGAACGTT |
| | TRC005 | | | mental | with age | | | ATG |
| | | | | | when Drd1a- | | | |
| | | | | | and Drd2- | | | |
| | | | | | expressing | | | |
| | | | | | medium spiny | | | |
| | | | | | gene | | | |
| | | | | | expression | | | |
| | | | | | data are | | | |
| | | | | | pooled | | | |
| | | | | | (analysis | | | |
| | | | | | not shown) | | | |
| TRCN0000096242 | pLKO.1 | NM_009483.1 | Kdm6a | Experi- | Upregulated | 22289 | CDS | GCTACGAATCTCTAATC |
| | | | | mental | with age | | | TTAA |
| | | | | | when Drd1a- | | | |
| | | | | | and Drd2- | | | |
| | | | | | expressing | | | |
| | | | | | medium spiny | | | |
| | | | | | gene | | | |
| | | | | | expression | | | |
| | | | | | data are | | | |
| | | | | | pooled | | | |
| | | | | | (analysis | | | |
| | | | | | not shown) | | | |
| TRCN0000331919 | pLKO_ | NM_009483.1 | Kdm6a | Experi- | Upregulated | 22289 | CDS | GCTACGAATCTCTAATC |
| | TRC005 | | | mental | with age | | | TTAA |
| | | | | | when Drd1a- | | | |
| | | | | | and Drd2- | | | |
| | | | | | expressing | | | |
| | | | | | medium spiny | | | |
| | | | | | gene | | | |
| | | | | | expression | | | |
| | | | | | data are | | | |
| | | | | | pooled | | | |
| | | | | | (analysis | | | |
| | | | | | not shown) | | | |
| TRCN0000085087 | pLKO.1 | NM_025444.1 | Taf13 | Experi- | Down- | 99730 | CDS | CGAAGACCTTGTCATAG |
| | | | | mental | regulated with | | | AGTT |
| | | | | | age in Drd1a- | | | |
| | | | | | and Drd2- | | | |
| | | | | | expressing | | | |
| | | | | | medium spiny | | | |
| | | | | | neurons, as | | | |
| | | | | | well as Drd2- | | | |
| | | | | | expressing | | | |
| | | | | | cortical cells | | | |
| | | | | | (Tables S1, | | | |
| | | | | | S2, and S4) | | | |
| TRCN0000085085 | pLKO.1 | NM_025444.1 | Taf13 | Experi- | Down- | 99730 | CDS | AGAATTGAAACGGGCTA |
| | | | | mental | regulated with | | | GAAA |
| | | | | | age in Drd1a- | | | |
| | | | | | and Drd2- | | | |
| | | | | | expressing | | | |
| | | | | | medium spiny | | | |
| | | | | | neurons, as | | | |
| | | | | | well as Drd2- | | | |
| | | | | | expressing | | | |
| | | | | | cortical cells | | | |
| | | | | | (Tables S1, | | | |
| | | | | | S2, and S4) | | | |
| TRCN0000317962 | pLKO_ | NM_025444.1 | Taf13 | Experi- | Down- | 99730 | CDS | CGAAGACCTTGTCATAG |
| | TRC005 | | | mental | regulated with | | | AGTT |
| | | | | | age in Drd1a- | | | |
| | | | | | and Drd2- | | | |
| | | | | | expressing | | | |
| | | | | | medium spiny | | | |
| | | | | | neurons, as | | | |
| | | | | | well as Drd2- | | | |
| | | | | | expressing | | | |
| | | | | | cortical cells | | | |
| | | | | | (Tables S1, | | | |
| | | | | | S2, and S4) | | | |
| TRCN0000317963 | pLKO_ | NM_025444.2 | Taf13 | Experi- | Down- | 99730 | CDS | AGAATTGAAACGGGCTA |
| | TRC005 | | | mental | regulated with | | | GAAA |
| | | | | | age in Drd1a- | | | |
| | | | | | and Drd2- | | | |
| | | | | | expressing | | | |
| | | | | | medium spiny | | | |
| | | | | | neurons, as | | | |
| | | | | | well as Drd2- | | | |
| | | | | | expressing | | | |
| | | | | | cortical cells | | | |
| | | | | | (Tables S1, | | | |
| | | | | | S2, and S4) | | | |
| TRCN0000287596 | pLKO_ | NM_026163.2 | Pkp2 | Experi- | Randomly | 67451 | CDS | GCCTTGAGAAACTTGG |
| | TRC005 | | | mental | chosen gene | | | TATTT |
| | | | | | target | | | |
| TRCN0000123350 | pLKO.1 | NM_026163.1 | Pkp2 | Experi- | Randomly | 67451 | CDS | GCCTTGAGAAACTTGGTA |
| | | | | mental | chosen gene | | | TTT |
| | | | | | target | | | |
| TRCN0000123351 | pLKO.1 | NM_026163.1 | Pkp2 | Experi- | Randomly | 67451 | CDS | CCTGAGTATGTCTACAA |
| | | | | mental | chosen gene | | | GCTA |
| | | | | | target | | | |
| TRCN0000287514 | pLKO_ | NM_026163.2 | Pkp2 | Experi- | Randomly | 67451 | CDS | CCTGAGTATGTCTACAA |
| | TRC005 | | | mental | chosen gene | | | GCTA |
| | | | | | target | | | |
| TRCN0000071993 | pLKO.1 | NM_053242.3 | Foxp2 | Experi- | Upregulated | 114142 | 3UTR | CGGAAGTTATTGATGT |
| | | | | mental | with age in | | | GGTAT |
| | | | | | Drd2- | | | |
| | | | | | expressing | | | |
| | | | | | medium spiny | | | |
| | | | | | neurons, as | | | |
| | | | | | well as | | | |
| | | | | | Drd2- | | | |
| | | | | | expressing | | | |
| | | | | | cortical cells | | | |
| | | | | | (Tables S2 | | | |
| | | | | | and S4) | | | |
| TRCN0000071994 | pLKO.1 | NM_053242.3 | Foxp2 | Experi- | Upregulated | 114142 | CDS | CGGACAGTCTTCAGTT |
| | | | | mental | with age in | | | CTGAA |
| | | | | | Drd2- | | | |
| | | | | | expressing | | | |
| | | | | | medium spiny | | | |
| | | | | | neurons, as | | | |
| | | | | | well as | | | |
| | | | | | Drd2- | | | |
| | | | | | expressing | | | |
| | | | | | cortical cells | | | |
| | | | | | (Tables S2 | | | |
| | | | | | and S4) | | | |
| TRCN0000071997 | pLKO.1 | NM_053242.3 | Foxp2 | Experi- | Upregulated | 114142 | CDS | GCGACATTCAGACAAA |
| | | | | mental | with age in | | | TACAA |
| | | | | | Drd2- | | | |
| | | | | | expressing | | | |
| | | | | | medium spiny | | | |
| | | | | | neurons, as | | | |
| | | | | | well as | | | |
| | | | | | Drd2- | | | |
| | | | | | expressing | | | |
| | | | | | cortical cells | | | |
| | | | | | (Tables S2 | | | |
| | | | | | and S4) | | | |
| TRCN0000076492 | pLKO.1 | NM_145451.1 | Gpx6 | Experi- | Upregulated | 75512 | CDS | AGCCATTCAACGTCAC |
| | | | | mental | with age in | | | GGTTT |
| | | | | | Drd1a- | | | |
| | | | | | and Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000076491 | pLKO.1 | NM_145451.1 | Gpx6 | Experi- | Upregulated | 75512 | CDS | GTGAACGGAGACAATGAA |
| | | | | mental | with age in | | | CAA |
| | | | | | Drd1a- | | | |
| | | | | | and Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000076488 | pLKO.1 | NM_145451.1 | Gpx6 | Experi- | Upregulated | 75512 | 3UTR | GCATGTGCAATCTACAG |
| | | | | mental | with age in | | | AGAT |
| | | | | | Drd1a- | | | |
| | | | | | and Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000125009 | pLKO.1 | NM_177346.1 | Gpr149 | Experi- | Upregulated | 229357 | 3UTR | CCCACTTTCTTCTAGTT |
| | | | | mental | with age in | | | ATAT |
| | | | | | Drd2- | | | |
| | | | | | expressing | | | |
| | | | | | medium spiny | | | |
| | | | | | neurons | | | |
| | | | | | (Table S2) | | | |
| TRCN0000125011 | pLKO.1 | NM_177346.1 | Gpr149 | Experi- | Upregulated | 229357 | CDS | GCGATATTAACTATGGA |
| | | | | mental | with age in | | | GAAA |
| | | | | | Drd2- | | | |
| | | | | | expressing | | | |
| | | | | | medium spiny | | | |
| | | | | | neurons | | | |
| | | | | | (Table S2) | | | |
| TRCN0000125010 | pLKO.1 | NM_177346.1 | Gpr149 | Experi- | Upregulated | 229357 | CDS | CCAGTGTTTGTCTTAT |
| | | | | mental | with age in | | | CCAAA |
| | | | | | Drd2- | | | |
| | | | | | expressing | | | |
| | | | | | medium spiny | | | |
| | | | | | neurons | | | |
| | | | | | (Table S2) | | | |
| TRCN0000317130 | pLKO_ | NM_009112.2 | S100a10 | Experi- | Upregulated | 20194 | CDS | CCGAGAGCTTTCTATCAC |
| | TRC005 | | | mental | with age in | | | TAGT |
| | | | | | Drd1a- and | | | |
| | | | | | Drd2- | | | |
| | | | | | expressing | | | |
| | | | | | medium spiny | | | |
| | | | | | neurons, as | | | |
| | | | | | well as | | | |
| | | | | | Drd2- | | | |
| | | | | | expressing | | | |
| | | | | | cortical cells | | | |
| | | | | | (Tables S1, | | | |
| | | | | | S2, and S4) | | | |
| TRCN0000097669 | pLKO.1 | NM_009112.1 | S100a10 | Experi- | Upregulated | 20194 | CDS | CCAGAGCTTTCTATCAC |
| | | | | mental | with age in | | | TAGT |
| | | | | | Drd1a- and | | | |
| | | | | | Drd2- | | | |
| | | | | | expressing | | | |
| | | | | | medium spiny | | | |
| | | | | | neurons, as | | | |
| | | | | | well as | | | |
| | | | | | Drd2- | | | |
| | | | | | expressing | | | |
| | | | | | cortical cells | | | |
| | | | | | (Tables S1, | | | |
| | | | | | S2, and S4) | | | |
| TRCN0000034382 | pLKO.1 | NM_008284.1 | Hras1 | Experi- | Downregulated | 15461 | CDS | CGGGTGAAAGATTCAGA |
| | | | | mental | with age in | | | TGAT |
| | | | | | Drd1a- | | | |
| | | | | | and Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000366695 | pLKO_ | NM_008284.2 | Hras1 | Experi- | Downregulated | 15461 | CDS | GTGAGATTCGGCAGCA |
| | TRC005 | | | mental | with age in | | | TAAAT |
| | | | | | Drd1a- | | | |
| | | | | | and Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000366696 | pLKO_ | NM_008284.2 | Hras1 | Experi- | Downregulated | 15461 | 3UTR | CACGTTGCATCACAGT |
| | TRC005 | | | mental | with age in | | | AAATT |
| | | | | | Drd1a- | | | |
| | | | | | and Drd2- | | | |
| | | | | | expressing | | | |
| | | | | | medium spiny | | | |
| | | | | | neurons | | | |
| | | | | | (Tables S1 | | | |
| | | | | | and S2) | | | |
| TRCN0000323443 | pLKO_ | NM_019635.2 | Stk3 | Experi- | Upregulated | 56274 | CDS | CCTGAGGTAATTCAAG |
| | TRC005 | | | mental | with age in | | | AAATA |
| | | | | | Drd1a- and | | | |
| | | | | | Drd2- | | | |
| | | | | | expressing | | | |
| | | | | | medium spiny | | | |
| | | | | | neurons, as | | | |
| | | | | | well as | | | |
| | | | | | Drd2- | | | |
| | | | | | expressing | | | |
| | | | | | cortical cells | | | |
| | | | | | (Tables S1, | | | |
| | | | | | S2, and S4) | | | |
| TRCN0000025880 | pLKO.1 | NM_019635.1 | Stk3 | Experi- | Upregulated | 56274 | CDS | CCTGAGGTAATTCAAG |
| | | | | mental | with age in | | | AAATA |
| | | | | | Drd1a- and | | | |
| | | | | | Drd2- | | | |
| | | | | | expressing | | | |
| | | | | | medium spiny | | | |
| | | | | | neurons, as | | | |
| | | | | | well as | | | |
| | | | | | Drd2- | | | |
| | | | | | expressing | | | |
| | | | | | cortical cells | | | |
| | | | | | (Tables S1, | | | |
| | | | | | S2, and S4) | | | |
| TRCN0000025951 | pLKO.1 | NM_019635.1 | Stk3 | Experi- | Upregulated | 56274 | CDS | CCTGAGGTAATTCAAG |
| | | | | mental | with age in | | | AAATA |
| | | | | | Drd1a- and | | | |
| | | | | | Drd2- | | | |
| | | | | | expressing | | | |
| | | | | | medium spiny | | | |
| | | | | | neurons, as | | | |
| | | | | | well as | | | |
| | | | | | Drd2- | | | |
| | | | | | expressing | | | |
| | | | | | cortical cells | | | |
| | | | | | (Tables S1, | | | |
| | | | | | S2, and S4) | | | |
| TRCN0000094178 | pLKO.1 | NM_053133.1 | Pcdhb8 | Experi- | Upregulated | 93879 | CDS | AGACTTGCAGTTCACA |
| | | | | mental | with age in | | | GATAT |
| | | | | | Drd2- | | | |
| | | | | | expressing | | | |
| | | | | | medium spiny | | | |
| | | | | | neurons | | | |
| | | | | | (Tables S2) | | | |
| TRCN0000094175 | pLKO.1 | NM_053133.1 | Pcdhb8 | Experi- | Upregulated | 93879 | CDS | CTGGCTCCAATGGCCTTA |
| | | | | mental | with age in | | | TTA |
| | | | | | Drd2- | | | |
| | | | | | expressing | | | |
| | | | | | medium spiny | | | |
| | | | | | neurons | | | |
| | | | | | (Tables S2) | | | |
| TRCN0000094176 | pLKO.1 | NM_053133.1 | Pcdhb8 | Experi- | Upregulated | 93879 | CDS | CACAGATATAAACGA |
| | | | | mental | with age in | | | CCATTT |
| | | | | | Drd2- | | | |
| | | | | | expressing | | | |
| | | | | | medium spiny | | | |
| | | | | | neurons | | | |
| | | | | | (Tables S2) | | | |
| TRCN0000077330 | pLKO.1 | NM_028810.1 | Rnd3 | Experi- | Downregulated | 74194 | CDS | GCACATTAGTGGAACTC |
| | | | | mental | with age in | | | TCAA |
| | | | | | all cell types | | | |
| | | | | | studied | | | |
| | | | | | (Tables S1, | | | |
| | | | | | S2, S3 and | | | |
| | | | | | S4) | | | |
| TRCN0000331730 | pLKO_ | NM_028810.2 | Rnd3 | Experi- | Downregulated | 74194 | CDS | GCACATTAGTGGAACT |
| | TRC005 | | | mental | with age in | | | CTCAA |
| | | | | | all cell types | | | |
| | | | | | studied | | | |
| | | | | | (Tables S1, | | | |
| | | | | | S2, S3 and | | | |
| | | | | | S4) | | | |
| TRCN0000081679 | pLKO.1 | NM_010118.1 | Eg2 | Experi- | Downregulated | 13654 | CDS | CCACTCTCTACCATCC |
| | | | | mental | with age in | | | GTAAT |
| | | | | | Drd1a- | | | |
| | | | | | expressing | | | |
| | | | | | medium spiny | | | |
| | | | | | neurons | | | |
| | | | | | (Tables S1) | | | |
| TRCN0000235775 | pLKO_ | NM_010118.3 | Eg2 | Experi- | Downregulated | 13654 | CDS | GAGATGGCATGATCAA |
| | TRC005 | | | mental | with age in | | | CATTG |
| | | | | | Drd1a- | | | |
| | | | | | expressing | | | |
| | | | | | medium spiny | | | |
| | | | | | neurons | | | |
| | | | | | (Tables S1) | | | |
| TRCN0000427699 | pLKO_ | NM_053131.1 | Pcdhb6 | Experi- | Upregulated | 93877 | CDS | GCTCACACTCTACCTGG |
| | TRC005 | | | mental | with age in | | | TCAT |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1, | | | |
| | | | | | and S2) | | | |
| TRCN0000434269 | pLKO_ | NM_053131.1 | Pcdhb6 | Experi- | Upregulated | 93877 | CDS | CAAATTCCTGAACCATT |
| | TRC005 | | | mental | with age in | | | ATTC |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1, | | | |
| | | | | | and S2) | | | |
| TRCN0000094302 | pLKO.1 | NM_053131.1 | Pcdhb6 | Experi- | Upregulated | 93877 | CDS | CCAGAATGCTTGGCTGT |
| | | | | mental | with age in | | | CATT |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1, | | | |
| | | | | | and S2) | | | |
| TRCN0000430303 | pLKO_ | NM_172126.2 | Adam1a | Experi- | Randomly | 280668 | CDS | TTCGCCAACATGTACGC |
| | TRC005 | | | mental | chosen target | | | TTAA |
| | | | | | gene | | | |
| TRCN000031725 | pLKO.1 | NM_172126.2 | Adam1a | Experi- | Randomly | 280668 | CDS | GCACAGTGTGATAGG |
| | | | | mental | chosen target | | | ATTT |
| | | | | | gene | | | |
| TRCN0000438367 | pLKO_ | NM_199021.3 | Dpp10 | Experi- | Upregulated | 269109 | CDS | GGCATCCAGTGTACTGC |
| | TRC005 | | | mental | with age in | | | ATAA |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1, | | | |
| | | | | | and S2) | | | |
| TRCN0000031459 | pLKO.1 | NM_199021.2 | Dpp10 | Experi- | Upregulated | 269109 | 3UTR | GCTTCTTTATTGAGCCA |
| | | | | mental | with age in | | | AATA |
| | | | | | Drd1a- and | | | |
| | | | | | Drd2- | | | |
| | | | | | expressing | | | |
| | | | | | medium spiny | | | |
| | | | | | neurons | | | |
| | | | | | (Tables S1, | | | |
| | | | | | and S2) | | | |
| TRCN0000104268 | pLKO.1 | NM_052835.1 | Rpl10 | Experi- | Proteins | 110954 | CDS | CCGAACCAAGTTGCAGA |
| | | | | mental | involved in | | | ACAA |
| | | | | | translation | | | |
| | | | | | have been | | | |
| | | | | | shown to be | | | |
| | | | | | associated | | | |
| | | | | | with | | | |
| | | | | | Huntingtin | | | |
| | | | | | protein | | | |
| | | | | | (Culver et | | | |
| | | | | | al., 2012) | | | |

### REFERENCES CITED ABOVE

Becanovic K, Pouladi M A, Lim R S, Kuhn A, Pavlidis P, Luthi-Carter R, Hayden M R, Leavitt B R, Transcriptional changes in Huntington disease identified using genome-wide expression profiling and cross-platform analysis.
Hum Mol Genet. 2010 Apr. 15; 19(8):1438-52.
Culver B P, Savas J N, Park S K, Choi J H, Zheng S. Zeitlin S O, Yates J R. 3rd, Tanecse N.
Proteomic analysis of wild-type and mutant huntingtin-associated proteins in mouse brains identifies unique interactions and involvement in protein synthesis.
J Biol Chem. 2012 June 22; 287(26):21599-614.
Heiman M, Schaefer A, Gong S, Peterson J D, Day M, Ramsey K E, Su‡rez-Fari-as M, Schwarz C, Stephan D A, Surmeier D J, Greengard P, Heintz N.
A translational profiling approach for the molecular characterization of CNS cell types.
Cell. 2008 Nov. 14; 135(4):738-48.
Humbert S, Bryson E A, Cordelieres F P, Connors N C, Datta S R, Finkbeiner S, Greenberg M E, Saudou F.
The IGF-1/Akt pathway is neuroprotective in Huntington's disease and involves Huntingtin phosphorylation by Aid.
Dev Cell 2002 June; 2(6):831-7.

TABLE 10
Log2 sequencing results from the SLIC screen time-points
4 replicates per time-point and genotype
95 viral elements targeting 76 distinct target sequences

| Hairpin Sequence | Hairpin IDs | Gene Name |
|---|---|---|
| CGCCAGTTAGCTCAATATCAT | TRCN0000066072 | Psmd2 (positive control) |
| AGACTTGCAGTTCACAGATAT | TRCN0000094178 | Pedhb8 |
| CCTGAGGTAATTCAAGAAATA | TRCN0000025951, | Stk3 |
| | TRCN0000323443 | |
| GCATGTGCAATCTACAGAGAT | TRCN0000076488 | Gpx6 |
| TAAAGTAGAGATGTCAGATAT | TRCN0000295762 | B2m |
| GTGAACGGAGACAATGAACAA | TRCN0000076491 | Gpx6 |
| CTGCGAAGTTTCGTTGGATTT | TRCN0000252745 | Kdm3a |
| CCTGACCAACAACATCACCCT | TRCN0000105789, | Pea15a |
| | TRCN0000288240 | |
| CAAAGACAACCTCTCCTACAT | TRCN0000105787, | Pea15a |
| | TRCN0000288175 | |
| CCCTCATAGTACCAAACGATT | TRCN0000104502, | Rps4x |
| | TRCN0000316606 | |
| AGCCATTCAACGTCACGGTTT | TRCN0000104426 | Rpl5 |
| CACAGATATAAACGACCATTT | TRCN0000076492 | Gpx6 |
| CACAGATATAAACGACCATTT | TRCN0000094176 | Pcdhb8 |
| CAAATTCCTGAACCATTATTC | TRCN0000434269 | Pcdhb6 |
| ACACCAAGCTAACCCGTATTC | TRCN0000307569 | Pea15a |
| CTATGCCAGGACTCTCGTAAA | TRCN0000311277 | Kdm6a |
| TGTCGGACAAGGCAGTAAATA | TRCN0000374677 | Arnt2 |
| CACGTTGCATCACAGTAAATT | TRCN0000366696 | Hras1 |
| CGCTAAATACTGGCAGGCGTT | TRCN0000072231, | LacZ (negative control) |
| | TRCN0000231710 | |
| GCCTTGAGAAACTTGGTATTT | TRCN0000123350, | Pkp2 |
| | TRCN0000287596 | |
| CCTATTTGATAGTCCTATATT | TRCN0000329357 | Sfmbt2 |
| CCTTCTTTCATGGACTACTTT | TRCN0000036990 | Stk3 |
| TTCGCCAACATGTACGCTTAA | TRCN0000430303 | Adam1a |
| CCGAACCAAGTTGCAGAACAA | TRCN0000104268 | Rpl10 |
| CACTCGGATATTTGATATGTG | TRCN0000072261, | Luciferase (negative control) |
| | TRCN0000231707 | |
| CCTGAGTATGTCTACAAGCTA | TRCN0000123351, | Pkp2 |
| | TRCN0000287514 | |
| CCAGTTTCTAATATGCTATAC | TRCN0000295705 | B2m |
| CCCTAATGAGTGGATAATAAA | TRCN0000337555 | Ddit41 |
| GGCATCCAGTGTACTGCATAA | TRCN0000438367 | Dpp10 |
| AGGCAAGTGACCGCCATTATC | TRCN0000435247 | Pcdhb14 |
| GCTCTTCCTTATAATTCCTTT | TRCN0000183360, | Phyh |
| | TRCN0000314262 | |
| TGCGGGTAGAAGGCTTCTTAA | TRCN0000252744 | Kdm3a |
| TCGCTTCTCCTCAGGCCTTAA | TRCN0000276918 | Ddit41 |
| CCACTCTCTACCATCCGTAAT | TRCN0000081679 | Egr2 |
| CATCAAATTCTGCTTGGACAA | TRCN0000221761 | Pgk1 |
| CCCACTTTCTTCTAGTTATAT | TRCN0000125009 | Gpr149 |
| CTGGCTCCAATGGCCTTATTA | TRCN0000094175 | Pcdhb8 |
| GCGATATTAACTATGGAGAAA | TRCN0000125011 | Gpr149 |
| CATTCCAAACTGTGACCGCAA | TRCN0000114797, | Igfbp4 |
| | TRCN0000320173 | |
| CCTACTCTGATGAGATCGAGT | TRCN0000096382, | Arnt2 |
| | TRCN0000323788 | |
| CCAGAGCTTTCTATCACTAGT | TRCN0000097669, | S100a10 |
| | TRCN0000317130 | |
| CGAAGACCTTGTCATAGAGTT | TRCN0000085087, | Taf13 |
| | TRCN0000317962 | |
| AGAATTGAAACGGGCTAGAAA | TRCN0000085085, | Taf13 |
| | TRCN0000317963 | |
| CCCTCTGACCACACCATATAA | TRCN0000329356 | Sfmbt2 |
| CCAGAATGCTTGGCTGTCATT | TRCN0000094302 | Pcdhb6 |
| GATTTCGACTACTGGGATTAT | TRCN0000176976, | Ddit41 |
| | TRCN0000276917 | |
| GAGGACATCAAAGCAAAGAAA | TRCN0000183203, | Phyh |
| | TRCN0000314263 | |
| GCACACAGTGTGATAGGATTT | TRCN0000031725 | Adam1a |
| CGCTATTATCATGCCATAGAT | TRCN0000096379, | Arnt2 |
| | TRCN0000323726 | |
| CACGATCAGAGCTGGTATTTA | TRCN0000231232 | Kdm3a |
| AGAATCGTCGTATGCAGTGAA | TRCN0000072250, | Luciferase (negative control) |
| | TRCN0000231730 | |
| GCGACATTCAGACAAATACAA | TRCN0000071997 | Foxp2 |
| GCACATTAGTGGAACTCTCAA | TRCN0000077330, | Rnd3 |
| | TRCN0000331730 | |
| GCTACGAATCTCTAATCTTAA | TRCN0000096242, | Kdm6a |
| | TRCN0000331919 | |
| CCAGTGTTTGTCTTATCCAAA | TRCN0000125010 | Gpr149 |
| ACAACAGCCACAACGTCTATA | TRCN0000464743, | GFP (negative control) |
| | TRCN0000464744, | |
| | TRCN0000464747, | |
| | TRCN0000072181, | |
| | TRCN0000231753 | |
| CGGACAGTCTTCAGTTCTGAA | TRCN0000071994 | Foxp2 |
| GAAGTTCCTGAGCAAGTTATT | TRCN0000252747 | Kdm3a |
| GTAGTGCAACCATCACGTATT | TRCN0000428544 | Pcdhb14 |
| TCAGTACTTATCAGCGAAATT | TRCN0000426134 | Pcdhb14 |
| TTCGTCAACCACCGGTGTTTC | TRCN0000329285 | Sfmbt2 |
| CGGAAGTTATTGATGTGGTAT | TRCN0000071993 | Foxp2 |
| GCTCACACTCTACCTGGTCAT | TRCN0000427699 | Pcdhb6 |
| CGGATGTGGTACGATTCATTA | TRCN0000329354 | Sfmt2 |
| AGTTAGCAGTGGAACGTTATG | TRCN0000305239 | Kdm6a |
| GAGATGGCATGATCAACATTG | TRCN0000235775 | Egr2 |
| GACAAGGATGAGAGCGAACAT | TRCN0000114798, | Igfbp4 |
| | TRCN0000320111 | |
| GCGATCACATGGTCCTGCTGG | TRCN0000207065 | GFP (negative control) |
| CTCAGTTCCAGTACGGCTCCA | TRCN0000072209, | RFP (negative control) |
| | TRCN0000231683 | |
| GCTTCTTTATTGAGCCAAATA | TRCN0000031459 | Dpp10 |
| GCTGCGGTTGTTGCGCCACTT | TRCN0000114800, | Igfbp4 |
| | TRCN0000350214 | |
| GCCGAACATACTGAACTGCTA | TRCN0000606424, | B2m |
| | TRCN0000288438 | |
| GTGAGATTCGGCAGCATAAAT | TRCN0000366695 | Hras1 |
| CGGGTGAAAGATTCAGATGAT | TRCN0000034382 | Hras1 |
| CATGATACTGGTAGTCATATT | TRCN0000419614 | Pcdhb14 |
| ACAGTTAACCACTTTTTGAAT | TRCN0000464725, | shRna negative control (non- |
| | TRCN0000464728, | shRNA transcript, negative |
| | TRCN0000464730, | control) |
| | TRCN0000464732, | |
| | TRCN0000464733, | |
| | TRCN0000464734, | |
| | TRCN0000464735, | |
| | TRCN0000464736, | |
| | TRCN0000464738, | |
| | TRCN0000241922, | |
| | TRCN0000464737, | |
| | TRCN0000464741, | |
| | TRCN0000464742, | |
| | TRCN0000241923, | |
| | TRCN0000231782, | |
| | TRCN0000464726, | |
| | TRCN0000464727, | |
| | TRCN0000464729, | |
| | TRCN0000464731, | |
| | TRCN0000464723, | |
| | TRCN0000464724 | |

TABLE 11
RIGER-assigned p values for depletion in the SLIC screen at 4 weeks.

| | | | | Normalized | | | p |
|---|---|---|---|---|---|---|---|
| | | # | Hairpin | enrichment | Gene | p | value |
| Gene | Hairpins | Hairpins | ranks | score | rank | value | rank |
| Gpx6 | GCATGTGCAATCTACAGAGAT. | 3 | 9, 3, 2 | 0.05882 | 1 | 0.0036 | 1 |
| | GTGAACGGAGACAATGAACAA, | | | | | | |
| | AGCCATTCAACGTCACGGTT | | | | | | |
| Pcdhb8 | CTGGCTCCAATGGCCTTATTA, | 3 | 13, 16, 4 | 0.2299 | 2 | 0.083 | 2 |
| | CACAGATATAAACGACCATTT, | | | | | | |
| | AGACTTGCAGTTCACAGATAT | | | | | | |
| Pkp2 | GCCTTGAGAAACTTGGTATTT, | 2 | 24, 30 | 0.5089 | 5 | 0.2377 | 3 |
| | CCTGAGTATGTCTACAAGCTA | | | | | | |
| Gpr149 | CCCACTTTCTTCTAGTTATAT, | 3 | 22, 37, 12 | 0.4171 | 3 | 0.2566 | 4 |
| | CCAGTGTTTGTCTTATCCAAA, | | | | | | |
| | GCGATATTAACTATGGAGAAA | | | | | | |
| Taf13 | AGAATTGAAACGGGCTAGAAA, | 2 | 17, 34 | 0.5312 | 6 | 0.2606 | 5 |
| | CGAAGACCTTGTCATAGAGTT | | | | | | |
| Phyh | GCTCTTCCTTATAATTCCTTT, | 2 | 19, 35 | 0.5536 | 7 | 0.2827 | 6 |
| | GAGGACATCAAAGCAAAGAAA | | | | | | |
| Hras1 | CGGGTGAAAGATTCAGATGAT, | 3 | 74, 185, 23 | 0.4652 | 4 | 0.3083 | 7 |
| | CACGTTGCATCACAGTAAATT, | | | | | | |
| | GTGAGATTCGGCAGCATAAAT | | | | | | |
| GFP | ACAACAGCCACAACGTCTATA, | 2 | 36, 25 | 0.5938 | 8 | 0.3282 | 8 |
| | GCGATCACATGGTCCTGCTGG | | | | | | |
| LUCIFERASE | AGAATCGTCGTATGCAGTGAA, | 2 | 20, 40 | 0.625 | 9 | 0.3619 | 9 |
| | CACTCGGATATTTGATATGTG | | | | | | |
| Foxp2 | CGGAAGTTATTGATGTGGTAT, | 3 | 41, 21, 33 | 0.6417 | 10 | 0.5203 | 10 |
| | CGGACAGTCTTCAGTTCTGAA, | | | | | | |
| | GCGACATTCAGACAAATACAA | | | | | | |
| Stk3 | CCTTCTTTCATGGACTACTTT, | 2 | 63, 7 | 0.875 | 15 | 0.7267 | 11 |
| | CCTGAGGTAATTCAAGAAATA | | | | | | |
| Igfbp4 | CATTCCAAACTGTGACCGCAA, | 3 | 54, 48, 8 | 0.8128 | 11 | 0.727 | 12 |
| | GACAAGGATGAGAGCGAACAT, | | | | | | |
| | GCTGCGGTTGTTGCGCCACTT | | | | | | |
| Ddit41 | GATTTCGACTACTGGGATTAT, | 3 | 46, 15, 53 | 0.8182 | 12 | 0.7335 | 13 |
| | TCGCTTCTCCTCAGGCCTTAA, | | | | | | |
| | CCCTAATGAGTGGATAATAAA | | | | | | |
| Pea15a | CAAAGACAACCTCTCCTACAT, | 3 | 39, 38, 44 | 0.8289 | 13 | 0.7465 | 14 |
| | CCTGACCAACAACATCACCCT, | | | | | | |
| | ACACCAAGCTAACCCGTATTC | | | | | | |
| Arnt2 | CGCTATTATCATGCCATAGAT, | 3 | 31, 42, 68 | 0.8396 | 14 | 0.759 | 15 |
| | CCTACTCTGATGAGATCGAGT, | | | | | | |
| | TGTCGGACAAGGCAGTAAATA | | | | | | |
| Pchhb6 | CCAGAATGCTTGGCTGTCATT, | 3 | 14, 61, 52 | 0.9091 | 16 | 0.8288 | 16 |
| | CAAATTCCTGAACCATTATTC, | | | | | | |
| | GCTCACACTCTACCTGGTCAT | | | | | | |
| Dpp10 | GCTTCTTTATTGAGCCAAATA, | 2 | 56, 43 | 0.942 | 17 | 0.832 | 17 |
| | GGCATCCAGTGTACTGCATAA | | | | | | |
| B2m | GCCGAACATACTGAACTGCTA, | 3 | 64, 11, 57 | 0.9733 | 18 | 0.8837 | 18 |
| | TAAAGTAGAGATGTCAGATAT, | | | | | | |
| | CCAGTTTCTAATATGCTATAC | | | | | | |
| Pcdhb14 | TCAGTACTTATCAGCGAAATT, | 4 | 75, 60, 72, | 1.2025 | 19 | 0.9765 | 19 |
| | CATGATACTGGTAGTCATATT, | | 10 | | | | |
| | GTAGTGCAACCATCACGTATT, | | | | | | |
| | AGGCAAGTGACCGCCATTATC | | | | | | |
| Kdm3a | CAGCATCAGAGCTGGTATTTA, | 4 | 47, 55, 58, | 1.2658 | 22 | 0.988 | 20 |
| | CTGCGAAGTTTCGTTGGATTT, | | 51 | | | | |
| | TGCGGGTAGAAGGCTTCTTAA, | | | | | | |
| | GAAGTTCCTGAGCAAGTTATT | | | | | | |
| Sfmbt2 | TTCGTCAACCACCGGTGTTTC, | 4 | 66, 49, 69, | 1.5443 | 24 | 1 | 21 |
| | CGGATGTGGTACGATTCATTA, | | 65 | | | | |
| | CCTATTTGATAGTCCTATATT, | | | | | | |
| | CCCTCTGACCACACCATATAA | | | | | | |
| Egr2 | CCACTCTCTACCATCCGTAAT, | 2 | 71, 59 | 1.2143 | 20 | 1.0001 | 22 |
| | GAGATGGCATGATCAACATTG | | | | | | |
| Kdm6a | GCTACGAATCTCTAATCTTAA, | 3 | 67, 70, 73 | 1.4813 | 23 | 1.0001 | 23 |
| | AGTTAGCAGTGGAACGTTATG, | | | | | | |
| | CTATGCCAGGACTCTCGTAAA | | | | | | |
| Adam1a | GCACACAGTGTGATAGGATTT, | 2 | 45, 76 | 1.2188 | 21 | 1.0001 | 24 |
| | TTCGCCAACATGTACGCTTAA | | | | | | |

TABLE 12
RIGER-assigned p values for depletion in the SLIC screen at 6 weeks.

| | | | | Normalized | | | p |
|---|---|---|---|---|---|---|---|
| | | # | Hairpin | enrichment | Gene | p | value |
| Gene | Hairpins | Hairpins | ranks | score | rank | value | rank |
| Gpx6 | GCATGTGCAATCTACAGAGAT. | 3 | 19, 6, 7 | 0.1444 | 1 | 0.032 | 1 |
| | GTGAACGGAGACAATGAACAA, | | | | | | |
| | AGCCATTCAACGTCACGGTT | | | | | | |
| Kdm3a | CAGCATCAGAGCTGGTATTTA, | 4 | 11, 9, 2, | 0.1835 | 2 | 0.0655 | 2 |
| | CTGCGAAGTTTCGTTGGATTT, | | 45 | | | | |
| | TGCGGGTAGAAGGCTTCTTAA, | | | | | | |
| | GAAGTTCCTGAGCAAGTTATT | | | | | | |
| Pcdhb8 | CTGGCTCCAATGGCCTTATTA, | 3 | 5, 16, 53 | 0.2834 | 3 | 0.1244 | 3 |
| | CACAGATATAAACGACCATTT, | | | | | | |
| | AGACTTGCAGTTCACAGATAT | | | | | | |
| Taf13 | AGAATTGAAACGGGCTAGAAA, | 2 | 13, 24 | 0.3795 | 4 | 0.1327 | 4 |
| | CGAAGACCTTGTCATAGAGTT | | | | | | |
| Adam1a | GCACACAGTGTGATAGGATTT, | 2 | 21, 40 | 0.6295 | 5 | 0.3669 | 5 |
| | TTCGCCAACATGTACGCTTAA | | | | | | |
| Phyh | GCTCTTCCTTATAATTCCTTT, | 2 | 25, 41 | 0.6607 | 7 | 0.4065 | 6 |
| | GAGGACATCAAAGCAAAGAAA | | | | | | |
| Pkp2 | GCCTTGAGAAACTTGGTATTT, | 2 | 27, 42 | 0.683 | 9 | 0.436 | 7 |
| | CCTGAGTATGTCTACAAGCTA | | | | | | |
| Pea15a | CAAAGACAACCTCTCCTACAT, | 3 | 30, 74, 29 | 0.634 | 6 | 0.5145 | 8 |
| | CCTGACCAACAACATCACCCT, | | | | | | |
| | ACACCAAGCTAACCCGTATTC | | | | | | |
| Hras1 | CGGGTGAAAGATTCAGATGAT, | 3 | 72, 34, 23 | 0.6684 | 8 | 0.5534 | 9 |
| | CACGTTGCATCACAGTAAATT, | | | | | | |
| | GTGAGATTCGGCAGCATAAAT | | | | | | |
| Ddit41 | GATTTCGACTACTGGGATTAT, | 3 | 36, 20, 65 | 0.6845 | 10 | 0.5719 | 10 |
| | TCGCTTCTCCTCAGGCCTTAA, | | | | | | |
| | CCCTAATGAGTGGATAATAAA | | | | | | |
| GFP | ACAACAGCCACAACGTCTATA, | 2 | 50, 35 | 0.8259 | 11 | 0.6461 | 11 |
| | GCGATCACATGGTCCTGCTGG | | | | | | |
| Gpr149 | CCCACTTTCTTCTAGTTATAT, | 3 | 46, 54, 18 | 0.8342 | 12 | 0.7527 | 12 |
| | CCAGTGTTTGTCTTATCCAAA, | | | | | | |
| | GCGATATTAACTATGGAGAAA | | | | | | |
| Igfbp4 | CATTCCAAACTGTGACCGCAA, | 3 | 61, 48, 15 | 0.8503 | 13 | 0.7693 | 13 |
| | GACAAGGATGAGAGCGAACAT, | | | | | | |
| | GCTGCGGTTGTTGCGCCACTT | | | | | | |
| Foxp2 | CGGAAGTTATTGATGTGGTAT, | 3 | 55, 38, 43 | 0.893 | 14 | 0.8132 | 14 |
| | CGGACAGTCTTCAGTTCTGAA, | | | | | | |
| | GCGACATTCAGACAAATACAA | | | | | | |
| Stk3 | CCTTCTTTCATGGACTACTTT, | 2 | 4, 70 | 0.9554 | 15 | 0.8491 | 15 |
| | CCTGAGGTAATTCAAGAAATA | | | | | | |
| Pchhb6 | CCAGAATGCTTGGCTGTCATT, | 3 | 10, 59, 60 | 1 | 16 | 0.9038 | 16 |
| | CAAATTCCTGAACCATTATTC, | | | | | | |
| | GCTCACACTCTACCTGGTCAT | | | | | | |
| Egr2 | CCACTCTCTACCATCCGTAAT, | 2 | 64, 39 | 1.0312 | 17 | 0.9305 | 17 |
| | GAGATGGCATGATCAACATTG | | | | | | |
| B2m | GCCGAACATACTGAACTGCTA, | 3 | 67, 63, 8 | 0.0535 | 18 | 0.9334 | 18 |
| | TAAAGTAGAGATGTCAGATAT, | | | | | | |
| | CCAGTTTCTAATATGCTATAC | | | | | | |
| Sfmbt2 | TTCGTCAACCACCGGTGTTTC, | 4 | 37, 49, 51, | 1.0696 | 20 | 0.9342 | 19 |
| | CGGATGTGGTACGATTCATTA, | | 44 | | | | |
| | CCTATTTGATAGTCCTATATT, | | | | | | |
| | CCCTCTGACCACACCATATAA | | | | | | |
| Pcdhb14 | TCAGTACTTATCAGCGAAATT, | 4 | 75, 69, 52, | 1.0759 | 21 | 0.9373 | 20 |
| | CATGATACTGGTAGTCATATT, | | 14 | | | | |
| | GTAGTGCAACCATCACGTATT, | | | | | | |
| | AGGCAAGTGACCGCCATTATC | | | | | | |
| Arnt2 | CGCTATTATCATGCCATAGAT, | 3 | 28, 66, 58 | 1.0802 | 22 | 0.9474 | 21 |
| | CCTACTCTGATGAGATCGAGT, | | | | | | |
| | TGTCGGACAAGGCAGTAAATA | | | | | | |
| LUCIFERASE | AGAATCGTCGTATGCAGTGAA, | 2 | 17, 73 | 1.0536 | 19 | 0.949 | 22 |
| | CACTCGGATATTTGATATGTG | | | | | | |
| Dpp10 | GCTTCTTTATTGAGCCAAATA, | 2 | 71, 31 | 1.0893 | 23 | 0.9726 | 23 |
| | GGCATCCAGTGTACTGCATAA | | | | | | |
| Kdm6a | GCTACGAATCTCTAATCTTAA, | 3 | 57, 68, 56 | 1.2139 | 24 | 0.9892 | 24 |
| | AGTTAGCAGTGGAACGTTATG, | | | | | | |
| | CTATGCCAGGACTCTCGTAAA | | | | | | |

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A method of screening for modulators of a disease comprising:
(a) administering to each of a first and second mammal of the same species at least one vector, each vector comprising a regulatory element operably linked to a nucleotide sequence that is transcribed in vivo,
wherein the first mammal is a model of a human disease and the second mammal is a normal control mammal not a model of a human disease, and
wherein the nucleotide sequence encodes a protein coding gene, or a short hairpin RNA, or a CRISPR/Cas system;
(b) harvesting DNA from the first mammal and the second mammal;
(c) identifying the vectors by sequencing the harvested DNA; and
(d) comparing the representation of each vector from the first mammal and the second mammal, whereby a differential representation in the first mammal indicates that the protein coding gene, or short hairpin RNA target, or CRISPR/Cas system target is a modulator of the disease.

2. The method of claim 1, wherein each vector comprises a unique barcode sequence, and the method further comprises identifying the barcodes during sequencing, whereby the identification of a barcode indicates the presence of a vector.

3. The method of claim 1, wherein the vectors are administered stereotaxically.

4. The method of claim 1, wherein the CRISPR/Cas system comprises:
(i) a first regulatory element operably linked to a nucleotide sequence encoding a CRISPR-Cas system polynucleotide sequence comprising at least one guide sequence, a tracr RNA, and a tracr mate sequence, wherein the at least one guide sequence hybridizes with a target sequence; and
(ii) a second regulatory element operably linked to a nucleotide sequence encoding a Type II Cas9 protein.

5. The method of claim 1, wherein the first and second mammals are transgenic non-human mammals comprising Cas9 and wherein the nucleotide sequence encoding a CRISPR/Cas system comprises at least one guide sequence, a tracr RNA, and a tracr mate sequence, wherein the at least one guide sequence hybridizes with a target sequence.

6. The method of claim 5, wherein expression of Cas9 is inducible.

7. The method of claim 1, wherein the vector is configured to be conditional, whereby the vector targets only certain cell types.

8. The method of claim 1, wherein the vector is a viral vector.

9. The method of claim 8, wherein the viral vector is a lentivirus, an adenovirus, or an adeno associated virus (AAV).

10. The method of claim 1, wherein the disease is Huntington's Disease.

11. The method of claim 1, wherein the first mammal is the R6/2 Huntington's disease model line.

12. A method of treating a nervous system disease comprising activating expression of Gpx6 in the central nervous system of a subject in need thereof suffering from the disease.

13. A method of treating a nervous system disease comprising expressing Gpx6 in the central nervous system of a subject in need thereof suffering from the disease.

14. A method of treating a nervous system disease comprising introducing into a subject in need thereof suffering from the disease a CRISPR-Cas9 based system configured to target Gpx6.

15. The method of claim 14, wherein the CRISPR/Cas system comprises a functional domain that activates transcription of the Gpx6 gene.

16. The method of claim 12, wherein the nervous system disease is Huntington's Disease or Parkinson's Disease.

17. The method of claim 12, further comprising administering to a subject in need thereof suffering from the disease at least one of the drugs selected from the group consisting of Tetrabenazine, neuroleptics, benzodiazepines, amantadine, anti Parkinson's drugs, valproic acid, antioxidants, and Gpx mimetics.

18. A method of determining a prognosis for a central nervous system disease comprising:
(e) obtaining a RNA sample from a patient suffering from a central nervous system disease;
(f) assaying the level of Gpx6 gene expression; and
(g) comparing the levels of Gpx6 gene expression to a control level determined by testing healthy subjects, wherein the prognosis is worse if Gpx6 gene expression is lower than the control level.

19. The method of claim 17 further comprising assaying the level of DARPP-32 gene expression; and comparing the levels of DARPP-32 gene expression to a control level determined by testing healthy subjects, wherein the prognosis is worse if DARPP-32 gene expression is lower than the control level.

20. An antibody comprising a heavy chain and a light chain, wherein the antibody binds to an antigenic region of the Gpx6 protein comprising SEQ ID No: 1.


SEQUENCE LISTING (321 sequences)

SEQ ID NO: 1 - protein, 9 aa, Mus musculus
SDIMEYLNQ

SEQ ID NO: 2 - protein, 6 aa, Artificial Sequence
  source: /note=“Description of Artificial Sequence Synthetic 6xHis tag”
HHHHHH

SEQ ID NO: 3 - protein, 7 aa, Simian virus 40
PKKKRKV

SEQ ID NO: 4 - protein, 16 aa, Unknown
  source: /note=“Description of Unknown Nucleoplasmin bipartite NLS sequence”
KRPAATKKAGQAKKKK

SEQ ID NO: 5 - protein, 9 aa, Unknown
  source: /note=“Description of Unknown c-myc NLS sequence”
PAAKRVKLD

SEQ ID NO: 6 - protein, 11 aa, Unknown
  source: /note=“Description of Unknown c-myc NLS sequence”
RQRRNELKRSP

SEQ ID NO: 7 - protein, 38 aa, Homo sapiens
NQSSNFGPMKGGNFGGRSSGPYGGGGQYFAKPRNQGGY

SEQ ID NO: 8 - protein, 42 aa, Unknown
  source: /note=“Description of Unknown IBB domain from importin-alpha sequence”
RMRIZFKNKGKDTAELRRRRVEVSVELRKAKKDEQILKRRNV

SEQ ID NO: 9 - protein, 8 aa, Unknown
  source: /note=“Description of Unknown myoma T protein sequence”
VSRKRPRP

SEQ ID NO: 10 - protein, 8 aa, Unknown
  source: /note=“Description of Unknown myoma T protein sequence”
PPKKARED

SEQ ID NO: 11 - protein, 8 aa, Homo sapiens
PQPKKKPL

SEQ ID NO: 12 - protein, 12 aa, Mus musculus
SALIKKKKKMAP

SEQ ID NO: 13 - protein, 5 aa, Influenza virus
DRLRR

SEQ ID NO: 14 - protein, 7 aa, Influenza virus
PKQKKRK

SEQ ID NO: 15 - protein, 10 aa, Hepatitis delta virus
RKLKKKIKKL

SEQ ID NO: 16 - protein, 10 aa, Mus musculus
REKKKFLKRR

SEQ ID NO: 17 - protein, 20 aa, Homo sapiens
KRKGDEVDGVDEVAKKKSKK

SEQ ID NO: 18 - protein, 17 aa, Homo sapiens
RKCLQAGMNLEARKTKK

SEQ ID NO: 19 - DNA, 21 bp, Artificial Sequence
  source: /note=“Description of Artificial Sequence Synthetic oligonucleotide”
cactcggatatttgatatgtg

SEQ ID NO: 20 - DNA, 21 bp, Artificial Sequence
  source: /note=“Description of Artificial Sequence Synthetic oligonucleotide”
agaatcgtcgtatgcagtgaa

SEQ ID NO: 21 - DNA, 21 bp, Artificial Sequence
  source: /note=“Description of Artificial Sequence Synthetic oligonucleotide”
cgccagttagctcaatatcat

SEQ ID NO: 22 - DNA, 21 bp, Artificial Sequence
  source: /note=“Description of Artificial Sequence Synthetic oligonucleotide”
gcgatcacatggtcctgctgg

SEQ ID NO: 23 - DNA, 21 bp, Artificial Sequence
  source: /note=“Description of Artificial Sequence Synthetic oligonucleotide”
cgctaaatactggcaggcgtt

SEQ ID NO: 24 - DNA, 21 bp, Artificial Sequence
  source: /note=“Description of Artificial Sequence Synthetic oligonucleotide”
ctcagttccagtacggctcca

SEQ ID NO: 25 - DNA, 21 bp, Artificial Sequence
  source: /note=“Description of Artificial Sequence Synthetic oligonucleotide”
acaacagccacaacgtctata

SEQ ID NO: 26 - DNA, 21 bp, Artificial Sequence
  source: /note=“Description of Artificial Sequence Synthetic oligonucleotide”
acagttaaccactttttgaat

SEQ ID NO: 27 - DNA, 21 bp, Artificial Sequence
  source: /note=“Description of Artificial Sequence Synthetic oligonucleotide”
gtagtgcaaccatcacgtatt

SEQ ID NO: 28 - DNA, 21 bp, Artificial Sequence
  source: /note=“Description of Artificial Sequence Synthetic oligonucleotide”
aggcaagtgaccgccattatc

SEQ ID NO: 29 - DNA, 21 bp, Artificial Sequence
  source: /note=“Description of Artificial Sequence Synthetic oligonucleotide”
catgatactggtagtcatatt

SEQ ID NO: 30 - DNA, 21 bp, Artificial Sequence
  source: /note=“Description of Artificial Sequence Synthetic oligonucleotide”
tcagtacttatcagcgaaatt

SEQ ID NO: 31 - DNA, 21 bp, Artificial Sequence
  source: /note=“Description of Artificial Sequence Synthetic oligonucleotide”
cattccaaactgtgaccgcaa

SEQ ID NO: 32 - DNA, 21 bp, Artificial Sequence
  source: /note=“Description of Artificial Sequence Synthetic oligonucleotide”
gctgcggttgttgcgccactt

SEQ ID NO: 33 - DNA, 21 bp, Artificial Sequence
  source: /note=“Description of Artificial Sequence Synthetic oligonucleotide”
gacaaggatgagagcgaacat

SEQ ID NO: 34 - DNA, 21 bp, Artificial Sequence
  source: /note=“Description of Artificial Sequence Synthetic oligonucleotide”
cattccaaactgtgaccgcaa

SEQ ID NO: 35 - DNA, 21 bp, Artificial Sequence
  source: /note=“Description of Artificial Sequence Synthetic oligonucleotide”
gctgcggttgttgcgccactt

SEQ ID NO: 36 - DNA, 21 bp, Artificial Sequence
  source: /note=“Description of Artificial Sequence Synthetic oligonucleotide”
gacaaggatgagagcgaacat

SEQ ID NO: 37 - DNA, 21 bp, Artificial Sequence
  source: /note=“Description of Artificial Sequence Synthetic oligonucleotide”
caaagacaacctctcctacat

SEQ ID NO: 38 - DNA, 21 bp, Artificial Sequence
  source: /note=“Description of Artificial Sequence Synthetic oligonucleotide”
cctgaccaacaacatcaccct

SEQ ID NO: 39 - DNA, 21 bp, Artificial Sequence
  source: /note=“Description of Artificial Sequence Synthetic oligonucleotide”
caaagacaacctctcctacat

SEQ ID NO: 40 - DNA, 21 bp, Artificial Sequence
  source: /note=“Description of Artificial Sequence Synthetic oligonucleotide”
cctgaccaacaacatcaccct

SEQ ID NO: 41 - DNA, 21 bp, Artificial Sequence
  source: /note=“Description of Artificial Sequence Synthetic oligonucleotide”
acaccaagctaacccgtattc

SEQ ID NO: 42 - DNA, 21 bp, Artificial Sequence
  source: /note=“Description of Artificial Sequence Synthetic oligonucleotide”
cgctattatcatgccatagat

SEQ ID NO: 43 - DNA, 21 bp, Artificial Sequence
  source: /note=“Description of Artificial Sequence Synthetic oligonucleotide”
cctactctgatgagatcgagt

SEQ ID NO: 44 - DNA, 21 bp, Artificial Sequence
  source: /note=“Description of Artificial Sequence Synthetic oligonucleotide”
cgctattatcatgccatagat

SEQ ID NO: 45 - DNA, 21 bp, Artificial Sequence
  source: /note=“Description of Artificial Sequence Synthetic oligonucleotide”
cctactctgatgagatcgagt

SEQ ID NO: 46 - DNA, 21 bp, Artificial Sequence
  source: /note=“Description of Artificial Sequence Synthetic oligonucleotide”
tgtcggacaaggcagtaaata

SEQ ID NO: 47 - DNA, 21 bp, Artificial Sequence
  source: /note=“Description of Artificial Sequence Synthetic oligonucleotide”
cacgatcagagctggtattta

SEQ ID NO: 48 - DNA, 21 bp, Artificial Sequence
  source: /note=“Description of Artificial Sequence Synthetic oligonucleotide”
tgcgggtagaaggcttcttaa

SEQ ID NO: 49 - DNA, 21 bp, Artificial Sequence
  source: /note=“Description of Artificial Sequence Synthetic oligonucleotide”
ctgcgaagtttcgttggattt

SEQ ID NO: 50 - DNA, 21 bp, Artificial Sequence
  source: /note=“Description of Artificial Sequence Synthetic oligonucleotide”
gaagttcctgagcaagttatt

SEQ ID NO: 51 - DNA, 21 bp, Artificial Sequence
  source: /note=“Description of Artificial Sequence Synthetic oligonucleotide”
ccagtttctaatatgctatac

SEQ ID NO: 52 - DNA, 21 bp, Artificial Sequence
  source: /note=“Description of Artificial Sequence Synthetic oligonucleotide”
taaagtagagatgtcagatat

SEQ ID NO: 53 - DNA, 21 bp, Artificial Sequence
  source: /note=“Description of Artificial Sequence Synthetic oligonucleotide”
gccgaacatactgaactgcta

SEQ ID NO: 54 - DNA, 21 bp, Artificial Sequence
  source: /note=“Description of Artificial Sequence Synthetic oligonucleotide”
gccgaacatactgaactgcta

SEQ ID NO: 55 - DNA, 21 bp, Artificial Sequence
  source: /note=“Description of Artificial Sequence Synthetic oligonucleotide”
ccctctgaccacaccatataa

SEQ ID NO: 56 - DNA, 21 bp, Artificial Sequence
  source: /note=“Description of Artificial Sequence Synthetic oligonucleotide”
cggatgtggtacgattcatta

SEQ ID NO: 57 - DNA, 21 bp, Artificial Sequence
  source: /note=“Description of Artificial Sequence Synthetic oligonucleotide”
cctatttgatagtcctatatt

SEQ ID NO: 58 - DNA, 21 bp, Artificial Sequence
  source: /note=“Description of Artificial Sequence Synthetic oligonucleotide”
ttcgtcaaccaccggtgtttc

SEQ ID NO: 59 - DNA, 21 bp, Artificial Sequence
  source: /note=“Description of Artificial Sequence Synthetic oligonucleotide”
ccctaatgagtggataataaa

SEQ ID NO: 60 - DNA, 21 bp, Artificial Sequence
  source: /note=“Description of Artificial Sequence Synthetic oligonucleotide”
gatttcgactactgggattat

SEQ ID NO: 61 - DNA, 21 bp, Artificial Sequence
  source: /note=“Description of Artificial Sequence Synthetic oligonucleotide”
gatttcgactactgggattat

SEQ ID NO: 62 - DNA, 21 bp, Artificial Sequence
  source: /note=“Description of Artificial Sequence Synthetic oligonucleotide”
tcgcttctcctcaggccttaa

SEQ ID NO: 63 - DNA, 21 bp, Artificial Sequence
  source: /note=“Description of Artificial Sequence Synthetic oligonucleotide”
gaggacatcaaagcaaagaaa

SEQ ID NO: 64 - DNA, 21 bp, Artificial Sequence
  source: /note=“Description of Artificial Sequence Synthetic oligonucleotide”
gctcttccttataattccttt

SEQ ID NO: 65 - DNA, 21 bp, Artificial Sequence
  source: /note=“Description of Artificial Sequence Synthetic oligonucleotide”
gaggacatcaaagcaaagaaa

SEQ ID NO: 66 - DNA, 21 bp, Artificial Sequence
  source: /note=“Description of Artificial Sequence Synthetic oligonucleotide”
gctcttccttataattccttt

SEQ ID NO: 67 - DNA, 21 bp, Artificial Sequence
  source: /note=“Description of Artificial Sequence Synthetic oligonucleotide”
catcaaattctgcttggacaa

SEQ ID NO: 68 - DNA, 21 bp, Artificial Sequence
  source: /note=“Description of Artificial Sequence Synthetic oligonucleotide”
ccctgactggagatgaagtaa

SEQ ID NO: 69 - DNA, 21 bp, Artificial Sequence
  source: /note=“Description of Artificial Sequence Synthetic oligonucleotide”
ccctcatagtaccaaacgatt

SEQ ID NO: 70 - DNA, 21 bp, Artificial Sequence
  source: /note=“Description of Artificial Sequence Synthetic oligonucleotide”
ctatgccaggactctcgtaaa

SEQ ID NO: 71 - DNA, 21 bp, Artificial Sequence
  source: /note=“Description of Artificial Sequence Synthetic oligonucleotide”
agttagcagtggaacgttatg

SEQ ID NO: 72 - DNA, 21 bp, Artificial Sequence
  source: /note=“Description of Artificial Sequence Synthetic oligonucleotide”
gctacgaatctctaatcttaa

SEQ ID NO: 73 - DNA, 21 bp, Artificial Sequence
  source: /note=“Description of Artificial Sequence Synthetic oligonucleotide”
gctacgaatctctaatcttaa

SEQ ID NO: 74 - DNA, 21 bp, Artificial Sequence
  source: /note=“Description of Artificial Sequence Synthetic oligonucleotide”
cgaagaccttgtcatagagtt

SEQ ID NO: 75 - DNA, 21 bp, Artificial Sequence
  source: /note=“Description of Artificial Sequence Synthetic oligonucleotide”
agaattgaaacgggctagaaa

SEQ ID NO: 76 - DNA, 21 bp, Artificial Sequence
  source: /note=“Description of Artificial Sequence Synthetic oligonucleotide”
cgaagaccttgtcatagagtt

SEQ ID NO: 77 - DNA, 21 bp, Artificial Sequence
  source: /note=“Description of Artificial Sequence Synthetic oligonucleotide”
agaattgaaacgggctagaaa

SEQ ID NO: 78 - DNA, 21 bp, Artificial Sequence
  source: /note=“Description of Artificial Sequence Synthetic oligonucleotide”
gccttgagaaacttggtattt

SEQ ID NO: 79 - DNA, 21 bp, Artificial Sequence
  source: /note=“Description of Artificial Sequence Synthetic oligonucleotide”
gccttgagaaacttggtattt

SEQ ID NO: 80 - DNA, 21 bp, Artificial Sequence
  source: /note=“Description of Artificial Sequence Synthetic oligonucleotide”
cctgagtatgtctacaagcta

SEQ ID NO: 81 - DNA, 21 bp, Artificial Sequence
  source: /note=“Description of Artificial Sequence Synthetic oligonucleotide”
cctgagtatgtctacaagcta

SEQ ID NO: 82 - DNA, 21 bp, Artificial Sequence
  source: /note=“Description of Artificial Sequence Synthetic oligonucleotide”
cggaagttattgatgtggtat

SEQ ID NO: 83 - DNA, 21 bp, Artificial Sequence
  source: /note=“Description of Artificial Sequence Synthetic oligonucleotide”
cggacagtcttcagttctgaa

SEQ ID NO: 84 - DNA, 21 bp, Artificial Sequence
  source: /note=“Description of Artificial Sequence Synthetic oligonucleotide”
gcgacattcagacaaatacaa

SEQ ID NO: 85 - DNA, 21 bp, Artificial Sequence
  source: /note=“Description of Artificial Sequence Synthetic oligonucleotide”
agccattcaacgtcacggttt

SEQ ID NO: 86 - DNA, 21 bp, Artificial Sequence
  source: /note=“Description of Artificial Sequence Synthetic oligonucleotide”
gtgaacggagacaatgaacaa

SEQ ID NO: 87 - DNA, 21 bp, Artificial Sequence
  source: /note=“Description of Artificial Sequence Synthetic oligonucleotide”
gcatgtgcaatctacagagat

SEQ ID NO: 88 - DNA, 21 bp, Artificial Sequence
  source: /note=“Description of Artificial Sequence Synthetic oligonucleotide”
cccactttcttctagttatat

SEQ ID NO: 89 - DNA, 21 bp, Artificial Sequence
  source: /note=“Description of Artificial Sequence Synthetic oligonucleotide”
gcgatattaactatggagaaa

SEQ ID NO: 90 - DNA, 21 bp, Artificial Sequence
  source: /note=“Description of Artificial Sequence Synthetic oligonucleotide”
ccagtgtttgtcttatccaaa

SEQ ID NO: 91 - DNA, 21 bp, Artificial Sequence
  source: /note=“Description of Artificial Sequence Synthetic oligonucleotide”
ccagagctttctatcactagt

SEQ ID NO: 92 - DNA, 21 bp, Artificial Sequence
  source: /note=“Description of Artificial Sequence Synthetic oligonucleotide”
ccagagctttctatcactagt

SEQ ID NO: 93 - DNA, 21 bp, Artificial Sequence
  source: /note=“Description of Artificial Sequence Synthetic oligonucleotide”
cgggtgaaagattcagatgat

SEQ ID NO: 94 - DNA, 21 bp, Artificial Sequence
  source: /note=“Description of Artificial Sequence Synthetic oligonucleotide”
gtgagattcggcagcataaat

SEQ ID NO: 95 - DNA, 21 bp, Artificial Sequence
  source: /note=“Description of Artificial Sequence Synthetic oligonucleotide”
cacgttgcatcacagtaaatt

SEQ ID NO: 96 - DNA, 21 bp, Artificial Sequence
  source: /note=“Description of Artificial Sequence Synthetic oligonucleotide”
cctgaggtaattcaagaaata

SEQ ID NO: 97 - DNA, 21 bp, Artificial Sequence
  source: /note=“Description of Artificial Sequence Synthetic oligonucleotide”
ccttctttcatggactacttt

SEQ ID NO: 98 - DNA, 21 bp, Artificial Sequence
  source: /note=“Description of Artificial Sequence Synthetic oligonucleotide”
cctgaggtaattcaagaaata

SEQ ID NO: 99 - DNA, 21 bp, Artificial Sequence
  source: /note=“Description of Artificial Sequence Synthetic oligonucleotide”
agacttgcagttcacagatat

SEQ ID NO: 100 - DNA, 21 bp, Artificial Sequence
  source: /note=“Description of Artificial Sequence Synthetic oligonucleotide”
ctggctccaatggccttatta

SEQ ID NO: 101 - DNA, 21 bp, Artificial Sequence
  source: /note=“Description of Artificial Sequence Synthetic oligonucleotide”
cacagatataaacgaccattt

SEQ ID NO: 102 - DNA, 21 bp, Artificial Sequence
  source: /note=“Description of Artificial Sequence Synthetic oligonucleotide”
gcacattagtggaactctcaa

SEQ ID NO: 103 - DNA, 21 bp, Artificial Sequence
  source: /note=“Description of Artificial Sequence Synthetic oligonucleotide”
gcacattagtggaactctcaa

SEQ ID NO: 104 - DNA, 21 bp, Artificial Sequence
  source: /note=“Description of Artificial Sequence Synthetic oligonucleotide”
ccactctctaccatccgtaat

SEQ ID NO: 105 - DNA, 21 bp, Artificial Sequence
  source: /note=“Description of Artificial Sequence Synthetic oligonucleotide”
gagatggcatgatcaacattg

SEQ ID NO: 106 - DNA, 21 bp, Artificial Sequence
  source: /note=“Description of Artificial Sequence Synthetic oligonucleotide”
gctcacactctacctggtcat

SEQ ID NO: 107 - DNA, 21 bp, Artificial Sequence
  source: /note=“Description of Artificial Sequence Synthetic oligonucleotide”
caaattcctgaaccattattc

SEQ ID NO: 108 - DNA, 21 bp, Artificial Sequence
  source: /note=“Description of Artificial Sequence Synthetic oligonucleotide”
ccagaatgcttggctgtcatt

SEQ ID NO: 109 - DNA, 21 bp, Artificial Sequence
  source: /note=“Description of Artificial Sequence Synthetic oligonucleotide”
ttcgccaacatgtacgcttaa

SEQ ID NO: 110 - DNA, 21 bp, Artificial Sequence
  source: /note=“Description of Artificial Sequence Synthetic oligonucleotide”
gcacacagtgtgataggattt

SEQ ID NO: 111 - DNA, 21 bp, Artificial Sequence
  source: /note=“Description of Artificial Sequence Synthetic oligonucleotide”
ggcatccagtgtactgcataa

SEQ ID NO: 112 - DNA, 21 bp, Artificial Sequence
  source: /note=“Description of Artificial Sequence Synthetic oligonucleotide”
gcttctttattgagccaaata

SEQ ID NO: 113 - DNA, 21 bp, Artificial Sequence
  source: /note=“Description of Artificial Sequence Synthetic oligonucleotide”
ccgaaccaagttgcagaacaa

SEQ ID NO: 114 - DNA, 21 bp, Artificial Sequence
  source: /note=“Description of Artificial Sequence Synthetic oligonucleotide”
cgccagttagctcaatatcat

SEQ ID NO: 115 - DNA, 21 bp, Artificial Sequence
  source: /note=“Description of Artificial Sequence Synthetic oligonucleotide”
agacttgcagttcacagatat

SEQ ID NO: 116 - DNA, 21 bp, Artificial Sequence
  source: /note=“Description of Artificial Sequence Synthetic oligonucleotide”
cctgaggtaattcaagaaata

SEQ ID NO: 117 - DNA, 21 bp, Artificial Sequence
  source: /note=“Description of Artificial Sequence Synthetic oligonucleotide”
gcatgtgcaatctacagagat

SEQ ID NO: 118 - DNA, 21 bp, Artificial Sequence
  source: /note=“Description of Artificial Sequence Synthetic oligonucleotide”
taaagtagagatgtcagatat

SEQ ID NO: 119 - DNA, 21 bp, Artificial Sequence
  source: /note=“Description of Artificial Sequence Synthetic oligonucleotide”
gtgaacggagacaatgaacaa

SEQ ID NO: 120 - DNA, 21 bp, Artificial Sequence
  source: /note=“Description of Artificial Sequence Synthetic oligonucleotide”
ctgcgaagtttcgttggattt

SEQ ID NO: 121 - DNA, 21 bp, Artificial Sequence
  source: /note=“Description of Artificial Sequence Synthetic oligonucleotide”
cctgaccaacaacatcaccct

SEQ ID NO: 122 - DNA, 21 bp, Artificial Sequence
  source: /note=“Description of Artificial Sequence Synthetic oligonucleotide”
caaagacaacctctcctacat

SEQ ID NO: 123 - DNA, 21 bp, Artificial Sequence
  source: /note=“Description of Artificial Sequence Synthetic oligonucleotide”
ccctgactggagatgaagtaa

SEQ ID NO: 124 - DNA, 21 bp, Artificial Sequence
  source: /note=“Description of Artificial Sequence Synthetic oligonucleotide”
ccctcatagtaccaaacgatt

SEQ ID NO: 125 - DNA, 21 bp, Artificial Sequence
  source: /note=“Description of Artificial Sequence Synthetic oligonucleotide”
agccattcaacgtcacggttt

SEQ ID NO: 126 - DNA, 21 bp, Artificial Sequence
  source: /note=“Description of Artificial Sequence Synthetic oligonucleotide”
cacagatataaacgaccattt

SEQ ID NO: 127 - DNA, 21 bp, Artificial Sequence
  source: /note=“Description of Artificial Sequence Synthetic oligonucleotide”
caaattcctgaaccattattc

SEQ ID NO: 128 - DNA, 21 bp, Artificial Sequence
  source: /note=“Description of Artificial Sequence Synthetic oligonucleotide”
acaccaagctaacccgtattc

SEQ ID NO: 129 - DNA, 21 bp, Artificial Sequence
  source: /note=“Description of Artificial Sequence Synthetic oligonucleotide”
ctatgccaggactctcgtaaa

SEQ ID NO: 130 - DNA, 21 bp, Artificial Sequence
  source: /note=“Description of Artificial Sequence Synthetic oligonucleotide”
tgtcggacaaggcagtaaata

SEQ ID NO: 131 - DNA, 21 bp, Artificial Sequence
  source: /note=“Description of Artificial Sequence Synthetic oligonucleotide”
cacgttgcatcacagtaaatt

SEQ ID NO: 132 - DNA, 21 bp, Artificial Sequence
  source: /note=“Description of Artificial Sequence Synthetic oligonucleotide”
cgctaaatactggcaggcgtt

SEQ ID NO: 133 - DNA, 21 bp, Artificial Sequence
  source: /note=“Description of Artificial Sequence Synthetic oligonucleotide”
gccttgagaaacttggtattt

SEQ ID NO: 134 - DNA, 21 bp, Artificial Sequence
  source: /note=“Description of Artificial Sequence Synthetic oligonucleotide”
cctatttgatagtcctatatt

SEQ ID NO: 135 - DNA, 21 bp, Artificial Sequence
  source: /note=“Description of Artificial Sequence Synthetic oligonucleotide”
ccttctttcatggactacttt

SEQ ID NO: 136 - DNA, 21 bp, Artificial Sequence
  source: /note=“Description of Artificial Sequence Synthetic oligonucleotide”
ttcgccaacatgtacgcttaa

SEQ ID NO: 137 - DNA, 21 bp, Artificial Sequence
  source: /note=“Description of Artificial Sequence Synthetic oligonucleotide”
ccgaaccaagttgcagaacaa

SEQ ID NO: 138 - DNA, 21 bp, Artificial Sequence
  source: /note=“Description of Artificial Sequence Synthetic oligonucleotide”
cactcggatatttgatatgtg

SEQ ID NO: 139 - DNA, 21 bp, Artificial Sequence
  source: /note=“Description of Artificial Sequence Synthetic oligonucleotide”
cctgagtatgtctacaagcta

SEQ ID NO: 140 - DNA, 21 bp, Artificial Sequence
  source: /note=“Description of Artificial Sequence Synthetic oligonucleotide”
ccagtttctaatatgctatac

SEQ ID NO: 141 - DNA, 21 bp, Artificial Sequence
  source: /note=“Description of Artificial Sequence Synthetic oligonucleotide”
ccctaatgagtggataataaa

SEQ ID NO: 142 - DNA, 21 bp, Artificial Sequence
  source: /note=“Description of Artificial Sequence Synthetic oligonucleotide”
ggcatccagtgtactgcataa

SEQ ID NO: 143 - DNA, 21 bp, Artificial Sequence
  source: /note=“Description of Artificial Sequence Synthetic oligonucleotide”
aggcaagtgaccgccattatc

SEQ ID NO: 144 - DNA, 21 bp, Artificial Sequence
  source: /note=“Description of Artificial Sequence Synthetic oligonucleotide”
gctcttccttataattccttt

SEQ ID NO: 145 - DNA, 21 bp, Artificial Sequence
  source: /note=“Description of Artificial Sequence Synthetic oligonucleotide”
tgcgggtagaaggcttcttaa

SEQ ID NO: 146 - DNA, 21 bp, Artificial Sequence
  source: /note=“Description of Artificial Sequence Synthetic oligonucleotide”
tcgcttctcctcaggccttaa

SEQ ID NO: 147 - DNA, 21 bp, Artificial Sequence
  source: /note=“Description of Artificial Sequence Synthetic oligonucleotide”
ccactctctaccatccgtaat

SEQ ID NO: 148 - DNA, 21 bp, Artificial Sequence
  source: /note=“Description of Artificial Sequence Synthetic oligonucleotide”
catcaaattctgcttggacaa

SEQ ID NO: 149 - DNA, 21 bp, Artificial Sequence
  source: /note=“Description of Artificial Sequence Synthetic oligonucleotide”
cccactttcttctagttatat

SEQ ID NO: 150 - DNA, 21 bp, Artificial Sequence
  source: /note=“Description of Artificial Sequence Synthetic oligonucleotide”
ctggctccaatggccttatta

SEQ ID NO: 151 - DNA, 21 bp, Artificial Sequence
  source: /note=“Description of Artificial Sequence Synthetic oligonucleotide”
gcgatattaactatggagaaa

SEQ ID NO: 152 - DNA, 21 bp, Artificial Sequence
  source: /note=“Description of Artificial Sequence Synthetic oligonucleotide”
cattccaaactgtgaccgcaa

SEQ ID NO: 153 - DNA, 21 bp, Artificial Sequence
  source: /note=“Description of Artificial Sequence Synthetic oligonucleotide”
cctactctgatgagatcgagt

SEQ ID NO: 154 - DNA, 21 bp, Artificial Sequence
  source: /note=“Description of Artificial Sequence Synthetic oligonucleotide”
ccagagctttctatcactagt

SEQ ID NO: 155 - DNA, 21 bp, Artificial Sequence
  source: /note=“Description of Artificial Sequence Synthetic oligonucleotide”
cgaagaccttgtcatagagtt

SEQ ID NO: 156 - DNA, 21 bp, Artificial Sequence
  source: /note=“Description of Artificial Sequence Synthetic oligonucleotide”
agaattgaaacgggctagaaa

SEQ ID NO: 157 - DNA, 21 bp, Artificial Sequence
  source: /note=“Description of Artificial Sequence Synthetic oligonucleotide”
ccctctgaccacaccatataa

SEQ ID NO: 158 - DNA, 21 bp, Artificial Sequence
  source: /note=“Description of Artificial Sequence Synthetic oligonucleotide”
ccagaatgcttggctgtcatt

SEQ ID NO: 159 - DNA, 21 bp, Artificial Sequence
  source: /note=“Description of Artificial Sequence Synthetic oligonucleotide”
gatttcgactactgggattat

SEQ ID NO: 160 - DNA, 21 bp, Artificial Sequence
  source: /note=“Description of Artificial Sequence Synthetic oligonucleotide”
gaggacatcaaagcaaagaaa

SEQ ID NO: 161 - DNA, 21 bp, Artificial Sequence
  source: /note=“Description of Artificial Sequence Synthetic oligonucleotide”
gcacacagtgtgataggattt

SEQ ID NO: 162 - DNA, 21 bp, Artificial Sequence
  source: /note=“Description of Artificial Sequence Synthetic oligonucleotide”
cgctattatcatgccatagat

SEQ ID NO: 163 - DNA, 21 bp, Artificial Sequence
  source: /note=“Description of Artificial Sequence Synthetic oligonucleotide”
cacgatcagagctggtattta

SEQ ID NO: 164 - DNA, 21 bp, Artificial Sequence
  source: /note=“Description of Artificial Sequence Synthetic oligonucleotide”
agaatcgtcgtatgcagtgaa

SEQ ID NO: 165 - DNA, 21 bp, Artificial Sequence
  source: /note=“Description of Artificial Sequence Synthetic oligonucleotide”
gcgacattcagacaaatacaa

SEQ ID NO: 166 - DNA, 21 bp, Artificial Sequence
  source: /note=“Description of Artificial Sequence Synthetic oligonucleotide”
gcacattagtggaactctcaa

SEQ ID NO: 167 - DNA, 21 bp, Artificial Sequence
  source: /note=“Description of Artificial Sequence Synthetic oligonucleotide”
gctacgaatctctaatcttaa

SEQ ID NO: 168 - DNA, 21 bp, Artificial Sequence
  source: /note=“Description of Artificial Sequence Synthetic oligonucleotide”
ccagtgtttgtcttatccaaa

SEQ ID NO: 169 - DNA, 21 bp, Artificial Sequence
  source: /note=“Description of Artificial Sequence Synthetic oligonucleotide”
acaacagccacaacgtctata

SEQ ID NO: 170 - DNA, 21 bp, Artificial Sequence
  source: /note=“Description of Artificial Sequence Synthetic oligonucleotide”
cggacagtcttcagttctgaa

SEQ ID NO: 171 - DNA, 21 bp, Artificial Sequence
  source: /note=“Description of Artificial Sequence Synthetic oligonucleotide”
gaagttcctgagcaagttatt

SEQ ID NO: 172 - DNA, 21 bp, Artificial Sequence
  source: /note=“Description of Artificial Sequence Synthetic oligonucleotide”
gtagtgcaaccatcacgtatt

SEQ ID NO: 173 - DNA, 21 bp, Artificial Sequence
  source: /note=“Description of Artificial Sequence Synthetic oligonucleotide”
tcagtacttatcagcgaaatt

SEQ ID NO: 174 - DNA, 21 bp, Artificial Sequence
  source: /note=“Description of Artificial Sequence Synthetic oligonucleotide”
ttcgtcaaccaccggtgtttc

SEQ ID NO: 175 - DNA, 21 bp, Artificial Sequence
  source: /note=“Description of Artificial Sequence Synthetic oligonucleotide”
cggaagttattgatgtggtat

SEQ ID NO: 176 - DNA, 21 bp, Artificial Sequence
  source: /note=“Description of Artificial Sequence Synthetic oligonucleotide”
gctcacactctacctggtcat

SEQ ID NO: 177 - DNA, 21 bp, Artificial Sequence
  source: /note=“Description of Artificial Sequence Synthetic oligonucleotide”
cggatgtggtacgattcatta

SEQ ID NO: 178 - DNA, 21 bp, Artificial Sequence
  source: /note=“Description of Artificial Sequence Synthetic oligonucleotide”
agttagcagtggaacgttatg

SEQ ID NO: 179 - DNA, 21 bp, Artificial Sequence
  source: /note=“Description of Artificial Sequence Synthetic oligonucleotide”
gagatggcatgatcaacattg

SEQ ID NO: 180 - DNA, 21 bp, Artificial Sequence
  source: /note=“Description of Artificial Sequence Synthetic oligonucleotide”
gacaaggatgagagcgaacat

SEQ ID NO: 181 - DNA, 21 bp, Artificial Sequence
  source: /note=“Description of Artificial Sequence Synthetic oligonucleotide”
gcgatcacatggtcctgctgg

SEQ ID NO: 182 - DNA, 21 bp, Artificial Sequence
  source: /note=“Description of Artificial Sequence Synthetic oligonucleotide”
ctcagttccagtacggctcca

SEQ ID NO: 183 - DNA, 21 bp, Artificial Sequence
  source: /note=“Description of Artificial Sequence Synthetic oligonucleotide”
gcttctttattgagccaaata

SEQ ID NO: 184 - DNA, 21 bp, Artificial Sequence
  source: /note=“Description of Artificial Sequence Synthetic oligonucleotide”
gctgcggttgttgcgccactt

SEQ ID NO: 185 - DNA, 21 bp, Artificial Sequence
  source: /note=“Description of Artificial Sequence Synthetic oligonucleotide”
gccgaacatactgaactgcta

SEQ ID NO: 186 - DNA, 21 bp, Artificial Sequence
  source: /note=“Description of Artificial Sequence Synthetic oligonucleotide”
gtgagattcggcagcataaat

SEQ ID NO: 187 - DNA, 21 bp, Artificial Sequence
  source: /note=“Description of Artificial Sequence Synthetic oligonucleotide”
cgggtgaaagattcagatgat

SEQ ID NO: 188 - DNA, 21 bp, Artificial Sequence
  source: /note=“Description of Artificial Sequence Synthetic oligonucleotide”
catgatactggtagtcatatt

SEQ ID NO: 189 - DNA, 21 bp, Artificial Sequence
  source: /note=“Description of Artificial Sequence Synthetic oligonucleotide”
acagttaaccactttttgaat

SEQ ID NO: 190 - DNA, 21 bp, Artificial Sequence
  source: /note=“Description of Artificial Sequence Synthetic oligonucleotide”
gcatgtgcaatctacagagat

SEQ ID NO: 191 - DNA, 21 bp, Artificial Sequence
  source: /note=“Description of Artificial Sequence Synthetic oligonucleotide”
gtgaacggagacaatgaacaa

SEQ ID NO: 192 - DNA, 21 bp, Artificial Sequence
  source: /note=“Description of Artificial Sequence Synthetic oligonucleotide”
agccattcaacgtcacggttt

SEQ ID NO: 193 - DNA, 21 bp, Artificial Sequence
  source: /note=“Description of Artificial Sequence Synthetic oligonucleotide”
ctggctccaatggccttatta

SEQ ID NO: 194 - DNA, 21 bp, Artificial Sequence
  source: /note=“Description of Artificial Sequence Synthetic oligonucleotide”
cacagatataaacgaccattt

SEQ ID NO: 195 - DNA, 21 bp, Artificial Sequence
  source: /note=“Description of Artificial Sequence Synthetic oligonucleotide”
agacttgcagttcacagatat

SEQ ID NO: 196 - DNA, 21 bp, Artificial Sequence
  source: /note=“Description of Artificial Sequence Synthetic oligonucleotide”
gccttgagaaacttggtattt

SEQ ID NO: 197 - DNA, 21 bp, Artificial Sequence
  source: /note=“Description of Artificial Sequence Synthetic oligonucleotide”
cctgagtatgtctacaagcta

SEQ ID NO: 198 - DNA, 21 bp, Artificial Sequence
  source: /note=“Description of Artificial Sequence Synthetic oligonucleotide”
cccactttcttctagttatat

SEQ ID NO: 199 - DNA, 21 bp, Artificial Sequence
  source: /note=“Description of Artificial Sequence Synthetic oligonucleotide”
ccagtgtttgtcttatccaaa

SEQ ID NO: 200 - DNA, 21 bp, Artificial Sequence
  source: /note=“Description of Artificial Sequence Synthetic oligonucleotide”
gcgatattaactatggagaaa

SEQ ID NO: 201 - DNA, 21 bp, Artificial Sequence
  source: /note=“Description of Artificial Sequence Synthetic oligonucleotide”
agaattgaaacgggctagaaa

SEQ ID NO: 202 - DNA, 21 bp, Artificial Sequence
  source: /note=“Description of Artificial Sequence Synthetic oligonucleotide”
cgaagaccttgtcatagagtt

SEQ ID NO: 203 - DNA, 21 bp, Artificial Sequence
  source: /note=“Description of Artificial Sequence Synthetic oligonucleotide”
gctcttccttataattccttt

SEQ ID NO: 204 - DNA, 21 bp, Artificial Sequence
  source: /note=“Description of Artificial Sequence Synthetic oligonucleotide”
gaggacatcaaagcaaagaaa

SEQ ID NO: 205 - DNA, 21 bp, Artificial Sequence
  source: /note=“Description of Artificial Sequence Synthetic oligonucleotide”
cgggtgaaagattcagatgat

SEQ ID NO: 206 - DNA, 21 bp, Artificial Sequence
  source: /note=“Description of Artificial Sequence Synthetic oligonucleotide”
cacgttgcatcacagtaaatt

SEQ ID NO: 207 - DNA, 21 bp, Artificial Sequence
  source: /note=“Description of Artificial Sequence Synthetic oligonucleotide”
gtgagattcggcagcataaat

SEQ ID NO: 208 - DNA, 21 bp, Artificial Sequence
  source: /note=“Description of Artificial Sequence Synthetic oligonucleotide”
acaacagccacaacgtctata

SEQ ID NO: 209 - DNA, 21 bp, Artificial Sequence
  source: /note=“Description of Artificial Sequence Synthetic oligonucleotide”
gcgatcacatggtcctgctgg

SEQ ID NO: 210 - DNA, 21 bp, Artificial Sequence
  source: /note=“Description of Artificial Sequence Synthetic oligonucleotide”
agaatcgtcgtatgcagtgaa

SEQ ID NO: 211 - DNA, 21 bp, Artificial Sequence
  source: /note=“Description of Artificial Sequence Synthetic oligonucleotide”
cactcggatatttgatatgtg

SEQ ID NO: 212 - DNA, 21 bp, Artificial Sequence
  source: /note=“Description of Artificial Sequence Synthetic oligonucleotide”
cggaagttattgatgtggtat

SEQ ID NO: 213 - DNA, 21 bp, Artificial Sequence
  source: /note=“Description of Artificial Sequence Synthetic oligonucleotide”
cggacagtcttcagttctgaa

SEQ ID NO: 214 - DNA, 21 bp, Artificial Sequence
  source: /note=“Description of Artificial Sequence Synthetic oligonucleotide”
gcgacattcagacaaatacaa

SEQ ID NO: 215 - DNA, 21 bp, Artificial Sequence
  source: /note=“Description of Artificial Sequence Synthetic oligonucleotide”
ccttctttcatggactacttt

SEQ ID NO: 216 - DNA, 21 bp, Artificial Sequence
  source: /note=“Description of Artificial Sequence Synthetic oligonucleotide”
cctgaggtaattcaagaaata

SEQ ID NO: 217 - DNA, 21 bp, Artificial Sequence
  source: /note=“Description of Artificial Sequence Synthetic oligonucleotide”
cattccaaactgtgaccgcaa

SEQ ID NO: 218 - DNA, 21 bp, Artificial Sequence
  source: /note=“Description of Artificial Sequence Synthetic oligonucleotide”
gacaaggatgagagcgaacat

SEQ ID NO: 219 - DNA, 21 bp, Artificial Sequence
  source: /note=“Description of Artificial Sequence Synthetic oligonucleotide”
gctgcggttgttgcgccactt

SEQ ID NO: 220 - DNA, 21 bp, Artificial Sequence
  source: /note=“Description of Artificial Sequence Synthetic oligonucleotide”
gatttcgactactgggattat

SEQ ID NO: 221 - DNA, 21 bp, Artificial Sequence
  source: /note=“Description of Artificial Sequence Synthetic oligonucleotide”
tcgcttctcctcaggccttaa

SEQ ID NO: 222 - DNA, 21 bp, Artificial Sequence
  source: /note=“Description of Artificial Sequence Synthetic oligonucleotide”
ccctaatgagtggataataaa

SEQ ID NO: 223 - DNA, 21 bp, Artificial Sequence
  source: /note=“Description of Artificial Sequence Synthetic oligonucleotide”
caaagacaacctctcctacat

SEQ ID NO: 224 - DNA, 21 bp, Artificial Sequence
  source: /note=“Description of Artificial Sequence Synthetic oligonucleotide”
cctgaccaacaacatcaccct

SEQ ID NO: 225 - DNA, 21 bp, Artificial Sequence
  source: /note=“Description of Artificial Sequence Synthetic oligonucleotide”
acaccaagctaacccgtattc

SEQ ID NO: 226 - DNA, 21 bp, Artificial Sequence
  source: /note=“Description of Artificial Sequence Synthetic oligonucleotide”
cgctattatcatgccatagat

SEQ ID NO: 227 - DNA, 21 bp, Artificial Sequence
  source: /note=“Description of Artificial Sequence Synthetic oligonucleotide”
cctactctgatgagatcgagt

SEQ ID NO: 228 - DNA, 21 bp, Artificial Sequence
  source: /note=“Description of Artificial Sequence Synthetic oligonucleotide”
tgtcggacaaggcagtaaata

SEQ ID NO: 229 - DNA, 21 bp, Artificial Sequence
  source: /note=“Description of Artificial Sequence Synthetic oligonucleotide”
ccagaatgcttggctgtcatt

SEQ ID NO: 230 - DNA, 21 bp, Artificial Sequence
  source: /note=“Description of Artificial Sequence Synthetic oligonucleotide”
caaattcctgaaccattattc

SEQ ID NO: 231 - DNA, 21 bp, Artificial Sequence
  source: /note=“Description of Artificial Sequence Synthetic oligonucleotide”
gctcacactctacctggtcat

SEQ ID NO: 232 - DNA, 21 bp, Artificial Sequence
  source: /note=“Description of Artificial Sequence Synthetic oligonucleotide”
gcttctttattgagccaaata

SEQ ID NO: 233 - DNA, 21 bp, Artificial Sequence
  source: /note=“Description of Artificial Sequence Synthetic oligonucleotide”
ggcatccagtgtactgcataa

SEQ ID NO: 234 - DNA, 21 bp, Artificial Sequence
  source: /note=“Description of Artificial Sequence Synthetic oligonucleotide”
gccgaacatactgaactgcta

SEQ ID NO: 235 - DNA, 21 bp, Artificial Sequence
  source: /note=“Description of Artificial Sequence Synthetic oligonucleotide”
taaagtagagatgtcagatat

SEQ ID NO: 236 - DNA, 21 bp, Artificial Sequence
  source: /note=“Description of Artificial Sequence Synthetic oligonucleotide”
ccagtttctaatatgctatac

SEQ ID NO: 237 - DNA, 21 bp, Artificial Sequence
  source: /note=“Description of Artificial Sequence Synthetic oligonucleotide”
tcagtacttatcagcgaaatt

SEQ ID NO: 238 - DNA, 21 bp, Artificial Sequence
  source: /note=“Description of Artificial Sequence Synthetic oligonucleotide”
catgatactggtagtcatatt

SEQ ID NO: 239 - DNA, 21 bp, Artificial Sequence
  source: /note=“Description of Artificial Sequence Synthetic oligonucleotide”
gtagtgcaaccatcacgtatt

SEQ ID NO: 240 - DNA, 21 bp, Artificial Sequence
  source: /note=“Description of Artificial Sequence Synthetic oligonucleotide”
aggcaagtgaccgccattatc

SEQ ID NO: 241 - DNA, 21 bp, Artificial Sequence
  source: /note=“Description of Artificial Sequence Synthetic oligonucleotide”
cacgatcagagctggtattta

SEQ ID NO: 242 - DNA, 21 bp, Artificial Sequence
  source: /note=“Description of Artificial Sequence Synthetic oligonucleotide”
ctgcgaagtttcgttggattt

SEQ ID NO: 243 - DNA, 21 bp, Artificial Sequence
  source: /note=“Description of Artificial Sequence Synthetic oligonucleotide”
tgcgggtagaaggcttcttaa

SEQ ID NO: 244 - DNA, 21 bp, Artificial Sequence
  source: /note=“Description of Artificial Sequence Synthetic oligonucleotide”
gaagttcctgagcaagttatt

SEQ ID NO: 245 - DNA, 21 bp, Artificial Sequence
  source: /note=“Description of Artificial Sequence Synthetic oligonucleotide”
ttcgtcaaccaccggtgtttc

SEQ ID NO: 246 - DNA, 21 bp, Artificial Sequence
  source: /note=“Description of Artificial Sequence Synthetic oligonucleotide”
cggatgtggtacgattcatta

SEQ ID NO: 247 - DNA, 21 bp, Artificial Sequence
  source: /note=“Description of Artificial Sequence Synthetic oligonucleotide”
cctatttgatagtcctatatt

SEQ ID NO: 248 - DNA, 21 bp, Artificial Sequence
  source: /note=“Description of Artificial Sequence Synthetic oligonucleotide”
ccctctgaccacaccatataa

SEQ ID NO: 249 - DNA, 21 bp, Artificial Sequence
  source: /note=“Description of Artificial Sequence Synthetic oligonucleotide”
ccactctctaccatccgtaat

SEQ ID NO: 250 - DNA, 21 bp, Artificial Sequence
  source: /note=“Description of Artificial Sequence Synthetic oligonucleotide”
gagatggcatgatcaacattg

SEQ ID NO: 251 - DNA, 21 bp, Artificial Sequence
  source: /note=“Description of Artificial Sequence Synthetic oligonucleotide”
gctacgaatctctaatcttaa

SEQ ID NO: 252 - DNA, 21 bp, Artificial Sequence
  source: /note=“Description of Artificial Sequence Synthetic oligonucleotide”
agttagcagtggaacgttatg

SEQ ID NO: 253 - DNA, 21 bp, Artificial Sequence
  source: /note=“Description of Artificial Sequence Synthetic oligonucleotide”
ctatgccaggactctcgtaaa

SEQ ID NO: 254 - DNA, 21 bp, Artificial Sequence
  source: /note=“Description of Artificial Sequence Synthetic oligonucleotide”
gcacacagtgtgataggattt

SEQ ID NO: 255 - DNA, 21 bp, Artificial Sequence
  source: /note=“Description of Artificial Sequence Synthetic oligonucleotide”
ttcgccaacatgtacgcttaa

SEQ ID NO: 256 - DNA, 21 bp, Artificial Sequence
  source: /note=“Description of Artificial Sequence Synthetic oligonucleotide”
gcatgtgcaatctacagagat

SEQ ID NO: 257 - DNA, 21 bp, Artificial Sequence
  source: /note=“Description of Artificial Sequence Synthetic oligonucleotide”
gtgaacggagacaatgaacaa

SEQ ID NO: 258 - DNA, 21 bp, Artificial Sequence
  source: /note=“Description of Artificial Sequence Synthetic oligonucleotide”
agccattcaacgtcacggttt

SEQ ID NO: 259 - DNA, 21 bp, Artificial Sequence
  source: /note=“Description of Artificial Sequence Synthetic oligonucleotide”
cacgatcagagctggtattta

SEQ ID NO: 260 - DNA, 21 bp, Artificial Sequence
  source: /note=“Description of Artificial Sequence Synthetic oligonucleotide”
tgcgggtagaaggcttcttaa

SEQ ID NO: 261 - DNA, 21 bp, Artificial Sequence
  source: /note=“Description of Artificial Sequence Synthetic oligonucleotide”
ctgcgaagtttcgttggattt

SEQ ID NO: 262 - DNA, 21 bp, Artificial Sequence
  source: /note=“Description of Artificial Sequence Synthetic oligonucleotide”
gaagttcctgagcaagttatt

SEQ ID NO: 263 - DNA, 21 bp, Artificial Sequence
  source: /note=“Description of Artificial Sequence Synthetic oligonucleotide”
ctggctccaatggccttatta

SEQ ID NO: 264 - DNA, 21 bp, Artificial Sequence
  source: /note=“Description of Artificial Sequence Synthetic oligonucleotide”
cacagatataaacgaccattt

SEQ ID NO: 265 - DNA, 21 bp, Artificial Sequence
  source: /note=“Description of Artificial Sequence Synthetic oligonucleotide”
agacttgcagttcacagatat

SEQ ID NO: 266 - DNA, 21 bp, Artificial Sequence
  source: /note=“Description of Artificial Sequence Synthetic oligonucleotide”
agaattgaaacgggctagaaa

SEQ ID NO: 267 - DNA, 21 bp, Artificial Sequence
  source: /note=“Description of Artificial Sequence Synthetic oligonucleotide”
cgaagaccttgtcatagagtt

SEQ ID NO: 268 - DNA, 21 bp, Artificial Sequence
  source: /note=“Description of Artificial Sequence Synthetic oligonucleotide”
gcacacagtgtgataggattt

SEQ ID NO: 269 - DNA, 21 bp, Artificial Sequence
  source: /note=“Description of Artificial Sequence Synthetic oligonucleotide”
ttcgccaacatgtacgcttaa

SEQ ID NO: 270 - DNA, 21 bp, Artificial Sequence
  source: /note=“Description of Artificial Sequence Synthetic oligonucleotide”
gaggacatcaaagcaaagaaa

SEQ ID NO: 271 - DNA, 21 bp, Artificial Sequence
  source: /note=“Description of Artificial Sequence Synthetic oligonucleotide”
gctcttccttataattccttt

SEQ ID NO: 272 - DNA, 21 bp, Artificial Sequence
  source: /note=“Description of Artificial Sequence Synthetic oligonucleotide”
gccttgagaaacttggtattt

SEQ ID NO: 273 - DNA, 21 bp, Artificial Sequence
  source: /note=“Description of Artificial Sequence Synthetic oligonucleotide”
cctgagtatgtctacaagcta

SEQ ID NO: 274 - DNA, 21 bp, Artificial Sequence
  source: /note=“Description of Artificial Sequence Synthetic oligonucleotide”
caaagacaacctctcctacat

SEQ ID NO: 275 - DNA, 21 bp, Artificial Sequence
  source: /note=“Description of Artificial Sequence Synthetic oligonucleotide”
cctgaccaacaacatcaccct

SEQ ID NO: 276 - DNA, 21 bp, Artificial Sequence
  source: /note=“Description of Artificial Sequence Synthetic oligonucleotide”
acaccaagctaacccgtattc

SEQ ID NO: 277 - DNA, 21 bp, Artificial Sequence
  source: /note=“Description of Artificial Sequence Synthetic oligonucleotide”
cgggtgaaagattcagatgat

SEQ ID NO: 278 - DNA, 21 bp, Artificial Sequence
  source: /note=“Description of Artificial Sequence Synthetic oligonucleotide”
gtgagattcggcagcataaat

SEQ ID NO: 279 - DNA, 21 bp, Artificial Sequence
  source: /note=“Description of Artificial Sequence Synthetic oligonucleotide”
cacgttgcatcacagtaaatt

SEQ ID NO: 280 - DNA, 21 bp, Artificial Sequence
  source: /note=“Description of Artificial Sequence Synthetic oligonucleotide”
gatttcgactactgggattat

SEQ ID NO: 281 - DNA, 21 bp, Artificial Sequence
  source: /note=“Description of Artificial Sequence Synthetic oligonucleotide”
tcgcttctcctcaggccttaa

SEQ ID NO: 282 - DNA, 21 bp, Artificial Sequence
  source: /note=“Description of Artificial Sequence Synthetic oligonucleotide”
ccctaatgagtggataataaa

SEQ ID NO: 283 - DNA, 21 bp, Artificial Sequence
  source: /note=“Description of Artificial Sequence Synthetic oligonucleotide”
acaacagccacaacgtctata

SEQ ID NO: 284 - DNA, 21 bp, Artificial Sequence
  source: /note=“Description of Artificial Sequence Synthetic oligonucleotide”
gcgatcacatggtcctgctgg

SEQ ID NO: 285 - DNA, 21 bp, Artificial Sequence
  source: /note=“Description of Artificial Sequence Synthetic oligonucleotide”
cccactttcttctagttatat

SEQ ID NO: 286 - DNA, 21 bp, Artificial Sequence
  source: /note=“Description of Artificial Sequence Synthetic oligonucleotide”
ccagtgtttgtcttatccaaa

SEQ ID NO: 287 - DNA, 21 bp, Artificial Sequence
  source: /note=“Description of Artificial Sequence Synthetic oligonucleotide”
gcgatattaactatggagaaa

SEQ ID NO: 288 - DNA, 21 bp, Artificial Sequence
  source: /note=“Description of Artificial Sequence Synthetic oligonucleotide”
cattccaaactgtgaccgcaa

SEQ ID NO: 289 - DNA, 21 bp, Artificial Sequence
  source: /note=“Description of Artificial Sequence Synthetic oligonucleotide”
gacaaggatgagagcgaacat

SEQ ID NO: 290 - DNA, 21 bp, Artificial Sequence
  source: /note=“Description of Artificial Sequence Synthetic oligonucleotide”
gctgcggttgttgcgccactt

SEQ ID NO: 291 - DNA, 21 bp, Artificial Sequence
  source: /note=“Description of Artificial Sequence Synthetic oligonucleotide”
cggaagttattgatgtggtat

SEQ ID NO: 292 - DNA, 21 bp, Artificial Sequence
  source: /note=“Description of Artificial Sequence Synthetic oligonucleotide”
cggacagtcttcagttctgaa

SEQ ID NO: 293 - DNA, 21 bp, Artificial Sequence
  source: /note=“Description of Artificial Sequence Synthetic oligonucleotide”
gcgacattcagacaaatacaa

SEQ ID NO: 294 - DNA, 21 bp, Artificial Sequence
  source: /note=“Description of Artificial Sequence Synthetic oligonucleotide”
cctgaggtaattcaagaaata

SEQ ID NO: 295 - DNA, 21 bp, Artificial Sequence
  source: /note=“Description of Artificial Sequence Synthetic oligonucleotide”
ccttctttcatggactacttt

SEQ ID NO: 296 - DNA, 21 bp, Artificial Sequence
  source: /note=“Description of Artificial Sequence Synthetic oligonucleotide”
ccagaatgcttggctgtcatt

SEQ ID NO: 297 - DNA, 21 bp, Artificial Sequence
  source: /note=“Description of Artificial Sequence Synthetic oligonucleotide”
gctcacactctacctggtcat

SEQ ID NO: 298 - DNA, 21 bp, Artificial Sequence
  source: /note=“Description of Artificial Sequence Synthetic oligonucleotide”
caaattcctgaaccattattc

SEQ ID NO: 299 - DNA, 21 bp, Artificial Sequence
  source: /note=“Description of Artificial Sequence Synthetic oligonucleotide”
ccactctctaccatccgtaat

SEQ ID NO: 300 - DNA, 21 bp, Artificial Sequence
  source: /note=“Description of Artificial Sequence Synthetic oligonucleotide”
gagatggcatgatcaacattg

SEQ ID NO: 301 - DNA, 21 bp, Artificial Sequence
  source: /note=“Description of Artificial Sequence Synthetic oligonucleotide”
gccgaacatactgaactgcta

SEQ ID NO: 302 - DNA, 21 bp, Artificial Sequence
  source: /note=“Description of Artificial Sequence Synthetic oligonucleotide”
ccagtttctaatatgctatac

SEQ ID NO: 303 - DNA, 21 bp, Artificial Sequence
  source: /note=“Description of Artificial Sequence Synthetic oligonucleotide”
taaagtagagatgtcagatat

SEQ ID NO: 304 - DNA, 21 bp, Artificial Sequence
  source: /note=“Description of Artificial Sequence Synthetic oligonucleotide”
ttcgtcaaccaccggtgtttc

SEQ ID NO: 305 - DNA, 21 bp, Artificial Sequence
  source: /note=“Description of Artificial Sequence Synthetic oligonucleotide”
cggatgtggtacgattcatta

SEQ ID NO: 306 - DNA, 21 bp, Artificial Sequence
  source: /note=“Description of Artificial Sequence Synthetic oligonucleotide”
ccctctgaccacaccatataa

SEQ ID NO: 307 - DNA, 21 bp, Artificial Sequence
  source: /note=“Description of Artificial Sequence Synthetic oligonucleotide”
cctatttgatagtcctatatt

SEQ ID NO: 308 - DNA, 21 bp, Artificial Sequence
  source: /note=“Description of Artificial Sequence Synthetic oligonucleotide”
catgatactggtagtcatatt

SEQ ID NO: 309 - DNA, 21 bp, Artificial Sequence
  source: /note=“Description of Artificial Sequence Synthetic oligonucleotide”
tcagtacttatcagcgaaatt

SEQ ID NO: 310 - DNA, 21 bp, Artificial Sequence
  source: /note=“Description of Artificial Sequence Synthetic oligonucleotide”
gtagtgcaaccatcacgtatt

SEQ ID NO: 311 - DNA, 21 bp, Artificial Sequence
  source: /note=“Description of Artificial Sequence Synthetic oligonucleotide”
aggcaagtgaccgccattatc

SEQ ID NO: 312 - DNA, 21 bp, Artificial Sequence
  source: /note=“Description of Artificial Sequence Synthetic oligonucleotide”
cgctattatcatgccatagat

SEQ ID NO: 313 - DNA, 21 bp, Artificial Sequence
  source: /note=“Description of Artificial Sequence Synthetic oligonucleotide”
cctactctgatgagatcgagt

SEQ ID NO: 314 - DNA, 21 bp, Artificial Sequence
  source: /note=“Description of Artificial Sequence Synthetic oligonucleotide”
tgtcggacaaggcagtaaata

SEQ ID NO: 315 - DNA, 21 bp, Artificial Sequence
  source: /note=“Description of Artificial Sequence Synthetic oligonucleotide”
agaatcgtcgtatgcagtgaa

SEQ ID NO: 316 - DNA, 21 bp, Artificial Sequence
  source: /note=“Description of Artificial Sequence Synthetic oligonucleotide”
cactcggatatttgatatgtg

SEQ ID NO: 317 - DNA, 21 bp, Artificial Sequence
  source: /note=“Description of Artificial Sequence Synthetic oligonucleotide”
gcttctttattgagccaaata

SEQ ID NO: 318 - DNA, 21 bp, Artificial Sequence
  source: /note=“Description of Artificial Sequence Synthetic oligonucleotide”
ggcatccagtgtactgcataa

SEQ ID NO: 319 - DNA, 21 bp, Artificial Sequence
  source: /note=“Description of Artificial Sequence Synthetic oligonucleotide”
gctacgaatctctaatcttaa

SEQ ID NO: 320 - DNA, 21 bp, Artificial Sequence
  source: /note=“Description of Artificial Sequence Synthetic oligonucleotide”
agttagcagtggaacgttatg

SEQ ID NO: 321 - DNA, 21 bp, Artificial Sequence
  source: /note=“Description of Artificial Sequence Synthetic oligonucleotide”
ctatgccaggactctcgtaaa